(19) United States
(12) Patent Application Publication
(10) Pub. No.: US 2016/0374690 A9
(43) Pub. Date: Dec. 29, 2016

(54) Devices and Methods for Occluding a Cerebral Aneurysm

(71) Applicant: Robert A. Connor, Forest Lake, MN (US)

(72) Inventor: Robert A. Connor, Forest Lake, MN (US)

(21) Appl. No.: 14/526,600

(22) Filed: Oct. 29, 2014

Related U.S. Application Data
(63) Continuation-in-part of application No. 12/989,048, filed on Oct. 21, 2010, now Pat. No. 8,974,487.
(60) Provisional application No. 61/897,245, filed on Oct. 30, 2013.

(51) Int. Cl. A61B 17/12

(52) U.S. Cl. CPC A61B 17/12118; A61B 17/12177; A61B 2017/1205; A61B 2017/00685

(57) ABSTRACT

This invention is a device to occlude a cerebral aneurysm which includes: a longitudinal lumen that is inserted into the parent blood vessel of an aneurysm; a flexible expandable member (such as a net or mesh) which is expanded within the aneurysm sack by the insertion of embolic members into that flexible expandable member; and a resilient expandable member (such as a cylindrical stent or ring stent) that is attached to a central portion of the flexible expandable member and expanded within the aneurysm sack in order to keep the flexible expandable member from slipping out of the aneurysm sack.

### CROSS-REFERENCE TO RELATED APPLICATIONS

This patent application is: (1) a Continuation In Part of U.S. patent application Ser. No. 12/989,048 entitled “Aneurysm Occlusion Device” by Robert A. Connor and Muhammad Tariq Janjua which has a 371 date of Oct. 21, 2010, a filing date of Apr. 24, 2009, and a priority date of May 1, 2008 which is the U.S. national phase filing of PCT/US 2009/002537 entitled “Aneurysm Occlusion Device” by Robert A. Connor and Muhammad Tariq Janjua filed on Apr. 24, 2009 which claimed the priority benefit of U.S. Provisional Patent Application No. 61/126,047 entitled “Flow of Soft Members into a Net to Embolize an Aneurysm” by Robert A. Connor which received a filing date of May 1, 2008 and claimed the priority benefit of U.S. Provisional Patent Application No. 61/126,027 entitled “Net Filled with Soft Members to Embolize an Aneurysm” by Robert A. Connor which received a filing date of May 1, 2008; and (2) also claims the priority benefit of U.S. Provisional Patent Application No. 61/897,245 entitled “Devices and Methods for Occluding a Cerebral Aneurysm” by Robert A. Connor filed on Oct. 30, 2013. The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to devices and methods for occluding a blood vessel aneurysm.

### Introduction to Cerebral Aneurysms

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain.

Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Sadly, even among those who survive, approximately one-half suffer significant and permanent deterioration of brain function.

### CATEGORIZATION AND REVIEW OF THE PRIOR ART

It can be challenging trying to classify prior art into discrete categories. This is the certainly the case in the field of devices and methods for treating aneurysms. There are numerous examples of potentially-relevant prior art. However, classification of the prior art into categories, even if imperfect, can be an invaluable tool for reviewing the relevant prior art. Towards this end, I herein identify 38 categories of relevant prior art and provide examples of prior art in each category (including patent or patent application number, inventor, publication date, and title). Some examples of prior art disclose multiple concepts and thus appear in more than one category. This review primarily focuses on structural endovascular approaches to treating aneurysms and does not include extravascular aneurysm clips or the many different pharmaceutical coatings for aneurysm treatment devices.

The 38 categories of prior art which are used for this review are as follows: (1) stent with circumferential variation in wall porosity in parent vessel; (2) stent with longitudinal variation in wall porosity in parent vessel; (3) stent with longitudinal or cross-sectional variation in size and/or flexibility in parent vessel; (4) stent in branching parent vessel; (5) stent with helical structure in parent vessel; (6) stent with special structure or flexibility in parent vessel; (7) stent with multiple layers in parent vessel; (8) stent with non-porous walls in parent vessel; (9) stent with integrated actuators and/or sensors in parent vessel; (10) stent with other complex structure in parent vessel; (11) stent in parent vessel to contain embolics within aneurysm sack; (12) multiple stents in parent vessel; (13) temporary balloon in parent vessel; (14) multi-balloon device; (15) aneurysm neck bridge or saddle in relatively-straight parent vessel; (16) aneurysm neck bridge or saddle in parent vessel with three-way junction; (17) aneurysm neck bridge or saddle with spherical structure in parent vessel; (18) localized aneurysm neck bridge in parent vessel; (19) aneurysm neck bridge spanning aneurysm sack and parent vessel; (20) aneurysm neck bridge inside aneurysm sack with radial protrusions; (21) aneurysm neck bridge inside aneurysm sack with convex shape; (22) single-chamber woven/mesh structure in aneurysm sack; (23) multi-chamber woven/mesh structure in aneurysm sack; (24) embolic coils with relatively-traditional structures in aneurysm sack; (25) embolic coils with complex structures in aneurysm sack; (26) embolic coils with inter-connecting members in aneurysm sack; (27) embolic coils with special coatings in aneurysm sack; (28) polymer or hydrogel longitudinal embolic members in aneurysm sack; (29) longitudinal embolic members with string-of-pearls structure in aneurysm sack; (30) accumulation of mass in aneurysm sack by spooling and/or axial rotation; (31) liner or balloon with non-porous walls in aneurysm sack; (32) liner, balloon, net, or mesh with porous walls in aneurysm sack; (33) liquid embolic composition into aneurysm sack; (34) gelatinous embolic composition into aneurysm sack; (35) embolic spheres and/or particles into aneurysm sack; (36) customized pre-molded member into aneurysm sack; (37) extravascular sleeve around aneurysm sack and parent vessel; and (38) other devices for aneurysm treatment.

1. Stent with Circumferential Variation in Wall Porosity in Parent Vessel:

The prior art discloses devices and methods for treating aneurysms comprising generally-cylindrical stents with circumferential variation in wall porosity which are implanted within the parent vessel of an aneurysm. In an example, a less porous portion of such a stent can comprise a “patch” which covers the aneurysm neck. In an example, a less-porous section of such a stent can be located so as to span an aneurysm neck and reduce blood flow to the aneurysm, while a more-porous section of the stent can be located so as to allow continued blood flow to a nearby (small) branching vessel. Prior art which appears to be within this category includes U.S. Pat. No. 5,951,599 (McCrory, Sep. 14, 1999, “Occlusion System for Endovascular Treatment of an Aneurysm”); U.S. Pat. No. 6,605,111 (Bose et al., Aug. 12, 2003, “Endovascular Thin Film Devices and Methods for Treating and Preventing Stroke”); U.S. Pat. No. 6,746,468 (Sepetka et al., Jun. 8, 2004, “Devices and Methods for Treating Vascular Malformations”); U.S. Pat. No. 6,969,401 (Marotta et al., Nov. 29, 2005, “Endovascular Prosthesis”); U.S. Pat. No. 7,147,659 (Jones, Dec. 12, 2006, “Expandable Stent Having a Dissolvable Portion”); U.S. Pat. No. 7,156,871 (Jones et al., Jan. 2, 2007, “Expandable Stent Having a Stabilized Portion”); U.S. Pat. No. 7,491,226 (Palmaz et al., Feb. 17, 2009, “Endoluminal Implantable Stent-Grafts”); U.S. Pat. No. 7,572,288 (Cox, Aug. 11, 2009, “Aneurysm Treatment Device and Method of Use”); U.S. Pat. No. 7,611,530 (Pomeranz et al., Nov. 3, 2009, “Expandable Stent Having Removable Slat Members”); U.S. Pat. No. 7,641,680 (Palmaz et al., Jan. 5, 2010, “Endoluminal Implantable Stent-Grafts”); and U.S. Pat. No. 7,769,603 (Jung et al., Aug. 3, 2010, “Stent Customization System and Method”).

Prior art which appears to be within this category also includes U.S. Pat. No. 7,780,645 (Jones, Aug. 24, 2010, “Method of Delivering Embolic Particles to an Aneurysm”); U.S. Pat. No. 8,038,706 (Eidenschink et al., Oct. 18, 2011, “Crown Stent Assembly”); U.S. Pat. No. 8,252,040 (Cox, Aug. 28, 2012, “Aneurysm Treatment Device and Method of Use”); U.S. Pat. No. 8,267,986 (Berez et al., Sep. 18, 2012, “Vascular Stenting for Aneurysms”); U.S. Pat. No. 8,353,943 (Kuppurathanam et al., Jan. 15, 2013, “Variable Weave Graft with Metal Strand Reinforcement for In Situ Fenestration”); U.S. Pat. No. 8,382,825 (Garcia et al., Feb. 26, 2013, “Flexible Vascular Occluding Device”); U.S. Pat. No. 8,398,701 (Berez et al., Mar. 19, 2013, “Flexible Vascular Occluding Device”); U.S. Pat. No. 8,409,267 (Berez et al., Apr. 2, 2013, “Vascular Stenting for Aneurysms”); U.S. Pat. No. 8,409,269 (Berez et al., Apr. 2, 2013, “Procedures for Vascular Occlusion”); U.S. Pat. No. 8,425,548 (Connor, Apr. 23, 2013, “Occluding Member Expansion and then Stent Expansion for Aneurysm Treatment”); U.S. Pat. No. 8,430,922 (Jung et al., Apr. 30, 2013, “Stent Customization System and Method”); U.S. Pat. No. 8,470,013 (Duggal et al., Jun. 25, 2013, “Systems and Methods for Aneurysm Treatment and Vessel Occlusion”); U.S. Pat. No. 8,475,517 (Jung et al., Jul. 2, 2013, “Stent Customization System and Method”); U.S. Pat. No. 8,500,788 (Berez et al., Aug. 6, 2013, “Vascular Stenting and Other Procedures”); U.S. Pat. No. 8,529,614 (Berez et al., Sep. 10, 2013, “Vascular Stenting and Other Procedures”); U.S. Pat. No. 8,529,614 (Berez et al., Sep. 10, 2013, “Vascular Stenting and Other Procedures”); U.S. Pat. No. 8,550,344 (Jung et al., Oct. 8, 2013, “Specialty Stents with Flow Control Features or the Like”); U.S. Pat. No. 8,551,155 (Jung et al., Oct. 8, 2013, “Stent Customization System and Method”); and U.S. Pat. No. 8,556,953 (Berez et al., Oct. 15, 2013, “Vascular Stenting for Aneurysms”).

Prior art which appears to be within this category also includes U.S. Pat. No. 8,562,667 (Cox, Oct. 22, 2013, “Aneurysm Treatment Device and Method of Use”); U.S. Pat. No. 8,577,693 (Jung et al., Nov. 5, 2013, “Specialty Stents with Flow Control Features or the Like”); U.S. Pat. No. 8,597,320 (Sepetka et al., Dec. 3, 2013, “Devices and Methods for Treating Vascular Malformations”); U.S. Pat. No. 8,597,342 (McKinsey et al., Dec. 3, 2013, “Textile Graft for In Situ Fenestration”); U.S. Pat. No. 8,715,312 (Burke et al., May 6, 2014, “Aneurysm Treatment Device and Method of Use”); U.S. Pat. No. 8,715,317 (Janardhan et al., May 6, 2014, “Flow Diverting Devices”); U.S. Pat. No. 8,721,706 (Jung et al., May 13, 2014, “Specialty Stents with Flow Control Features or the Like”); U.S. Pat. No. 8,747,432 (Janardhan et al., Jun. 10, 2014, “Woven Vascular Treatment Devices”); U.S. Pat. No. 8,753,371 (Janardhan et al., Jun. 17, 2014, “Woven Vascular Treatment Systems”); U.S. Pat. No. 8,784,446 (Janardhan et al., Jul. 22, 2014, “Circumferentially Offset Variable Porosity Devices”); U.S. Pat. No. 8,813,625 (Janardhan et al., Aug. 26, 2014, “Methods of Manufacturing Variable Porosity Flow Diverting Devices”); and U.S. Pat. No. 8,845,679 (Janardhan et al., Sep. 30, 2014, “Variable Porosity Flow Diverting Devices”).

Prior art which appears to be within this category also includes U.S. patent applications: 20020169473 (Sepetka et al., Nov. 14, 2002, “Devices and Methods for Treating Vascular Malformations”); 20030018294 (Cox, Jan. 23, 2003, “Aneurysm Treatment Device and Method of Use”); 20030109917 (Rudin et al., Jun. 12, 2003, “Stent Vascular Intervention Device and Method”); 20030139802 (Wulfman et al., Jul. 24, 2003, “Medical Device”); 20040181253 (Sepetka et al., Sep. 16, 2004, “Devices and Methods for Treating Vascular Malformations”); 20050267568 (Berez et al., Dec. 1, 2005, “Flexible Vascular Occluding Device”); 20060206200 (Garcia et al., Sep. 14, 2006, “Flexible Vascular Occluding Device”); 20060206201 (Garcia et al., Sep. 14, 2006, “Flexible Vascular Occluding Device”); 20070021816 (Rudin, Jan. 25, 2007, “Stent Vascular Intervention Device and Methods for Treating Aneurysms”); 20070067015 (Jones et al., Mar. 22, 2007, “Expandable Stent Having a Stabilized Portion”); and 20070239261 (Bose et al., Oct. 11, 2007, “Aneurysm Occlusion System and Method”).

Prior art which appears to be within this category also includes U.S. patent applications: 20080114391 (Dieck et al., May 15, 2008, “Aneurysm Covering Devices and Delivery Devices”); 20080114436 (Dieck et al., May 15, 2008, “Aneurysm Covering Devices and Delivery Devices”); 20090069880 (Vonderwalde et al., Mar. 12, 2009, “Implantable Graft Assembly and Aneurysm Treatment”); 20090270974 (Berez et al., Oct. 29, 2009, “Vascular Stenting for Aneurysms”); 20090287241 (Berez et al., Nov. 19, 2009, “Methods and Apparatus for Luminal Stenting”); 20090287288 (Berez et al., Nov. 19, 2009, “Methods and Apparatus for Luminal Stenting”); 20090292348 (Berez et al., Nov. 26, 2009, “Vascular Stenting and Other Procedures”); 20090319017 (Berez et al., Dec. 24, 2009, “Vascular Stenting for Aneurysms”); 20100010624 (Berez et al., Jan. 14, 2010, “Vascular Stenting for Aneurysms”); 20100082091 (Berez et al., Apr. 1, 2010, “Vascular Stenting and Other Procedures”); and 20100106240 (Duggal et al., Apr. 29, 2010, “Systems and Methods for Aneurysm Treatment and Vessel Occlusion”).

Prior art which appears to be within this category also includes U.S. patent applications: 20100274276 (Chow et al., Oct. 28, 2010, “Aneurysm Treatment System, Device and Method”); 20110082491 (Sepetka et al., Apr. 7, 2011, “Devices and Methods for Treating Vascular Malformations”); 20110137332 (Sepetka et al., Jun. 9, 2011, “Devices and Methods for Treating Vascular Malformations”); 20110152998 (Berez et al., Jun. 23, 2011, “Procedures for Vascular Occlusion”); 20110166592 (Garcia et al., Jul. 7, 2011, “Flexible Vascular Occluding Device”); 20110224776 (Sepetka et al., Sep. 15, 2011, “Devices and Methods for Treating Vascular Malformations”); 20120004682 (Connor, Jan. 5, 2012, “Occluding Member Expansion and Then Stent Expansion for Aneurysm Treatment”); 20130123901 (Connor et al., May 16, 2013, “Stent with In Situ Determination of Wall Areas with Differences in Porosity”); 20130172975 (Berez et al., Jul. 4, 2013, “Methods and Apparatus for Luminal Stenting”); 20130204288 (Johnson et al., Aug. 8, 2013, “Modifiable Occlusion Device”); 20130204288 (Johnson et al., Aug. 8, 2013, “Modifiable Occlusion Device”); and 20130231732 (Vonderwalde et al., Sep. 5, 2013, “Implantable Graft Assembly and Aneurysm Treatment”).

Prior art which appears to be within this category also includes U.S. patent applications: 20130238083 (Duggal et al., Sep. 12, 2013, “Systems and Methods for Aneurysm Treatment and Vessel Occlusion”); 20130282096 (Berez et al., Oct. 24, 2013, “Procedures for Vascular Occlusion”); 20140018843 (Berez et al., Jan. 16, 2014, “Methods and Apparatus for Luminal Stenting”); 20140039606 (Rudakov et al., Feb. 6, 2014, “Medical Device”); 20140074149 (Garcia et al., Mar. 13, 2014, “Flexible Vascular Occluding Device”); 20140094896 (Berez et al., Apr. 3, 2014, “Vascular Stenting for Aneurysms”); 20140114342 (Berez et al., Apr. 24, 2014, “Flexible Vascular Occluding Device”); 20140128901 (Kang et al., May 8, 2014, “Implant for Aneurysm Treatment”); 20140172071 (Berez et al., Jun. 19, 2014, “Vascular Stenting for Aneurysms”); 20140260928 (Janardhan et al., Sep. 18, 2014, “Methods of Using Non-Cylindrical Mandrels”); 20140265096 (Janardhan et al., Sep. 18, 2014, “Non-Cylindrical Mandrels”); and 20140288633 (Burke et al., Sep. 25, 2014, “Aneurysm Treatment Device and Method of Use”); and Yet unpublished U.S. patent application Ser. No. 13/889,451 (Connor et al., 2013, “Method of Radially-Asymmetric Stent Expansion”).

2. Stent with Longitudinal Variation in Wall Porosity in Parent Vessel:

The prior art also discloses devices and methods for treating aneurysms comprising generally-cylindrical stents with longitudinal variation in wall porosity which are implanted within the parent vessel of an aneurysm. In an example, a less-porous section of such a stent can be located so as to span an aneurysm neck and reduce blood flow to the aneurysm, while a more-porous section of the stent can be located so as to allow continued blood flow to a nearby (small) branching vessel. Prior art which appears to be within this category includes U.S. Pat. No. 5,693,088 (Lazarus, Dec. 2, 1997, “Intraluminal Vascular Graft”); U.S. Pat. No. 5,769,884 (Solovay, Jun. 23, 1998, “Controlled Porosity Endovascular Implant”); U.S. Pat. No. 5,951,599 (McCrory, Sep. 14, 1999, “Occlusion System for Endovascular Treatment of an Aneurysm”); U.S. Pat. No. 6,093,199 (Brown et al., Jul. 25, 2000, “Intra-Luminal Device for Treatment of Body Cavities and Lumens and Method of Use”); U.S. Pat. No. 6,258,115 (Dubrul, Jul. 10, 2001, “Bifurcated Stent and Distal Protection System”); U.S. Pat. No. 6,312,463 (Rourke et al., Nov. 6, 2001, “Micro-Porous Mesh Stent with Hybrid Structure”); U.S. Pat. No. 6,585,758 (Chouinard et al., Jul. 1, 2003, “Multi-Section Filamentary Endoluminal Stent”); U.S. Pat. No. 6,676,701 (Rourke et al., Jan. 13, 2004, “Micro-Porous Mesh Stent with Hybrid Structure”); U.S. Pat. No. 6,746,468 (Sepetka et al., Jun. 8, 2004, “Devices and Methods for Treating Vascular Malformations”); U.S. Pat. No. 6,913,618 (Denardo et al., Jul. 5, 2005, “Intravascular Flow Modifier and Reinforcement Device”); U.S. Pat. No. 7,041,129 (Rourke et al., May 9, 2006, “Micro-Porous Mesh Stent with Hybrid Structure”); U.S. Pat. No. 7,288,112 (Denardo et al., Oct. 30, 2007, “Intravascular Flow Modifier and Reinforcement Device”); and U.S. Pat. No. 7,306,624 (Yodfat et al., Dec. 11, 2007, “Implantable Intraluminal Device and Method of Using Same in Treating Aneurysms”).

Prior art which appears to be within this category also includes U.S. Pat. No. 7,491,226 (Palmaz et al., Feb. 17, 2009, “Endoluminal Implantable Stent-Grafts”); U.S. Pat. No. 7,572,290 (Yodfat et al., Aug. 11, 2009, “Implantable Intraluminal Device and Method of Using Same in Treating Aneurysms”); U.S. Pat. No. 7,641,680 (Palmaz et al., Jan. 5, 2010, “Endoluminal Implantable Stent-Grafts”); U.S. Pat. No. 7,695,509 (Rourke et al., Apr. 13, 2010, “Micro-Porous Mesh Stent with Hybrid Structure”); U.S. Pat. No. 7,763,011 (Ortiz et al., Jul. 27, 2010, “Variable Density Braid Stent”); U.S. Pat. No. 7,769,603 (Jung et al., Aug. 3, 2010, “Stent Customization System and Method”); U.S. Pat. No. 7,811,300 (Feller et al., Oct. 12, 2010, “Thin Film Devices for Temporary or Permanent Occlusion of a Vessel”); U.S. Pat. No. 7,857,843 (Henderson, Dec. 28, 2010, “Differentially Expanded Vascular Graft”); U.S. Pat. No. 7,862,608 (Hogendijk et al., Jan. 4, 2011, “Vascular Prosthesis and Methods of Use”); U.S. Pat. No. 7,942,925 (Yodfat et al., May 17, 2011, “Implantable Intraluminal Device and Method of Using Same in Treating Aneurysms”); U.S. Pat. No. 8,007,529 (Yan, Aug. 30, 2011, “Medicated Porous Metal Prosthesis”); U.S. Pat. No. 8,267,986 (Berez et al., Sep. 18, 2012, “Vascular Stenting for Aneurysms”); and U.S. Pat. No. 8,353,943 (Kuppurathanam et al., Jan. 15, 2013, “Variable Weave Graft with Metal Strand Reinforcement for In Situ Fenestration”).

Prior art which appears to be within this category also includes U.S. Pat. No. 8,409,267 (Berez et al., Apr. 2, 2013, “Vascular Stenting for Aneurysms”); U.S. Pat. No. 8,409,269 (Berez et al., Apr. 2, 2013, “Procedures for Vascular Occlusion”); U.S. Pat. No. 8,419,787 (Yodfat et al., Apr. 16, 2013, “Implantable Intraluminal Device and Method of Using Same in Treating Aneurysms”); U.S. Pat. No. 8,430,922 (Jung et al., Apr. 30, 2013, “Stent Customization System and Method”); U.S. Pat. No. 8,470,013 (Duggal et al., Jun. 25, 2013, “Systems and Methods for Aneurysm Treatment and Vessel Occlusion”); U.S. Pat. No. 8,475,517 (Jung et al., Jul. 2, 2013, “Stent Customization System and Method”); U.S. Pat. No. 8,491,646 (Schreck, Jul. 23, 2013, “Stent Graft”); U.S. Pat. No. 8,500,788 (Berez et al., Aug. 6, 2013, “Vascular Stenting and Other Procedures”); U.S. Pat. No. 8,506,618 (Chouinard et al., Aug. 13, 2013, “Multi-Section Filamentary Endoluminal Stent”); U.S. Pat. No. 8,506,619 (Ortiz et al., Aug. 13, 2013, “Variable Density Braid Stent”); U.S. Pat. No. 8,529,614 (Berez et al., Sep. 10, 2013, “Vascular Stenting and Other Procedures”); U.S. Pat. No. 8,529,614 (Berez et al., Sep. 10, 2013, “Vascular Stenting and Other Procedures”); U.S. Pat. No. 8,535,590 (Milner et al., Sep. 17, 2013, “Spray System and Method of Making Phase Separated Polymer Membrane Structures”); and U.S. Pat. No. 8,550,344 (Jung et al., Oct. 8, 2013, “Specialty Stents with Flow Control Features or the Like”).

Prior art which appears to be within this category also includes U.S. Pat. No. 8,551,155 (Jung et al., Oct. 8, 2013, “Stent Customization System and Method”); U.S. Pat. No. 8,556,953 (Berez et al., Oct. 15, 2013, “Vascular Stenting for Aneurysms”); U.S. Pat. No. 8,577,693 (Jung et al., Nov. 5, 2013, “Specialty Stents with Flow Control Features or the Like”); U.S. Pat. No. 8,597,320 (Sepetka et al., Dec. 3, 2013, “Devices and Methods for Treating Vascular Malformations”); U.S. Pat. No. 8,715,317 (Janardhan et al., May 6, 2014, “Flow Diverting Devices”); U.S. Pat. No. 8,721,706 (Jung et al., May 13, 2014, “Specialty Stents with Flow Control Features or the Like”); U.S. Pat. No. 8,747,432 (Janardhan et al., Jun. 10, 2014, “Woven Vascular Treatment Devices”); U.S. Pat. No. 8,753,371 (Janardhan et al., Jun. 17, 2014, “Woven Vascular Treatment Systems”); U.S. Pat. No. 8,784,446 (Janardhan et al., Jul. 22, 2014, “Circumferentially Offset Variable Porosity Devices”); U.S. Pat. No. 8,813,625 (Janardhan et al., Aug. 26, 2014, “Methods of Manufacturing Variable Porosity Flow Diverting Devices”); and U.S. Pat. No. 8,845,679 (Janardhan et al., Sep. 30, 2014, “Variable Porosity Flow Diverting Devices”).

Prior art which appears to be within this category also includes U.S. patent applications: 20020169473 (Sepetka et al., Nov. 14, 2002, “Devices and Methods for Treating Vascular Malformations”); 20030074049 (Hoganson et al., Apr. 17, 2003, “Covered Stents and Systems for Deploying Covered Stents”); 20030100945 (Yodfat et al., May 29, 2003, “Implantable Intraluminal Device and Method of Using Same in Treating Aneurysms”); 20030195606 (Davidson et al., Oct. 16, 2003, “Bifurcation Stent System and Method”); 20040111142 (Rourke et al., Jun. 10, 2004, “Micro-Porous Mesh Stent with Hybrid Structure”); 20040181253 (Sepetka et al., Sep. 16, 2004, “Devices and Methods for Treating Vascular Malformations”); 20040193246 (Ferrera, Sep. 30, 2004, “Methods and Apparatus for Treating Aneurysms and Other Vascular Defects”); 20050010281 (Yodfat et al., Jan. 13, 2005, “Implantable Intraluminal Device and Method of Using Same in Treating Aneurysms”); and 20050090888 (Hines et al., Apr. 28, 2005, “Pleated Stent Assembly”).

Prior art which appears to be within this category also includes U.S. patent applications: 20050283220 (Gobran et al., Dec. 22, 2005, “Blood Flow Diverters for the Treatment of Intracranial Aneurysms”); 20070032855 (Davidson et al., Feb. 8, 2007, “Extendible Stent Apparatus”); 20070060994 (Gobran et al., Mar. 15, 2007, “Blood Flow Diverters for the Treatment of Intracranial Aneurysms”); 20070150045 (Ferrera, Jun. 28, 2007, “Methods and Apparatus for Treating Aneurysms and Other Vascular Defects”); 20070219619 (Dieck et al., Sep. 20, 2007, “Partially Covered Stent Devices and Methods of Use”); 20080004653 (Sherman et al., Jan. 3, 2008, “Thin Film Devices for Occlusion of a Vessel”); 20080039933 (Yodfat et al., Feb. 14, 2008, “Implantable Intraluminal Device and Method of Using Same in Treating Aneurysms”); 20090069880 (Vonderwalde et al., Mar. 12, 2009, “Implantable Graft Assembly and Aneurysm Treatment”); 20090270970 (Yodfat et al., Oct. 29, 2009, “Implantable Intraluminal Device and Method of Using Same in Treating Aneurysms”); 20090270974 (Berez et al., Oct. 29, 2009, “Vascular Stenting for Aneurysms”); and 20090287241 (Berez et al., Nov. 19, 2009, “Methods and Apparatus for Luminal Stenting”).

Prior art which appears to be within this category also includes U.S. patent applications: 20090287288 (Berez et al., Nov. 19, 2009, “Methods and Apparatus for Luminal Stenting”); 20090292348 (Berez et al., Nov. 26, 2009, “Vascular Stenting and Other Procedures”); 20090319017 (Berez et al., Dec. 24, 2009, “Vascular Stenting for Aneurysms”); 20090319023 (Hildebrand et al., Dec. 24, 2009, “Stents and Stent Grafts”); 20100010624 (Berez et al., Jan. 14, 2010, “Vascular Stenting for Aneurysms”); 20100082091 (Berez et al., Apr. 1, 2010, “Vascular Stenting and Other Procedures”); 20100106240 (Duggal et al., Apr. 29, 2010, “Systems and Methods for Aneurysm Treatment and Vessel Occlusion”); 20100152834 (Hannes et al., Jun. 17, 2010, “Implant for Influencing Blood Flow”); 20100198334 (Yodfat et al., Aug. 5, 2010, “Implantable Intraluminal Device and Method of Using Same in Treating Aneurysms”); 20100274346 (Chouinard et al., Oct. 28, 2010, “Multi-Section Filamentary Endoluminal Stent”); 20110046716 (Parkinson et al., Feb. 24, 2011, “Stent”); 20110054589 (Bashiri et al., Mar. 3, 2011, “Stent with Variable Cross Section Braiding Filament and Method for Making Same”); 20110082491 (Sepetka et al., Apr. 7, 2011, “Devices and Methods for Treating Vascular Malformations”); 20110137332 (Sepetka et al., Jun. 9, 2011, “Devices and Methods for Treating Vascular Malformations”); and 20110152998 (Berez et al., Jun. 23, 2011, “Procedures for Vascular Occlusion”).

Prior art which appears to be within this category also includes U.S. patent applications: 20110224776 (Sepetka et al., Sep. 15, 2011, “Devices and Methods for Treating Vascular Malformations”); 20120253377 (Slazas et al., Oct. 4, 2012, “Modifiable Occlusion Device”); 20120271399 (Perkins et al., Oct. 25, 2012, “High Metal to Vessel Ratio Landing Zone Stent-Graft and Method”); 20120303112 (Armstrong et al., Nov. 29, 2012, “Stent”); 20130123901 (Connor et al., May 16, 2013, “Stent with In Situ Determination of Wall Areas with Differences in Porosity”); 20130131780 (Armstrong et al., May 23, 2013, “Lattice”); 20130172975 (Berez et al., Jul. 4, 2013, “Methods and Apparatus for Luminal Stenting”); 20130190805 (Slazas et al., Jul. 25, 2013, “Method of Fabricating Modifiable Occlusion Device”); 20130197617 (Armstrong et al., Aug. 1, 2013, “Stent”); 20130197624 (Armstrong et al., Aug. 1, 2013, “Stent”); 20130204347 (Armstrong et al., Aug. 8, 2013, “Lattice”); 20130204351 (Cox et al., Aug. 8, 2013, “Aneurysm Graft Devices and Methods”); 20130211497 (Charlebois et al., Aug. 15, 2013, “Medical Prostheses Having Bundled and Non-Bundled Regions”); and 20130226276 (Newell et al., Aug. 29, 2013, “Methods and Apparatus for Luminal Stenting”).

Prior art which appears to be within this category also includes U.S. patent applications: 20130226278 (Newell et al., Aug. 29, 2013, “Methods and Apparatus for Luminal Stenting”); 20130231732 (Vonderwalde et al., Sep. 5, 2013, “Implantable Graft Assembly and Aneurysm Treatment”); 20130238083 (Duggal et al., Sep. 12, 2013, “Systems and Methods for Aneurysm Treatment and Vessel Occlusion”); 20130245745 (Vong et al., Sep. 19, 2013, “Stent and Stent Delivery Device”); 20130261728 (Perkins et al., Oct. 3, 2013, “High Metal to Vessel Ratio Stent and Method”); 20130261732 (Perkins et al., Oct. 3, 2013, “Integrated Mesh High Metal to Vessel Ratio Stent and Method”); 20130282096 (Berez et al., Oct. 24, 2013, “Procedures for Vascular Occlusion”); 20140018843 (Berez et al., Jan. 16, 2014, “Methods and Apparatus for Luminal Stenting”); 20140025151 (Gao, Jan. 23, 2014, “Retrievable Stent for Intracranial Aneurysms”); 20140039606 (Rudakov et al., Feb. 6, 2014, “Medical Device”); 20140058498 (Hannes et al., Feb. 27, 2014, “Implant Comprising a Non-Woven Fabric”); 20140083969 (Porter, Mar. 27, 2014, “Method of Manufacturing a Variably Reinforced Elongate Medical Device”); and 20140094896 (Berez et al., Apr. 3, 2014, “Vascular Stenting for Aneurysms”).

Prior art which appears to be within this category also includes U.S. patent applications: 20140121744 (Kusleika, May 1, 2014, “Methods and Systems for Increasing a Density of a Region of a Vascular Device”); 20140121745 (Kusleika et al., May 1, 2014, “Systems for Attaining a Predetermined Porosity of a Vascular Device”); 20140121746 (Kusleika et al., May 1, 2014, “Methods for Attaining a Predetermined Porosity of a Vascular Device”); 20140128901 (Kang et al., May 8, 2014, “Implant for Aneurysm Treatment”); 20140172071 (Berez et al., Jun. 19, 2014, “Vascular Stenting for Aneurysms”); 20140200648 (Newell et al., Jul. 17, 2014, “Methods and Apparatus for Luminal Stenting”); 20140249620 (Carman et al., Sep. 4, 2014, “Ultra-Low Fractional Area Coverage Flow Diverter for Treating Aneurysms and Vascular Diseases”); 20140260928 (Janardhan et al., Sep. 18, 2014, “Methods of Using Non-Cylindrical Mandrels”); and 20140265096 (Janardhan et al., Sep. 18, 2014, “Non-Cylindrical Mandrels”).

3. Stent with Longitudinal or Cross-Sectional Variation in Size and/or Flexibility in Parent Vessel:

The prior art also discloses devices and methods for treating aneurysms comprising generally-cylindrical stents with longitudinal or cross-sectional variation in their size and/or flexibility which are implanted within the parent vessel of an aneurysm. Prior art which appears to be within this category includes U.S. Pat. No. 5,836,966 (St. Germain, Nov. 17, 1998, “Variable Expansion Force Stent”); U.S. Pat. No. 5,868,780 (Lashinski et al., Feb. 9, 1999, “Stents for Supporting Lumens in Living Tissue”); U.S. Pat. No. 5,922,019 (Hankh et al., Jul. 13, 1999, “Conical Stent”); U.S. Pat. No. 5,938,697 (Killion et al., Aug. 17, 1999, “Stent Having Variable Properties”); U.S. Pat. No. 5,957,975 (Lafont et al., Sep. 28, 1999, “Stent Having a Programmed Pattern of In Vivo Degradation”); U.S. Pat. No. 5,980,514 (Kupiecki et al., Nov. 9, 1999, “Aneurysm Closure Device Assembly”); U.S. Pat. No. 6,027,526 (Limon et al., Feb. 22, 2000, “Stent Having Varied Amounts of Structural Strength Along its Length”); U.S. Pat. No. 6,071,298 (Lashinski et al., Jun. 6, 2000, “Stents for Supporting Lumens in Living Tissue”); U.S. Pat. No. 6,096,034 (Kupiecki et al., Aug. 1, 2000, “Aneurysm Closure Device Assembly”); U.S. Pat. No. 6,099,559 (Notting, Aug. 8, 2000, “Endoluminal Support Assembly with Capped Ends”); U.S. Pat. No. 6,159,238 (Killion et al., Dec. 12, 2000, “Stent Having Variable Properties and Method of Its Use”); U.S. Pat. No. 6,168,592 (Kupiecki et al., Jan. 2, 2001, “Aneurysm Closure Device Assembly”); U.S. Pat. No. 6,273,910 (Limon, Aug. 14, 2001, “Stent with Varying Strut Geometry”); U.S. Pat. No. 6,273,911 (Cox et al., Aug. 14, 2001, “Variable Strength Stent”); and U.S. Pat. No. 6,344,041 (Kupiecki et al., Feb. 5, 2002, “Aneurysm Closure Device Assembly”).

Prior art which appears to be within this category also includes U.S. Pat. No. 6,468,302 (Cox et al., Oct. 22, 2002, “Variable Strength Stent”); U.S. Pat. No. 6,475,233 (Trozera, Nov. 5, 2002, “Stent Having Tapered Struts”); U.S. Pat. No. 6,475,236 (Roubin et al., Nov. 5, 2002, “Non-Foreshortening Intraluminal Prosthesis”); U.S. Pat. No. 6,485,509 (Killion et al., Nov. 26, 2002, “Stent Having Variable Properties and Method of its Use”); U.S. Pat. No. 6,497,722 (Von Oepen et al., Dec. 24, 2002, “Methods and Apparatus for In-Vivo Tailored Stents Indicated for Use in Tortuous Anatomy”); U.S. Pat. No. 6,511,505 (Cox et al., Jan. 28, 2003, “Variable Strength Stent”); U.S. Pat. No. 6,569,193 (Cox et al., May 27, 2003, “Tapered Self-Expanding Stent”); U.S. Pat. No. 6,585,758 (Chouinard et al., Jul. 1, 2003, “Multi-Section Filamentary Endoluminal Stent”); U.S. Pat. No. 6,602,284 (Cox et al., Aug. 5, 2003, “Variable Strength Stent”); U.S. Pat. No. 6,645,237 (Klumb et al., Nov. 11, 2003, “Expandable Coiled Endoluminal Prosthesis”); U.S. Pat. No. 6,652,576 (Stalker, Nov. 25, 2003, “Variable Stiffness Stent”); U.S. Pat. No. 6,660,032 (Klumb et al., Dec. 9, 2003, “Expandable Coil Endoluminal Prosthesis”); U.S. Pat. No. 6,669,723 (Killion et al., Dec. 30, 2003, “Stent Having Variable Properties and Method of its Use”); U.S. Pat. No. 6,746,475 (Rivelli, Jun. 8, 2004, “Stent with Variable Stiffness”); and U.S. Pat. No. 6,796,997 (Penn et al., Sep. 28, 2004, “Expandable Stent”).

Prior art which appears to be within this category also includes U.S. Pat. No. 6,814,754 (Greenhalgh, Nov. 9, 2004, “Woven Tubular Graft with Regions of Varying Flexibility”); U.S. Pat. No. 6,860,899 (Rivelli, Mar. 1, 2005, “Method for Treating Neurovascular Aneurysms”); U.S. Pat. No. 6,899,730 (Rivelli, May 31, 2005, “Catheter-Stent Device”); U.S. Pat. No. 7,001,422 (Escamilla et al., Feb. 21, 2006, “Expandable Stent and Delivery System”); U.S. Pat. No. 7,060,091 (Killion et al., Jun. 13, 2006, “Stent Having Variable Properties and Method of Its Use”); U.S. Pat. No. 7,112,216 (Gregorich, Sep. 26, 2006, “Stent with Tapered Flexibility”); U.S. Pat. No. 7,226,475 (Lenz et al., Jun. 5, 2007, “Stent with Variable Properties”); U.S. Pat. No. 7,241,308 (Andreas et al., Jul. 10, 2007, “Stent Deployment Systems and Methods”); U.S. Pat. No. 7,309,351 (Escamilla et al., Dec. 18, 2007, “Expandable Stent with Markers and Stent Delivery System”); U.S. Pat. No. 7,326,236 (Andreas et al., Feb. 5, 2008, “Devices and Methods for Controlling and Indicating the Length of an Interventional Element”); U.S. Pat. No. 7,402,169 (Killion et al., Jul. 22, 2008, “Stent Having Variable Properties and Method of its Use”); U.S. Pat. No. 7,520,893 (Rivelli, Apr. 21, 2009, “Method for Treating Neurovascular Aneurysms”); U.S. Pat. No. 7,780,719 (Killion et al., Aug. 24, 2010, “Stent Having Variable Properties and Method of its Use”); U.S. Pat. No. 7,892,273 (George et al., Feb. 22, 2011, “Custom Length Stent Apparatus”); U.S. Pat. No. 7,918,881 (Andreas et al., Apr. 5, 2011, “Stent Deployment Systems and Methods”); and U.S. Pat. No. 7,935,142 (Gregorich, May 3, 2011, “Stent with Tapered Flexibility”); U.S. Pat. No. 7,959,662 (Erbel et al., Jun. 14, 2011, “Endovascular Prosthesis”).

Prior art which appears to be within this category also includes U.S. Pat. No. 8,357,178 (Grandfield et al., Jan. 22, 2013, “Vascular and Bodily Duct Treatment Devices and Methods”); U.S. Pat. No. 8,357,179 (Grandfield et al., Jan. 22, 2013, “Vascular and Bodily Duct Treatment Devices and Methods”); U.S. Pat. No. 8,506,618 (Chouinard et al., Aug. 13, 2013, “Multi-Section Filamentary Endoluminal Stent”); U.S. Pat. No. 8,529,596 (Grandfield et al., Sep. 10, 2013, “Vascular and Bodily Duct Treatment Devices and Methods”); U.S. Pat. No. 8,715,317 (Janardhan et al., May 6, 2014, “Flow Diverting Devices”); U.S. Pat. No. 8,734,502 (Orr, May 27, 2014, “Tapered Stent and Flexible Prosthesis”); U.S. Pat. No. 8,747,432 (Janardhan et al., Jun. 10, 2014, “Woven Vascular Treatment Devices”); U.S. Pat. No. 8,753,371 (Janardhan et al., Jun. 17, 2014, “Woven Vascular Treatment Systems”); U.S. Pat. No. 8,771,341 (Strauss et al., Jul. 8, 2014, “Protuberant Aneurysm Bridging Device and Method of Use”); U.S. Pat. No. 8,784,446 (Janardhan et al., Jul. 22, 2014, “Circumferentially Offset Variable Porosity Devices”); U.S. Pat. No. 8,795,345 (Grandfield et al., Aug. 5, 2014, “Vascular and Bodily Duct Treatment Devices and Methods”); U.S. Pat. No. 8,808,361 (Strauss et al., Aug. 19, 2014, “Protuberant Aneurysm Bridging Device and Method of Use”); U.S. Pat. No. 8,813,625 (Janardhan et al., Aug. 26, 2014, “Methods of Manufacturing Variable Porosity Flow Diverting Devices”); and U.S. Pat. No. 8,845,679 (Janardhan et al., Sep. 30, 2014, “Variable Porosity Flow Diverting Devices”).

Prior art which appears to be within this category also includes U.S. patent applications: 20030074056 (Killion et al., Apr. 17, 2003, “Stent Having Variable Properties and Method of its Use”); 20040220663 (Rivelli, J R., Nov. 4, 2004, “Stent with Variable Stiffness”); 20040243216 (Gregorich, Dec. 2, 2004, “Stent with Tapered Flexibility”); 20040249439 (Richter et al., Dec. 9, 2004, “Method and Apparatus for Stenting”); 20050149159 (Andreas et al., Jul. 7, 2005, “Devices and Methods for Controlling and Indicating the Length of an Interventional Element”); 20050149164 (Rivelli, Jul. 7, 2005, “Method for Treating Neurovascular Aneurysms”); 20080319525 (Tieu et al., Dec. 25, 2008, “Self-Expanding Prosthesis”); 20100016833 (Ogle et al., Jan. 21, 2010, “Devices for the Treatment of Vascular Aneurysm”); 20100042200 (Richter et al., Feb. 18, 2010, “Method and Apparatus for Stenting”); 20100114302 (Tzafriri et al., May 6, 2010, “Endovascular Devices with Axial Perturbations”); and 20100274346 (Chouinard et al., Oct. 28, 2010, “Multi-Section Filamentary Endoluminal Stent”).

Prior art which appears to be within this category also includes U.S. patent applications: 20110009940 (Grandfield et al., Jan. 13, 2011, “Vascular and Bodily Duct Treatment Devices and Methods”); 20110009941 (Grandfield et al., Jan. 13, 2011, “Vascular and Bodily Duct Treatment Devices and Methods”); 20110009950 (Grandfield et al., Jan. 13, 2011, “Vascular and Bodily Duct Treatment Devices and Methods”); 20110184456 (Grandfield et al., Jul. 28, 2011, “Vascular and Bodily Duct Treatment Devices and Methods”); 20120209311 (Grandfield et al., Aug. 16, 2012, “Vascular and Bodily Duct Treatment Devices and Methods”); 20120215250 (Grandfield et al., Aug. 23, 2012, “Vascular and Bodily Duct Treatment Devices and Methods”); 20130066415 (Hocking, Mar. 14, 2013, “Stent”); and 20130116774 (Strauss et al., May 9, 2013, “Protuberant Aneurysm Bridging Device and Method of Use”).

Prior art which appears to be within this category also includes U.S. patent applications: 20130211498 (Buckley et al., Aug. 15, 2013, “Endoprosthesis with Varying Compressibility and Methods of Use”); 20130226276 (Newell et al., Aug. 29, 2013, “Methods and Apparatus for Luminal Stenting”); 20130226278 (Newell et al., Aug. 29, 2013, “Methods and Apparatus for Luminal Stenting”); 20130289714 (Strauss et al., Oct. 31, 2013, “Protuberant Aneurysm Bridging Device and Method of Use”); 20140025154 (Liang et al., Jan. 23, 2014, “Methods and Apparatus for Luminal Stenting”); 20140031918 (Newell et al., Jan. 30, 2014, “Luminal Stenting”); 20140046338 (Grandfield et al., Feb. 13, 2014, “Vascular and Bodily Duct Treatment Devices and Methods”); 20140058436 (Rosenbluth et al., Feb. 27, 2014, “Blood Flow Disruption Devices and Methods for the Treatment of Vascular Defects”); 20140083969 (Porter, Mar. 27, 2014, “Method of Manufacturing a Variably Reinforced Elongate Medical Device”); and 20140114343 (Lee et al., Apr. 24, 2014, “Stent for the Coil Embolization of a Cerebral Aneurysm”).

Prior art which appears to be within this category also includes U.S. patent applications: 20140128957 (Losordo et al., May 8, 2014, “Shaped Occluding Devices and Methods of Using the Same”); 20140200648 (Newell et al., Jul. 17, 2014, “Methods and Apparatus for Luminal Stenting”); 20140243951 (Orr, Aug. 28, 2014, “Tapered Stent and Flexible Prosthesis”); 20140249616 (Strauss et al., Sep. 4, 2014, “Protuberant Aneurysm Bridging Device Deployment Method”); 20140260928 (Janardhan et al., Sep. 18, 2014, “Methods of Using Non-Cylindrical Mandrels”); and 20140265096 (Janardhan et al., Sep. 18, 2014, “Non-Cylindrical Mandrels”).

### 4. Stent in Branching Parent Vessel:

The prior art also discloses devices and methods for treating aneurysms comprising stents which are specifically designed for implantation within a branching parent vessel. Some of the examples in this category were originally focused on aortic aneurysms rather than cerebral aneurysms, but are included herein because of the generalizability of some of their features. Prior art which appears to be within this category includes U.S. Pat. No. 5,723,004 (Dereume et al., Mar. 3, 1998, “Expandable Supportive Endoluminal Grafts”); U.S. Pat. No. 5,948,018 (Dereume et al., Sep. 7, 1999, “Expandable Supportive Endoluminal Grafts”); U.S. Pat. No. 6,165,212 (Dereume et al., Dec. 26, 2000, “Expandable Supportive Endoluminal Grafts”); U.S. Pat. No. 6,210,429 (Vardi et al., Apr. 3, 2001, “Extendible Stent Apparatus”); U.S. Pat. No. 6,309,413 (Dereume et al., Oct. 30, 2001, “Expandable Supportive Endoluminal Grafts”); U.S. Pat. No. 6,395,018 (Castaneda, May 28, 2002, “Endovascular Graft and Process for Bridging a Defect in a Main Vessel Near One of More Branch Vessels”); U.S. Pat. No. 6,599,316 (Vardi et al., Jul. 29, 2003, “Extendible Stent Apparatus”); U.S. Pat. No. 6,835,203 (Vardi et al., Dec. 28, 2004, “Extendible Stent Apparatus”); U.S. Pat. No. 6,962,602 (Vardi et al., Nov. 8, 2005, “Method for Employing an Extendible Stent Apparatus”); U.S. Pat. No. 6,994,721 (Israel, Feb. 7, 2006, “Stent Assembly”); U.S. Pat. No. 7,306,624 (Yodfat et al., Dec. 11, 2007, “Implantable Intraluminal Device and Method of Using Same in Treating Aneurysms”); U.S. Pat. No. 7,413,573 (Hartley et al., Aug. 19, 2008, “Fenestrated Stent Grafts”); U.S. Pat. No. 7,537,609 (Davidson et al., May 26, 2009, “Extendible Stent Apparatus”); and U.S. Pat. No. 7,572,290 (Yodfat et al., Aug. 11, 2009, “Implantable Intraluminal Device and Method of Using Same in Treating Aneurysms”).

Prior art which appears to be within this category also includes U.S. Pat. No. 7,645,298 (Hartley et al., Jan. 12, 2010, “Stent Graft Fenestration”); U.S. Pat. No. 7,766,955 (Vardi et al., Aug. 3, 2010, “Extendible Stent Apparatus”); U.S. Pat. No. 7,769,603 (Jung et al., Aug. 3, 2010, “Stent Customization System and Method”); U.S. Pat. No. 7,776,079 (Gumm, Aug. 17, 2010, “Conical Balloon for Deployment into Side Branch”); U.S. Pat. No. 7,850,725 (Vardi et al., Dec. 14, 2010, “Extendible Stent Apparatus”); U.S. Pat. No. 7,892,279 (Davidson et al., Feb. 22, 2011, “Extendible Stent Apparatus”); U.S. Pat. No. 7,942,925 (Yodfat et al., May 17, 2011, “Implantable Intraluminal Device and Method of Using Same in Treating Aneurysms”); U.S. Pat. No. 8,012,192 (Eidenschink et al., Sep. 6, 2011, “Multi-Stent Delivery System”); U.S. Pat. No. 8,016,878 (Meyer et al., Sep. 13, 2011, “Bifurcation Stent Pattern”); U.S. Pat. No. 8,048,140 (Purdy, Nov. 1, 2011, “Fenestrated Intraluminal Stent System”); U.S. Pat. No. 8,052,736 (Doig et al., Nov. 8, 2011, “Universal Modular Stent Graft Assembly to Accommodate Flow to Collateral Branches”); U.S. Pat. No. 8,100,960 (Bruszewski, Jan. 24, 2012, “Bloused Stent-Graft and Fenestration Method”); U.S. Pat. No. 8,172,895 (Anderson et al., May 8, 2012, “Design and Assembly of Fenestrated Stent Grafts”); U.S. Pat. No. 8,226,706 (Hartley et al., Jul. 24, 2012, “Stent Graft with Integral Side Arm”); U.S. Pat. No. 8,257,430 (Covalin et al., Sep. 4, 2012, “Interconnected Leg Extensions for an Endoluminal Prosthesis”); U.S. Pat. No. 8,257,431 (Henderson et al., Sep. 4, 2012, “Multi-Furcated ePTFE Grafts and Stent-Graft Prostheses and Methods of Making the Same”); U.S. Pat. No. 8,394,136 (Hartley et al., Mar. 12, 2013, “Stent Graft with Internal Tube”); and U.S. Pat. No. 8,419,787 (Yodfat et al., Apr. 16, 2013, “Implantable Intraluminal Device and Method of Using Same in Treating Aneurysms”).

Prior art which appears to be within this category also includes U.S. Pat. No. 8,430,922 (Jung et al., Apr. 30, 2013, “Stent Customization System and Method”); U.S. Pat. No. 8,475,517 (Jung et al., Jul. 2, 2013, “Stent Customization System and Method”); U.S. Pat. No. 8,523,934 (Purdy, Sep. 3, 2013, “Fenestrated Intraluminal Stent System”); U.S. Pat. No. 8,550,344 (Jung et al., Oct. 8, 2013, “Specialty Stents with Flow Control Features or the Like”); U.S. Pat. No. 8,551,155 (Jung et al., Oct. 8, 2013, “Stent Customization System and Method”); U.S. Pat. No. 8,577,693 (Jung et al., Nov. 5, 2013, “Specialty Stents with Flow Control Features or the Like”); U.S. Pat. No. 8,657,865 (Gumm, Feb. 25, 2014, “Conical Balloon for Deployment Into Side Branch”); U.S. Pat. No. 8,715,336 (Chu et al., May 6, 2014, “Methods and Apparatus for Treatment of Aneurysms Adjacent to Branch Arteries”); U.S. Pat. No. 8,721,706 (Jung et al., May 13, 2014, “Specialty Stents with Flow Control Features or the Like”); U.S. Pat. No. 8,728,145 (Chuter et al., May 20, 2014, “Low Profile Non-Symmetrical Stents and Stent-Grafts”); U.S. Pat. No. 8,747,455 (Greenberg, Jun. 10, 2014, “Branched Stent Graft System”); U.S. Pat. No. 8,769,796 (Bourang et al., Jul. 8, 2014, “Selective Stent Crimping”); U.S. Pat. No. 8,771,342 (Vardi, Jul. 8, 2014, “Methods for Deploying Stents in Bifurcations”); U.S. Pat. No. 8,795,347 (Bourang et al., Aug. 5, 2014, “Methods and Systems for Treating a Bifurcation with Provisional Side Branch Stenting”); U.S. Pat. No. 8,808,347 (Bourang et al., Aug. 19, 2014, “Stent Alignment During Treatment of a Bifurcation”); U.S. Pat. No. 8,821,564 (Schreck et al., Sep. 2, 2014, “Stent Graft”); and U.S. Pat. No. 8,828,071 (Bourang et al., Sep. 9, 2014, “Methods and Systems for Ostial Stenting of a Bifurcation”).

Prior art which appears to be within this category also includes U.S. patent applications: 20010016766 (Vardi et al., Aug. 23, 2001, “Extendible Stent Apparatus”); 20010037137 (Vardi et al., Nov. 1, 2001, “Extendible Stent Apparatus”); 20020116047 (Vardi et al., Aug. 22, 2002, “Extendible Stent Apparatus and Method for Deploying the Same”); 20020156516 (Vardi et al., Oct. 24, 2002, “Method for Employing an Extendible Stent Apparatus”); 20030074049 (Hoganson et al., Apr. 17, 2003, “Covered Stents and Systems for Deploying Covered Stents”); 20030100945 (Yodfat et al., May 29, 2003, “Implantable Intraluminal Device and Method of Using Same in Treating Aneurysms”); 20030195606 (Davidson et al., Oct. 16, 2003, “Bifurcation Stent System and Method”); 20040015227 (Vardi et al., Jan. 22, 2004, “Extendible Stent Apparatus”); 20050010281 (Yodfat et al., Jan. 13, 2005, “Implantable Intraluminal Device and Method of Using Same in Treating Aneurysms”); and 20050131518 (Hartley et al., Jun. 16, 2005, “Fenestrated Stent Grafts”).

Prior art which appears to be within this category also includes U.S. patent applications: 20060085061 (Vardi et al., Apr. 20, 2006, “Extendible Stent Apparatus and Method for Deploying the Same”); 20060241740 (Vardi et al., Oct. 26, 2006, “Extendible Stent Apparatus”); 20070032855 (Davidson et al., Feb. 8, 2007, “Extendible Stent Apparatus”); 20070100301 (Gumm, May 3, 2007, “Conical Balloon for Deployment into Side Branch”); 20070299498 (Perez et al., Dec. 27, 2007, “Methods and Devices for Aiding In Situ Assembly of Repair Devices”); 20080039933 (Yodfat et al., Feb. 14, 2008, “Implantable Intraluminal Device and Method of Using Same in Treating Aneurysms”); 20080312732 (Hartley et al., Dec. 18, 2008, “Fenestrated Stent Grafts”); 20090132028 (Vardi et al., May 21, 2009, “Extendible Stent Apparatus and Method for Deploying the Same”); 20090248135 (Bruszewski et al., Oct. 1, 2009, “Eversible Branch Stent-Graft and Deployment Method”); 20090270970 (Yodfat et al., Oct. 29, 2009, “Implantable Intraluminal Device and Method of Using Same in Treating Aneurysms”); and 20100198334 (Yodfat et al., Aug. 5, 2010, “Implantable Intraluminal Device and Method of Using Same in Treating Aneurysms”).

Prior art which appears to be within this category also includes U.S. patent applications: 20100305681 (Gumm, Dec. 2, 2010, “Conical Balloon for Deployment into Side Branch”); 20100312326 (Chuter et al., Dec. 9, 2010, “Apparatus and Methods for Deployment of a Modular Stent-Graft System”); 20110082533 (Vardi et al., Apr. 7, 2011, “Extendible Stent Apparatus”); 20110288627 (Hartley et al., Nov. 24, 2011, “Stent Graft with Integral Side Arm”); 20110307044 (Bourang et al., Dec. 15, 2011, “Methods and Systems for Ostial Stenting of a Bifurcation”); 20110307045 (Bourang et al., Dec. 15, 2011, “Methods and Systems for Treating a Bifurcation with Provisional Side Branch Stenting”); 20110307046 (Bourang et al., Dec. 15, 2011, “Selective Stent Crimping”); 20110307052 (Bourang et al., Dec. 15, 2011, “Stent Alignment During Treatment of a Bifurcation”); 20110313512 (Hartley et al., Dec. 22, 2011, “Side Branch Stent Graft”); 20120053670 (Purdy, Mar. 1, 2012, “Fenestrated Intraluminal Stent System”); and 20120130479 (Chuter et al., May 24, 2012, “Low Profile Non-Symmetrical Stents and Stent-Grafts”).

Prior art which appears to be within this category also includes U.S. patent applications: 20120143237 (Cam et al., Jun. 7, 2012, “Vascular Remodeling Device”); 20120143317 (Cam et al., Jun. 7, 2012, “Vascular Remodeling Device”); 20120253448 (Hartley et al., Oct. 4, 2012, “Stent Graft with Integral Side Arm”); 20120296361 (Cam et al., Nov. 22, 2012, “Vascular Remodeling Device”); 20120296362 (Cam et al., Nov. 22, 2012, “Vascular Remodeling Device”); 20130046371 (Greenberg et al., Feb. 21, 2013, “Endoluminal Prosthesis Having Multiple Branches or Fenestrations and Methods of Deployment”); 20130053944 (Fogarty et al., Mar. 7, 2013, “Endoluminal Prosthesis Assembly”); 20130150946 (Hartley et al., Jun. 13, 2013, “Fenestrated Stent Grafts”); 20130204351 (Cox et al., Aug. 8, 2013, “Aneurysm Graft Devices and Methods”); 20130204354 (Adams, O., Aug. 8, 2013, “Branched Stent/Graft and Method of Fabrication”); and 20130211505 (Robison, Aug. 15, 2013, “Devices and Methods for Approximating the Cross-Sectional Profile of Vasculature Having Branches”).

Prior art which appears to be within this category also includes U.S. patent applications: 20130211507 (LaDuca et al., Aug. 15, 2013, “Apparatus and Method for Deploying an Implantable Device Within the Body”); 20130345785 (Hartley et al., Dec. 26, 2013, “Fenestrated Stent Grafts”); 20140100647 (Bourang, Apr. 10, 2014, “System and Methods for Treating a Bifurcation with a Fully Crimped Stent”); and 20140222130 (Kusleika, S., Aug. 7, 2014, “Vascular Device for Aneurysm Treatment and Providing Blood Flow into a Perforator Vessel”).

5. Stent with Helical Structure in Parent Vessel:

The prior art also discloses devices and methods for treating aneurysms comprising stents with one or more helical structures which are implanted within the parent vessel of an aneurysm. Prior art which appears to be within this category includes U.S. Pat. No. 5,980,514 (Kupiecki et al., Nov. 9, 1999, “Aneurysm Closure Device Assembly”); U.S. Pat. No. 6,063,111 (Hieshima et al., May 16, 2000, “Stent Aneurysm Treatment System and Method”); U.S. Pat. No. 6,096,034 (Kupiecki et al., Aug. 1, 2000, “Aneurysm Closure Device Assembly”); U.S. Pat. No. 6,168,592 (Kupiecki et al., Jan. 2, 2001, “Aneurysm Closure Device Assembly”); U.S. Pat. No. 6,344,041 (Kupiecki et al., Feb. 5, 2002, “Aneurysm Closure Device Assembly”); U.S. Pat. No. 6,645,237 (Klumb et al., Nov. 11, 2003, “Expandable Coiled Endoluminal Prosthesis”); U.S. Pat. No. 6,660,032 (Klumb et al., Dec. 9, 2003, “Expandable Coil Endoluminal Prosthesis”); U.S. Pat. No. 6,746,468 (Sepetka et al., Jun. 8, 2004, “Devices and Methods for Treating Vascular Malformations”); U.S. Pat. No. 6,746,475 (Rivelli, Jun. 8, 2004, “Stent with Variable Stiffness”); U.S. Pat. No. 6,860,899 (Rivelli, Mar. 1, 2005, “Method for Treating Neurovascular Aneurysms”); U.S. Pat. No. 6,899,730 (Rivelli, May 31, 2005, “Catheter-Stent Device”); U.S. Pat. No. 7,481,821 (Fogarty et al., Jan. 27, 2009, “Embolization Device and a Method of Using the Same”); and U.S. Pat. No. 7,520,893 (Rivelli, Apr. 21, 2009, “Method for Treating Neurovascular Aneurysms”).

Prior art which appears to be within this category also includes U.S. Pat. No. 7,572,288 (Cox, Aug. 11, 2009, “Aneurysm Treatment Device and Method of Use”); U.S. Pat. No. 7,803,180 (Burpee et al., Sep. 28, 2010, “Flexible Stent”); U.S. Pat. No. 7,862,608 (Hogendijk et al., Jan. 4, 2011, “Vascular Prosthesis and Methods of Use”); U.S. Pat. No. 8,057,495 (Pal et al., Nov. 15, 2011, “Aneurysm Occlusion Device”); U.S. Pat. No. 8,252,040 (Cox, Aug. 28, 2012, “Aneurysm Treatment Device and Method of Use”); U.S. Pat. No. 8,262,686 (Fogarty et al., Sep. 11, 2012, “Embolization Device and a Method of Using the Same”); U.S. Pat. No. 8,562,636 (Fogarty et al., Oct. 22, 2013, “Embolization Device and a Method of Using the Same”); U.S. Pat. No. 8,562,667 (Cox, Oct. 22, 2013, “Aneurysm Treatment Device and Method of Use”); U.S. Pat. No. 8,597,320 (Sepetka et al., Dec. 3, 2013, “Devices and Methods for Treating Vascular Malformations”); and U.S. Pat. No. 8,715,312 (Burke et al., May 6, 2014, “Aneurysm Treatment Device and Method of Use”).

Prior art which appears to be within this category also includes U.S. patent applications: 20020133190 (Horton et al., Sep. 19, 2002, “In Situ Formable and Self-Forming Intravascular Flow Modifier (IFM), Catheter and IFM Assembly, and Method for Deployment of Same”); 20020169473 (Sepetka et al., Nov. 14, 2002, “Devices and Methods for Treating Vascular Malformations”); 20030018294 (Cox, Jan. 23, 2003, “Aneurysm Treatment Device and Method of Use”); 20040181253 (Sepetka et al., Sep. 16, 2004, “Devices and Methods for Treating Vascular Malformations”); 20040193246 (Ferrera, Sep. 30, 2004, “Methods and Apparatus for Treating Aneurysms and Other Vascular Defects”); 20040210249 (Fogarty et al., Oct. 21, 2004, “Embolization Device and a Method of Using the Same”); and 20040220663 (Rivelli, J R., Nov. 4, 2004, “Stent with Variable Stiffness”).

Prior art which appears to be within this category also includes U.S. patent applications: 20040260384 (Allen, Dec. 23, 2004, “Superelastic Coiled Stent”); 20050015110 (Fogarty et al., Jan. 20, 2005, “Embolization Device and a Method of Using the Same”); 20050149164 (Rivelli, Jul. 7, 2005, “Method for Treating Neurovascular Aneurysms”); 20050171597 (Boatman et al., Aug. 4, 2005, “Helical Stent for Branched Vessel Prosthesis”); 20060136033 (Hermann et al., Jun. 22, 2006, “Coiled Stent Delivery System and Method”); 20070083257 (Pal et al., Apr. 12, 2007, “Aneurysm Occlusion Device”); 20070129786 (Beach et al., Jun. 7, 2007, “Helical Stent”); 20070150045 (Ferrera, Jun. 28, 2007, “Methods and Apparatus for Treating Aneurysms and Other Vascular Defects”); 20080114391 (Dieck et al., May 15, 2008, “Aneurysm Covering Devices and Delivery Devices”); 20080114436 (Dieck et al., May 15, 2008, “Aneurysm Covering Devices and Delivery Devices”); 20090105748 (Fogarty et al., Apr. 23, 2009, “Embolization Device and a Method of Using the Same”); 20110082491 (Sepetka et al., Apr. 7, 2011, “Devices and Methods for Treating Vascular Malformations”); and 20110137332 (Sepetka et al., Jun. 9, 2011, “Devices and Methods for Treating Vascular Malformations”).

Prior art which appears to be within this category also includes U.S. patent applications: 20110166641 (Bales et al., Jul. 7, 2011, “Highly Flexible Stent and Method of Manufacture”); 20110224776 (Sepetka et al., Sep. 15, 2011, “Devices and Methods for Treating Vascular Malformations”); 20120179192 (Fogarty et al., Jul. 12, 2012, “Embolization Device and a Method of Using the Same”); 20120303108 (Fogarty et al., Nov. 29, 2012, “Embolization Device and a Method of Using the Same”); 20120330402 (Vad et al., Dec. 27, 2012, “Helical Stent”); 20130090719 (Bales et al., Apr. 11, 2013, “Highly Flexible Stent and Method of Manufacture”); 20130090721 (Bales et al., Apr. 11, 2013, “Highly Flexible Stent and Method of Manufacture”); 20130116659 (Porter, May 9, 2013, “Medical Device with Bi-Component Polymer Fiber Sleeve”); 20130123899 (Leopold et al., May 16, 2013, “Vascular Prosthesis and Methods of Use”); 20140031920 (Malek, Jan. 30, 2014, “Endovascular Stent”); 20140088690 (Fogarty et al., Mar. 27, 2014, “Embolization Device and a Method of Using the Same”); and 20140288633 (Burke et al., Sep. 25, 2014, “Aneurysm Treatment Device and Method of Use”).

6. Stent with Special Structure or Flexibility in Parent Vessel:

The prior art also discloses devices and methods for treating aneurysms comprising stents with special structure or flexibility (which is not specified in one of the other categories herein) which are implanted within the parent vessel of an aneurysm. Prior art which appears to be within this category includes U.S. Pat. No. 5,411,549 (Peters, May 2, 1995, “Selectively Expandable, Retractable and Removable Stent”); U.S. Pat. No. 5,603,722 (Phan et al., Feb. 18, 1997, “Intravascular Stent”); U.S. Pat. No. 5,964,797 (Ho, Oct. 12, 1999, “Electrolytically Deployable Braided Vaso-Occlusion Device”); U.S. Pat. No. 6,190,406 (Duerig et al., Feb. 20, 2001, “Intravascular Stent Having Tapered Struts”); U.S. Pat. No. 6,258,117 (Camrud et al., Jul. 10, 2001, “Multi-Section Stent”); U.S. Pat. No. 6,342,068 (Thompson, Jan. 29, 2002, “Three-Dimensional Braided Stent”); U.S. Pat. No. 6,416,543 (Hilaire et al., Jul. 9, 2002, “Expandable Stent with Variable Thickness”); U.S. Pat. No. 6,485,510 (Camrud et al., Nov. 26, 2002, “Multi-Section Stent”); U.S. Pat. No. 6,520,985 (Burpee et al., Feb. 18, 2003, “Stent with Reduced Shortening”); U.S. Pat. No. 6,520,987 (Plante, Feb. 18, 2003, “Expandable Intravascular Stent”); U.S. Pat. No. 6,585,753 (Eder et al., Jul. 1, 2003, “Expandable Coil Stent”); U.S. Pat. No. 6,833,003 (Jones et al., Dec. 21, 2004, “Expandable Stent and Delivery System”); U.S. Pat. No. 7,033,385 (Eder et al., Apr. 25, 2006, “Expandable Coil Stent”); U.S. Pat. No. 7,037,330 (Rivelli et al., May 2, 2006, “Neurovascular Stent and Method”); U.S. Pat. No. 7,052,513 (Thompson, May 30, 2006, “Three-Dimensional Braided Covered Stent”); U.S. Pat. No. 7,195,648 (Jones et al., Mar. 27, 2007, “Intravascular Stent Device”); and U.S. Pat. No. 7,211,109 (Thompson, May 1, 2007, “Braided Composite Prosthesis”).

Prior art which appears to be within this category also includes U.S. Pat. No. 7,309,352 (Eder et al., Dec. 18, 2007, “Expandable Coil Stent”); U.S. Pat. No. 7,491,229 (Eder et al., Feb. 17, 2009, “Expandable Coil Stent”); U.S. Pat. No. 7,695,507 (Rivelli et al., Apr. 13, 2010, “Neurovascular Stent and Method”); U.S. Pat. No. 7,803,179 (Denison, Sep. 28, 2010, “Intravascular Stents”); U.S. Pat. No. 7,955,382 (Flanagan et al., Jun. 7, 2011, “Endoprosthesis with Adjustable Surface Features”); U.S. Pat. No. 7,988,721 (Morris et al., Aug. 2, 2011, “Axially-Radially Nested Expandable Device”); U.S. Pat. No. 8,012,197 (Bashiri et al., Sep. 6, 2011, “Hybrid Balloon Expandable/Self-Expanding Stent”); U.S. Pat. No. 8,016,876 (Gregorich et al., Sep. 13, 2011, “Stent Configurations”); U.S. Pat. No. 8,070,792 (Gregorich et al., Dec. 6, 2011, “Stent”); U.S. Pat. No. 8,282,679 (Denison, Oct. 9, 2012, “Intravascular Stents”); U.S. Pat. No. 8,414,637 (Chouinard, Apr. 9, 2013, “Stent”); U.S. Pat. No. 8,512,395 (Meyer et al., Aug. 20, 2013, “Stent with Horseshoe Shaped Bridges”); U.S. Pat. No. 8,529,616 (Boyle et al., Sep. 10, 2013, “Implantable Expandable Medical Devices Having Regions of Differential Mechanical Properties and Methods of Making Same”); U.S. Pat. No. 8,623,071 (Lundkvist et al., Jan. 7, 2014, “Radiopaque Super-Elastic Intravascular Stent”); U.S. Pat. No. 8,663,309 (Chobotov, Mar. 4, 2014, “Asymmetric Stent Apparatus and Method”); U.S. Pat. No. 8,671,815 (Hancock et al., Mar. 18, 2014, “Self-Expanding Pseudo-Braided Intravascular Device”); U.S. Pat. No. 8,671,815 (Hancock et al., Mar. 18, 2014, “Self-Expanding Pseudo-Braided Intravascular Device”); U.S. Pat. No. 8,728,146 (Gregorich et al., May 20, 2014, “Stent Configurations”); U.S. Pat. No. 8,740,966 (Brocker et al., Jun. 3, 2014, “Low Profile Non-Symmetrical Stent”); and U.S. Pat. No. 8,801,772 (Shobayashi et al., Aug. 12, 2014, “Stent to Be Used in Tubular Organ In Vivo”).

Prior art which appears to be within this category also includes U.S. patent applications: 20040186551 (Kao et al., Sep. 23, 2004, “Multiple Independent Nested Stent Structures and Methods for Their Preparation and Deployment”); 20060224230 (Rivelli et al., Oct. 5, 2006, “Neurovascular Stent and Method”); 20090082846 (Chobotov, Mar. 26, 2009, “Asymmetric Stent Apparatus and Method”); 20090171437 (Brocker et al., Jul. 2, 2009, “Low Profile Non-Symmetrical Stent”); 20090177268 (Lundkvist et al., Jul. 9, 2009, “Radiopaque Super-Elastic Intravascular Stent”); 20090299390 (Dehnad, Dec. 3, 2009, “Multistrand Coil for Interventional Therapy”); 20100004726 (Hancock et al., Jan. 7, 2010, “Self-Expanding Pseudo-Braided Intravascular Device”); 20100152837 (Lundkvist et al., Jun. 17, 2010, “Radiopaque Super-Elastic Intravascular Stent”); 20100222864 (Rivelli et al., Sep. 2, 2010, “Neurovascular Stent and Method”); 20100324660 (Denison, Dec. 23, 2010, “Intravascular Stents”); and 20110264192 (Hartley et al., Oct. 27, 2011, “Curve Forming Stent Graft”).

Prior art which appears to be within this category also includes U.S. patent applications: 20120004719 (Gregorich et al., Jan. 5, 2012, “Stent Configurations”); 20120016462 (Gregorich et al., Jan. 19, 2012, “Stent”); 20120041540 (Shobayashi, Feb. 16, 2012, “Stent to be Used in Tubular Organ In Vivo”); 20120055614 (Hancock et al., Mar. 8, 2012, “Self-Expanding Pseudo-Braided Intravascular Device”); 20120165920 (Meyer et al., Jun. 28, 2012, “Stent”); 20120172972 (Meyer et al., Jul. 5, 2012, “Multi Stage Opening Stent Designs”); 20120323309 (Cattaneo, Dec. 20, 2012, “Stent with Flaps”); 20130060322 (Leynov et al., Mar. 7, 2013, “Expandable Framework with Overlapping Connectors”); 20130066413 (Jin et al., Mar. 14, 2013, “Surgical Apparatus for Aneurysms”); 20130146173 (Krivoruchko et al., Jun. 13, 2013, “Stent With Improved Flexibility and Method for Making Same”); 20140058500 (Lundkvist et al., Feb. 27, 2014, “Radiopaque Super-Elastic Intravascular Stent”); 20140082924 (Lundkvist et al., Mar. 27, 2014, “Radiopaque Super-Elastic Intravascular Stent”); 20140188208 (Hancock et al., Jul. 3, 2014, “Self-Expanding Pseudo-Braided Intravascular Device”); and 20140277370 (Brocker et al., Sep. 18, 2014, “Low Profile Non-Symmetrical Stent”).

7. Stent with Multiple Layers in Parent Vessel:

The prior art also discloses devices and methods for treating aneurysms comprising stents with multiple layers which are implanted within the parent vessel of an aneurysm. In an example, a first layer of such a stent can be porous, but provide structural support, while a second layer can be non-porous. In another example, embolic members or compositions can be inserted between two stent layers. Prior art which appears to be within this category includes U.S. Pat. No. 5,645,559 (Hachtman et al., Jul. 8, 1997, “Multiple Layer Stent”); U.S. Pat. No. 5,769,882 (Fogarty et al., Jun. 23, 1998, “Methods and apparatus for conformably sealing prostheses within body lumens”); U.S. Pat. No. 6,086,610 (Duerig et al., Jul. 11, 2000, “Composite Self Expanding Stent Device Having a Restraining Element”); U.S. Pat. No. 6,149,681 (Houser et al., Nov. 21, 2000, “Radially Expanding Prostheses and Systems for Their Deployment”); U.S. Pat. No. 6,270,523 (Herweck et al., Aug. 7, 2001, “Expandable Shielded Vessel Support”); U.S. Pat. No. 6,331,191 (Chobotov, Dec. 18, 2001, “Layered endovascular graft”); U.S. Pat. No. 6,398,803 (Layne et al., Jun. 4, 2002, “Partial Encapsulation of Stents”); U.S. Pat. No. 6,558,414 (Layne, May 6, 2003, “Partial Encapsulation of Stents Using Strips and Bands”); U.S. Pat. No. 6,579,314 (Lombardi et al., Jun. 17, 2003, “Covered Stent with Encapsulated Ends”); U.S. Pat. No. 6,656,214 (Fogarty et al., Dec. 2, 2003, “Methods and apparatus for conformably sealing prostheses within body lumens”); U.S. Pat. No. 6,673,103 (Golds et al., Jan. 6, 2004, “Mesh and Stent for Increased Flexibility”); U.S. Pat. No. 6,699,277 (Freidberg et al., Mar. 2, 2004, “Stent with Cover Connectors”); U.S. Pat. No. 6,719,783 (Lentz et al., Apr. 13, 2004, “PTFE Vascular Graft and Method of Manufacture”); U.S. Pat. No. 6,770,087 (Layne et al., Aug. 3, 2004, “Partial Encapsulation of Stents”); U.S. Pat. No. 6,786,920 (Shannon et al., Sep. 7, 2004, “Stented Radially Expandable Tubular PTFE Grafts”); and U.S. Pat. No. 6,790,225 (Shannon et al., Sep. 14, 2004, “Stented Radially Expandable Tubular PTFE Grafts”).

Prior art which appears to be within this category also includes U.S. Pat. No. 6,929,658 (Freidberg et al., Aug. 16, 2005, “Stent with Cover Connectors”); U.S. Pat. No. 7,081,129 (Chobotov, Jul. 25, 2006, “Endovascular Graft”); U.S. Pat. No. 7,083,640 (Lombardi et al., Aug. 1, 2006, “Covered Stent with Encapsulated Ends”); U.S. Pat. No. 7,186,263 (Golds et al., Mar. 6, 2007, “Mesh Graft and Stent for Increased Flexibility”); U.S. Pat. No. 7,588,597 (Frid, Sep. 15, 2009, “Three-Dimensional Braided Structure Stent”); U.S. Pat. No. 7,615,071 (Chobotov, Nov. 10, 2009, “Endovascular Graft”); U.S. Pat. No. 7,666,220 (Evans et al., Feb. 23, 2010, “System and Methods for Endovascular Aneurysm Treatment”); U.S. Pat. No. 7,704,274 (Boyle et al., Apr. 27, 2010, “Implantable Graft and Methods of Making Same”); U.S. Pat. No. 7,758,892 (Chen et al., Jul. 20, 2010, “Medical Devices Having Multiple Layers”); U.S. Pat. No. 7,914,639 (Layne et al., Mar. 29, 2011, “Partial Encapsulation of Stents”); U.S. Pat. No. 8,211,160 (Garrison et al., Jul. 3, 2012, “Stent Graft Assembly and Method”); U.S. Pat. No. 8,388,677 (Henmann, Mar. 5, 2013, “Anti-Thrombogenic and Anti-Restenotic Vascular Medical Devices”); U.S. Pat. No. 8,535,367 (Kim et al., Sep. 17, 2013, “Devices and Methods for Treatment of Vascular Aneurysms”); U.S. Pat. No. 8,647,377 (Kim et al., Feb. 11, 2014, “Devices and Methods for Treatment of Vascular Aneurysms”); and U.S. Pat. No. 8,784,477 (Bregulla et al., Jul. 22, 2014, “Stent Graft with Two Layer ePTFE Layer System with High Plasticity and High Rigidity”).

Prior art which appears to be within this category also includes U.S. patent applications: 20030074049 (Hoganson et al., Apr. 17, 2003, “Covered Stents and Systems for Deploying Covered Stents”); 20050107863 (Brown, May 19, 2005, “Micro Structure Stent Configurations”); 20060229714 (Lombardi et al., Oct. 12, 2006, “Covered Stent with Encapsulated Ends”); 20060292206 (Kim et al., Dec. 28, 2006, “Devices and Methods for Treatment of Vascular Aneurysms”); 20070050008 (Kim et al., Mar. 1, 2007, “Devices and Methods for Treatment of Vascular Aneurysms”); 20070055355 (Kim et al., Mar. 8, 2007, “Devices and Methods for Treatment of Vascular Aneurysms”); 20070061005 (Kim et al., Mar. 15, 2007, “Devices and Methods for Treatment of Vascular Aneurysms”); and 20070207186 (Scanlon et al., Sep. 6, 2007, “Tear and Abrasion Resistant Expanded Material and Reinforcement”).

Prior art which appears to be within this category also includes U.S. patent applications: 20080125852 (Garrison et al., May 29, 2008, “Stent Graft Assembly and Method”); 20090318949 (Ganpath et al., Dec. 24, 2009, “Sealing Apparatus and Methods of Use”); 20100131002 (Connor et al., May 27, 2010, “Stent with a Net Layer to Embolize an Aneurysm”); 20100179645 (Chen et al., Jul. 15, 2010, “Medical Devices Having Multiple Layers”); 20100280452 (Chen et al., Nov. 4, 2010, “Medical Devices Having Multiple Layers”); 20120172977 (Bregulla et al., Jul. 5, 2012, “Stent Graft with Two Layer ePTFE Layer System with High Plasticity and High Rigidity”); 20120259404 (Tieu et al., Oct. 11, 2012, “Stent”); 20120303112 (Armstrong et al., Nov. 29, 2012, “Stent”); 20120330343 (Kim et al., Dec. 27, 2012, “Devices and Methods for Treatment of Vascular Aneurysms”); 20130018220 (Vad et al., Jan. 17, 2013, “Method for Electrospinning a Graft Layer”); 20130103135 (Vinluan, Apr. 25, 2013, “Fenestrated Inflatable Graft”); and 20130131780 (Armstrong et al., May 23, 2013, “Lattice”).

Prior art which appears to be within this category also includes U.S. patent applications: 20130131786 (Chobotov, May 23, 2013, “Endovascular Graft”); 20130197617 (Armstrong et al., Aug. 1, 2013, “Stent”); 20130197624 (Armstrong et al., Aug. 1, 2013, “Stent”); 20130204347 (Armstrong et al., Aug. 8, 2013, “Lattice”); 20130245745 (Vong et al., Sep. 19, 2013, “Stent and Stent Delivery Device”); 20140058436 (Rosenbluth et al., Feb. 27, 2014, “Blood Flow Disruption Devices and Methods for the Treatment of Vascular Defects”); 20140081374 (Kim et al., Mar. 20, 2014, “Devices and Methods for Treatment of Vascular Aneurysms”); 20140130965 (Banks et al., May 15, 2014, “Method of Manufacturing a Stent-Graft Prosthesis with Two Layers of Expanded Polytetrafluoroethylene”); and 20140180397 (Gerberding et al., Jun. 26, 2014, “Multilayer Stent”).

8. Stent with Non-Porous Walls in Parent Vessel:

The prior art also discloses devices and methods for treating aneurysms comprising stents with non-porous walls which are implanted within the parent vessel of an aneurysm. Prior art which appears to be within this category includes U.S. Pat. No. 5,405,379 (Lane, Apr. 11, 1995, “Self Expanding Vascular Endoprosthesis for Aneurysms”); U.S. Pat. No. 5,723,004 (Dereume et al., Mar. 3, 1998, “Expandable Supportive Endoluminal Grafts”); U.S. Pat. No. 5,948,018 (Dereume et al., Sep. 7, 1999, “Expandable Supportive Endoluminal Grafts”); U.S. Pat. No. 6,165,212 (Dereume et al., Dec. 26, 2000, “Expandable Supportive Endoluminal Grafts”); U.S. Pat. No. 6,309,413 (Dereume et al., Oct. 30, 2001, “Expandable Supportive Endoluminal Grafts”); U.S. Pat. No. 8,003,180 (Goffena et al., Aug. 23, 2011, “Thin-Wall Polytetrafluoroethylene Tube”); U.S. Pat. No. 8,480,727 (Clarke, Jul. 9, 2013, “Endovascular Implant Having an Integral Graft Component and Method of Manufacture”); and U.S. Pat. No. 8,715,336 (Chu et al., May 6, 2014, “Methods and Apparatus for Treatment of Aneurysms Adjacent to Branch Arteries”).

Prior art which appears to be within this category also includes U.S. patent applications: 20050131516 (Greenhalgh, Jun. 16, 2005, “Integral Support Stent Graft Assembly”); 20050171597 (Boatman et al., Aug. 4, 2005, “Helical Stent for Branched Vessel Prosthesis”); 20080195137 (Alleyne et al., Aug. 14, 2008, “Devices and Methods for Aneurysm Treatment”); 20130053872 (Hansen, Feb. 28, 2013, “Device and Method for Preventing Blood Flow into an Aneurysm”); 20130116659 (Porter, May 9, 2013, “Medical Device with Bi-Component Polymer Fiber Sleeve”); 20130289713 (Pearson et al., Oct. 31, 2013, “Circumferentially Constraining Sutures for a Stent-Graft”); 20140130965 (Banks et al., May 15, 2014, “Method of Manufacturing a Stent-Graft Prosthesis with Two Layers of Expanded Polytetrafluoroethylene”); and 20140194973 (Chobotov, Jul. 10, 2014, “Sac Liner for Aneurysm Repair”).

9. Stent with Integrated Actuators and/or Sensors in Parent Vessel:

Although not yet common, the prior art also discloses devices and methods for treating aneurysms which could be called smart stents—featuring integrated microscale actuators (such as MEMS) for controlled expansion and/or sensors to collect biological data. In an example, such smart stents can be implanted within the parent vessel of an aneurysm. Prior art which appears to be within this category includes U.S. Pat. No. 7,235,098 (Palmaz, Jun. 26, 2007, “Medical Devices Having MEMS Functionality and Methods of Making Same”); U.S. Pat. No. 8,019,413 (Ferren et al., Sep. 13, 2011, “Lumen-Traveling Biological Interface Device and Method of Use”); U.S. Pat. No. 8,024,036 (Ferren et al., Sep. 20, 2011, “Lumen-Traveling Biological Interface Device and Method of Use”); U.S. Pat. No. 8,512,219 (Ferren et al., Aug. 20, 2013, “BioelectroMagnetic Interface System”); and U.S. Pat. No. 8,784,475 (Martinson et al., Jul. 22, 2014, “Instrumented Implantable Stents, Vascular Grafts and Other Medical Devices”).

Prior art which appears to be within this category also includes U.S. patent applications: 20050065592 (Holzer, Mar. 24, 2005, “System and Method of Aneurism Monitoring and Treatment”); 20080004692 (Henson et al., Jan. 3, 2008, “Dynamically Adjustable Vascular Stent”); 20120271200 (Martinson et al., Oct. 25, 2012, “Instrumented Implantable Stents, Vascular Grafts and Other Medical Devices”); 20130041454 (Dobson et al., Feb. 14, 2013, “Sensor Actuated Stent”); and 20130166017 (Cartledge et al., Jun. 27, 2013, “Actively Controllable Stent, Stent Graft, Heart Valve and Method of Controlling Same”); and Yet unpublished U.S. patent application Ser. No. 12/387,637 (Connor et al., 2009, “Stent with Two-Stage Expansion to Reduce Restenosis”).

10. Stent with Other Complex Structure in Parent Vessel:

The prior art also discloses devices and methods for treating aneurysms comprising stents with other complex structures which are implanted within the parent vessel of an aneurysm. Prior art which appears to be within this category includes U.S. Pat. No. 5,370,691 (Samson, Dec. 6, 1994, “Intravascular Inflatable Stent”); U.S. Pat. No. 5,873,907 (Frantzen, Feb. 23, 1999, “Electrolytic Stent Delivery System and Methods of Use”); U.S. Pat. No. 6,007,573 (Wallace et al., Dec. 28, 1999, “Intracranial Stent and Method of Use”); U.S. Pat. No. 6,015,433 (Roth, Jan. 18, 2000, “Rolled Stent with Waveform Perforation Pattern”); U.S. Pat. No. 6,096,175 (Roth, Aug. 1, 2000, “Thin Film Stent”); U.S. Pat. No. 6,406,490 (Roth, Jun. 18, 2002, “Rolled Stent with Waveform Perforation Pattern”); U.S. Pat. No. 6,432,128 (Wallace et al., Aug. 13, 2002, “Intracranial Stent and Method of Use”); U.S. Pat. No. 6,527,919 (Roth, Mar. 4, 2003, “Thin Film Stent”); U.S. Pat. No. 6,579,305 (Lashinski, Jun. 17, 2003, “Method and Apparatus for Delivery Deployment and Retrieval of a Stent Comprising Shape-Memory Material”); U.S. Pat. No. 6,669,719 (Wallace et al., Dec. 30, 2003, “Intracranial Stent and Method of Use”); U.S. Pat. No. 6,916,337 (Roth, Jul. 12, 2005, “Rolled Stent with Waveform Perforation Pattern”); U.S. Pat. No. 7,037,327 (Salmon et al., May 2, 2006, “Stent with Self-Expanding End Sections”); U.S. Pat. No. 7,118,656 (Roth, Oct. 10, 2006, “Thin Film Stent”); U.S. Pat. No. 7,141,063 (White et al., Nov. 28, 2006, “Stent with Micro-Latching Hinge Joints”); and U.S. Pat. No. 7,147,660 (Chobotov et al., Dec. 12, 2006, “Advanced Endovascular Graft”).

Prior art which appears to be within this category also includes U.S. Pat. No. 7,147,661 (Chobotov et al., Dec. 12, 2006, “Radially Expandable Stent”); U.S. Pat. No. 7,294,137 (Rivelli et al., Nov. 13, 2007, “Device for Multi-Modal Treatment of Vascular Lesions”); U.S. Pat. No. 7,306,598 (Truckai et al., Dec. 11, 2007, “Polymer Matrix Devices for Treatment of Vascular Malformations”); U.S. Pat. No. 7,323,005 (Wallace et al., Jan. 29, 2008, “Intracranial Stent and Method of Use”); U.S. Pat. No. 7,384,426 (Wallace et al., Jun. 10, 2008, “Intracranial Stent and Method of Use”); U.S. Pat. No. 7,455,753 (Roth, Nov. 25, 2008, “Thin Film Stent”); U.S. Pat. No. 7,547,321 (Silvestri et al., Jun. 16, 2009, “Removable Stent and Method of Using the Same”); U.S. Pat. No. 7,651,525 (Dolan, Jan. 26, 2010, “Intraluminal Stent Assembly and Method of Deploying the Same”); U.S. Pat. No. 7,901,445 (Wallace et al., Mar. 8, 2011, “Intracranial Stent and Method of Use”); U.S. Pat. No. 7,914,574 (Schmid et al., Mar. 29, 2011, “Axially Nested Slide and Lock Expandable Device”); U.S. Pat. No. 7,947,071 (Schmid et al., May 24, 2011, “Expandable Slide and Lock Stent”); U.S. Pat. No. 8,016,853 (Griffen et al., Sep. 13, 2011, “Sacrificial Anode Stent System”); U.S. Pat. No. 8,038,708 (Case et al., Oct. 18, 2011, “Implantable Device with Remodelable Material and Covering Material”); and U.S. Pat. No. 8,147,534 (Berez et al., Apr. 3, 2012, “System and Method for Delivering and Deploying an Occluding Device within a Vessel”).

Prior art which appears to be within this category also includes U.S. Pat. No. 8,236,042 (Berez et al., Aug. 7, 2012, “System and Method for Delivering and Deploying an Occluding Device within a Vessel”); U.S. Pat. No. 8,257,421 (Berez et al., Sep. 4, 2012, “System and Method for Delivering and Deploying an Occluding Device within a Vessel”); U.S. Pat. No. 8,267,985 (Garcia et al., Sep. 18, 2012, “System and Method for Delivering and Deploying an Occluding Device within a Vessel”); U.S. Pat. No. 8,273,101 (Garcia et al., Sep. 25, 2012, “System and Method for Delivering and Deploying an Occluding Device within a Vessel”); U.S. Pat. No. 8,277,500 (Schmid et al., Oct. 2, 2012, “Slide-And-Lock Stent”); U.S. Pat. No. 8,292,944 (Schmid et al., Oct. 23, 2012, “Slide-And-Lock Stent”); U.S. Pat. No. 8,377,112 (Griffin et al., Feb. 19, 2013, “Sacrificial Anode Stent System”); U.S. Pat. No. 8,444,686 (Holman et al., May 21, 2013, “Catheter with Removable Balloon Protector and Stent Delivery System with Removable Stent Protector”); U.S. Pat. No. 8,636,760 (Garcia et al., Jan. 28, 2014, “System and Method for Delivering and Deploying an Occluding Device Within a Vessel”); U.S. Pat. No. 8,709,065 (Chobotov, Apr. 29, 2014, “Advanced Endovascular Graft”); and U.S. Pat. No. 8,795,346 (Alkhatib, Aug. 5, 2014, “Semi Rigid Edge Protection Design for Stent Delivery System”).

Prior art which appears to be within this category also includes U.S. patent applications: 20020151965 (Roth, Oct. 17, 2002, “Rolled Stent with Waveform Perforation Pattern”); 20030159920 (Roth, Aug. 28, 2003, “Thin Film Stent”); 20040002752 (Griffin et al., Jan. 1, 2004, “Sacrificial Anode Stent System”); 20050192661 (Griffen et al., Sep. 1, 2005, “Sacrificial Anode Stent System”); 20050251247 (Roth, Nov. 10, 2005, “Rolled Stent with Waveform Perforation Pattern”); 20060271149 (Berez et al., Nov. 30, 2006, “System and Method for Delivering and Deploying an Occluding Device within a Vessel”); 20060271153 (Garcia et al., Nov. 30, 2006, “System and Method for Delivering and Deploying an Occluding Device within a Vessel”); 20070031584 (Roth, Feb. 8, 2007, “Thin Film Stent”); and 20080319521 (Norris et al., Dec. 25, 2008, “Compressible Resilient Fabric Devices and Methods”).

Prior art which appears to be within this category also includes U.S. patent applications: 20090192536 (Berez et al., Jul. 30, 2009, “System and Method for Delivering and Deploying an Occluding Device Within a Vessel”); 20090198318 (Berez et al., Aug. 6, 2009, “System and Method for Delivering and Deploying an Occluding Device Within a Vessel”); 20090287292 (Becking et al., Nov. 19, 2009, “Braid Implant Delivery Systems”); 20090318947 (Garcia et al., Dec. 24, 2009, “System and Method for Delivering and Deploying an Occluding Device Within a Vessel”); 20100152828 (Pakbaz et al., Jun. 17, 2010, “Devices and Methods for Accessing and Treating an Aneurysm”); 20100179640 (Reith, Jul. 15, 2010, “Radially Expandable System for Use in Body Tubes”); 20110004294 (Bialas, R., Jan. 6, 2011, “Fatigue-Resistant Stent”); 20110066221 (White et al., Mar. 17, 2011, “Hybrid Intraluminal Device”); 20110144740 (Molaei et al., Jun. 6, 2011, “Medical Devices Including Metallic Film and at Least One Filament”); and 20110230957 (Bonsignore et al., Sep. 22, 2011, “Alternating Circumferential Bridge Stent Design and Methods for Use Thereof”).

Prior art which appears to be within this category also includes U.S. patent applications: 20110319928 (Griffin et al., Dec. 29, 2011, “Sacrificial Anode Stent System”); 20120221095 (Berez et al., Aug. 30, 2012, “System and Method for Delivering and Deploying an Occluding Device within a Vessel”); 20120277784 (Berez et al., Nov. 1, 2012, “System and Method for Delivering and Deploying an Occluding Device within a Vessel”); 20120283765 (Berez et al., Nov. 8, 2012, “System and Method for Delivering and Deploying an Occluding Device within a Vessel”); 20120283815 (Berez et al., Nov. 8, 2012, “System and Method for Delivering and Deploying an Occluding Device within a Vessel”); 20120316638 (Grad et al., Dec. 13, 2012, “Method and Device for Treating Cerebrovascular Pathologies and Delivery System Therefor”); 20120323547 (Baloch et al., Dec. 20, 2012, “Method for Intracranial Aneurysm Analysis and Endovascular Intervention Planning”); and 20130053872 (Hansen, Feb. 28, 2013, “Device and Method for Preventing Blood Flow into an Aneurysm”).

Prior art which appears to be within this category also includes U.S. patent applications: 20130166010 (Vad, Jun. 27, 2013, “Hybrid Balloon-Expandable/Self-Expanding Prosthesis for Deployment in a Body Vessel and Method of Making”); 20130172925 (Garcia et al., Jul. 4, 2013, “System and Method for Delivering and Deploying an Occluding Device within a Vessel”); 20130172976 (Garcia et al., Jul. 4, 2013, “System and Method for Delivering and Deploying an Occluding Device within a Vessel”); 20130211492 (Schneider et al., Aug. 15, 2013, “Implant for Influencing the Blood Flow in Arteriovenous Defects”); 20130253631 (Schmid et al., Sep. 26, 2013, “Slide-And-Lock Stent”); 20130261730 (Bose et al., Oct. 3, 2013, “Aneurysm Occlusion System and Method”); 20130289713 (Pearson et al., Oct. 31, 2013, “Circumferentially Constraining Sutures for a Stent-Graft”); 20140172067 (Brown et al., Jun. 19, 2014, “Luminal Stenting”); 20140180387 (Khenansho et al., Jun. 26, 2014, “Stent Delivery System”); 20140214071 (Thomas, Jul. 31, 2014, “Embolic Coil Delivery System and Method of Using Same”); 20140249614 (Levi et al., Sep. 4, 2014, “Thin Film Vascular Stent and Biocompatible Surface Treatment”); and 20140277391 (Layman et al., Sep. 18, 2014, “Stent and Method of Use”).

11. Stent in Parent Vessel to Contain Embolics within Aneurysm Sack:

The prior art also discloses devices and methods for treating aneurysms comprising the implantation of a stent within the parent vessel of an aneurysm in order to contain embolics within an aneurysm sack. This is sometimes called “jailing.” In an example, the stent wall can have an opening through which embolic members (such as coils) are inserted into the aneurysm sack. Prior art which appears to be within this category includes U.S. Pat. No. 5,928,260 (Chin et al., Jul. 27, 1999, “Removable Occlusion System for Aneurysm Neck”); U.S. Pat. No. 5,951,599 (McCrory, Sep. 14, 1999, “Occlusion System for Endovascular Treatment of an Aneurysm”); U.S. Pat. No. 5,980,514 (Kupiecki et al., Nov. 9, 1999, “Aneurysm Closure Device Assembly”); U.S. Pat. No. 6,017,977 (Evans et al., Jan. 25, 2000, “Methods for Embolizing Blood Vessels”); U.S. Pat. No. 6,096,034 (Kupiecki et al., Aug. 1, 2000, “Aneurysm Closure Device Assembly”); U.S. Pat. No. 6,168,592 (Kupiecki et al., Jan. 2, 2001, “Aneurysm Closure Device Assembly”); U.S. Pat. No. 6,281,263 (Evans et al., Aug. 28, 2001, “Methods for Embolizing Blood Vessels”); U.S. Pat. No. 6,335,384 (Evans et al., Jan. 1, 2002, “Methods for Embolizing Blood Vessels”); U.S. Pat. No. 6,344,041 (Kupiecki et al., Feb. 5, 2002, “Aneurysm Closure Device Assembly”); and U.S. Pat. No. 6,344,048 (Chin et al., Feb. 5, 2002, “Removable Occlusion System for Aneurysm Neck”).

Prior art which appears to be within this category also includes U.S. Pat. No. 6,375,668 (Gifford et al., Apr. 23, 2002, “Devices and Methods for Treating Vascular Malformations”); U.S. Pat. No. 6,746,468 (Sepetka et al., Jun. 8, 2004, “Devices and Methods for Treating Vascular Malformations”); U.S. Pat. No. 6,780,196 (Chin et al., Aug. 24, 2004, “Removable Occlusion System for Aneurysm Neck”); U.S. Pat. No. 6,913,618 (Denardo et al., Jul. 5, 2005, “Intravascular Flow Modifier and Reinforcement Device”); U.S. Pat. No. 7,001,422 (Escamilla et al., Feb. 21, 2006, “Expandable Stent and Delivery System”); U.S. Pat. No. 7,229,461 (Chin et al., Jun. 12, 2007, “Removable Occlusion System for Aneurysm Neck”); U.S. Pat. No. 7,288,112 (Denardo et al., Oct. 30, 2007, “Intravascular Flow Modifier and Reinforcement Device”); U.S. Pat. No. 7,309,351 (Escamilla et al., Dec. 18, 2007, “Expandable Stent with Markers and Stent Delivery System”); U.S. Pat. No. 7,563,270 (Gumm, Jul. 21, 2009, “Rotating Stent Delivery System for Side Branch Access and Protection and Method of Using Same”); and U.S. Pat. No. 7,569,066 (Gerberding et al., Aug. 4, 2009, “Methods and Devices for the Treatment of Aneurysms”).

Prior art which appears to be within this category also includes U.S. Pat. No. 7,875,044 (Feller et al., Jan. 25, 2011, “Remodeling Device for Aneurysms”); U.S. Pat. No. 8,016,853 (Griffen et al., Sep. 13, 2011, “Sacrificial Anode Stent System”); U.S. Pat. No. 8,377,112 (Griffin et al., Feb. 19, 2013, “Sacrificial Anode Stent System”); U.S. Pat. No. 8,444,667 (Porter, May 21, 2013, “Device for Closure of a Vascular Defect and Method for Treating the Same”); U.S. Pat. No. 8,470,013 (Duggal et al., Jun. 25, 2013, “Systems and Methods for Aneurysm Treatment and Vessel Occlusion”); U.S. Pat. No. 8,535,367 (Kim et al., Sep. 17, 2013, “Devices and Methods for Treatment of Vascular Aneurysms”); U.S. Pat. No. 8,597,320 (Sepetka et al., Dec. 3, 2013, “Devices and Methods for Treating Vascular Malformations”); U.S. Pat. No. 8,647,377 (Kim et al., Feb. 11, 2014, “Devices and Methods for Treatment of Vascular Aneurysms”); U.S. Pat. No. 8,747,430 (Porter, Jun. 10, 2014, “Device for Closure of a Vascular Defect and Method for Treating the Same”); U.S. Pat. No. 8,771,341 (Strauss et al., Jul. 8, 2014, “Protuberant Aneurysm Bridging Device and Method of Use”); and U.S. Pat. No. 8,808,361 (Strauss et al., Aug. 19, 2014, “Protuberant Aneurysm Bridging Device and Method of Use”).

Prior art which appears to be within this category also includes U.S. patent applications: 20020042628 (Chin et al., Apr. 11, 2002, “Removable Occlusion System for Aneurysm Neck”); 20020169473 (Sepetka et al., Nov. 14, 2002, “Devices and Methods for Treating Vascular Malformations”); 20030014075 (Rosenbluth et al., Jan. 16, 2003, “Methods, Materials and Apparatus for Deterring or Preventing Endoleaks Following Endovascular Graft Implantation”); 20040002752 (Griffin et al., Jan. 1, 2004, “Sacrificial Anode Stent System”); 20040181253 (Sepetka et al., Sep. 16, 2004, “Devices and Methods for Treating Vascular Malformations”); 20040193246 (Ferrera, Sep. 30, 2004, “Methods and Apparatus for Treating Aneurysms and Other Vascular Defects”); 20050004660 (Rosenbluth et al., Jan. 6, 2005, “Methods, Materials and Apparatus for Deterring or Preventing Endoleaks Following Endovascular Graft Implantation”); 20050021077 (Chin et al., Jan. 27, 2005, “Removable Occlusion System for Aneurysm Neck”); and 20050192661 (Griffen et al., Sep. 1, 2005, “Sacrificial Anode Stent System”).

Prior art which appears to be within this category also includes U.S. patent applications: 20060206196 (Porter, Sep. 14, 2006, “Device for Closure of a Vascular Defect and Method for Treating the Same”); 20060292206 (Kim et al., Dec. 28, 2006, “Devices and Methods for Treatment of Vascular Aneurysms”); 20070050008 (Kim et al., Mar. 1, 2007, “Devices and Methods for Treatment of Vascular Aneurysms”); 20070055355 (Kim et al., Mar. 8, 2007, “Devices and Methods for Treatment of Vascular Aneurysms”); 20070061005 (Kim et al., Mar. 15, 2007, “Devices and Methods for Treatment of Vascular Aneurysms”); 20070150041 (Evans et al., Jun. 28, 2007, “Methods and Systems for Aneurysm Treatment Using Filling Structures”); 20070150045 (Ferrera, Jun. 28, 2007, “Methods and Apparatus for Treating Aneurysms and Other Vascular Defects”); 20080004692 (Henson et al., Jan. 3, 2008, “Dynamically Adjustable Vascular Stent”); 20080033341 (Grad, Feb. 7, 2008, “Methods and Devices for Reducing or Blocking Blood Flow To a Selected Blood Vessel or Part Thereof”); 20080161936 (Feller et al., Jul. 3, 2008, “Remodeling Device for Aneurysms”); and 20090069880 (Vonderwalde et al., Mar. 12, 2009, “Implantable Graft Assembly and Aneurysm Treatment”).

Prior art which appears to be within this category also includes U.S. patent applications: 20090125053 (Ferrera et al., May 14, 2009, “Aneurysm Neck Bridging Processes with Revascularization Systems Methods and Products Thereby”); 20090318948 (Linder et al., Dec. 24, 2009, “Device, System and Method for Aneurysm Embolization”); 20100004671 (Gerberding et al., Jan. 7, 2010, “Methods and Devices for the Treatment of Aneurysms”); 20100023105 (Levy et al., Jan. 28, 2010, “Vascular Remodeling Device”); 20100106240 (Duggal et al., Apr. 29, 2010, “Systems and Methods for Aneurysm Treatment and Vessel Occlusion”); 20100131002 (Connor et al., May 27, 2010, “Stent with a Net Layer to Embolize an Aneurysm”); 20110046716 (Parkinson et al., Feb. 24, 2011, “Stent”); 20110082491 (Sepetka et al., Apr. 7, 2011, “Devices and Methods for Treating Vascular Malformations”); 20110137332 (Sepetka et al., Jun. 9, 2011, “Devices and Methods for Treating Vascular Malformations”); 20110184453 (Levy et al., Jul. 28, 2011, “Vascular Remodeling Device”); 20110224776 (Sepetka et al., Sep. 15, 2011, “Devices and Methods for Treating Vascular Malformations”); and 20110319928 (Griffin et al., Dec. 29, 2011, “Sacrificial Anode Stent System”).

Prior art which appears to be within this category also includes U.S. patent applications: 20120245674 (Molaei et al., Sep. 27, 2012, “Vascular Remodeling Device”); 20120245675 (Molaei et al., Sep. 27, 2012, “Vascular Remodeling Device”); 20120283768 (Cox et al., Nov. 8, 2012, “Method and Apparatus for the Treatment of Large and Giant Vascular Defects”); 20120330343 (Kim et al., Dec. 27, 2012, “Devices and Methods for Treatment of Vascular Aneurysms”); 20130116774 (Strauss et al., May 9, 2013, “Protuberant Aneurysm Bridging Device and Method of Use”); 20130231732 (Vonderwalde et al., Sep. 5, 2013, “Implantable Graft Assembly and Aneurysm Treatment”); 20130238083 (Duggal et al., Sep. 12, 2013, “Systems and Methods for Aneurysm Treatment and Vessel Occlusion”); 20130268053 (Molaei et al., Oct. 10, 2013, “Vascular Remodeling Device”); 20130289714 (Strauss et al., Oct. 31, 2013, “Protuberant Aneurysm Bridging Device and Method of Use”); 20140025151 (Gao, Jan. 23, 2014, “Retrievable Stent for Intracranial Aneurysms”); 20140081374 (Kim et al., Mar. 20, 2014, “Devices and Methods for Treatment of Vascular Aneurysms”); 20140121752 (Losordo et al., May 1, 2014, “Wing Bifurcation Reconstruction Device”); and 20140249616 (Strauss et al., Sep. 4, 2014, “Protuberant Aneurysm Bridging Device Deployment Method”).

### 12. Multiple Stents in Parent Vessel:

The prior art also discloses devices and methods for treating aneurysms comprising the coordinated and concurrent implantation of multiple stents (or stent modules) within the parent vessel of an aneurysm. Prior art which appears to be within this category includes U.S. Pat. No. 7,137,993 (Acosta et al., Nov. 21, 2006, “Apparatus and Methods for Delivery of Multiple Distributed Stents”); U.S. Pat. No. 7,147,655 (Chermoni, Dec. 12, 2006, “Balloon Catheter for Multiple Adjustable Stent Deployment”); U.S. Pat. No. 7,294,146 (Chew et al., Nov. 13, 2007, “Apparatus and Methods for Delivery of Variable Length Stents”); U.S. Pat. No. 7,905,913 (Chew et al., Mar. 15, 2011, “Apparatus and Methods for Delivery of Multiple Distributed Stents”); U.S. Pat. No. 7,922,755 (Acosta et al., Apr. 12, 2011, “Apparatus and Methods for Delivery of Multiple Distributed Stents”); U.S. Pat. No. 7,963,987 (Melsheimer et al., Jun. 21, 2011, “Sequential Implant Delivery System”); U.S. Pat. No. 8,016,870 (Chew et al., Sep. 13, 2011, “Apparatus and Methods for Delivery of Variable Length Stents”); U.S. Pat. No. 8,016,871 (Chew et al., Sep. 13, 2011, “Apparatus and Methods for Delivery of Multiple Distributed Stents”); and U.S. Pat. No. 8,246,672 (Osborne, Aug. 21, 2012, “Endovascular Graft with Separately Positionable and Removable Frame Units”).

Prior art which appears to be within this category also includes U.S. patent applications: 20030065375 (Eskuri, Apr. 3, 2003, “Nested Stent Apparatus”); 20040215331 (Chew et al., Oct. 28, 2004, “Apparatus and Methods for Delivery of Variable Length Stents”); 20040249435 (Andreas et al., Dec. 9, 2004, “Stent Deployment Systems and Methods”); 20070088368 (Acosta; Pablo et al., Apr. 19, 2007, “Apparatus and Methods for Delivery of Multiple Distributed Stents”); 20090088832 (Chew et al., Apr. 2, 2009, “Apparatus and Methods for Delivery of Variable Length Stents”); 20100318173 (Kolandaivelu et al., Dec. 16, 2010, “Endovascular Devices/Catheter Platforms and Methods for Achieving Congruency in Sequentially Deployed Devices”); and 20110152996 (Acosta et al., Jun. 23, 2011, “Apparatus and Methods for Delivery of Multiple Distributed Stents”).

### 13. Temporary Balloon in Parent Vessel:

The prior art also discloses devices and methods for treating aneurysms comprising the temporarily insertion and expansion of a balloon within the parent vessel of an aneurysm. In an example, temporary insertion and expansion of such as balloon can help to contain a liquid embolic composition within the aneurysm sack while the embolic composition is congealing. As another example, such a balloon can help to contain embolic coils within the aneurysm sack while the coils are being deployed. Prior art which appears to be within this category also includes U.S. Pat. No. 5,776,097 (Massoud, Jul. 7, 1998, “Method and Device for Treating Intracranial Vascular Aneurysms”); U.S. Pat. No. 5,785,679 (Abolfathi et al., Jul. 28, 1998, “Methods and Apparatus for Treating Aneurysms and Arterio-Venous Fistulas”); U.S. Pat. No. 5,795,331 (Cragg et al., Aug. 18, 1998, “Balloon Catheter for Occluding Aneurysms of Branch Vessels”); U.S. Pat. No. 6,074,407 (Levine et al., Jun. 13, 2000, “Delivery Catheter for Occlusive Implants”); U.S. Pat. No. 6,096,021 (Helm et al., Aug. 1, 2000, “Flow Arrest, Double Balloon Technique for Occluding Aneurysms or Blood Vessels”); U.S. Pat. No. 8,221,447 (Solar et al., Jul. 17, 2012, “Aneurysm Coil Delivery System”); and U.S. Pat. No. 8,361,106 (Solar et al., Jan. 29, 2013, “Aneurysm Coil Delivery System”).

Prior art which appears to be within this category also includes U.S. patent applications: 20070219578 (Solar et al., Sep. 20, 2007, “Aneurysm Coil Delivery System”); 20080033341 (Grad, Feb. 7, 2008, “Methods and Devices for Reducing or Blocking Blood Flow To a Selected Blood Vessel or Part Thereof”); 20120078285 (Griffin, Mar. 29, 2012, “Balloon Catheter for Intravascular Therapies”); 20120116352 (Rangi, May 10, 2012, “Balloon Assisted Occlusion of Aneurysms”); 20120283764 (Solar et al., Nov. 8, 2012, “Aneurysm Coil Delivery System”); and 20130310687 (Takizawa et al., Nov. 21, 2013, “Blood Vessel Embolization Method Using Balloon Catheter and Balloon Catheter for Blood Vessel Embolization Method”).

### 14. Multi-Balloon Device:

The prior art also discloses devices and methods for treating aneurysms which comprise multiple balloons and/or balloon chambers. In an example, multiple balloons or balloon chambers can be used to differentially expand different portions of a stent within the parent vessel of an aneurysm in order to create structural asymmetry and/or differences in wall porosity. Prior art which appears to be within this category includes U.S. Pat. No. 5,226,889 (Sheiban, Jul. 13, 1993, “Double Balloon Catheter for Stent Implantation”); U.S. Pat. No. 5,304,132 (Jang, Apr. 19, 1994, “Limacon Geometry Balloon Angioplasty Catheter Systems and Method of Making Same”); U.S. Pat. No. 5,536,252 (Imran et al., Jul. 16, 1996, “Angioplasty Catheter with Multiple Coaxial Balloons”); U.S. Pat. No. 5,833,657 (Reinhardt et al., Nov. 10, 1998, “Single-Walled Balloon Catheter with Non-Linear Compliance Characteristic”); U.S. Pat. No. 6,123,712 (Di Caprio et al., Sep. 26, 2000, “Balloon Catheter with Stent Securement Means”); U.S. Pat. No. 6,136,011 (Stambaugh, Oct. 24, 2000, “Stent Delivery System and Method of Use”); and U.S. Pat. No. 6,419,685 (Di Caprio et al., 2002, “Balloon Catheter with Stent Securement Means”).

Prior art which appears to be within this category also includes U.S. Pat. No. 6,471,672 (Brown et al., Oct. 29, 2002, “Selective High Pressure Dilation Balloon”); U.S. Pat. No. 6,506,201 (Di Caprio et al., 2003, “Balloon Catheter with Stent Securement Means”); U.S. Pat. No. 6,605,056 (Eidenschink et al., 2003, “Conformable Balloon”); U.S. Pat. No. 7,052,510 (Richter, May 30, 2006, “Two Balloon Staged Stent Expansion”); U.S. Pat. No. 7,300,459 (Heuser, Nov. 27, 2007, “Stent with Covering and Differential Dilation”); U.S. Pat. No. 7,776,079 (Gumm, Aug. 17, 2010, “Conical Balloon for Deployment into Side Branch”); U.S. Pat. No. 8,460,240 (Towler, Jun. 11, 2013, “Inflatable Toroidal-Shaped Balloons”); U.S. Pat. No. 8,657,865 (Gumm, Feb. 25, 2014, “Conical Balloon for Deployment Into Side Branch”); and U.S. Pat. No. 8,709,062 (Dusbabek et al., Apr. 29, 2014, “Stent Delivery System Having Stent Securement Apparatus”).

Prior art which appears to be within this category also includes U.S. patent applications: 20030014007 (Eidenschink et al., Jan. 16, 2003, “Conformable Balloon”); 20050209674 (Kutscher et al., Sep. 22, 2005, “Balloon Assembly (V)”); 20070100301 (Gumm, May 3, 2007, “Conical Balloon for Deployment into Side Branch”); 20100063531 (Rudakov et al., Mar. 11, 2010, “Medical Device with Non-Circumferential Surface Portion”); 20100305681 (Gumm, Dec. 2, 2010, “Conical Balloon for Deployment into Side Branch”); 20110238105 (Gelbart et al., Sep. 29, 2011, “Vivo Inflatable Structures for Example to Expand Stents”); 20120116352 (Rangi, May 10, 2012, “Balloon Assisted Occlusion of Aneurysms”); 20130060317 (Dusbabek et al., Mar. 7, 2013, “Stent Delivery System Having Stent Securement Apparatus”); 20130231695 (Malek, Sep. 5, 2013, “Embolic Coil”); 20140222128 (Dusbabek et al., Aug. 7, 2014, “Stent Delivery System Having Stent Securement Apparatus”); and 20140277361 (Farhat et al., Sep. 18, 2014, “Methods and Apparatus for Luminal Stenting”); and Yet unpublished U.S. patent application Ser. No. 13/889,451 (Connor et al., 2013, “Method of Radially-Asymmetric Stent Expansion”).

### 15. Aneurysm Neck Bridge or Saddle in Relatively-Straight Parent Vessel:

The prior art also discloses devices and methods for treating aneurysms comprising an aneurysm neck bridge (or saddle) which is implanted within a relatively-straight portion of a parent vessel. Prior art which appears to be within this category includes U.S. Pat. No. 6,007,573 (Wallace et al., Dec. 28, 1999, “Intracranial Stent and Method of Use”); U.S. Pat. No. 6,093,199 (Brown et al., Jul. 25, 2000, “Intra-Luminal Device for Treatment of Body Cavities and Lumens and Method of Use”); U.S. Pat. No. 6,139,564 (Teoh, Oct. 31, 2000, “Minimally Occlusive Flow Disruptor Stent for Bridging Aneurysm Necks”); U.S. Pat. No. 6,231,597 (Deem et al., May 15, 2001, “Apparatus and Methods for Selectively Stenting a Portion of a Vessel Wall”); U.S. Pat. No. 6,309,367 (Boock, Oct. 30, 2001, “Aneurysm Shield”); U.S. Pat. No. 6,432,128 (Wallace et al., Aug. 13, 2002, “Intracranial Stent and Method of Use”); U.S. Pat. No. 6,605,111 (Bose et al., Aug. 12, 2003, “Endovascular Thin Film Devices and Methods for Treating and Preventing Stroke”); U.S. Pat. No. 6,613,074 (Mitelberg et al., Sep. 2, 2003, “Endovascular Aneurysm Embolization Device”); U.S. Pat. No. 6,669,719 (Wallace et al., Dec. 30, 2003, “Intracranial Stent and Method of Use”); U.S. Pat. No. 6,802,851 (Jones et al., Oct. 12, 2004, “Stent Aneurysm Embolization Method Using Collapsible Member and Embolic Coils”); U.S. Pat. No. 6,811,560 (Jones et al., Nov. 2, 2004, “Stent Aneurysm Embolization Method and Device”); U.S. Pat. No. 7,231,260 (Wallace et al., Jun. 12, 2007, “Intravascular Self-Anchoring Electrode Body with Arcuate Springs, Spring Loops, or Arms”); U.S. Pat. No. 7,241,301 (Thramann et al., Jul. 10, 2007, “Aneurysm Stent with Growth Factor”); U.S. Pat. No. 7,306,622 (Jones et al., Dec. 11, 2007, “Stent Embolization Device”); and U.S. Pat. No. 7,323,005 (Wallace et al., Jan. 29, 2008, “Intracranial Stent and Method of Use”).

Prior art which appears to be within this category also includes U.S. Pat. No. 7,384,426 (Wallace et al., Jun. 10, 2008, “Intracranial Stent and Method of Use”); U.S. Pat. No. 7,572,288 (Cox, Aug. 11, 2009, “Aneurysm Treatment Device and Method of Use”); U.S. Pat. No. 7,608,088 (Jones et al., Oct. 27, 2009, “Stent Aneurysm Embolization Device”); U.S. Pat. No. 7,621,928 (Thramann et al., Nov. 24, 2009, “Aneurysm Stent”); U.S. Pat. No. 7,901,445 (Wallace et al., Mar. 8, 2011, “Intracranial Stent and Method of Use”); U.S. Pat. No. 8,038,706 (Eidenschink et al., Oct. 18, 2011, “Crown Stent Assembly”); U.S. Pat. No. 8,187,315 (Clauson et al., May 29, 2012, “Partial Stent for Treatment of a Vascular Aneurysm”); U.S. Pat. No. 8,252,040 (Cox, Aug. 28, 2012, “Aneurysm Treatment Device and Method of Use”); U.S. Pat. No. 8,267,986 (Berez et al., Sep. 18, 2012, “Vascular Stenting for Aneurysms”); U.S. Pat. No. 8,382,825 (Garcia et al., Feb. 26, 2013, “Flexible Vascular Occluding Device”); U.S. Pat. No. 8,398,701 (Berez et al., Mar. 19, 2013, “Flexible Vascular Occluding Device”); U.S. Pat. No. 8,409,267 (Berez et al., Apr. 2, 2013, “Vascular Stenting for Aneurysms”); U.S. Pat. No. 8,409,269 (Berez et al., Apr. 2, 2013, “Procedures for Vascular Occlusion”); U.S. Pat. No. 8,425,548 (Connor, Apr. 23, 2013, “Occluding Member Expansion and then Stent Expansion for Aneurysm Treatment”); U.S. Pat. No. 8,470,013 (Duggal et al., Jun. 25, 2013, “Systems and Methods for Aneurysm Treatment and Vessel Occlusion”); U.S. Pat. No. 8,500,788 (Berez et al., Aug. 6, 2013, “Vascular Stenting and Other Procedures”); U.S. Pat. No. 8,529,614 (Berez et al., Sep. 10, 2013, “Vascular Stenting and Other Procedures”); U.S. Pat. No. 8,529,614 (Berez et al., Sep. 10, 2013, “Vascular Stenting and Other Procedures”); U.S. Pat. No. 8,556,953 (Berez et al., Oct. 15, 2013, “Vascular Stenting for Aneurysms”); U.S. Pat. No. 8,562,667 (Cox, Oct. 22, 2013, “Aneurysm Treatment Device and Method of Use”); and U.S. Pat. No. 8,715,312 (Burke et al., May 6, 2014, “Aneurysm Treatment Device and Method of Use”).

Prior art which appears to be within this category also includes U.S. patent applications: 20030018294 (Cox, Jan. 23, 2003, “Aneurysm Treatment Device and Method of Use”); 20030109917 (Rudin et al., Jun. 12, 2003, “Stent Vascular Intervention Device and Method”); 20030139802 (Wulfman et al., Jul. 24, 2003, “Medical Device”); 20050033349 (Jones et al., Feb. 10, 2005, “Stent Aneurysm Embolization Device”); 20050267568 (Berez et al., Dec. 1, 2005, “Flexible Vascular Occluding Device”); 20060206200 (Garcia et al., Sep. 14, 2006, “Flexible Vascular Occluding Device”); 20060206201 (Garcia et al., Sep. 14, 2006, “Flexible Vascular Occluding Device”); 20070021816 (Rudin, Jan. 25, 2007, “Stent Vascular Intervention Device and Methods for Treating Aneurysms”); 20070219610 (Israel, Sep. 20, 2007, “Stent with Flap”); 20070225794 (Thramann et al., Sep. 27, 2007, “Aneurysm Stent”); 20090228029 (Lee, Sep. 10, 2009, “Aneurysm Shield Anchoring Device”); and 20090270974 (Berez et al., Oct. 29, 2009, “Vascular Stenting for Aneurysms”).

Prior art which appears to be within this category also includes U.S. patent applications: 20090287241 (Berez et al., Nov. 19, 2009, “Methods and Apparatus for Luminal Stenting”); 20090287288 (Berez et al., Nov. 19, 2009, “Methods and Apparatus for Luminal Stenting”); 20090292348 (Berez et al., Nov. 26, 2009, “Vascular Stenting and Other Procedures”); 20090319017 (Berez et al., Dec. 24, 2009, “Vascular Stenting for Aneurysms”); 20100010624 (Berez et al., Jan. 14, 2010, “Vascular Stenting for Aneurysms”); 20100063531 (Rudakov et al., Mar. 11, 2010, “Medical Device with Non-Circumferential Surface Portion”); 20100082091 (Berez et al., Apr. 1, 2010, “Vascular Stenting and Other Procedures”); 20100106240 (Duggal et al., Apr. 29, 2010, “Systems and Methods for Aneurysm Treatment and Vessel Occlusion”); 20110022149 (Cox et al., Jan. 27, 2011, “Methods and Devices for Treatment of Vascular Defects”); 20110152998 (Berez et al., Jun. 23, 2011, “Procedures for Vascular Occlusion”); and 20110166592 (Garcia et al., Jul. 7, 2011, “Flexible Vascular Occluding Device”).

Prior art which appears to be within this category also includes U.S. patent applications: 20110184451 (Sahl, Jul. 28, 2011, “Membrane Implant for Treatment of Cerebral Artery Aneurysms”); 20120004682 (Connor, Jan. 5, 2012, “Occluding Member Expansion and Then Stent Expansion for Aneurysm Treatment”); 20120165919 (Cox et al., Jun. 28, 2012, “Methods and Devices for Treatment of Vascular Defects”); 20130103074 (Riina et al., Apr. 25, 2013, “Method and Apparatus for Restricting Flow Through an Opening in the Side Wall of a Body Lumen, and/or for Reinforcing a Weakness in the Side Wall of a Body Lumen, While Still Maintaining Substantially Normal Flow Through the Body Lumen”); 20130172975 (Berez et al., Jul. 4, 2013, “Methods and Apparatus for Luminal Stenting”); 20130238083 (Duggal et al., Sep. 12, 2013, “Systems and Methods for Aneurysm Treatment and Vessel Occlusion”); 20130274862 (Cox et al., Oct. 17, 2013, “Methods and Devices for Treatment of Vascular Defects”); and 20130274863 (Cox et al., Oct. 17, 2013, “Methods and Devices for Treatment of Vascular Defects”).

Prior art which appears to be within this category also includes U.S. patent applications: 20130274866 (Cox et al., Oct. 17, 2013, “Methods and Devices for Treatment of Vascular Defects”); 20130274868 (Cox et al., Oct. 17, 2013, “Methods and Devices for Treatment of Vascular Defects”); 20130282096 (Berez et al., Oct. 24, 2013, “Procedures for Vascular Occlusion”); 20140018843 (Berez et al., Jan. 16, 2014, “Methods and Apparatus for Luminal Stenting”); 20140052233 (Cox et al., Feb. 20, 2014, “Methods and Devices for Treatment of Vascular Defects”); 20140074149 (Garcia et al., Mar. 13, 2014, “Flexible Vascular Occluding Device”); 20140094896 (Berez et al., Apr. 3, 2014, “Vascular Stenting for Aneurysms”); 20140114342 (Berez et al., Apr. 24, 2014, “Flexible Vascular Occluding Device”); 20140128901 (Kang et al., May 8, 2014, “Implant for Aneurysm Treatment”); 20140172071 (Berez et al., Jun. 19, 2014, “Vascular Stenting for Aneurysms”); and 20140288633 (Burke et al., Sep. 25, 2014, “Aneurysm Treatment Device and Method of Use”).

16. Aneurysm Neck Bridge or Saddle in Parent Vessel with Three-Way Junction:

The prior art also discloses devices and methods for treating aneurysms comprising an aneurysm neck bridge (or saddle) which is implanted within a parent vessel with a three-way junction. Prior art which appears to be within this category includes U.S. Pat. No. 5,951,599 (McCrory, Sep. 14, 1999, “Occlusion System for Endovascular Treatment of an Aneurysm”); U.S. Pat. No. 6,309,367 (Boock, Oct. 30, 2001, “Aneurysm Shield”); U.S. Pat. No. 6,969,401 (Marotta et al., Nov. 29, 2005, “Endovascular Prosthesis”); U.S. Pat. No. 7,232,461 (Ramer, Jun. 19, 2007, “Neck Covering Device for an Aneurysm”); U.S. Pat. No. 7,572,288 (Cox, Aug. 11, 2009, “Aneurysm Treatment Device and Method of Use”); U.S. Pat. No. 8,252,040 (Cox, Aug. 28, 2012, “Aneurysm Treatment Device and Method of Use”); U.S. Pat. No. 8,388,650 (Gerberding et al., Mar. 5, 2013, “Systems and Methods for Supporting or Occluding a Physiological Opening or Cavity”); U.S. Pat. No. 8,470,013 (Duggal et al., Jun. 25, 2013, “Systems and Methods for Aneurysm Treatment and Vessel Occlusion”); U.S. Pat. No. 8,562,667 (Cox, Oct. 22, 2013, “Aneurysm Treatment Device and Method of Use”); and U.S. Pat. No. 8,715,312 (Burke et al., May 6, 2014, “Aneurysm Treatment Device and Method of Use”).

Prior art which appears to be within this category also includes U.S. patent applications: 20030018294 (Cox, Jan. 23, 2003, “Aneurysm Treatment Device and Method of Use”); 20030109917 (Rudin et al., Jun. 12, 2003, “Stent Vascular Intervention Device and Method”); 20040111112 (Hoffmann, Jun. 10, 2004, “Method and Apparatus for Retaining Embolic Material”); 20050096728 (Ramer, May 5, 2005, “Neck Covering Device for an Aneurysm”); 20070021816 (Rudin, Jan. 25, 2007, “Stent Vascular Intervention Device and Methods for Treating Aneurysms”); 20080114391 (Dieck et al., May 15, 2008, “Aneurysm Covering Devices and Delivery Devices”); 20080114436 (Dieck et al., May 15, 2008, “Aneurysm Covering Devices and Delivery Devices”); 20080221600 (Dieck et al., Sep. 11, 2008, “Isolation Devices for the Treatment of Aneurysms”); 20080319533 (Lehe, Dec. 25, 2008, “Aneurysm Occlusion Assist Device”); and 20100094335 (Gerberding et al., Apr. 15, 2010, “Systems and Methods for Supporting or Occluding a Physiological Opening or Cavity”).

Prior art which appears to be within this category also includes U.S. patent applications: 20100106240 (Duggal et al., Apr. 29, 2010, “Systems and Methods for Aneurysm Treatment and Vessel Occlusion”); 20100198250 (Ricci et al., Aug. 5, 2010, “Endovascular Prosthesis Delivery System”); 20110022149 (Cox et al., Jan. 27, 2011, “Methods and Devices for Treatment of Vascular Defects”); 20110245862 (Dieck et al., Oct. 6, 2011, “Isolation Devices for the Treatment of Aneurysms”); 20120143237 (Cam et al., Jun. 7, 2012, “Vascular Remodeling Device”); 20120143317 (Cam et al., Jun. 7, 2012, “Vascular Remodeling Device”); 20120165919 (Cox et al., Jun. 28, 2012, “Methods and Devices for Treatment of Vascular Defects”); 20120245674 (Molaei et al., Sep. 27, 2012, “Vascular Remodeling Device”); 20120245675 (Molaei et al., Sep. 27, 2012, “Vascular Remodeling Device”); 20120296361 (Cam et al., Nov. 22, 2012, “Vascular Remodeling Device”); 20120296362 (Cam et al., Nov. 22, 2012, “Vascular Remodeling Device”); 20120316632 (Gao, Dec. 13, 2012, “Retrievable Covered Stent for Bifurcation Aneurysms”); and 20130090682 (Bachman et al., Apr. 11, 2013, “Devices, Systems and Methods for Enclosing an Anatomical Opening”).

Prior art which appears to be within this category also includes U.S. patent applications: 20130103074 (Riina et al., Apr. 25, 2013, “Method and Apparatus for Restricting Flow Through an Opening in the Side Wall of a Body Lumen, and/or for Reinforcing a Weakness in the Side Wall of a Body Lumen, While Still Maintaining Substantially Normal Flow Through the Body Lumen”); 20130204290 (Clarke et al., Aug. 8, 2013, “Systems and Methods for Enclosing an Anatomical Opening”); 20130238083 (Duggal et al., Sep. 12, 2013, “Systems and Methods for Aneurysm Treatment and Vessel Occlusion”); 20130268046 (Gerberding et al., Oct. 10, 2013, “Systems and Methods for Supporting or Occluding a Physiological Opening or Cavity”); 20130268053 (Molaei et al., Oct. 10, 2013, “Vascular Remodeling Device”); 20130274862 (Cox et al., Oct. 17, 2013, “Methods and Devices for Treatment of Vascular Defects”); 20130274863 (Cox et al., Oct. 17, 2013, “Methods and Devices for Treatment of Vascular Defects”); and 20130274866 (Cox et al., Oct. 17, 2013, “Methods and Devices for Treatment of Vascular Defects”).

Prior art which appears to be within this category also includes U.S. patent applications: 20130274868 (Cox et al., Oct. 17, 2013, “Methods and Devices for Treatment of Vascular Defects”); 20130304109 (Abrams et al., Nov. 14, 2013, “Systems and Methods for Enclosing an Anatomical Opening, Including Coil-Tipped Aneurysm Devices”); 20140039606 (Rudakov et al., Feb. 6, 2014, “Medical Device”); 20140052233 (Cox et al., Feb. 20, 2014, “Methods and Devices for Treatment of Vascular Defects”); 20140058420 (Hannes et al., Feb. 27, 2014, “Implant Especially for the Occlusion of Bifurcation Aneurysms”); 20140121752 (Losordo et al., May 1, 2014, “Wing Bifurcation Reconstruction Device”); 20140180377 (Bose et al., Jun. 24, 2014, “Aneurysm Occlusion System and Method”); 20140236216 (Gerberding, Aug. 21, 2014, “Systems and Methods for Enclosing an Anatomical Opening, Including Shock Absorbing Aneurysm Devices”); and 20140288633 (Burke et al., Sep. 25, 2014, “Aneurysm Treatment Device and Method of Use”).

17. Aneurysm Neck Bridge or Saddle with Spherical Structure in Parent Vessel:

The prior art also discloses devices and methods for treating aneurysms comprising an aneurysm neck bridge (or saddle) with a relatively-spherical structure which is implanted within the parent vessel of an aneurysm. Prior art which appears to be within this category includes U.S. Pat. No. 5,928,260 (Chin et al., Jul. 27, 1999, “Removable Occlusion System for Aneurysm Neck”); U.S. Pat. No. 6,344,048 (Chin et al., Feb. 5, 2002, “Removable Occlusion System for Aneurysm Neck”); U.S. Pat. No. 6,428,558 (Jones et al., Aug. 6, 2002, “Aneurysm Embolization Device”); U.S. Pat. No. 6,780,196 (Chin et al., Aug. 24, 2004, “Removable Occlusion System for Aneurysm Neck”); and U.S. Pat. No. 7,229,461 (Chin et al., Jun. 12, 2007, “Removable Occlusion System for Aneurysm Neck”).

Prior art which appears to be within this category also includes U.S. patent applications: 20020042628 (Chin et al., Apr. 11, 2002, “Removable Occlusion System for Aneurysm Neck”); 20050021077 (Chin et al., Jan. 27, 2005, “Removable Occlusion System for Aneurysm Neck”); 20080221600 (Dieck et al., Sep. 11, 2008, “Isolation Devices for the Treatment of Aneurysms”); 20100023105 (Levy et al., Jan. 28, 2010, “Vascular Remodeling Device”); 20110022149 (Cox et al., Jan. 27, 2011, “Methods and Devices for Treatment of Vascular Defects”); 20110184452 (Huynh et al., Jul. 28, 2011, “Vascular Remodeling Device”); 20110184453 (Levy et al., Jul. 28, 2011, “Vascular Remodeling Device”); 20120165919 (Cox et al., Jun. 28, 2012, “Methods and Devices for Treatment of Vascular Defects”); 20130103074 (Riina et al., Apr. 25, 2013, “Method and Apparatus for Restricting Flow Through an Opening in the Side Wall of a Body Lumen, and/or for Reinforcing a Weakness in the Side Wall of a Body Lumen, While Still Maintaining Substantially Normal Flow Through the Body Lumen”); 20130274862 (Cox et al., Oct. 17, 2013, “Methods and Devices for Treatment of Vascular Defects”); 20130274863 (Cox et al., Oct. 17, 2013, “Methods and Devices for Treatment of Vascular Defects”); 20130274866 (Cox et al., Oct. 17, 2013, “Methods and Devices for Treatment of Vascular Defects”); 20130274868 (Cox et al., Oct. 17, 2013, “Methods and Devices for Treatment of Vascular Defects”); and 20140052233 (Cox et al., Feb. 20, 2014, “Methods and Devices for Treatment of Vascular Defects”).

### 18. Localized Aneurysm Neck Bridge in Parent Vessel:

Although not common, the prior art also discloses devices and methods for treating aneurysms comprising a localized aneurysm neck bridge within the parent vessel of an aneurysm which is attached to the aneurysm neck by non-compressive methods such as adhesion or barbs. Saddle-like devices in the parent vessel which are held in place against the aneurysm neck by more-extensive compressive structures in the parent vessel are included in a different category. Prior art which appears to be within this category includes U.S. Pat. No. 5,941,249 (Maynard, Aug. 24, 1999, “Distributed Activator for a Two-Dimensional Shape Memory Alloy”); U.S. Pat. No. 6,409,749 (Maynard, Jun. 25, 2002, “Aneurism Patch Including Distributed Activator for a Two-Dimensional Shape Memory Alloy”); U.S. Pat. No. 6,746,468 (Sepetka et al., Jun. 8, 2004, “Devices and Methods for Treating Vascular Malformations”); U.S. Pat. No. 7,241,301 (Thramann et al., Jul. 10, 2007, “Aneurysm Stent with Growth Factor”); U.S. Pat. No. 7,569,066 (Gerberding et al., Aug. 4, 2009, “Methods and Devices for the Treatment of Aneurysms”); U.S. Pat. No. 7,621,928 (Thramann et al., Nov. 24, 2009, “Aneurysm Stent”); and U.S. Pat. No. 8,597,320 (Sepetka et al., Dec. 3, 2013, “Devices and Methods for Treating Vascular Malformations”).

Prior art which appears to be within this category also includes U.S. patent applications: 20020169473 (Sepetka et al., Nov. 14, 2002, “Devices and Methods for Treating Vascular Malformations”); 20040181253 (Sepetka et al., Sep. 16, 2004, “Devices and Methods for Treating Vascular Malformations”); 20070225794 (Thramann et al., Sep. 27, 2007, “Aneurysm Stent”); 20100004671 (Gerberding et al., Jan. 7, 2010, “Methods and Devices for the Treatment of Aneurysms”); 20110082491 (Sepetka et al., Apr. 7, 2011, “Devices and Methods for Treating Vascular Malformations”); 20110137332 (Sepetka et al., Jun. 9, 2011, “Devices and Methods for Treating Vascular Malformations”); 20110224776 (Sepetka et al., Sep. 15, 2011, “Devices and Methods for Treating Vascular Malformations”); 20130103074 (Riina et al., Apr. 25, 2013, “Method and Apparatus for Restricting Flow Through an Opening in the Side Wall of a Body Lumen, and/or for Reinforcing a Weakness in the Side Wall of a Body Lumen, While Still Maintaining Substantially Normal Flow Through the Body Lumen”); and 20130197570 (Ebata et al., Aug. 1, 2013, “Device for Closing Luminal Cavity and Method Therefor”).

### 19. Aneurysm Neck Bridge Spanning Aneurysm Sack and Parent Vessel:

The prior art also discloses devices and methods for treating aneurysms comprising an aneurysm neck bridge which compresses the aneurysm neck from both inside and outside the aneurysm sack. In an example, such a device can have a pre-compression hourglass shape, wherein the intrasacular portion of the hourglass shape compresses the aneurysm neck from inside the sack and the parent vessel portion of the hourglass shape compresses the aneurysm neck from outside the sack. In an example, this type of device can act like an internal aneurysm clip or clamp.

Prior art which appears to be within this category includes U.S. Pat. No. 6,168,622 (Mazzocchi, Jan. 2, 2001, “Method and Apparatus for Occluding Aneurysms”); U.S. Pat. No. 6,506,204 (Mazzocchi, Jan. 14, 2003, “Method and Apparatus for Occluding Aneurysms”); U.S. Pat. No. 6,746,468 (Sepetka et al., Jun. 8, 2004, “Devices and Methods for Treating Vascular Malformations”); U.S. Pat. No. 6,855,154 (Abdel-Gawwad, Feb. 15, 2005, “Endovascular Aneurysm Treatment Device and Method”); U.S. Pat. No. 7,195,636 (Avellanet et al., Mar. 27, 2007, “Aneurysm Neck Cover for Sealing an Aneurysm”); U.S. Pat. No. 7,241,301 (Thramann et al., Jul. 10, 2007, “Aneurysm Stent with Growth Factor”); U.S. Pat. No. 7,569,066 (Gerberding et al., Aug. 4, 2009, “Methods and Devices for the Treatment of Aneurysms”); U.S. Pat. No. 7,621,928 (Thramann et al., Nov. 24, 2009, “Aneurysm Stent”); U.S. Pat. No. 7,744,652 (Morsi, Jun. 29, 2010, “Aneurysm Sealing Device”); U.S. Pat. No. 7,993,364 (Morsi, Aug. 9, 2011, “Aneurysm Flow Barrier”); U.S. Pat. No. 8,062,379 (Morsi, Nov. 22, 2011, “Aneurysm Sealing Device”); and U.S. Pat. No. 8,075,585 (Lee et al., Dec. 13, 2011, “Device and Method for Treatment of a Vascular Defect”); U.S. Pat. No. 8,292,914 (Morsi, Oct. 23, 2012, “Aneurysm Flow Barrier”).

Prior art which appears to be within this category also includes U.S. Pat. No. 8,357,180 (Feller et al., Jan. 22, 2013, “Thin Film Metallic Device for Plugging Aneurysms or Vessels”); U.S. Pat. No. 8,372,114 (Hines, Feb. 12, 2013, “Over-The-Wire Exclusion Device and System for Delivery”); U.S. Pat. No. 8,398,670 (Amplatz et al., Mar. 19, 2013, “Multi-Layer Braided Structures for Occluding Vascular Defects and for Occluding Fluid Flow Through Portions of the Vasculature of the Body”); U.S. Pat. No. 8,444,667 (Porter, May 21, 2013, “Device for Closure of a Vascular Defect and Method for Treating the Same”); U.S. Pat. No. 8,597,320 (Sepetka et al., Dec. 3, 2013, “Devices and Methods for Treating Vascular Malformations”); U.S. Pat. No. 8,668,716 (Hines, Mar. 11, 2014, “Over-the-Wire Exclusion Device and System for Delivery”); U.S. Pat. No. 8,668,717 (Hines, Mar. 11, 2014, “Over-the-Wire Exclusion Device and System for Delivery”); and U.S. Pat. No. 8,747,430 (Porter, Jun. 10, 2014, “Device for Closure of a Vascular Defect and Method for Treating the Same”).

Prior art which appears to be within this category also includes U.S. patent applications: 20010000797 (Mazzocchi, May 3, 2001, “Method and Apparatus for Occluding Aneurysms”); 20020169473 (Sepetka et al., Nov. 14, 2002, “Devices and Methods for Treating Vascular Malformations”); 20030093108 (Avellanet et al., May 15, 2003, “Aneurysm Neck Cover for Sealing an Aneurysm”); 20030195553 (Wallace, et al., Oct. 16, 2003, “System and Method for Retaining Vaso-Occlusive Devices Within an Aneurysm”); 20040181253 (Sepetka et al., Sep. 16, 2004, “Devices and Methods for Treating Vascular Malformations”); 20050228434 (Amplatz et al., Oct. 13, 2005, “Multi-Layer Braided Structures for Occluding Vascular Defects”); and 20060206196 (Porter, Sep. 14, 2006, “Device for Closure of a Vascular Defect and Method for Treating the Same”).

Prior art which appears to be within this category also includes U.S. patent applications: 20060241690 (Amplatz et al., Oct. 26, 2006, “Multi-Layer Braided Structures for Occluding Vascular Defects and for Occluding Fluid Flow Through Portions of the Vasculature of the Body”); 20070088387 (Eskridge et al., Apr. 19, 2007, “Implantable Aneurysm Closure Systems and Methods”); 20070106311 (Wallace et al., May 10, 2007, “System and Method for Retaining Vaso-Occlusive Devices Within an Aneurysm”); 20070225794 (Thramann et al., Sep. 27, 2007, “Aneurysm Stent”); 20070265656 (Amplatz et al., Nov. 15, 2007, “Multi-Layer Braided Structures for Occluding Vascular Defects”); 20080097495 (Feller et al., Apr. 24, 2008, “Thin Film Metallic Device for Plugging Aneurysms or Vessels”); and 20080200945 (Amplatz et al., Aug. 21, 2008, “Device for Occluding Vascular Defects”).

Prior art which appears to be within this category also includes U.S. patent applications: 20090062841 (Amplatz et al., Mar. 5, 2009, “Device for Occluding Vascular Defects”); 20090299326 (Morsi, Dec. 3, 2009, “Endovascular Aneurysm Treatment Device and Method”); 20100004671 (Gerberding et al., Jan. 7, 2010, “Methods and Devices for the Treatment of Aneurysms”); 20110082491 (Sepetka et al., Apr. 7, 2011, “Devices and Methods for Treating Vascular Malformations”); 20110137332 (Sepetka et al., Jun. 9, 2011, “Devices and Methods for Treating Vascular Malformations”); 20110196413 (Wallace et al., Aug. 11, 2011, “System and Method for Retaining Vaso-Occlusive Devices Within an Aneurysm”); 20110224776 (Sepetka et al., Sep. 15, 2011, “Devices and Methods for Treating Vascular Malformations”); and 20120046676 (Morsi, Feb. 23, 2012, “Aneurysm Flow Barrier”).

Prior art which appears to be within this category also includes U.S. patent applications: 20120253369 (Morsi, Oct. 4, 2012, “Advanced Endovascular Clip and Method of Using Same”); 20130012979 (Amplatz et al., Jan. 10, 2013, “Multi-Layer Braided Structures for Occluding Vascular Defects and for Occluding Fluid Flow Through Portions of the Vasculature of the Body”); 20130197547 (Fukuoka et al., Aug. 1, 2013, “Device for Closing Luminal Cavity and Method Therefor”); 20130267986 (Hines, Oct. 10, 2013, “Over-The-Wire Exclusion Device and System for Delivery”); 20130345738 (Eskridge, Dec. 26, 2013, “Endovascular Closure Device”); and 20140005698 (Eskridge, Jan. 2, 2014, “Endovascular Closure System”).

20. Aneurysm Neck Bridge Inside Aneurysm Sack with Radial Protrusions:

The prior art also discloses devices and methods for treating aneurysms comprising an aneurysm neck bridge with radially-extending loops, petals, or undulations which is implanted inside the aneurysm sack. In example, such a neck bridge can be held against the inside surface of the aneurysm neck by pressure from embolic members which are inserted into the sack. In an example, such a neck bridge can be held against the inside surface of the aneurysm neck by one or more tensile members which are in contact with the walls of the aneurysm sack.

Prior art which appears to be within this category includes U.S. Pat. No. 5,733,294 (Forber et al., Mar. 31, 1998, “Self Expanding Cardiovascular Occlusion Device, Method of Using and Method of Making the Same”); U.S. Pat. No. 5,935,148 (Villar et al., Aug. 10, 1999, “Detachable, Varying Flexibility, Aneurysm Neck Bridge”); U.S. Pat. No. 6,036,720 (Abrams et al., Mar. 14, 2000, “Sheet Metal Aneurysm Neck Bridge”); U.S. Pat. No. 6,063,070 (Eder, May 16, 2000, “Detachable Aneurysm Neck Bridge (II)”); U.S. Pat. No. 6,063,104 (Villar et al., May 16, 2000, “Detachable, Varying Flexibility, Aneurysm Neck Bridge”); U.S. Pat. No. 6,383,174 (Eder, May 7, 2002, “Detachable Aneurysm Neck Bridge (II)”); and U.S. Pat. No. 6,746,468 (Sepetka et al., Jun. 8, 2004, “Devices and Methods for Treating Vascular Malformations”).

Prior art which appears to be within this category also includes U.S. Pat. No. 7,410,482 (Murphy et al., Aug. 12, 2008, “Detachable Aneurysm Neck Bridge”); U.S. Pat. No. 7,713,264 (Murphy et al., May 11, 2010, “Detachable Aneurysm Neck Bridge”); U.S. Pat. No. 8,267,923 (Murphy et al., Sep. 18, 2012, “Detachable Aneurysm Neck Bridge”); U.S. Pat. No. 8,372,062 (Murphy et al., Feb. 12, 2013, “Detachable Aneurysm Neck Bridge”); U.S. Pat. No. 8,449,532 (Murphy et al., May 28, 2013, “Detachable Aneurysm Neck Bridge”); U.S. Pat. No. 8,529,556 (Murphy et al., Sep. 10, 2013, “Detachable Aneurysm Neck Bridge”); and U.S. Pat. No. 8,597,320 (Sepetka et al., Dec. 3, 2013, “Devices and Methods for Treating Vascular Malformations”).

Prior art which appears to be within this category also includes U.S. patent applications: 20020169473 (Sepetka et al., Nov. 14, 2002, “Devices and Methods for Treating Vascular Malformations”); 20040093014 (Ho et al., May 13, 2004, “Bioactive Components for Incorporation with Vaso-Occlusive Members”); 20040098027 (Teoh et al., May 20, 2004, “Expandable Body Cavity Liner Device”); 20040181253 (Sepetka et al., Sep. 16, 2004, “Devices and Methods for Treating Vascular Malformations”); 20070088387 (Eskridge et al., Apr. 19, 2007, “Implantable Aneurysm Closure Systems and Methods”); 20100222804 (Murphy et al., Sep. 2, 2010, “Detachable Aneurysm Neck Bridge”); 20110022149 (Cox et al., Jan. 27, 2011, “Methods and Devices for Treatment of Vascular Defects”); 20110082491 (Sepetka et al., Apr. 7, 2011, “Devices and Methods for Treating Vascular Malformations”); 20110137332 (Sepetka et al., Jun. 9, 2011, “Devices and Methods for Treating Vascular Malformations”); and 20110224776 (Sepetka et al., Sep. 15, 2011, “Devices and Methods for Treating Vascular Malformations”).

Prior art which appears to be within this category also includes U.S. patent applications: 20110282378 (Murphy et al., Nov. 17, 2011, “Detachable Aneurysm Neck Bridge”); 20120165919 (Cox et al., Jun. 28, 2012, “Methods and Devices for Treatment of Vascular Defects”); 20120310270 (Murphy et al., Feb. 12, 2013, “Detachable Aneurysm Neck Bridge”); 20130035712 (Theobald et al., Feb. 7, 2013, “Cerebral Aneurysm Closure Device”); 20130190800 (Murphy et al., Jul. 25, 2013, “Detachable Aneurysm Neck Bridge”); 20130274862 (Cox et al., Oct. 17, 2013, “Methods and Devices for Treatment of Vascular Defects”); 20130274863 (Cox et al., Oct. 17, 2013, “Methods and Devices for Treatment of Vascular Defects”); 20130274866 (Cox et al., Oct. 17, 2013, “Methods and Devices for Treatment of Vascular Defects”); 20130274868 (Cox et al., Oct. 17, 2013, “Methods and Devices for Treatment of Vascular Defects”); 20140052233 (Cox et al., Feb. 20, 2014, “Methods and Devices for Treatment of Vascular Defects”); and 20140207162 (Tran et al., Jul. 24, 2014, “Occlusive Cinching Devices and Methods of Use”).

21. Aneurysm Neck Bridge Inside Aneurysm Sack with Convex Shape:

The prior art also discloses devices and methods for treating aneurysms comprising an aneurysm neck bridge with a bowl shape, cup shape, or other generally-convex shape which is implanted inside the aneurysm sack. In example, such a neck bridge can be held against the inside surface of the aneurysm neck by pressure from embolic members which are inserted into the sack. In an example, such a neck bridge can be held against the inside surface of the aneurysm neck by one or more tensile members which are in contact with the walls of the aneurysm sack.

Prior art which appears to be within this category includes U.S. Pat. No. 6,168,615 (Ken et al., Jan. 2, 2001, “Method and Apparatus for Occlusion and Reinforcement of Aneurysms”); U.S. Pat. No. 6,454,780 (Wallace, Sep. 24, 2002, “Aneurysm Neck Obstruction Device”); U.S. Pat. No. 6,605,111 (Bose et al., Aug. 12, 2003, “Endovascular Thin Film Devices and Methods for Treating and Preventing Stroke”); U.S. Pat. No. 6,802,851 (Jones et al., Oct. 12, 2004, “Stent Aneurysm Embolization Method Using Collapsible Member and Embolic Coils”); U.S. Pat. No. 6,811,560 (Jones et al., Nov. 2, 2004, “Stent Aneurysm Embolization Method and Device”); U.S. Pat. No. 7,083,632 (Avellanet et al., Aug. 1, 2006, “Aneurysm Embolic Device with an Occlusive Member”); U.S. Pat. No. 7,128,736 (Abrams et al., Oct. 31, 2006, “Detachable Aneurysm Neck Closure Patch”); and U.S. Pat. No. 7,241,301 (Thramann et al., Jul. 10, 2007, “Aneurysm Stent with Growth Factor”).

Prior art which appears to be within this category also includes U.S. Pat. No. 7,306,622 (Jones et al., Dec. 11, 2007, “Stent Embolization Device”); U.S. Pat. No. 7,569,066 (Gerberding et al., Aug. 4, 2009, “Methods and Devices for the Treatment of Aneurysms”); U.S. Pat. No. 7,572,288 (Cox, Aug. 11, 2009, “Aneurysm Treatment Device and Method of Use”); U.S. Pat. No. 7,608,088 (Jones et al., Oct. 27, 2009, “Stent Aneurysm Embolization Device”); U.S. Pat. No. 7,621,928 (Thramann et al., Nov. 24, 2009, “Aneurysm Stent”); U.S. Pat. No. 8,252,040 (Cox, Aug. 28, 2012, “Aneurysm Treatment Device and Method of Use”); U.S. Pat. No. 8,444,667 (Porter, May 21, 2013, “Device for Closure of a Vascular Defect and Method for Treating the Same”); U.S. Pat. No. 8,562,667 (Cox, Oct. 22, 2013, “Aneurysm Treatment Device and Method of Use”); U.S. Pat. No. 8,715,312 (Burke et al., May 6, 2014, “Aneurysm Treatment Device and Method of Use”); and U.S. Pat. No. 8,747,430 (Porter, Jun. 10, 2014, “Device for Closure of a Vascular Defect and Method for Treating the Same”).

Prior art which appears to be within this category also includes U.S. patent applications: 20030018294 (Cox, Jan. 23, 2003, “Aneurysm Treatment Device and Method of Use”); 20030083676 (Wallace, May 1, 2003, “Aneurysm Neck Obstruction Device”); 20030093097 (Avellanet et al., May 15, 2003, “Aneurysm Embolic Device with an Occlusive Member”); 20030181927 (Wallace, Sep. 25, 2003, “Aneurysm Neck Obstruction Device”); 20030195553 (Wallace, et al., Oct. 16, 2003, “System and Method for Retaining Vaso-Occlusive Devices Within an Aneurysm”); 20040098027 (Teoh et al., May 20, 2004, “Expandable Body Cavity Liner Device”); 20050033349 (Jones et al., Feb. 10, 2005, “Stent Aneurysm Embolization Device”); 20060155323 (Porter et al., Jul. 13, 2006, “Intra-Aneurysm Devices”); and 20060206196 (Porter, Sep. 14, 2006, “Device for Closure of a Vascular Defect and Method for Treating the Same”).

Prior art which appears to be within this category also includes U.S. patent applications: 20060235464 (Avellanet et al., Oct. 19, 2006, “Aneurysm Embolic Device with an Occlusive Member”); 20070088387 (Eskridge et al., Apr. 19, 2007, “Implantable Aneurysm Closure Systems and Methods”); 20070106311 (Wallace et al., May 10, 2007, “System and Method for Retaining Vaso-Occlusive Devices Within an Aneurysm”); 20070225794 (Thramann et al., Sep. 27, 2007, “Aneurysm Stent”); 20080147100 (Wallace, Jun. 19, 2008, “Aneurysm Neck Obstruction Device”); 20080281350 (Sepetka et al., Nov. 13, 2008, “Aneurysm Occlusion Devices”); 20100004671 (Gerberding et al., Jan. 7, 2010, “Methods and Devices for the Treatment of Aneurysms”); 20110022149 (Cox et al., Jan. 27, 2011, “Methods and Devices for Treatment of Vascular Defects”); 20110144669 (Becking et al., Jun. 16, 2011, “Aneurysm Cover Device for Embolic Delivery and Retention”); and 20110196413 (Wallace et al., Aug. 11, 2011, “System and Method for Retaining Vaso-Occlusive Devices Within an Aneurysm”).

Prior art which appears to be within this category also includes U.S. patent applications: 20120143237 (Cam et al., Jun. 7, 2012, “Vascular Remodeling Device”); 20120143317 (Cam et al., Jun. 7, 2012, “Vascular Remodeling Device”); 20120165919 (Cox et al., Jun. 28, 2012, “Methods and Devices for Treatment of Vascular Defects”); 20130274862 (Cox et al., Oct. 17, 2013, “Methods and Devices for Treatment of Vascular Defects”); 20130274863 (Cox et al., Oct. 17, 2013, “Methods and Devices for Treatment of Vascular Defects”); 20130274866 (Cox et al., Oct. 17, 2013, “Methods and Devices for Treatment of Vascular Defects”); 20130274868 (Cox et al., Oct. 17, 2013, “Methods and Devices for Treatment of Vascular Defects”); 20130325053 (Porter et al., Dec. 5, 2013, “Intra-Aneurysm Devices”); 20140052233 (Cox et al., Feb. 20, 2014, “Methods and Devices for Treatment of Vascular Defects”); 20140135810 (Divino et al., May 15, 2014, “Occlusive Devices”); 20140135811 (Divino et al., May 15, 2014, “Occlusive Devices”); 20140135812 (Divino et al., May 15, 2014, “Occlusive Devices”); and 20140288633 (Burke et al., Sep. 25, 2014, “Aneurysm Treatment Device and Method of Use”).

### 22. Single-Chamber Woven/Mesh Structure in Aneurysm Sack:

The prior art also discloses devices and methods for treating aneurysms comprising single-chamber woven and/or mesh structures which are expanded within the aneurysm sack. In an example, such structures can be formed from arcuate metal wires or filaments. Generally, such structures are expanded into a hollow, but compression-resilient, geometric shape within the aneurysm sack. Relatively spherical, ellipsoid, and toroidal shapes are common To paraphrase Jerry Lee Lewis—“Goodness. Gracious. Great balls of wire!”

Prior art which appears to be within this category includes U.S. Pat. No. 5,766,219 (Horton, Jun. 16, 1998, “Anatomically Shaped Vasoocclusive Device and Method for Deploying Same”); U.S. Pat. No. 5,911,731 (Pham et al., Jun. 15, 1999, “Anatomically Shaped Vasoocclusive Devices”); U.S. Pat. No. 6,346,117 (Greenhalgh, Feb. 12, 2002, “Bag for Use in the Intravascular Treatment of Saccular Aneurysms”); U.S. Pat. No. 6,375,668 (Gifford et al., Apr. 23, 2002, “Devices and Methods for Treating Vascular Malformations”); U.S. Pat. No. 6,391,037 (Greenhalgh, May 21, 2002, “Bag for Use in the Intravascular Treatment of Saccular Aneurysms”); U.S. Pat. No. 6,746,468 (Sepetka et al., Jun. 8, 2004, “Devices and Methods for Treating Vascular Malformations”); U.S. Pat. No. 8,142,456 (Rosqueta et al., Mar. 27, 2012, “Braid-Ball Embolic Devices”); U.S. Pat. No. 8,597,320 (Sepetka et al., Dec. 3, 2013, “Devices and Methods for Treating Vascular Malformations”); U.S. Pat. No. 8,597,323 (Plaza et al., Dec. 3, 2013, “Delivery and Detachment Systems and Methods for Vascular Implants”); U.S. Pat. No. 8,696,701 (Becking et al., Apr. 15, 2014, “Braid-Ball Embolic Devices”); and U.S. Pat. No. 8,747,597 (Rosqueta et al., Jun. 10, 2014, “Methods for Making Braid-Ball Occlusion Devices”).

Prior art which appears to be within this category also includes U.S. patent applications: 20020169473 (Sepetka et al., Nov. 14, 2002, “Devices and Methods for Treating Vascular Malformations”); 20040098027 (Teoh et al., May 20, 2004, “Expandable Body Cavity Liner Device”); 20040181253 (Sepetka et al., Sep. 16, 2004, “Devices and Methods for Treating Vascular Malformations”); 20060052816 (Bates et al., Mar. 9, 2006, “Device for Treating an Aneurysm”); 20060155323 (Porter et al., Jul. 13, 2006, “Intra-Aneurysm Devices”); 20090275974 (Marchand et al., Nov. 5, 2009, “Filamentary Devices for Treatment of Vascular Defects”); 20090287291 (Becking et al., Nov. 19, 2009, “Embolic Device Delivery Systems”); 20090287294 (Rosqueta et al., Nov. 19, 2009, “Braid-Ball Embolic Devices”); 20090318941 (Sepetka et al., Dec. 24, 2009, “Self-Expandable Endovascular Device for Aneurysm Occlusion”); and 20100069948 (Veznedaroglu et al., Mar. 18, 2010, “Self-Expandable Aneurysm Filling Device System and Method of Placement”).

Prior art which appears to be within this category also includes U.S. patent applications: 20110022149 (Cox et al., Jan. 27, 2011, “Methods and Devices for Treatment of Vascular Defects”); 20110082491 (Sepetka et al., Apr. 7, 2011, “Devices and Methods for Treating Vascular Malformations”); 20110137332 (Sepetka et al., Jun. 9, 2011, “Devices and Methods for Treating Vascular Malformations”); 20110152993 (Marchand et al., Jun. 23, 2011, “Multiple Layer Filamentary Devices for Treatment of Vascular Defects”); 20110202085 (Loganathan et al., Aug. 18, 2011, “Braid Ball Embolic Device Features”); 20110208227 (Becking, Aug. 25, 2011, “Filamentary Devices for Treatment of Vascular Defects”); 20110224776 (Sepetka et al., Sep. 15, 2011, “Devices and Methods for Treating Vascular Malformations”); 20110265943 (Rosqueta et al., Nov. 3, 2011, “Methods for Making Braid-Ball Occlusion Devices”); 20110319926 (Becking et al., Dec. 29, 2011, “Braid Ball Embolic Device Features”); and 20120071911 (Sadasivan et al., Mar. 22, 2012, “Spherical Helix Embolic Coils for the Treatment of Cerebral Aneurysms”).

Prior art which appears to be within this category also includes U.S. patent applications: 20120165919 (Cox et al., Jun. 28, 2012, “Methods and Devices for Treatment of Vascular Defects”); 20120197283 (Marchand et al., Aug. 2, 2012, “Multiple Layer Filamentary Devices for Treatment of Vascular Defects”); 20120283768 (Cox et al., Nov. 8, 2012, “Method and Apparatus for the Treatment of Large and Giant Vascular Defects”); 20120316598 (Becking et al., Dec. 13, 2012, “Multiple Layer Filamentary Devices for Treatment of Vascular Defects”); 20120330347 (Becking et al., Dec. 27, 2012, “Multiple Layer Filamentary Devices for Treatment of Vascular Defects”); 20130066360 (Becking et al., Mar. 14, 2013, “Braid-Ball Embolic Devices”); 20130085522 (Becking et al., Apr. 4, 2013, “Braid-Ball Embolic Devices”); and 20130103074 (Riina et al., Apr. 25, 2013, “Method and Apparatus for Restricting Flow Through an Opening in the Side Wall of a Body Lumen, and/or for Reinforcing a Weakness in the Side Wall of a Body Lumen, While Still Maintaining Substantially Normal Flow Through the Body Lumen”).

Prior art which appears to be within this category also includes U.S. patent applications: 20130123830 (Becking et al., May 16, 2013, “Multiple Layer Filamentary Devices for Treatment of Vascular Defects”); 20130245667 (Marchand et al., Sep. 19, 2013, “Filamentary Devices and Treatment of Vascular Defects”); 20130274862 (Cox et al., Oct. 17, 2013, “Methods and Devices for Treatment of Vascular Defects”); 20130274863 (Cox et al., Oct. 17, 2013, “Methods and Devices for Treatment of Vascular Defects”); 20130274866 (Cox et al., Oct. 17, 2013, “Methods and Devices for Treatment of Vascular Defects”); 20130274868 (Cox et al., Oct. 17, 2013, “Methods and Devices for Treatment of Vascular Defects”); 20130325053 (Porter et al., Dec. 5, 2013, “Intra-Aneurysm Devices”); 20140012307 (Franano et al., Jan. 9, 2014, “Detachable Metal Balloon Delivery Device and Method”); 20140012363 (Franano et al., Jan. 9, 2014, “Ballstent Device and Methods of Use”); and 20140052233 (Cox et al., Feb. 20, 2014, “Methods and Devices for Treatment of Vascular Defects”).

Prior art which appears to be within this category also includes U.S. patent applications: 20140135810 (Divino et al., May 15, 2014, “Occlusive Devices”); 20140135811 (Divino et al., May 15, 2014, “Occlusive Devices”); 20140135812 (Divino et al., May 15, 2014, “Occlusive Devices”); 20140142611 (Plaza et al., May 22, 2014, “Delivery and Detachment Systems and Methods for Vascular Implants”); 20140172001 (Becking et al., Jun. 19, 2014, “Two-Stage Deployment Aneurysm Embolization Devices”); and 20140200607 (Sepetka et al., Jul. 17, 2014, “Occlusive Device”).

### 23. Multi-Chamber Woven/Mesh Structure in Aneurysm Sack:

The prior art also discloses devices and methods for treating aneurysms comprising multi-chamber woven and/or mesh structures which are expanded within the aneurysm sack. In an example, such structures can be formed from arcuate metal wires or filaments. Generally, such structures are expanded into one or more hollow, but compression-resilient, geometric shapes within the aneurysm sack. Prior art which appears to be within this category includes U.S. Pat. No. 6,168,622 (Mazzocchi, Jan. 2, 2001, “Method and Apparatus for Occluding Aneurysms”); U.S. Pat. No. 6,375,668 (Gifford et al., Apr. 23, 2002, “Devices and Methods for Treating Vascular Malformations”); U.S. Pat. No. 6,506,204 (Mazzocchi, Jan. 14, 2003, “Method and Apparatus for Occluding Aneurysms”); U.S. Pat. No. 6,746,468 (Sepetka et al., Jun. 8, 2004, “Devices and Methods for Treating Vascular Malformations”); U.S. Pat. No. 7,993,364 (Morsi, Aug. 9, 2011, “Aneurysm Flow Barrier”); U.S. Pat. No. 8,066,036 (Monetti et al., Nov. 29, 2011, “Three-Dimensional Complex Coil”); U.S. Pat. No. 8,292,914 (Morsi, Oct. 23, 2012, “Aneurysm Flow Barrier”); and U.S. Pat. No. 8,597,320 (Sepetka et al., Dec. 3, 2013, “Devices and Methods for Treating Vascular Malformations”).

Prior art which appears to be within this category also includes U.S. patent applications: 20010000797 (Mazzocchi, May 3, 2001, “Method and Apparatus for Occluding Aneurysms”); 20020169473 (Sepetka et al., Nov. 14, 2002, “Devices and Methods for Treating Vascular Malformations”); 20040181253 (Sepetka et al., Sep. 16, 2004, “Devices and Methods for Treating Vascular Malformations”); 20060052816 (Bates et al., Mar. 9, 2006, “Device for Treating an Aneurysm”); 20070175536 (Monetti et al., Aug. 2, 2007, “Three-Dimensional Complex Coil”); 20090297582 (Meyer et al., Dec. 3, 2009, “Vascular Occlusion Devices and Methods”); 20090299326 (Morsi, Dec. 3, 2009, “Endovascular Aneurysm Treatment Device and Method”); 20110082491 (Sepetka et al., Apr. 7, 2011, “Devices and Methods for Treating Vascular Malformations”); 20110098814 (Monstadt et al., Apr. 28, 2011, “Medical Implant”); 20110137332 (Sepetka et al., Jun. 9, 2011, “Devices and Methods for Treating Vascular Malformations”); and 20110224776 (Sepetka et al., Sep. 15, 2011, “Devices and Methods for Treating Vascular Malformations”).

Prior art which appears to be within this category also includes U.S. patent applications: 20120046676 (Morsi, Feb. 23, 2012, “Aneurysm Flow Barrier”); 20120071911 (Sadasivan et al., Mar. 22, 2012, “Spherical Helix Embolic Coils for the Treatment of Cerebral Aneurysms”); 20120283768 (Cox et al., Nov. 8, 2012, “Method and Apparatus for the Treatment of Large and Giant Vascular Defects”); 20120330341 (Becking et al., Dec. 27, 2012, “Folded-Flat Aneurysm Embolization Devices”); 20130089576 (Maitland et al., Apr. 11, 2013, “Implantable Embolic Scaffolds that Promote Healing”); 20130204289 (Dasnurkar et al., Aug. 8, 2013, “Devices and Methods for Occluding Vascular Abnormalities”); 20140012303 (Heipl, Jan. 9, 2014, “Braided Medical Device and Manufacturing Method Thereof”); 20140047694 (Monstadt et al., Feb. 20, 2014, “Medical Implant”); 20140135810 (Divino et al., May 15, 2014, “Occlusive Devices”); 20140135811 (Divino et al., May 15, 2014, “Occlusive Devices”); 20140135812 (Divino et al., May 15, 2014, “Occlusive Devices”); and 20140296358 (Maitland et al., Oct. 2, 2014, “Implantable Embolic Scaffolds That Promote Healing”).

24. Embolic Coils with Relatively-Traditional Structures in Aneurysm Sack:

The prior art also includes some relatively-early embolic coils and also embolic coils with relatively-traditional structures which are implanted within an aneurysm sack. Some of these examples in the prior art represent early, pioneering use of embolic coils for occluding aneurysms. Some of these examples in the prior art use relatively-traditional embolic coils, but use them in novel ways or in novel combinations with other device components. Prior art which appears to be within this category includes U.S. Pat. No. 4,994,069 (Ritchart et al., Feb. 19, 1991, “Vaso-Occlusion Coil and Method”); U.S. Pat. No. 5,423,829 (Pham et al., Jun. 13, 1995, “Electrolytically Severable Joint for Endovascular Embolic Devices”); U.S. Pat. No. 5,624,449 (Pham et al., Apr. 29, 1997, “Electrolytically Severable Joint for Endovascular Embolic Devices”); U.S. Pat. No. 5,639,277 (Mariant et al., Jun. 17, 1997, “Embolic Coils with Offset Helical and Twisted Helical Shapes”); U.S. Pat. No. 5,690,666 (Berenstein et al., Nov. 25, 1997, “Ultrasoft Embolism Coils and Process for Using Them”); U.S. Pat. No. 5,718,711 (Berenstein et al., Feb. 17, 1998, “Ultrasoft Embolism Devices and Process for Using Them”); U.S. Pat. No. 5,743,905 (Eder et al., Apr. 28, 1998, “Partially Insulated Occlusion Device”); U.S. Pat. No. 5,749,894 (Engelson, May 12, 1998, “Aneurysm Closure Method”); U.S. Pat. No. 5,800,453 (Gia, Sep. 1, 1998, “Detachable Embolic Coil Assembly Using Interlocking Hooks and Slots”); U.S. Pat. No. 5,826,587 (Berenstein et al., Oct. 27, 1998, “Ultrasoft Embolism Coils and Process for Using Them”); and U.S. Pat. No. 5,916,235 (Guglielmi, Jun. 29, 1999, “Apparatus and Method for the Use of Detachable Coils in Vascular Aneurysms and Body Cavities”).

Prior art which appears to be within this category also includes U.S. Pat. No. 6,024,754 (Engelson, Feb. 15, 2000, “Aneurysm Closure Method”); U.S. Pat. No. 6,099,546 (Gia, Aug. 8, 2000, “Detachable Embolic Coil Assembly Using Interlocking Hooks and Slots”); U.S. Pat. No. 6,123,714 (Gia et al., Sep. 26, 2000, “System for Detaching an Occlusive Device Within a Body Using a Solderless, Electrolytically Severable Joint”); U.S. Pat. No. 6,350,270 (Roue, Feb. 26, 2002, “Aneurysm Liner”); U.S. Pat. No. 6,409,721 (Wheelock et al., Jun. 25, 2002, “Process for Forming an Occlusion in a Body Cavity”); U.S. Pat. No. 6,458,119 (Berenstein et al., Oct. 1, 2002, “Ultrasoft Embolism Devices and Process for Using Them”); U.S. Pat. No. 6,585,748 (Jeffree, Jul. 1, 2003, “Device for Treating Aneurysms”); U.S. Pat. No. 6,589,230 (Gia et al., Jul. 8, 2003, “System for Detaching an Occlusive Device Within a Mammalian Body Using a Solderless, Electrolytically Severable Joint”); U.S. Pat. No. 6,855,153 (Saadat, Feb. 15, 2005, “Embolic Balloon”); U.S. Pat. No. 7,153,323 (Teoh et al., Dec. 26, 2006, “Aneurysm Liner with Multi-Segment Extender”); U.S. Pat. No. 7,238,194 (Monstadt et al., Jul. 3, 2007, “Device for Implanting Occlusion Spirals”); U.S. Pat. No. 7,294,137 (Rivelli et al., Nov. 13, 2007, “Device for Multi-Modal Treatment of Vascular Lesions”); U.S. Pat. No. 7,410,482 (Murphy et al., Aug. 12, 2008, “Detachable Aneurysm Neck Bridge”); and U.S. Pat. No. 7,691,124 (Balgobin, Apr. 6, 2010, “Delivery of Therapeutic Devices”).

Prior art which appears to be within this category also includes U.S. Pat. No. 7,695,488 (Berenstein et al., Apr. 13, 2010, “Expandable Body Cavity Liner Device”); U.S. Pat. No. 7,708,754 (Balgobin et al., May 4, 2010, “Stretch Resistant Embolic Coil Delivery System with Mechanical Release Mechanism”); U.S. Pat. No. 7,713,264 (Murphy et al., May 11, 2010, “Detachable Aneurysm Neck Bridge”); U.S. Pat. No. 7,799,052 (Balgobin et al., Sep. 21, 2010, “Stretch Resistant Embolic Coil Delivery System with Mechanical Release Mechanism”); U.S. Pat. No. 7,811,305 (Balgobin et al., Oct. 12, 2010, “Stretch Resistant Embolic Coil Delivery System with Spring Release Mechanism”); U.S. Pat. No. 7,819,891 (Balgobin et al., Oct. 26, 2010, “Stretch Resistant Embolic Coil Delivery System with Spring Release Mechanism”); U.S. Pat. No. 7,819,892 (Balgobin et al., Oct. 26, 2010, “Embolic Coil Delivery System with Spring Wire Release Mechanism”); U.S. Pat. No. 7,985,238 (Balgobin et al., Jul. 26, 2011, “Embolic Coil Delivery System with Spring Wire Release Mechanism”); U.S. Pat. No. 8,021,416 (Abrams, Sep. 20, 2011, “Methods for Delivering a Prosthesis to a Site in a Body”); and U.S. Pat. No. 8,221,447 (Solar et al., Jul. 17, 2012, “Aneurysm Coil Delivery System”).

Prior art which appears to be within this category also includes U.S. Pat. No. 8,267,923 (Murphy et al., Sep. 18, 2012, “Detachable Aneurysm Neck Bridge”); U.S. Pat. No. 8,361,106 (Solar et al., Jan. 29, 2013, “Aneurysm Coil Delivery System”); U.S. Pat. No. 8,372,062 (Murphy et al., Feb. 12, 2013, “Detachable Aneurysm Neck Bridge”); U.S. Pat. No. 8,425,542 (Moftakhar et al., Apr. 23, 2013, “Aneurysm Occlusion Device Containing Bioactive and Biocompatible Copolymer Shell and Biocompatible Metallic Frame Member”); U.S. Pat. No. 8,449,532 (Murphy et al., May 28, 2013, “Detachable Aneurysm Neck Bridge”); U.S. Pat. No. 8,529,556 (Murphy et al., Sep. 10, 2013, “Detachable Aneurysm Neck Bridge”); U.S. Pat. No. 8,529,619 (Abrams, Sep. 10, 2013, “Methods for Delivering a Prosthesis to a Site in a Body”); and U.S. Pat. No. 8,597,321 (Monstadt et al., Dec. 3, 2013, “Device for the Implantation of Occlusion Spirals”).

Prior art which appears to be within this category also includes U.S. patent applications: 20030187473 (Berenstein et al., Oct. 2, 2003, “Expandable Body Cavity Liner Device”); 20040098027 (Teoh et al., May 20, 2004, “Expandable Body Cavity Liner Device”); 20070219578 (Solar et al., Sep. 20, 2007, “Aneurysm Coil Delivery System”); 20080281350 (Sepetka et al., Nov. 13, 2008, “Aneurysm Occlusion Devices”); 20090062834 (Moftakhar et al., Mar. 5, 2009, “Aneurysm Occlusion Device Containing Bioactive and Biocompatible Copolymer Shell and Biocompatible Metallic Frame Member”); 20100168781 (Berenstein et al., Jul. 1, 2010, “Expandable Body Cavity Liner Device”); 20100222804 (Murphy et al., Sep. 2, 2010, “Detachable Aneurysm Neck Bridge”); and 20110282378 (Murphy et al., Nov. 17, 2011, “Detachable Aneurysm Neck Bridge”).

Prior art which appears to be within this category also includes U.S. patent applications: 20120283764 (Solar et al., Nov. 8, 2012, “Aneurysm Coil Delivery System”); 20120310270 (Murphy et al., Feb. 12, 2013, “Detachable Aneurysm Neck Bridge”); 20120310271 (Kwon, Dec. 6, 2012, “Embolus-Forming In-Vivo Indwelling Coil and Method for Manufacturing an Embolus-Forming In-Vivo Indwelling Coil”); 20130190800 (Murphy et al., Jul. 25, 2013, “Detachable Aneurysm Neck Bridge”); 20130261730 (Bose et al., Oct. 3, 2013, “Aneurysm Occlusion System and Method”); 20140081313 (Elliott, J., Mar. 20, 2014, “Embolic Coils and Related Components, Systems, and Methods”); and 20140180377 (Bose et al., Jun. 24, 2014, “Aneurysm Occlusion System and Method”).

25. Embolic Coils with Complex Structures in Aneurysm Sack:

There are a large number of coil structures and configurations which are disclosed in the prior art. Some of these are not easily matched to a reasonable number of general device categories. Accordingly, this category is a general one for prior art which discloses coils with complex structures which do not fit well into other coil categories. Prior art which appears to be within this category includes U.S. Pat. No. 5,350,397 (Palermo et al., Sep. 27, 1994, “Axially Detachable Embolic Coil Assembly”); U.S. Pat. No. 5,382,259 (Phelps et al., Jan. 17, 1995, “Vasoocclusion Coil with Attached Tubular Woven or Braided Fibrous Covering”); U.S. Pat. No. 5,522,822 (Phelps et al., Jun. 4, 1996, “Vasoocclusion Coil with Attached Tubular Woven or Braided Fibrous Covering”); U.S. Pat. No. 5,582,619 (Ken, Dec. 10, 1996, “Stretch Resistant Vaso-Occlusive Coils”); U.S. Pat. No. 5,624,461 (Mariant, Apr. 29, 1997, “Three Dimensional In-Filling Vaso-Occlusive Coils”); U.S. Pat. No. 5,649,949 (Wallace et al., Jul. 22, 1997, “Variable Cross-Section Conical Vasoocclusive Coils”); U.S. Pat. No. 5,733,329 (Wallace et al., Mar. 31, 1998, “Vaso-Occlusive Coil with Conical End”); U.S. Pat. No. 5,749,891 (Ken et al., May 12, 1998, “Multiple Layered Vaso-Occlusive Coils”); U.S. Pat. No. 5,766,160 (Samson et al., Jun. 16, 1998, “Variable Stiffness Coils”); U.S. Pat. No. 5,800,455 (Palermo et al., Sep. 1, 1998, “Detachable Embolic Coil Assembly”); U.S. Pat. No. 5,833,705 (Ken et al., Nov. 10, 1998, “Stretch Resistant Vaso-Occlusive Coils”); U.S. Pat. No. 5,853,418 (Ken et al., Dec. 29, 1998, “Stretch Resistant Vaso-Occlusive Coils (II)”); U.S. Pat. No. 5,891,130 (Palermo et al., Apr. 6, 1999, “Axially Detachable Embolic Coil Assembly”); and U.S. Pat. No. 5,911,731 (Pham et al., Jun. 15, 1999, “Anatomically Shaped Vasoocclusive Devices”).

Prior art which appears to be within this category also includes U.S. Pat. No. 5,925,059 (Palermo et al., Jul. 20, 1999, “Detachable Embolic Coil Assembly”); U.S. Pat. No. 5,941,888 (Wallace et al., Aug. 24, 1999, “Vaso-Occlusive Member Assembly with Multiple Detaching Points”); U.S. Pat. No. 5,957,948 (Mariant, Sep. 28, 1999, “Three Dimensional In-Filling Vaso-Occlusive Coils”); U.S. Pat. No. 5,980,514 (Kupiecki et al., Nov. 9, 1999, “Aneurysm Closure Device Assembly”); U.S. Pat. No. 6,004,338 (Ken et al., Dec. 21, 1999, “Stretch Resistant Vaso-Occlusive Coils”); U.S. Pat. No. 6,013,084 (Ken et al., Jan. 11, 2000, “Stretch Resistant Vaso-Occlusive Coils (II)”); U.S. Pat. No. 6,024,765 (Wallace et al., Feb. 15, 2000, “Vaso-Occlusive Coil with Conical End”); U.S. Pat. No. 6,033,423 (Ken et al., Mar. 7, 2000, “Multiple Layered Vaso-Occlusive Coils”); U.S. Pat. No. 6,096,034 (Kupiecki et al., Aug. 1, 2000, “Aneurysm Closure Device Assembly”); U.S. Pat. No. 6,143,007 (Mariant et al., Nov. 7, 2000, “Method for Making an Occlusive Device”); U.S. Pat. No. 6,159,165 (Ferrera et al., Dec. 12, 2000, “Three Dimensional Spherical Micro-Coils Manufactured from Radiopaque Nickel-Titanium Microstrand”); U.S. Pat. No. 6,168,592 (Kupiecki et al., Jan. 2, 2001, “Aneurysm Closure Device Assembly”); U.S. Pat. No. 6,187,027 (Mariant et al., Feb. 13, 2001, “Vaso-Occlusive Devices with Heat Secured Polymer Fiber”); and U.S. Pat. No. 6,193,728 (Ken et al., Feb. 27, 2001, “Stretch Resistant Vaso-Occlusive Coils (II)”).

Prior art which appears to be within this category also includes U.S. Pat. No. 6,221,066 (Ferrera et al., Apr. 24, 2001, “Shape Memory Segmented Detachable Coil”); U.S. Pat. No. 6,231,586 (Mariant, May 15, 2001, “Three Dimensional In-Filling Vaso-Occlusive Coils”); U.S. Pat. No. 6,254,592 (Samson et al., Jul. 3, 2001, “Variable Stiffness Coils”); U.S. Pat. No. 6,280,457 (Wallace et al., Aug. 28, 2001, “Polymer Covered Vaso-Occlusive Devices and Methods of Producing Such Devices”); U.S. Pat. No. 6,344,041 (Kupiecki et al., Feb. 5, 2002, “Aneurysm Closure Device Assembly”); U.S. Pat. No. 6,371,972 (Wallace et al., Apr. 16, 2002, “Vaso-Occlusive Member Assembly with Multiple Detaching Points”); U.S. Pat. No. 6,533,801 (Wallace et al., Mar. 18, 2003, “Vaso-Occlusive Member Assembly with Multiple Detaching Points”); U.S. Pat. No. 6,551,305 (Ferrera et al., Apr. 22, 2003, “Shape Memory Segmented Detachable Coil”); U.S. Pat. No. 6,605,101 (Schaefer et al., Aug. 12, 2003, “Microcoil Vaso-Occlusive Device with Multi-Axis Secondary Configuration”); and U.S. Pat. No. 6,616,617 (Ferrera et al., Sep. 9, 2003, “Vasoocclusive Device for Treatment of Aneurysms”).

Prior art which appears to be within this category also includes U.S. Pat. No. 6,623,493 (Wallace et al., Sep. 23, 2003, “Vaso-Occlusive Member Assembly with Multiple Detaching Points”); U.S. Pat. No. 6,660,020 (Wallace et al., Dec. 9, 2003, “Vaso-Occlusive Coil with Conical End”); U.S. Pat. No. 6,723,108 (Jones et al., Apr. 20, 2004, “Foam Matrix Embolization Device”); U.S. Pat. No. 6,979,344 (Jones et al., Dec. 27, 2005, “Foam Matrix Embolization Device”); U.S. Pat. No. 6,984,240 (Ken et al., Jan. 10, 2006, “Detachable Multidiameter Vasoocclusive Coil”); U.S. Pat. No. 7,029,486 (Schaefer et al., Apr. 18, 2006, “Microcoil Vaso-Occlusive Device with Multi-Axis Secondary Configuration”); U.S. Pat. No. 7,033,374 (Schaefer et al., Apr. 25, 2006, “Microcoil Vaso-Occlusive Device with Multi-Axis Secondary Configuration”); U.S. Pat. No. 7,326,225 (Ferrera et al., Feb. 5, 2008, “Vasoocclusive Device for Treatment of Aneurysms”); U.S. Pat. No. 7,331,974 (Schaefer et al., Feb. 19, 2008, “Microcoil Vaso-Occlusive Device with Multi-Axis Secondary Configuration”); U.S. Pat. No. 7,485,123 (Porter, Feb. 3, 2009, “Complex Vaso-Occlusive Coils”); and U.S. Pat. No. 7,695,484 (Wallace et al., Apr. 13, 2010, “Polymer Covered Vaso-Occlusive Devices and Methods of Producing Such Devices”).

Prior art which appears to be within this category also includes U.S. Pat. No. 7,896,899 (Patterson et al., Mar. 1, 2011, “Metallic Coils Enlaced with Biological or Biodegradable or Synthetic Polymers or Fibers for Embolization of a Body Cavity”); U.S. Pat. No. 8,002,789 (Ramzipoor et al., Aug. 23, 2011, “Stretch-Resistant Vaso-Occlusive Devices with Flexible Detachment Junctions”); U.S. Pat. No. 8,066,036 (Monetti et al., Nov. 29, 2011, “Three-Dimensional Complex Coil”); U.S. Pat. No. 8,172,862 (Wallace et al., May 8, 2012, “Polymer Covered Vaso-Occlusive Devices and Methods of Producing Such Devices”); U.S. Pat. No. 8,202,292 (Kellett, Jun. 19, 2012, “Vaso-Occlusive Coil Delivery System”); U.S. Pat. No. 8,226,660 (Teoh et al., Jul. 24, 2012, “Vaso-Occlusive Coils with Non-Overlapping Sections”); U.S. Pat. No. 8,267,955 (Patterson et al., Sep. 18, 2012, “Metallic Coils Enlaced with Fibers for Embolization of a Body Cavity”); U.S. Pat. No. 8,308,751 (Gerberding, Nov. 13, 2012, “Foldable Vaso-Occlusive Member”); U.S. Pat. No. 8,323,306 (Schaefer et al., Dec. 4, 2012, “Microcoil Vaso-Occlusive Device with Multi-Axis Secondary Configuration”); U.S. Pat. No. 8,361,104 (Jones et al., Jan. 29, 2013, “Vascular Occlusion Device with an Embolic Mesh Ribbon”); and U.S. Pat. No. 8,444,668 (Jones et al., May 21, 2013, “Expandable Vascular Occlusion Device”).

Prior art which appears to be within this category also includes U.S. patent applications: 20010009996 (Ferrera et al., Jul. 26, 2001, “Shape Memory Segmented Detachable Coil”); 20010056281 (Wallace et al., Dec. 27, 2001, “Vaso-Occlusive Member Assembly with Multiple Detaching Points”); 20020002382 (Wallace et al., Jan. 3, 2002, “Polymer Covered Vaso-Occlusive Devices and Methods of Producing Such Devices”); 20020058962 (Wallace et al., May 16, 2002, “Vaso-Occlusive Member Assembly with Multiple Detaching Points”); 20020107534 (Schaefer et al., Aug. 8, 2002, “Microcoil Vaso-Occlusive Device with Multi-Axis Secondary Configuration”); 20020128671 (Wallace et al., Sep. 12, 2002, “Polymer Covered Vaso-Occlusive Devices and Methods of Producing Such Devices”); 20020151926 (Wallace et al., Oct. 17, 2002, “Vasco-Occlusive Coil with Conical End”); 20030018356 (Schaefer et al., Jan. 23, 2003, “Microcoil Vaso-Occlusive Device with Multi-Axis Secondary Configuration”); and 20030130689 (Wallace et al., Jul. 10, 2003, “Vaso-Occlusive Member Assembly with Multiple Detaching Points”).

Prior art which appears to be within this category also includes U.S. patent applications: 20040045554 (Schaefer et al., Mar. 11, 2004, “Microcoil Vaso-Occlusive Device with Multi-Axis Secondary Configuration”); 20040158282 (Jones et al., Aug. 12, 2004, “Foam Matrix Embolization Device”); 20040243168 (Ferrera et al., Dec. 2, 2004, “Vasoocclusive Device for Treatment of Aneurysms”); 20050033350 (Ken et al., Feb. 10, 2005, “Detachable Multidiameter Vasoocclusive Coil”); 20050192618 (Porter, Sep. 1, 2005, “Complex Vaso-Occlusive Coils”); 20050192621 (Wallace et al., Sep. 1, 2005, “Polymer Covered Vaso-Occlusive Devices and Methods of Producing Such Devices”); 20050277978 (Greenhalgh, Dec. 15, 2005, “Three-Dimensional Coils for Treatment of Vascular Aneurysms”); 20060036281 (Patterson; William R. et al., Feb. 16, 2006, “Metallic Coils Enlaced with Biological or Biodegradable or Synthetic Polymers or Fibers for Embolization of a Body Cavity”); and 20060184195 (Schaefer et al., Aug. 17, 2006, “Microcoil Vaso-Occlusive Device with Multi-Axis Secondary Configuration”).

Prior art which appears to be within this category also includes U.S. patent applications: 20060184196 (Schaefer et al., Aug. 17, 2006, “Microcoil Vaso-Occlusive Device with Multi-Axis Secondary Configuration”); 20070016233 (Ferrera et al., Jan. 18, 2007, “Vasoocclusive Device for Treatment of Aneurysms”); 20070175536 (Monetti et al., Aug. 2, 2007, “Three-Dimensional Complex Coil”); 20080103585 (Monstadt et al., May 1, 2008, “Micro-Spiral Implantation Device”); 20090149864 (Porter, Jun. 11, 2009, “Complex Vaso-Occlusive Coils”); 20090254111 (Monstadt et al., Oct. 8, 2009, “Device for Implanting Occlusion Spirals Comprising an Interior Securing Element”); 20100036412 (Porter et al., Feb. 11, 2010, “Vaso-Occlusive Devices with Textured Surfaces”); 20100174301 (Wallace et al., Jul. 8, 2010, “Polymer Covered Vaso-Occlusive Devices and Methods of Producing Such Devices”); 20110092997 (Kang, Apr. 21, 2011, “Micro-Coil Assembly”); and 20110098814 (Monstadt et al., Apr. 28, 2011, “Medical Implant”).

Prior art which appears to be within this category also includes U.S. patent applications: 20110118777 (Patterson et al., May 19, 2011, “Metallic Coils Enlaced with Fibers for Embolization of a Body Cavity”); 20110184454 (Barry et al., Jul. 28, 2011, “Embolic Implants”); 20110184455 (Keeley et al., Jul. 28, 2011, “Embolization Device Constructed from Expansile Polymer”); 20120089174 (Chen et al., Apr. 12, 2012, “Vaso-Occlusive Device”); 20120116441 (Yamanaka et al., May 10, 2012, “Embolization Coil”); 20120116442 (Monstadt et al., May 10, 2012, “Micro-Spiral Implantation Device”); 20120172921 (Yamanaka et al., Jul. 5, 2012, “Embolization Coil”); 20120209309 (Chen et al., Aug. 16, 2012, “Vaso-Occlusive Device”); 20120239074 (Aboytes et al., Sep. 20, 2012, “Devices and Methods for the Treatment of Vascular Defects”); 20120259354 (Kellett, Oct. 11, 2012, “Vaso-Occlusive Coil Delivery System”); 20130018409 (Le et al., Jan. 17, 2013, “Packing Coil”); and 20130066357 (Aboytes et al., Mar. 14, 2013, “Devices and Methods for the Treatment of Vascular Defects”).

Prior art which appears to be within this category also includes U.S. patent applications: 20130116722 (Aboytes et al., May 9, 2013, “Devices and Methods for the Treatment of Vascular Defects”); 20130131711 (Bowman, May 23, 2013, “Embolic Device with Shaped Wire”); 20130253572 (Molaei et al., Sep. 26, 2013, “Occlusive Devices and Methods of Use”); 20140031858 (Bhagchandani et al., Jan. 30, 2014, “Systems and Methods for Delivering Multiple Embolization Coils”); 20140047694 (Monstadt et al., Feb. 20, 2014, “Medical Implant”); 20140128907 (Hui et al., May 8, 2014, “Occlusive Coil”); 20140163604 (Monstadt, Jun. 12, 2014, “Device for Implanting Electrically Isolated Occlusion Helixes”); 20140207180 (Ferrera, Jul. 24, 2014, “Systems and Devices for Cerebral Aneurysm Repair”); and 20140277100 (Kang, Sep. 18, 2014, “Micro-Coil Assembly”).

26. Embolic Coils with Inter-Connecting Members in Aneurysm Sack:

The prior art also discloses devices and methods comprising embolic coils with inter-connecting members which are implanted within an aneurysm sack. In an example, a longitudinal coil can be interconnected by relatively-inelastic strands at different locations along its longitudinal axis. Accordingly, the coil is constrained by these strands when it is deployed within the aneurysm sack and forms specific desired configurations which differ from those formed by an unconstrained coil. Prior art which appears to be within this category includes U.S. Pat. No. 5,443,478 (Purdy, Aug. 22, 1995, “Multi-Element Intravascular Occlusion Device”); U.S. Pat. No. 5,582,619 (Ken, Dec. 10, 1996, “Stretch Resistant Vaso-Occlusive Coils”); U.S. Pat. No. 5,766,219 (Horton, Jun. 16, 1998, “Anatomically Shaped Vasoocclusive Device and Method for Deploying Same”); U.S. Pat. No. 5,833,705 (Ken et al., Nov. 10, 1998, “Stretch Resistant Vaso-Occlusive Coils”); U.S. Pat. No. 5,853,418 (Ken et al., Dec. 29, 1998, “Stretch Resistant Vaso-Occlusive Coils (II)”); U.S. Pat. No. 5,935,145 (Villar et al., Aug. 10, 1999, “Vaso-Occlusive Device with Attached Polymeric Materials”); U.S. Pat. No. 6,004,338 (Ken et al., Dec. 21, 1999, “Stretch Resistant Vaso-Occlusive Coils”); U.S. Pat. No. 6,013,084 (Ken et al., Jan. 11, 2000, “Stretch Resistant Vaso-Occlusive Coils (II)”); and U.S. Pat. No. 6,193,728 (Ken et al., Feb. 27, 2001, “Stretch Resistant Vaso-Occlusive Coils (II)”).

Prior art which appears to be within this category also includes U.S. Pat. No. 6,287,318 (Villar et al., Sep. 11, 2001, “Vaso-Occlusive Device with Attached Polymeric Materials”); U.S. Pat. No. 6,616,617 (Ferrera et al., Sep. 9, 2003, “Vasoocclusive Device for Treatment of Aneurysms”); U.S. Pat. No. 7,326,225 (Ferrera et al., Feb. 5, 2008, “Vasoocclusive Device for Treatment of Aneurysms”); U.S. Pat. No. 7,708,755 (Davis et al., May 4, 2010, “Stretch Resistant Embolic Coil Delivery System with Combined Mechanical and Pressure Release Mechanism”); U.S. Pat. No. 7,749,242 (Tran et al., Jul. 6, 2010, “Expanding Vaso-Occlusive Device”); U.S. Pat. No. 7,766,933 (Davis et al., Aug. 3, 2010, “Stretch Resistant Design for Embolic Coils with Stabilization Bead”); U.S. Pat. No. 7,883,526 (Jones et al., Feb. 8, 2011, “Embolic Coil Having Stretch Resistant Member with an Attached End and an End with Movement Freedom”); U.S. Pat. No. 7,896,899 (Patterson et al., Mar. 1, 2011, “Metallic Coils Enlaced with Biological or Biodegradable or Synthetic Polymers or Fibers for Embolization of a Body Cavity”); U.S. Pat. No. 7,938,845 (Aganon et al., May 10, 2011, “Anchor Assemblies in Stretch-Resistant Vaso-Occlusive Coils”); and U.S. Pat. No. 8,034,073 (Davis et al., Oct. 11, 2011, “Stretch Resistant Embolic Coil”).

Prior art which appears to be within this category also includes U.S. Pat. No. 8,211,141 (Davis et al., Jul. 3, 2012, “Stretch Resistant Design for Embolic Coils with Stabilization Bead”); U.S. Pat. No. 8,267,955 (Patterson et al., Sep. 18, 2012, “Metallic Coils Enlaced with Fibers for Embolization of a Body Cavity”); U.S. Pat. No. 8,308,751 (Gerberding, Nov. 13, 2012, “Foldable Vaso-Occlusive Member”); U.S. Pat. No. 8,328,860 (Strauss et al., Dec. 11, 2012, “Implant Including a Coil and a Stretch-Resistant Member”); and U.S. Pat. No. 8,486,101 (Tran et al., Jul. 16, 2013, “Expanding Vaso-Occlusive Device”).

Prior art which appears to be within this category also includes U.S. patent applications: 20060036281 (Patterson; William R. et al., Feb. 16, 2006, “Metallic Coils Enlaced with Biological or Biodegradable or Synthetic Polymers or Fibers for Embolization of a Body Cavity”); 20070016233 (Ferrera et al., Jan. 18, 2007, “Vasoocclusive Device for Treatment of Aneurysms”); 20110118777 (Patterson et al., May 19, 2011, “Metallic Coils Enlaced with Fibers for Embolization of a Body Cavity”); 20110213406 (Aganon et al., Sep. 1, 2011, “Anchor Assemblies in Stretch-Resistant Vaso-Occlusive Coils”); 20110313443 (Lorenzo et al., Dec. 22, 2011, “Occlusive Device with Stretch Resistant Member and Anchor Filament”); and 20130331883 (Strauss et al., Dec. 12, 2013, “Implant Including a Coil and a Stretch-Resistant Member”).

27. Embolic Coils with Special Coatings in Aneurysm Sack:

The prior art also discloses devices and methods for treating aneurysms which comprise embolic coils with special coatings for implantation within an aneurysm sack. Since this review focuses primarily on the mechanical and structural features of aneurysm treatment devices, this category focuses primarily on coil coatings which have special mechanical properties. There is a large body of prior art featuring stent and coil coatings with special pharmaceutical properties (apart from interesting mechanical properties) which are not included in this review.

Prior art which appears to be within this category includes U.S. Pat. No. 5,690,667 (Gia, Nov. 25, 1997, “Vasoocclusion Coil Having a Polymer Tip”); U.S. Pat. No. 5,702,361 (Evans et al., Dec. 30, 1997, “Method for Embolizing Blood Vessels”); U.S. Pat. No. 7,244,261 (Lorenzo et al., Jul. 17, 2007, “Activatable Bioactive Vascular Occlusive Device”); U.S. Pat. No. 7,247,159 (Lorenzo et al., Jul. 24, 2007, “Activatable Bioactive Vascular Occlusive Device”); U.S. Pat. No. 7,294,123 (Jones et al., Nov. 13, 2007, “Activatable Bioactive Vascular Occlusive Device and Method of Use”); U.S. Pat. No. 7,300,661 (Henson et al., Nov. 27, 2007, “Adding Microscopic Porosity to the Surface of a Microcoil to be Used for Medical Implantation”); U.S. Pat. No. 7,361,367 (Henson et al., Apr. 22, 2008, “Adding Microscopic Porosity to the Surface of a Microcoil to be Used for Medical Implantation”); U.S. Pat. No. 7,442,382 (Henson et al., Oct. 28, 2008, “Adding Microscopic Porosity to the Surface of a Microcoil to be Used for Medical Implantation”); U.S. Pat. No. 8,273,100 (Martinez, Sep. 25, 2012, “Three Element Coaxial Vaso-Occlusive Device”); and U.S. Pat. No. 8,764,788 (Martinez, Jul. 1, 2014, “Multi-Layer Coaxial Vaso-Occlusive Device”).

Prior art which appears to be within this category also includes U.S. patent applications: 20030093111 (Ken et al., May 15, 2003, “Device for Vaso-Occlusion and Interventional Therapy”); 20040093014 (Ho et al., May 13, 2004, “Bioactive Components for Incorporation with Vaso-Occlusive Members”); 20040098028 (Martinez, May 20, 2004, “Three Element Coaxial Vaso-Occlusive Device”); 20050171572 (Martinez, Aug. 4, 2005, “Multi-Layer Coaxial Vaso-Occlusive Device”); 20060251695 (Henson et al., Nov. 9, 2006, “Adding Microscopic Porosity to the Surface of a Microcoil to be Used for Medical Implantation”); 20060251700 (Henson et al., Nov. 9, 2006, “Adding Microscopic Porosity to the Surface of a Microcoil to be Used for Medical Implantation”); 20080031919 (Henson et al., Feb. 7, 2008, “Adding Microscopic Porosity to the Surface of a Microcoil to be Used for Medical Implantation”); 20080152686 (Henson et al., Jun. 26, 2008, “Adding Microscopic Porosity to the Surface of a Microcoil to be Used for Medical Implantation”); 20110054511 (Henson et al., Mar. 3, 2011, “Adding Microscopic Porosity to the Surface of a Microcoil to be Used for Medical Implantation”); 20110245863 (Martinez, Oct. 6, 2011, “Multi-Layer Coaxial Vaso-Occlusive Device”); 20120323268 (Martinez, Dec. 20, 2012, “Three Element Coaxial Vaso-Occlusive Device”); 20130066359 (Murphy et al., Mar. 14, 2013, “Vaso-Occlusive Device”); and 20130072959 (Wu et al., Mar. 21, 2013, “Non-Fragmenting Low Friction Bioactive Absorbable Coils for Brain Aneurysm Therapy”).

### 28. Polymer or Hydrogel Longitudinal Embolic Members in Aneurysm Sack:

The prior art also includes devices and methods comprising longitudinal embolic members (such as coils, filaments, or meshes) which are made from polymers or hydrogels and implanted within an aneurysm sack. These embolic members can be advantageously more flexible, more compressive, more expansive, and/or more bulky than metal embolic members. Prior art which appears to be within this category includes U.S. Pat. No. 5,749,894 (Engelson, May 12, 1998, “Aneurysm Closure Method”); U.S. Pat. No. 6,015,424 (Rosenbluth et al., Jan. 18, 2000, “Apparatus and Method for Vascular Embolization”); U.S. Pat. No. 6,024,754 (Engelson, Feb. 15, 2000, “Aneurysm Closure Method”); U.S. Pat. No. 6,312,421 (Boock, Nov. 6, 2001, “Aneurysm Embolization Material and Device”); U.S. Pat. No. 6,375,669 (Rosenbluth et al., Apr. 23, 2002, “Apparatus and Method for Vascular Embolization”); U.S. Pat. No. 6,423,085 (Murayama et al., Jul. 23, 2002, “Biodegradable Polymer Coils for Intraluminal Implants”); U.S. Pat. No. 6,602,269 (Wallace et al., Aug. 5, 2003, “Embolic Devices Capable of In-Situ Reinforcement”); and U.S. Pat. No. 6,723,108 (Jones et al., Apr. 20, 2004, “Foam Matrix Embolization Device”).

Prior art which appears to be within this category also includes U.S. Pat. No. 6,979,344 (Jones et al., Dec. 27, 2005, “Foam Matrix Embolization Device”); U.S. Pat. No. 8,002,789 (Ramzipoor et al., Aug. 23, 2011, “Stretch-Resistant Vaso-Occlusive Devices with Flexible Detachment Junctions”); U.S. Pat. No. 8,273,100 (Martinez, Sep. 25, 2012, “Three Element Coaxial Vaso-Occlusive Device”); U.S. Pat. No. 8,313,504 (Do et al., Nov. 20, 2012, “Foam Matrix Embolization Device”); U.S. Pat. No. 8,377,091 (Cruise et al., Feb. 19, 2013, “Embolization Device Constructed from Expansile Polymer”); U.S. Pat. No. 8,470,035 (Cruise et al., Jun. 25, 2013, “Hydrogel Filaments for Biomedical Uses”); U.S. Pat. No. 8,764,788 (Martinez, Jul. 1, 2014, “Multi-Layer Coaxial Vaso-Occlusive Device”); and U.S. Pat. No. 8,771,294 (Sepetka et al., Jul. 8, 2014, “Aneurysm Treatment Devices and Methods”).

Prior art which appears to be within this category also includes U.S. patent applications: 20020143348 (Wallace et al., Oct. 3, 2002, “Embolic Devices Capable of In-Situ Reinforcement”); 20040098028 (Martinez, May 20, 2004, “Three Element Coaxial Vaso-Occlusive Device”); 20040115164 (Pierce et al., Jun. 17, 2004, “Soft Filament Occlusive Device Delivery System”); 20040158282 (Jones et al., Aug. 12, 2004, “Foam Matrix Embolization Device”); 20040161451 (Pierce et al., Aug. 19, 2004, “Soft Filament Occlusive Device Delivery System”); and 20050119687 (Dacey et al., Jun. 2, 2005, “Methods of, and Materials for, Treating Vascular Defects with Magnetically Controllable Hydrogels”).

Prior art which appears to be within this category also includes U.S. patent applications: 20050171572 (Martinez, Aug. 4, 2005, “Multi-Layer Coaxial Vaso-Occlusive Device”); 20060058834 (Do et al., Mar. 16, 2006, “Foam Matrix Embolization Device”); 20060116709 (Sepetka et al., Jun. 1, 2006, “Aneurysm Treatment Devices and Methods”); 20060116709 (Sepetka et al., Jun. 1, 2006, “Aneurysm Treatment Devices and Methods”); 20060116712 (Sepetka et al., Jun. 1, 2006, “Aneurysm Treatment Devices and Methods”); 20060116713 (Sepetka et al., Jun. 1, 2006, “Aneurysm Treatment Devices and Methods”); 20060276831 (Porter et al., Dec. 7, 2006, “Porous Materials for Use in Aneurysms”); 20070299464 (Cruise; Gregory M. et al., Dec. 27, 2007, “Embolization Device Constructed from Expansile Polymer”); 20080249608 (Dave, Oct. 9, 2008, “Bioabsorbable Polymer Bioabsorbable Composite Stents”); 20090164013 (Cruise et al., Jun. 25, 2009, “Hydrogel Filaments for Biomedical Uses”); and 20090227976 (Calabria et al., Sep. 10, 2009, “Multiple Biocompatible Polymeric Strand Aneurysm Embolization System and Method”).

Prior art which appears to be within this category also includes U.S. patent applications: 20110184455 (Keeley et al., Jul. 28, 2011, “Embolization Device Constructed from Expansile Polymer”); 20110245863 (Martinez, Oct. 6, 2011, “Multi-Layer Coaxial Vaso-Occlusive Device”); 20120283769 (Cruise et al., Nov. 8, 2012, “Embolization Device Constructed from Expansile Polymer”); 20120289995 (Constant et al., Nov. 15, 2012, “Embolic Devices”); 20120323268 (Martinez, Dec. 20, 2012, “Three Element Coaxial Vaso-Occlusive Device”); 20130085518 (Trommeter et al., Apr. 4, 2013, “Multi-Fiber Shape Memory Device”); 20130131716 (Cruise et al., May 23, 2013, “Embolization Device Constructed from Expansile Polymer”); and 20130302251 (Constant et al., Nov. 14, 2013, “Embolic Devices”).

29. Longitudinal Embolic Members with String-of-Pearls Structure in Aneurysm Sack:

The prior art also discloses devices and methods for treating aneurysms which comprise longitudinal embolic members with structure similar to a “string-of-pearls” for implantation within an aneurysm sack. In various examples, “string-of-pearls” structures can comprise an (evenly-spaced) series of wider (spherical, oblong, or rectangular) embolic members which are connected by a thinner (central) coil or string. Prior art which appears to be within this category includes U.S. Pat. No. 6,238,403 (Greene et al., May 29, 2001, “Filamentous Embolic Device with Expansible Elements”); U.S. Pat. No. 6,299,619 (Greene et al., Oct. 9, 2001, “Methods for Embolizing a Target Vascular Site”); U.S. Pat. No. 6,602,261 (Greene et al., Aug. 5, 2003, “Filamentous Embolic Device with Expansile Elements”); U.S. Pat. No. 6,616,617 (Ferrera et al., Sep. 9, 2003, “Vasoocclusive Device for Treatment of Aneurysms”); U.S. Pat. No. 7,014,645 (Greene et al., Mar. 21, 2006, “Method of Manufacturing Expansile Filamentous Embolization Devices”); U.S. Pat. No. 7,070,609 (West, Jul. 4, 2006, “Aneurysm Embolization Device and Deployment System”); U.S. Pat. No. 7,326,225 (Ferrera et al., Feb. 5, 2008, “Vasoocclusive Device for Treatment of Aneurysms”); U.S. Pat. No. 7,481,821 (Fogarty et al., Jan. 27, 2009, “Embolization Device and a Method of Using the Same”); U.S. Pat. No. 7,491,214 (Greene et al., Feb. 17, 2009, “Filamentous Embolization Device with Expansible Elements”); and U.S. Pat. No. 7,842,054 (Greene et al., Nov. 30, 2010, “Method of Manufacturing Expansile Filamentous Embolization Devices”).

Prior art which appears to be within this category also includes U.S. Pat. No. 8,262,686 (Fogarty et al., Sep. 11, 2012, “Embolization Device and a Method of Using the Same”); U.S. Pat. No. 8,562,636 (Fogarty et al., Oct. 22, 2013, “Embolization Device and a Method of Using the Same”); U.S. Pat. No. 8,715,317 (Janardhan et al., May 6, 2014, “Flow Diverting Devices”); U.S. Pat. No. 8,747,432 (Janardhan et al., Jun. 10, 2014, “Woven Vascular Treatment Devices”); U.S. Pat. No. 8,753,371 (Janardhan et al., Jun. 17, 2014, “Woven Vascular Treatment Systems”); U.S. Pat. No. 8,771,294 (Sepetka et al., Jul. 8, 2014, “Aneurysm Treatment Devices and Methods”); U.S. Pat. No. 8,784,446 (Janardhan et al., Jul. 22, 2014, “Circumferentially Offset Variable Porosity Devices”); U.S. Pat. No. 8,813,625 (Janardhan et al., Aug. 26, 2014, “Methods of Manufacturing Variable Porosity Flow Diverting Devices”); and U.S. Pat. No. 8,845,679 (Janardhan et al., Sep. 30, 2014, “Variable Porosity Flow Diverting Devices”).

Prior art which appears to be within this category also includes U.S. patent applications: 20020120276 (Greene et al., Aug. 29, 2002, “Filamentous Embolic Device with Expansile Elements”); 20020177855 (Greene et al., Nov. 28, 2002, “Method of Manufacturing Expansile Filamentous Embolization Devices”); 20040059370 (Greene et al., Mar. 25, 2004, “Filamentous Embolization Device with Expansible Elements”); 20040193246 (Ferrera, Sep. 30, 2004, “Methods and Apparatus for Treating Aneurysms and Other Vascular Defects”); 20040210249 (Fogarty et al., Oct. 21, 2004, “Embolization Device and a Method of Using the Same”); 20050015110 (Fogarty et al., Jan. 20, 2005, “Embolization Device and a Method of Using the Same”); and 20050267510 (Razack, Dec. 1, 2005, “Device for the Endovascular Treatment of Intracranial Aneurysms”).

Prior art which appears to be within this category also includes U.S. patent applications: 20060116709 (Sepetka et al., Jun. 1, 2006, “Aneurysm Treatment Devices and Methods”); 20060116709 (Sepetka et al., Jun. 1, 2006, “Aneurysm Treatment Devices and Methods”); 20060116712 (Sepetka et al., Jun. 1, 2006, “Aneurysm Treatment Devices and Methods”); 20060116713 (Sepetka et al., Jun. 1, 2006, “Aneurysm Treatment Devices and Methods”); 20060149299 (Greene et al., Jul. 6, 2006, “Method of Manufacturing Expansile Filamentous Embolization Devices”); 20070016233 (Ferrera et al., Jan. 18, 2007, “Vasoocclusive Device for Treatment of Aneurysms”); 20070150045 (Ferrera, Jun. 28, 2007, “Methods and Apparatus for Treating Aneurysms and Other Vascular Defects”); 20090105748 (Fogarty et al., Apr. 23, 2009, “Embolization Device and a Method of Using the Same”); 20090232869 (Greene et al., Sep. 17, 2009, “Filamentous Embolization Device with Expansible Elements”); and 20120179192 (Fogarty et al., Jul. 12, 2012, “Embolization Device and a Method of Using the Same”).

Prior art which appears to be within this category also includes U.S. patent applications: 20120239074 (Aboytes et al., Sep. 20, 2012, “Devices and Methods for the Treatment of Vascular Defects”); 20120303108 (Fogarty et al., Nov. 29, 2012, “Embolization Device and a Method of Using the Same”); 20130066357 (Aboytes et al., Mar. 14, 2013, “Devices and Methods for the Treatment of Vascular Defects”); 20130116722 (Aboytes et al., May 9, 2013, “Devices and Methods for the Treatment of Vascular Defects”); 20130231695 (Malek, Sep. 5, 2013, “Embolic Coil”); 20140088690 (Fogarty et al., Mar. 27, 2014, “Embolization Device and a Method of Using the Same”); 20140135810 (Divino et al., May 15, 2014, “Occlusive Devices”); 20140135811 (Divino et al., May 15, 2014, “Occlusive Devices”); 20140135812 (Divino et al., May 15, 2014, “Occlusive Devices”); 20140260928 (Janardhan et al., Sep. 18, 2014, “Methods of Using Non-Cylindrical Mandrels”); and 20140265096 (Janardhan et al., Sep. 18, 2014, “Non-Cylindrical Mandrels”).

30. Accumulation of Mass in Aneurysm Sack by Spooling and/or Axial Rotation:

Although not common, the prior art also includes a few examples of devices and methods for treating aneurysms comprising accumulating embolic mass in the aneurysm sack by the rotational spooling or dispensation of a longitudinal embolic member within the aneurysm sack. In an example, the accumulation of mass can occur as a longitudinal embolic member is spooled around a rotating central member within the sack. In an example, the accumulation of mass can occur as a longitudinal embolic member is dispensed within the sack from a rotating central member. Prior art which appears to be within this category includes U.S. patent applications 20110166588 (Connor et al., Jul. 7, 2011, “Aneurysm Embolization by Rotational Accumulation of Mass”) and 20120303052 (Connor, Nov. 29, 2012, “Aneurysm Occlusion by Rotational Dispensation of Mass”).

31. Liner or Balloon with Non-Porous Walls in Aneurysm Sack:

The prior art also discloses devices and methods for treating aneurysms including the use of a flexible occluding intrasacular aneurysm liner or balloon which has non-porous walls and which is deployed within the aneurysm sack. Such a flexible liner or balloon can be filled with solid embolic members, a gelatinous embolic composition, or a liquid embolic composition—which can contribute to its expansion within the aneurysm sack. Prior art which appears to be within this category includes U.S. Pat. No. 5,334,210 (Gianturco, Aug. 2, 1994, “Vascular Occlusion Assembly”); U.S. Pat. No. 6,350,270 (Roue, Feb. 26, 2002, “Aneurysm Liner”); U.S. Pat. No. 6,511,468 (Cragg et al., Jan. 28, 2003, “Device and Method for Controlling Injection of Liquid Embolic Composition”); U.S. Pat. No. 7,338,511 (Mirigian et al., Mar. 4, 2008, “Solid Embolic Material with Variable Expansion”); U.S. Pat. No. 7,695,488 (Berenstein et al., Apr. 13, 2010, “Expandable Body Cavity Liner Device”); U.S. Pat. No. 7,976,527 (Cragg et al., Jul. 12, 2011, “Device and Method for Controlling Injection of Liquid Embolic Composition”); U.S. Pat. No. 8,021,416 (Abrams, Sep. 20, 2011, “Methods for Delivering a Prosthesis to a Site in a Body”); U.S. Pat. No. 8,425,541 (Masters et al., Apr. 23, 2013, “Aneurysm Occlusion Device Containing Bioactive and Biocompatible Copolymer Shell and a Liquid Embolic Agent”); U.S. Pat. No. 8,425,542 (Moftakhar et al., Apr. 23, 2013, “Aneurysm Occlusion Device Containing Bioactive and Biocompatible Copolymer Shell and Biocompatible Metallic Frame Member”); U.S. Pat. No. 8,454,649 (Cragg et al., Jun. 4, 2013, “Device and Method for Controlling Injection of Liquid Embolic Composition”); and U.S. Pat. No. 8,529,619 (Abrams, Sep. 10, 2013, “Methods for Delivering a Prosthesis to a Site in a Body”).

Prior art which appears to be within this category also includes U.S. patent applications: 20030187473 (Berenstein et al., Oct. 2, 2003, “Expandable Body Cavity Liner Device”); 20090062834 (Moftakhar et al., Mar. 5, 2009, “Aneurysm Occlusion Device Containing Bioactive and Biocompatible Copolymer Shell and Biocompatible Metallic Frame Member”); 20090118761 (Masters et al., May 7, 2009, “Aneurysm Occlusion Device Containing Bioactive and Biocompatible Copolymer Shell and a Liquid Embolic Agent”); 20100168781 (Berenstein et al., Jul. 1, 2010, “Expandable Body Cavity Liner Device”); 20130211443 (Cragg et al., Aug. 15, 2013, “Device and Method for Controlling Injection of Liquid Embolic Composition”); and 20140039536 (Cully et al., Feb. 6, 2014, “Space-Filling Device”).

32. Liner, Balloon, Net, or Mesh with Porous Walls in Aneurysm Sack:

The prior art also includes devices and methods for treating aneurysms comprising a flexible occluding intrasacular aneurysm liner, balloon, net, or mesh with relatively-porous walls which is deployed within the aneurysm sack. Such a flexible liner, balloon, net, or mesh can be filled with solid embolic members or compositions, which can contribute to its expansion within the aneurysm sack. In an example, the walls of an aneurysm liner, balloon, net, or mesh can be permeable to inflow of blood from the sack or to outflow from liquid (e.g. saline or contrast media) which is injected within it, but be impermeable to outflow of embolic members. Prior art which appears to be within this category includes U.S. Pat. No. 4,364,392 (Strother et al., Dec. 21, 1982, “Detachable Balloon Catheter”); U.S. Pat. No. 6,346,117 (Greenhalgh, Feb. 12, 2002, “Bag for Use in the Intravascular Treatment of Saccular Aneurysms”); U.S. Pat. No. 6,391,037 (Greenhalgh, May 21, 2002, “Bag for Use in the Intravascular Treatment of Saccular Aneurysms”); U.S. Pat. No. 6,547,804 (Porter et al., Apr. 15, 2003, “Selectively Permeable Highly Distensible Occlusion Balloon”); U.S. Pat. No. 6,585,748 (Jeffree, Jul. 1, 2003, “Device for Treating Aneurysms”); U.S. Pat. No. 6,855,153 (Saadat, Feb. 15, 2005, “Embolic Balloon”); U.S. Pat. No. 7,153,323 (Teoh et al., Dec. 26, 2006, “Aneurysm Liner with Multi-Segment Extender”); U.S. Pat. No. 7,695,488 (Berenstein et al., Apr. 13, 2010, “Expandable Body Cavity Liner Device”); U.S. Pat. No. 8,021,416 (Abrams, Sep. 20, 2011, “Methods for Delivering a Prosthesis to a Site in a Body”); and U.S. Pat. No. 8,529,619 (Abrams, Sep. 10, 2013, “Methods for Delivering a Prosthesis to a Site in a Body”).

Prior art which appears to be within this category also includes U.S. patent applications: 20030187473 (Berenstein et al., Oct. 2, 2003, “Expandable Body Cavity Liner Device”); 20040098027 (Teoh et al., May 20, 2004, “Expandable Body Cavity Liner Device”); 20060079923 (Chhabra et al., Apr. 13, 2006, “Aneurysm Treatment Using Semi-Compliant Balloon”); 20090112249 (Miles et al., Apr. 30, 2009, “Medical Device for Modification of Left Atrial Appendage and Related Systems and Methods”); 20100168781 (Berenstein et al., Jul. 1, 2010, “Expandable Body Cavity Liner Device”); 20110046658 (Connor et al., Feb. 24, 2011, “Aneurysm Occlusion Device”); 20140135810 (Divino et al., May 15, 2014, “Occlusive Devices”); 20140135811 (Divino et al., May 15, 2014, “Occlusive Devices”); and 20140135812 (Divino et al., May 15, 2014, “Occlusive Devices”); and also PCT/US2009/002537 (Connor et al, 2009, “Aneurysm Occlusion Device”).

33. Liquid Embolic Composition into Aneurysm Sack:

The prior art also discloses devices and methods for treating aneurysms comprising injection of a liquid embolic composition into an aneurysm sack. Most of the time, unless contained within a liner or balloon, this liquid embolic composition is formulated to congeal within the sack. Often this congealing process is accelerated by the concurrent delivery of a congealing agent or energy. Sometimes a balloon is temporarily expanded within the parent vessel to prevent the liquid embolic composition from leaking out of the aneurysm sack before it congeals. Prior art which appears to be within this category includes U.S. Pat. No. 5,776,097 (Massoud, Jul. 7, 1998, “Method and Device for Treating Intracranial Vascular Aneurysms”); U.S. Pat. No. 5,785,679 (Abolfathi et al., Jul. 28, 1998, “Methods and Apparatus for Treating Aneurysms and Arterio-Venous Fistulas”); U.S. Pat. No. 5,795,331 (Cragg et al., Aug. 18, 1998, “Balloon Catheter for Occluding Aneurysms of Branch Vessels”); U.S. Pat. No. 6,096,021 (Helm et al., Aug. 1, 2000, “Flow Arrest, Double Balloon Technique for Occluding Aneurysms or Blood Vessels”); U.S. Pat. No. 6,140,452 (Felt et al., Oct. 31, 2000, “Biomaterial for In Situ Tissue Repair”); U.S. Pat. No. 6,306,177 (Felt et al., Oct. 23, 2001, “Biomaterial System for In Situ Tissue Repair”); U.S. Pat. No. 6,454,738 (Tran et al., Sep. 24, 2002, “Methods for Delivering In Vivo Uniform Dispersed Embolic Compositions of High Viscosity”); and U.S. Pat. No. 6,511,468 (Cragg et al., Jan. 28, 2003, “Device and Method for Controlling Injection of Liquid Embolic Composition”).

Prior art which appears to be within this category also includes U.S. Pat. No. 6,547,804 (Porter et al., Apr. 15, 2003, “Selectively Permeable Highly Distensible Occlusion Balloon”); U.S. Pat. No. 6,569,190 (Whalen et al., May 27, 2003, “Methods for Treating Aneurysms”); U.S. Pat. No. 6,629,947 (Sahatjian et al., Oct. 7, 2003, “Systems and Methods for Delivering Flowable Substances for Use as Implants and Surgical Sealants”); U.S. Pat. No. 6,958,061 (Truckai et al., Oct. 25, 2005, “Microspheres with Sacrificial Coatings for Vaso-Occlusive Systems”); U.S. Pat. No. 6,964,657 (Cragg et al., Nov. 15, 2005, “Catheter System and Method for Injection of a Liquid Embolic Composition and a Solidification Agent”); U.S. Pat. No. 7,083,632 (Avellanet et al., Aug. 1, 2006, “Aneurysm Embolic Device with an Occlusive Member”); U.S. Pat. No. 7,083,643 (Whalen et al., Aug. 1, 2006, “Methods for Treating Aneurysms”); and U.S. Pat. No. 7,294,137 (Rivelli et al., Nov. 13, 2007, “Device for Multi-Modal Treatment of Vascular Lesions”).

Prior art which appears to be within this category also includes U.S. Pat. No. 7,338,511 (Mirigian et al., Mar. 4, 2008, “Solid Embolic Material with Variable Expansion”); U.S. Pat. No. 7,374,568 (Whalen et al., May 20, 2008, “Methods for Embolizing Aneurysmal Sites with a High Viscosity Embolizing Composition”); U.S. Pat. No. 7,414,038 (Kinugasa et al., Aug. 19, 2008, “Embolic Materials”); U.S. Pat. No. 7,666,220 (Evans et al., Feb. 23, 2010, “System and Methods for Endovascular Aneurysm Treatment”); U.S. Pat. No. 7,976,527 (Cragg et al., Jul. 12, 2011, “Device and Method for Controlling Injection of Liquid Embolic Composition”); U.S. Pat. No. 8,262,607 (Porter, Sep. 11, 2012, “Liquid Embolic Composition Delivery Devices and Methods”); U.S. Pat. No. 8,425,541 (Masters et al., Apr. 23, 2013, “Aneurysm Occlusion Device Containing Bioactive and Biocompatible Copolymer Shell and a Liquid Embolic Agent”); and U.S. Pat. No. 8,454,649 (Cragg et al., Jun. 4, 2013, “Device and Method for Controlling Injection of Liquid Embolic Composition”).

Prior art which appears to be within this category also includes U.S. patent applications: 20020018752 (Krall et al., Feb. 14, 2002, “Polymerizable Compositions and Methods of Use”); 20020082620 (Lee, Jun. 27, 2002, “Bioactive Materials for Aneurysm Repair”); 20030093097 (Avellanet et al., May 15, 2003, “Aneurysm Embolic Device with an Occlusive Member”); 20030135264 (Whalen et al., Jul. 17, 2003, “Methods for Treating Aneurysms”); 20030223955 (Whalen et al., Dec. 4, 2003, “Methods for Embolizing Aneurysmal Sites with a High Viscosity Embolizing Composition”); 20040098027 (Teoh et al., May 20, 2004, “Expandable Body Cavity Liner Device”); 20060235464 (Avellanet et al., Oct. 19, 2006, “Aneurysm Embolic Device with an Occlusive Member”); and 20090118761 (Masters et al., May 7, 2009, “Aneurysm Occlusion Device Containing Bioactive and Biocompatible Copolymer Shell and a Liquid Embolic Agent”).

Prior art which appears to be within this category also includes U.S. patent applications: 20090318949 (Ganpath et al., Dec. 24, 2009, “Sealing Apparatus and Methods of Use”); 20130108574 (Chevalier et al., May 2, 2013, “Radiopaque, Non-Biodegradable, Water-Insoluble Iodinated Benzyl Ethers of Poly(Vinyl Alcohol), Preparation Method Thereof, Injectable Embolizing Compositions Containing Thereof and Use Thereof”); 20130211443 (Cragg et al., Aug. 15, 2013, “Device and Method for Controlling Injection of Liquid Embolic Composition”); and 20130310687 (Takizawa et al., Nov. 21, 2013, “Blood Vessel Embolization Method Using Balloon Catheter and Balloon Catheter for Blood Vessel Embolization Method”).

34. Gelatinous Embolic Composition into Aneurysm Sack:

The prior art also discloses devices and methods for treating aneurysms comprising injection of a gelatinous embolic composition into an aneurysm sack. Often, this gelatinous embolic composition is formulated to further solidify within the aneurysm sack. Sometimes this solidification process is accelerated by the concurrent delivery of a solidifying agent or energy. Prior art which appears to be within this category includes U.S. Pat. No. 5,624,685 (Takahashi et al., Apr. 29, 1997, “High Polymer Gel and Vascular Lesion Embolizing Material Comprising the Same”); U.S. Pat. No. 5,702,361 (Evans et al., Dec. 30, 1997, “Method for Embolizing Blood Vessels”); U.S. Pat. No. 5,749,894 (Engelson, May 12, 1998, “Aneurysm Closure Method”); U.S. Pat. No. 5,888,546 (Ji et al., Mar. 30, 1999, “Embolic Material for Endovascular Occlusion of Abnormal Vasculature and Method for Using the Same”); U.S. Pat. No. 5,894,022 (Ji et al., Apr. 13, 1999, “Embolic Material for Endovascular Occlusion of Abnormal Vasculature and Method of Using the Same”); U.S. Pat. No. 6,017,977 (Evans et al., Jan. 25, 2000, “Methods for Embolizing Blood Vessels”); U.S. Pat. No. 6,024,754 (Engelson, Feb. 15, 2000, “Aneurysm Closure Method”); and U.S. Pat. No. 6,238,403 (Greene et al., May 29, 2001, “Filamentous Embolic Device with Expansible Elements”).

Prior art which appears to be within this category also includes U.S. Pat. No. 6,281,263 (Evans et al., Aug. 28, 2001, “Methods for Embolizing Blood Vessels”); U.S. Pat. No. 6,299,619 (Greene et al., Oct. 9, 2001, “Methods for Embolizing a Target Vascular Site”); U.S. Pat. No. 6,335,384 (Evans et al., Jan. 1, 2002, “Methods for Embolizing Blood Vessels”); U.S. Pat. No. 6,379,373 (Sawhney et al., Apr. 30, 2002, “Methods and Apparatus for Intraluminal Deposition of Hydrogels”); U.S. Pat. No. 6,463,317 (Kucharczyk et al., Oct. 8, 2002, “Device and Method for the Endovascular Treatment of Aneurysms”); U.S. Pat. No. 6,602,261 (Greene et al., Aug. 5, 2003, “Filamentous Embolic Device with Expansile Elements”); U.S. Pat. No. 6,689,148 (Sawhney et al., Feb. 10, 2004, “Methods and Apparatus for Intraluminal Deposition of Hydrogels”); U.S. Pat. No. 6,818,018 (Sawhney, Nov. 16, 2004, “In Situ Polymerizable Hydrogels”); and U.S. Pat. No. 7,014,645 (Greene et al., Mar. 21, 2006, “Method of Manufacturing Expansile Filamentous Embolization Devices”).

Prior art which appears to be within this category also includes U.S. Pat. No. 7,220,270 (Sawhney et al., May 22, 2007, “Methods and Apparatus for Intraluminal Deposition of Hydrogels”); U.S. Pat. No. 7,491,214 (Greene et al., Feb. 17, 2009, “Filamentous Embolization Device with Expansible Elements”); U.S. Pat. No. 7,842,054 (Greene et al., Nov. 30, 2010, “Method of Manufacturing Expansile Filamentous Embolization Devices”); U.S. Pat. No. 8,439,942 (Moran et al., May 14, 2013, “Embolization Device”); U.S. Pat. No. 8,535,367 (Kim et al., Sep. 17, 2013, “Devices and Methods for Treatment of Vascular Aneurysms”); U.S. Pat. No. 8,647,377 (Kim et al., Feb. 11, 2014, “Devices and Methods for Treatment of Vascular Aneurysms”); and U.S. Pat. No. 8,840,867 (Sophie et al., Sep. 23, 2014, “Embolizing Sclerosing Hydrogel”).

Prior art which appears to be within this category also includes U.S. patent applications: 20020082636 (Sawhney et al., Jun. 27, 2002, “Methods and Apparatus for Intraluminal Deposition of Hydrogels”); 20020120276 (Greene et al., Aug. 29, 2002, “Filamentous Embolic Device with Expansile Elements”); 20020177855 (Greene et al., Nov. 28, 2002, “Method of Manufacturing Expansile Filamentous Embolization Devices”); 20030014075 (Rosenbluth et al., Jan. 16, 2003, “Methods, Materials and Apparatus for Deterring or Preventing Endoleaks Following Endovascular Graft Implantation”); 20040059370 (Greene et al., Mar. 25, 2004, “Filamentous Embolization Device with Expansible Elements”); 20050004660 (Rosenbluth et al., Jan. 6, 2005, “Methods, Materials and Apparatus for Deterring or Preventing Endoleaks Following Endovascular Graft Implantation”); 20050080445 (Sawhney et al., Apr. 14, 2005, “Methods and Apparatus for Intraluminal Deposition of Hydrogels”); and 20050133046 (Becker et al., Jun. 23, 2005, “Compositions and Methods for Improved Occlusion of Vascular Defects”).

Prior art which appears to be within this category also includes U.S. patent applications: 20060149299 (Greene et al., Jul. 6, 2006, “Method of Manufacturing Expansile Filamentous Embolization Devices”); 20060292206 (Kim et al., Dec. 28, 2006, “Devices and Methods for Treatment of Vascular Aneurysms”); 20070050008 (Kim et al., Mar. 1, 2007, “Devices and Methods for Treatment of Vascular Aneurysms”); 20070055355 (Kim et al., Mar. 8, 2007, “Devices and Methods for Treatment of Vascular Aneurysms”); 20070061005 (Kim et al., Mar. 15, 2007, “Devices and Methods for Treatment of Vascular Aneurysms”); 20070150041 (Evans et al., Jun. 28, 2007, “Methods and Systems for Aneurysm Treatment Using Filling Structures”); and 20070167747 (Borgert et al., Jul. 19, 2007, “Catheter, Apparatus and Method for Therapeutic Embolization”).

Prior art which appears to be within this category also includes U.S. patent applications: 20090232869 (Greene et al., Sep. 17, 2009, “Filamentous Embolization Device with Expansible Elements”); 20100063472 (Becker et al., Mar. 11, 2010, “Compositions and Methods for Improved Occlusion of Vascular Defects”); 20110182998 (Reb et al., Jul. 28, 2011, “Microspheres Useful for Therapeutic Vascular Embolization”); 20110286925 (Lerouge et al., Nov. 24, 2011, “Embolizing Sclerosing Hydrogel”); 20120238644 (Gong et al., Sep. 20, 2012, “Fragmented Hydrogels”); 20120265287 (Sharma et al., Oct. 18, 2012, “In-Situ Forming Foams for Treatment of Aneurysms”); 20120330343 (Kim et al., Dec. 27, 2012, “Devices and Methods for Treatment of Vascular Aneurysms”); and 20130045182 (Gong et al., Feb. 21, 2013, “Polysaccharide Based Hydrogels”).

Prior art which appears to be within this category also includes U.S. patent applications: 20130095087 (Shalaby et al., Apr. 18, 2013, “Absorbable In Situ Gel-Forming System, Method of Making and Use Thereof”); 20130131711 (Bowman, May 23, 2013, “Embolic Device with Shaped Wire”); 20130245606 (Stam et al., Sep. 19, 2013, “Hydrogel Based Occlusion Systems”); 20130252900 (Reb et al., Sep. 26, 2013, “Microspheres Useful for Therapeutic Vascular Embolization”); 20130344159 (Moine et al., Dec. 26, 2013, “Implantable Swellable Bio-Resorbable Polymer”); 20140052168 (Sawhney, Feb. 20, 2014, “Methods of Using In Situ Hydration of Hydrogel Articles for Sealing or Augmentation of Tissue or Vessels”); and 20140081374 (Kim et al., Mar. 20, 2014, “Devices and Methods for Treatment of Vascular Aneurysms”).

35. Embolic Spheres and/or Particles into Aneurysm Sack:

The prior art also includes devices and methods for treating aneurysms which comprise the delivery of a plurality of separate embolic spherical, polygonal, and/or other generally-convex shaped members into the sack of an aneurysm. These spheres, polygons, and/or other convex particles can be inserted into an aneurysm liner, net, or mesh which helps to contain them within the sack. These spheres, polygons, and/or other convex embolic members can be soft or hard, compressible or resilient, solid or hollow. Expandable intrasacular woven wire devices (e.g. ball-shaped stents) are included in a different category. Prior art which appears to be within this category includes U.S. Pat. No. 4,364,392 (Strother et al., Dec. 21, 1982, “Detachable Balloon Catheter”); U.S. Pat. No. 6,958,061 (Truckai et al., Oct. 25, 2005, “Microspheres with Sacrificial Coatings for Vaso-Occlusive Systems”); U.S. Pat. No. 7,311,861 (Lanphere et al., Dec. 25, 2007, “Embolization”); U.S. Pat. No. 7,449,236 (Lanphere et al., Nov. 11, 2008, “Porous Polymeric Particle Comprising Polyvinyl Alcohol and Having Interior to Surface Porosity-Gradient”); U.S. Pat. No. 7,588,780 (Buiser et al., Sep. 15, 2009, “Embolization”); U.S. Pat. No. 7,588,825 (Bell et al., Sep. 15, 2009, “Embolic Compositions”); U.S. Pat. No. 7,666,333 (Lanphere et al., Feb. 23, 2010, “Embolization”); U.S. Pat. No. 7,695,488 (Berenstein et al., Apr. 13, 2010, “Expandable Body Cavity Liner Device”); U.S. Pat. No. 7,736,671 (DiCarlo et al., Jun. 15, 2010, “Embolization”); U.S. Pat. No. 7,842,377 (Lanphere et al., Nov. 30, 2010, “Porous Polymeric Particle Comprising Polyvinyl Alcohol and Having Interior to Surface Porosity-Gradient”); U.S. Pat. No. 7,976,823 (Lanphere et al., Jul. 12, 2011, “Ferromagnetic Particles and Methods”); and U.S. Pat. No. 8,617,132 (Golzarian et al., Dec. 31, 2013, “Bioresorbable Embolization Microspheres”).

Prior art which appears to be within this category also includes U.S. patent applications: 20030187473 (Berenstein et al., Oct. 2, 2003, “Expandable Body Cavity Liner Device”); 20040091543 (Bell et al., May 13, 2004, “Embolic Compositions”); 20040098027 (Teoh et al., May 20, 2004, “Expandable Body Cavity Liner Device”); 20060276831 (Porter et al., Dec. 7, 2006, “Porous Materials for Use in Aneurysms”); 20080033366 (Matson et al., Feb. 7, 2008, “Compressible Intravascular Embolization Particles and Related Methods and Delivery Systems”); 20090112249 (Miles et al., Apr. 30, 2009, “Medical Device for Modification of Left Atrial Appendage and Related Systems and Methods”); 20090318948 (Linder et al., Dec. 24, 2009, “Device, System and Method for Aneurysm Embolization”); and 20100168781 (Berenstein et al., Jul. 1, 2010, “Expandable Body Cavity Liner Device”).

Prior art which appears to be within this category also includes U.S. patent applications: 20110046658 (Connor et al., Feb. 24, 2011, “Aneurysm Occlusion Device”); 20110082427 (Golzarian et al., Apr. 7, 2011, “Bioresorbable Embolization Microspheres”); 20110182998 (Reb et al., Jul. 28, 2011, “Microspheres Useful for Therapeutic Vascular Embolization”); 20130190795 (Matson et al., Jul. 25, 2013, “Compressible Intravascular Embolization Particles and Related Methods and Delivery Systems”); 20130252900 (Reb et al., Sep. 26, 2013, “Microspheres Useful for Therapeutic Vascular Embolization”); 20140099374 (Golzarian et al., Apr. 10, 2014, “Bioresorbable Embolization Microspheres”); 20140135810 (Divino et al., May 15, 2014, “Occlusive Devices”); 20140135811 (Divino et al., May 15, 2014, “Occlusive Devices”); and 20140135812 (Divino et al., May 15, 2014, “Occlusive Devices”); and also PCT/US2009/002537 (Connor et al, 2009, “Aneurysm Occlusion Device”).

36. Customized Pre-Molded Member into Aneurysm Sack:

The prior art also discloses devices and methods for treating aneurysms which comprise customized (e.g. pre-molded or pre-shaped) occlusion devices which are specifically configured to match the configuration of a specific aneurysm (in a specific patient). Such customized occlusion devices are often designed based on the results of three-dimensional imaging of the aneurysm. Prior art which appears to be within this category includes U.S. Pat. No. 6,165,193 (Greene et al., Dec. 26, 2000, “Vascular Embolization with an Expansible Implant”); U.S. Pat. No. 6,165,193 (Greene et al., Dec. 26, 2000, “Vascular Embolization with an Expansible Implant”); U.S. Pat. No. 6,500,190 (Greene et al., Dec. 31, 2002, “Vascular Embolization with an Expansible Implant”); U.S. Pat. No. 7,029,487 (Greene et al., Apr. 18, 2006, “Vascular Embolization with an Expansible Implant”); U.S. Pat. No. 7,201,762 (Greene et al., Apr. 10, 2007, “Vascular Embolization with an Expansible Implant”); and U.S. Pat. No. 7,483,558 (Greene et al., Jan. 27, 2009, “Vascular Embolization with an Expansible Implant”).

Prior art which appears to be within this category also includes U.S. Pat. No. 7,799,047 (Greene et al., Sep. 21, 2010, “Vascular Embolization with an Expansible Implant”); U.S. Pat. No. 8,067,071 (Farnsworth et al., Nov. 29, 2011, “Composite Self-Cohered Web Materials”); U.S. Pat. No. 8,133,256 (Wilson et al., Mar. 13, 2012, “Shape Memory Polymer Foams for Endovascular Therapies”); U.S. Pat. No. 8,377,241 (Farnsworth et al., Feb. 19, 2013, “Method of Making Porous Self-Cohered Web Materials”); U.S. Pat. No. 8,449,592 (Wilson et al., May 28, 2013, “Stent with Expandable Foam”); U.S. Pat. No. 8,473,030 (Greenan et al., Jun. 25, 2013, “Vessel Position and Configuration Imaging Apparatus and Methods”); and U.S. Pat. No. 8,597,745 (Farnsworth et al., Dec. 3, 2013, “Composite Self-Cohered Web Materials”).

Prior art which appears to be within this category also includes U.S. patent applications: 20010001835 (Greene et al., May 24, 2001, “Vascular Embolization with an Expansible Implant”); 20030083737 (Greene et al., May 1, 2003, “Vascular Embolization with an Expansible Implant”); 20030088311 (Greene et al., May 8, 2003, “Vascular Embolization with an Expansible Implant”); 20050075405 (Wilson et al., Apr. 7, 2005, “Shape Memory Polymer Foams for Endovascular Therapies”); 20050095428 (Dicarlo et al., May 5, 2005, “Embolic Compositions”); 20060036045 (Wilson et al., Feb. 16, 2006, “Shape Memory Polymers”); 20070135907 (Wilson et al., Jun. 14, 2007, “Stent with Expandable Foam”); 20070176333 (Greene et al., Aug. 2, 2007, “Vascular Embolization with an Expansible Implant”); and 20090112250 (Greene et al., Apr. 30, 2009, “Vascular Embolization with an Expansible Implant”).

Prior art which appears to be within this category also includes U.S. patent applications: 20090318941 (Sepetka et al., Dec. 24, 2009, “Self-Expandable Endovascular Device for Aneurysm Occlusion”); 20110005062 (Greene et al., Jan. 13, 2011, “Vascular Embolization with an Expansible Implant”); 20110039967 (Wilson et al., Feb. 17, 2011, “Shape Memory Polymers”); 20110089592 (Farnsworth et al., Apr. 21, 2011, “Method of Making Porous Self-Cohered Web Materials”); 20110137405 (Wilson et al., Jun. 9, 2011, “Stent with Expandable Foam”); 20110144686 (Wilson et al., Jun. 16, 2011, “Shape Memory Polymer Foams for Endovascular Therapies”); 20130045182 (Gong et al., Feb. 21, 2013, “Polysaccharide Based Hydrogels”); 20130253086 (Wilson et al., Sep. 26, 2013, “Shape Memory Polymers”); 20130253634 (Wilson et al., Sep. 26, 2013, “Stent with Expandable Foam”); 20130289690 (Thapliyal, Oct. 31, 2013, “Personalized Prosthesis and Methods of Use”); and 20140018902 (Myr, Jan. 16, 2014, “Tailor-Made Stent Graft and Procedure for Minimally Invasive Aneurysm Repair with Novel Tailor-Made Balloon, Novel Guidewire, and Novel Capsulated Bioglue”).

### 37. Extravascular Sleeve Around Aneurysm Sack and Parent Vessel:

The prior art also discloses devices and methods for treating aneurysms comprising an extravascular sleeve which is implanted around an aneurysm sack and the parent vessel of the aneurysm. Prior art which appears to be within this category includes U.S. Pat. No. 7,818,084 (Boyden et al., Oct. 19, 2010, “Methods and Systems for Making a Blood Vessel Sleeve”); U.S. Pat. No. 8,095,382 (Boyden et al., Jan. 10, 2012, “Methods and Systems for Specifying a Blood Vessel Sleeve”); U.S. Pat. No. 8,147,537 (Boyden et al., Apr. 3, 2012, “Rapid-Prototyped Custom-Fitted Blood Vessel Sleeve”); U.S. Pat. No. 8,163,003 (Boyden et al., Apr. 24, 2012, “Active Blood Vessel Sleeve Methods and Systems”); U.S. Pat. No. 8,478,437 (Boyden et al., Jul. 2, 2013, “Methods and Systems for Making a Blood Vessel Sleeve”); and U.S. Pat. No. 8,491,459 (Yun, Jul. 23, 2013, “Expandable Vessel Harness for Treating Vessel Aneurysms”); and U.S. patent application 20130218191 (Heltai, Aug. 22, 2013, “Method for Deploying a Sleeve and Tubing Device for Restricting and Constricting Aneurysms and a Sleeve and Tubing Device and System”).

### 38. Other Devices for Aneurysm Treatment:

Finally, there are a variety of devices and methods for treating aneurysms which are relevant to this application, but which do not fit neatly into the above categories. I have included them in this miscellaneous category for completeness. Prior art which appears to be within this miscellaneous category includes U.S. Pat. No. 6,603,994 (Wallace et al., Aug. 5, 2003, “Apparatus and Method for Internally Inducing a Magnetic Field in an Aneurysm to Embolize Aneurysm with Magnetically-Controllable Substance”); U.S. Pat. No. 7,182,744 (Yamasaki et al., Feb. 27, 2007, “Method and Apparatus for Aneurismal Treatment”); U.S. Pat. No. 7,294,137 (Rivelli et al., Nov. 13, 2007, “Device for Multi-Modal Treatment of Vascular Lesions”); U.S. Pat. No. 7,744,610 (Hausen, Jun. 29, 2010, “System for Closing a Tissue Structure from Inside”); and U.S. Pat. No. 8,728,094 (Roorda et al., May 20, 2014, “Percutaneous Aneurysm Inversion and Ligation”). Prior art which appears to be within this category also includes U.S. patent applications: 20020087077 (Wallace et al., Jul. 4, 2002, “Apparatus and Method for Internally Inducing a Magnetic Field in an Aneurysm to Embolize Aneurysm with Magnetically-Controllable Substance”); 20090299448 (Timko et al., Dec. 3, 2009, “Aneurysm Treatment System”); 20120310611 (Sadasivan et al., Dec. 6, 2012, “System and Method for Simulating Deployment Configuration of an Expandable Device”); and 20130023903 (Roorda et al., Jan. 24, 2013, “Percutaneous Aneurysm Inversion and Ligation”).

### SUMMARY OF THIS INVENTION

This invention is a device and method to occlude a cerebral aneurysm. This invention can be embodied in a device to occlude a cerebral aneurysm which includes: a longitudinal lumen that is inserted into the parent blood vessel of an aneurysm; a flexible expandable member (such as a net or mesh) which is expanded within the aneurysm sack by the insertion of embolic members into that flexible expandable member; and a resilient expandable member (such as a cylindrical stent or ring stent) that is attached to a central portion of the flexible expandable member and expanded within the aneurysm sack in order to keep the flexible expandable member from slipping out of the aneurysm sack.

More specifically, this invention can be embodied in: (a) a longitudinal lumen that is configured to be inserted into a blood vessel; (b) a flexible expandable member that is configured to travel through the longitudinal lumen, be inserted into an aneurysm, and then be expanded within the aneurysm sack; wherein this flexible expandable member is selected from the group consisting of a net, a mesh, a lattice, and a balloon with holes; wherein this flexible expandable member is sufficiently flexible to substantively conform to the contours of the walls of the aneurysm sack after the flexible expandable member is expanded within the aneurysm; (c) a resilient expandable member that is configured to travel through the longitudinal lumen, be inserted into the aneurysm, and then be expanded within the aneurysm sack; wherein this resilient expandable member resists contraction after it has been expanded; wherein a plane formed by the expanding circumference of this resilient expandable member is substantially parallel to the plane that centrally spans the circumference of the aneurysm neck; wherein a plane formed by the expanding circumference of this resilient expandable member spans the aneurysm sack at the sack's largest circumference parallel to the plane that centrally spans the circumference of the aneurysm neck; and wherein expansion of the resilient expandable member resiliently holds a central portion of the flexible expandable member against the walls of the aneurysm so that the flexible expandable member does not slip out of the aneurysm sack; and (d) a plurality of individual embolic members that are configured to travel through the longitudinal lumen, be inserted into the flexible expandable member within the aneurysm, and accumulate within the flexible expandable member; wherein the flexible expandable member does not allow the embolic members to escape out from the flexible expandable member; and wherein accumulation of the plurality of embolic members inside the flexible expandable member causes the flexible expandable member to expand.

Of the 38 categories of prior art which were identified in the proceeding section, the most relevant categories to this particular embodiment are: category 32, “liner, balloon, net, or mesh with porous walls in aneurysm sack”; and category 35, “embolic spheres and/or particles into aneurysm sack.” Of the 79 figures which are discussed in the following sections, the most relevant figures to this particular embodiment are FIGS. 31, 32, and 33 which show three sequential views (during deployment) of a Saturn-shaped device for aneurysm occlusion.

### BRIEF INTRODUCTION TO THE FIGURES

FIGS. 1 through 79 show different examples of how this invention can be embodied in devices and methods for occluding an aneurysm, but they do not limit the full generalizability of the claims.

FIGS. 1 through 3 show three sequential views (during deployment) of an embolic coil which forms interconnected contiguous loops within an aneurysm sack.

FIGS. 4 through 6 show three sequential views (during deployment) of an embolic coil with selective connection detachment for adjustable-size interconnected loops.

FIGS. 7 through 9 show three sequential views (during deployment) of a device and method to occlude an aneurysm which can be described as an embolic coil with selective connection formation for adjustable-size interconnected loops.

FIGS. 10 through 12 show three sequential views (during deployment) of a device and method to occlude an aneurysm which can be described as connecting and looping embolic members to occlude an aneurysm.

FIGS. 13 through 15 show three sequential views (during deployment) of a device and method to occlude an aneurysm which can be described as inserting and rotating embolic members to occlude an aneurysm.

FIGS. 16 through 18 show an example of a device and method to occlude an aneurysm which can be described as coil rotation to form a densely-packed mass within an aneurysm.

FIGS. 19 through 21 show three sequential views (during deployment) of a device and method to occlude an aneurysm which can be described as “string-of-pearls” rotation to form a densely-packed mass within an aneurysm.

FIGS. 22 through 24 show three sequential views (during deployment) of a device and method to occlude an aneurysm which can be described as aneurysm coil jailing using an expandable torus.

FIGS. 25 through 27 show three sequential views (during deployment) of a device and method to occlude an aneurysm which can be described as aneurysm occlusion using loops which are substantially-parallel to the aneurysm neck.

FIGS. 28 through 30 show three sequential views (during deployment) of a device and method to occlude an aneurysm which can be described as aneurysm occlusion using multiple centrally-aligned ellipsoids.

FIGS. 31 through 33 show three sequential views (during deployment) of a device and method to occlude an aneurysm which can be described as a “Saturn-shaped” device for aneurysm occlusion.

FIGS. 34 through 36 show three sequential views (during deployment) of a device and method to occlude an aneurysm which can be described as using concentric resilient and non-resilient intrasacular members for aneurysm occlusion.

FIGS. 37 through 39 show three sequential views (during deployment) of a device and method to occlude an aneurysm which can be described as an aneurysm parent vessel stent with a semi-cylindrical central portion.

FIGS. 40 through 42 show three sequential views (during deployment) of a device and method to occlude an aneurysm which can be described as an aneurysm parent vessel stent with a saddle-shaped central segment.

FIGS. 43 through 45 show three sequential views (during deployment) of a device and method to occlude an aneurysm which can be described as a bowtie or barbell shaped stent to occlude an aneurysm.

FIGS. 46 through 48 show three sequential views (during deployment) of a device and method to occlude an aneurysm which can be described as an aqueduct-shaped stent to occlude an aneurysm.

FIGS. 49 through 51 show three sequential views (during deployment) of a device and method to occlude an aneurysm which can be described as a stent with end rings and a central helical segment to occlude an aneurysm.

FIGS. 52 through 54 show three sequential views (during deployment) of a device and method to occlude an aneurysm which can be described as a three-stent, two-balloon device for occluding an aneurysm.

FIGS. 55 through 57 show three sequential views (during deployment) of a device and method to occlude an aneurysm which can be described as a device with two parallel stents for occluding an aneurysm.

FIGS. 58 through 65 show eight sequential views (during deployment) of a device and method to occlude an aneurysm which can be described as a three-stent device (“tri-stent device”) for occluding an aneurysm at a three-way vessel junction.

FIGS. 66 through 68 show alternative perspectives of example of a device and method to occlude an aneurysm which can be described as a three-petal device (or “tri-petal device”) for occluding an aneurysm at a vessel junction.

FIGS. 69 through 73 show five sequential views (during deployment) of this three-petal device (or “tri-petal device”) for occluding an aneurysm at a vessel junction.

FIG. 74 shows an example of a tri-petal member whose petals comprise sections of a virtual three-dimensional triangular object with two arcuate convex sides.

FIG. 75 shows an example of a tri-petal member whose petals comprise equal sections of a virtual sphere.

FIG. 76 shows an example of a tri-petal member whose petals comprise sections of a virtual sphere, but wherein these sections are not equal.

FIG. 77 shows an example of a tri-petal member with a saddle-shaped first petal which spans the aneurysm neck and two smaller petals which span the junction at polar coordinates of 120 degrees and 240 degrees.

FIG. 78 shows an example of a tri-petal member whose petals comprise portions of a virtual sphere including a wide common core.

FIG. 79 shows an example of a tri-petal member whose petals comprise a virtual sphere with three arcuate “bites” taken of it.

### DETAILED DESCRIPTION OF THE FIGURES

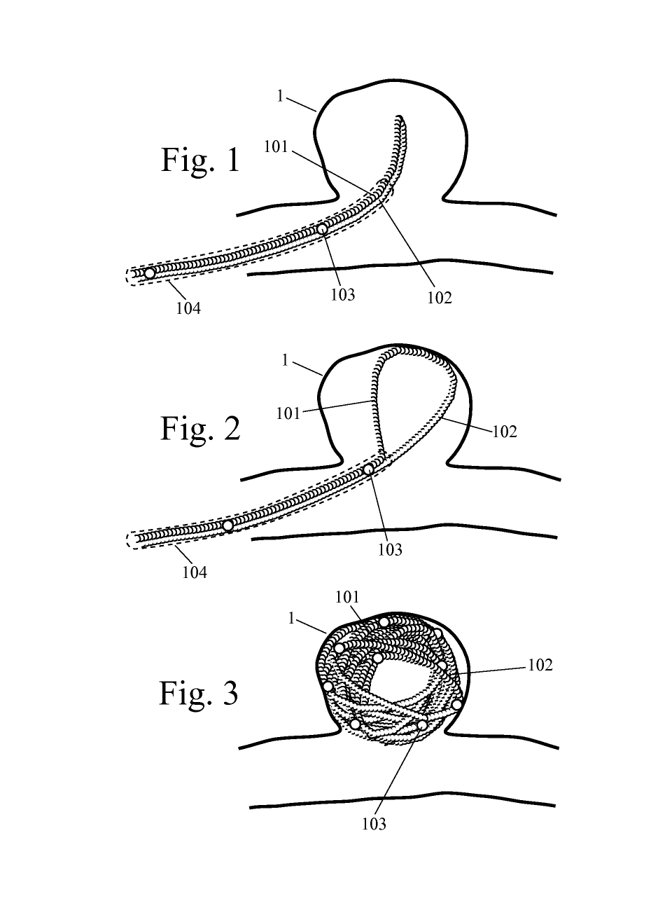

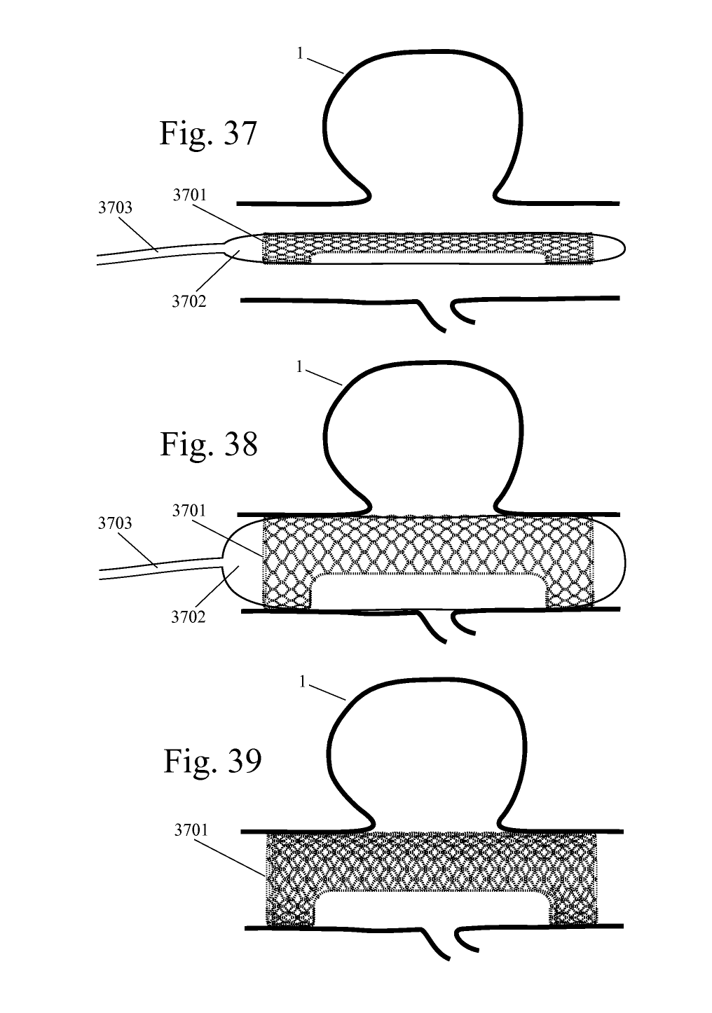

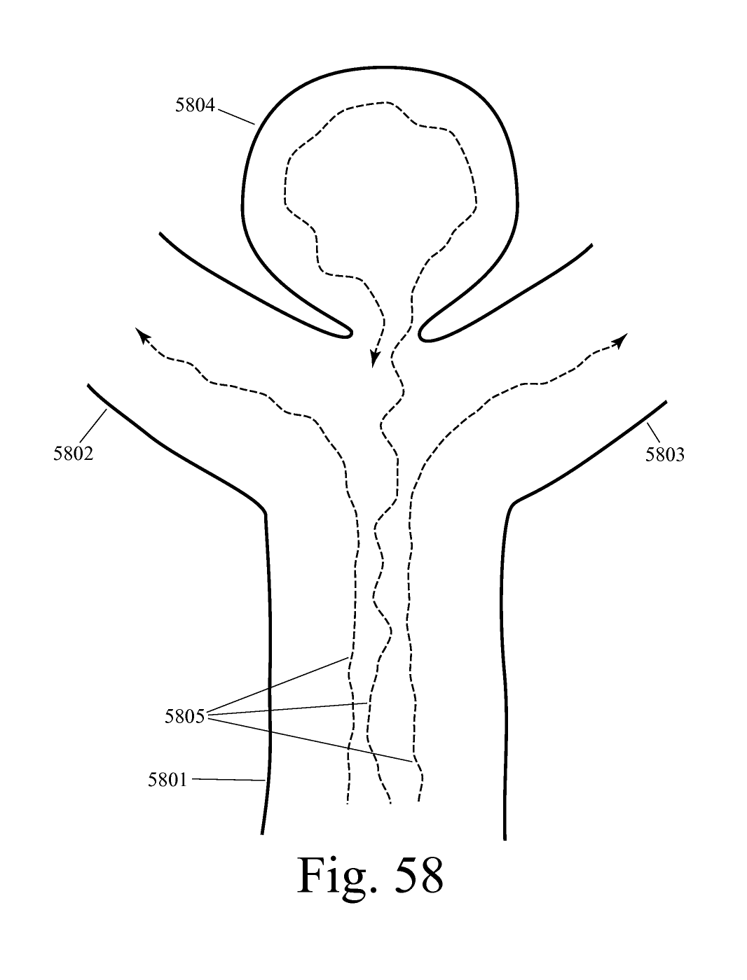

FIGS. 1 through 79 show different examples of devices and methods that can be used to occlude an aneurysm of a blood vessel, but they do not limit the full generalizability of the claims. FIGS. 1 through 36 show a number of intrasacular devices and methods, wherein these examples involve the insertion of a device into the aneurysm sac. FIGS. 37 through 57 show a number of parent vessel devices and methods, wherein these examples involve the insertion of a device into a longitudinal blood vessel from which an aneurysm has formed. FIGS. 58 through 79 show a number of three-way vessel junction devices and methods, wherein these examples involve the insertion of a device into a three-way junction of blood vessels from which an aneurysm has formed.

### A. Intrasacular Devices and Methods

FIGS. 1 through 36 show a number of intrasacular devices and methods for occluding an aneurysm, wherein these examples involve the insertion of a device into the aneurysm sac. FIGS. 1 through 3 show an example of a device and method to occlude an aneurysm which can be described as an embolic coil for forming interconnected contiguous loops in an aneurysm.

More specifically, FIGS. 1 through 3 show an example of a device to occlude an aneurysm comprising: (a) a first longitudinal section of a flexible longitudinal embolic member that is configured to be inserted into an aneurysm; (b) a second longitudinal section of a flexible longitudinal embolic member that is configured to be inserted into the aneurysm, wherein the first and second longitudinal sections are connected and/or are contiguous sections of the same flexible longitudinal embolic member; (c) a plurality of connections between the first and second sections, wherein these connections connect the first and second longitudinal sections at a plurality of selected locations along their longitudinal axes; and (d) a longitudinal lumen that is configured to be inserted into a blood vessel, wherein the first and second longitudinal sections travel through the lumen in order to be inserted into the aneurysm; wherein at least portions of the first and second longitudinal sections are configured in parallel within the lumen; wherein portions of the first and second longitudinal sections which are not connected by connections move apart from each other after exiting the lumen and connections move closer to each other after exiting the lumen in order to form a plurality of loops within the aneurysm; wherein part of the perimeter of a loop is comprised of a portion of the first longitudinal section and part of the perimeter of a loop is comprised of a portion of the second longitudinal section; wherein a loop has a contiguous 360-degree perimeter with ends which are connected to each other; and wherein loops are interconnected at the connections.

FIGS. 1 through 3 also show an example of a device to occlude an aneurysm comprising: (a) a longitudinal lumen that is configured to be inserted into a blood vessel, wherein this blood vessel is the parent vessel from which an aneurysm has formed; (b) a first longitudinal flexible embolic member that is configured to travel through the longitudinal lumen and be inserted into the aneurysm sac; (c) a second longitudinal flexible embolic member that is configured to travel through the longitudinal lumen and be inserted into the aneurysm sac; wherein the longitudinal axes of the first and second embolic members are substantially parallel as these embolic members travel through the longitudinal lumen; and wherein the first and second embolic members are contiguous and/or connected to each other at their distal ends; and (d) a plurality of connections which connect the first and second embolic members at a plurality of locations along the lengths of the embolic members; wherein the segments of the first and second embolic members that are not connected by the connections move away from each other after they exit the longitudinal lumen, thereby forming loops within the aneurysm sac; wherein these loops are connected by the connections; and wherein accumulation of these loops within the aneurysm sac substantially occludes the aneurysm.

In an example, the longitudinal lumen can be a removable catheter. In an example, the embolic members can be coils. In an example, the connections can connect the embolic members at uniformly-spaced locations along their lengths so as to form equal-size loops within the aneurysm sac and wherein these equal-size loops substantially span the circumference of the aneurysm sac. In an example, the connections can connect the embolic members at uniformly-spaced locations along their lengths so as to form equal-size loops within the aneurysm sac and these equal-size loops substantially span the circumference of the aneurysm sac without protruding into the parent vessel. In an example, the connections can connect the embolic members at non-uniformly-spaced locations along their lengths so as to form loops of different sizes within the aneurysm sac and these different size loops substantially occlude the interior as well as the circumference of the aneurysm sac.

We now discuss the specific components of FIGS. 1 through 3 in detail. FIGS. 1 through 3 show three sequential views of the same example of a device and method to occlude an aneurysm. To provide anatomical context, FIG. 1 shows a longitudinal cross-sectional view of an aneurysm sac 1 which has formed on a longitudinal blood vessel. FIG. 1 also shows an occlusive device comprising: a longitudinal lumen 104 that has been inserted into the longitudinal blood vessel; a first longitudinal flexible embolic member 101 that travels through lumen 104 into aneurysm sac 1; a second longitudinal flexible embolic member 102 that travels through lumen 104 into aneurysm sac 1; and a plurality of connections (including 103) which connect first and second embolic members 101 and 102 at a plurality of locations along their longitudinal lengths. In this example, flexible embolic members 101 and 102 are two different segments (or sides) of the same continuous embolic member. In this example, this continuous member has two parallel segments or sides (comprising embolic members 101 and 102) within longitudinal lumen 104. In another example, embolic member 101 and embolic 102 can be different embolic members that are connected in some other manner at their distal ends.

In this example, flexible embolic members 101 and 102 are substantially parallel as they travel through longitudinal lumen 104. However, as shown in FIG. 2, portions of embolic members 101 and 102 which are not connected to each other separate from each other after they exit longitudinal lumen 104 within aneurysm sac 1. In an example, this separation can be partly caused by pressure from contact with the wall of aneurysm sac 1. In an example, this separation can be partly caused by embolic members 101 and 102 having a shape memory with a shape that is restored after these embolic members exit longitudinal lumen 104. In the example that is shown in FIGS. 1 and 2, segments of embolic members 101 and 102 which are not connected by connections (such as 103) move away from each other after they exit longitudinal lumen 104.

FIG. 3 shows the accumulation of a plurality of interconnected, contiguous loops within aneurysm sac 1 as flexible longitudinal embolic members 101 and 102 continue to be pushed into aneurysm sac 1. These loops are pair-wise connected to each other by the plurality of connections (including connection 103). As shown in FIG. 3, accumulation of this plurality of loops within aneurysm sac 1 forms an embolic mass which substantially occludes the aneurysm. In this example, the interconnected and contiguous nature of these loops helps to prevent loops from prolapsing out of aneurysm sac 1 into the parent blood vessel. This can result in less prolapse of coils into the parent vessel than is the case with coils in the prior art which disperse and accumulate in a free-form spiraling manner within the aneurysm sac. Also, FIG. 3 shows longitudinal lumen 104 as having been removed.

In the example shown in FIGS. 1 through 3, the connections (such as 103) between embolic members 101 and 102 are relatively evenly-spaced along the longitudinal lengths of embolic members 101 and 102. In an example, the spacing of these connections can be selected for a specific aneurysm with a specific size and shape in order to most efficiently occlude that specific aneurysm. In an example, the spacing of connections can differ between devices which are configured to occlude narrow-neck aneurysms and devices which are configured to occlude wide-neck aneurysms. In an example, the spacing of these connections can be pre-selected to vary along the length of embolic members 101 and 102 in order to most efficiently occlude an aneurysm at different times or stages during the occlusion procedure. For example, connections can be separated by longer distances at the most distal portions of embolic members 101 and 102 and become progressively shorter at more proximal portions of embolic members 101 and 102. In an example, the most distal connections can be spaced to form loops which span the entire circumference of the aneurysm sac but successive loops can become smaller and smaller to better fill the central space of the aneurysm sac.

FIGS. 4 through 6 show an example of a device and method to occlude an aneurysm which can be described as an embolic coil with selective connection detachment for adjustable-size interconnected loops. More specifically, FIGS. 4 through 6 show an example of a device to occlude an aneurysm comprising: (a) a first longitudinal section of a flexible longitudinal embolic member that is configured to be inserted into an aneurysm; (b) a second longitudinal section of a flexible longitudinal embolic member that is configured to be inserted into the aneurysm, wherein the first and second longitudinal sections are connected and/or are contiguous sections of the same flexible longitudinal embolic member; (c) selectively-detachable connections between the first and second sections, wherein these selectively-detachable connections connect the first and second longitudinal sections at a plurality of selected locations along their longitudinal axes, and wherein a subset of the selectively-detachable connections are selectively detached during insertion of the first and second longitudinal sections into the aneurysm; and (d) a longitudinal lumen that is configured to be inserted into a blood vessel, wherein the first and second longitudinal sections travel through the lumen in order to be inserted into the aneurysm; wherein at least a portion of the first and second longitudinal sections are substantially parallel to each other within the lumen; wherein portions of the first and second longitudinal sections which are not connected by selectively-detachable connections move apart from each other after exiting the lumen and selectively-detachable connections which are not detached move closer to each other after exiting the lumen in order to form a plurality of loops within the aneurysm; wherein part of the perimeter of a loop is comprised of a portion of the first longitudinal section and part of the perimeter of a loop is comprised of a portion of the second longitudinal section; wherein a loop has a contiguous 360-degree perimeter with ends which are connected to each other; wherein loops are interconnected at the selectively-detachable connections which are not detached; and wherein the loop size of at least one loop is selectively determined by selective detachment of a subset of the selectively-detachable connections during insertion of the first and second longitudinal sections.

FIGS. 4 through 6 also show an example of a device to occlude an aneurysm comprising: (a) a longitudinal lumen that is configured to be inserted into a blood vessel, wherein this blood vessel is the parent vessel from which an aneurysm has formed; (b) a first longitudinal flexible embolic member that is configured to travel through the longitudinal lumen and be inserted into the aneurysm sac; (c) a second longitudinal flexible embolic member that is configured to travel through the longitudinal lumen and be inserted into the aneurysm sac; wherein the longitudinal axes of the first and second embolic members are substantially parallel as these embolic members travel through the longitudinal lumen; and wherein the first and second embolic members are contiguous and/or connected to each other at their distal ends; and (d) a plurality of disconnectable connections which connect the first and second embolic members at a plurality of locations along the lengths of the embolic members; wherein these a subset of these connections can be selected and disconnected during the insertion of the longitudinal members; wherein the segments of the first and second embolic members that are not connected by the remaining connections move away from each other after they exit the longitudinal lumen, thereby forming loops within the aneurysm sac; wherein these loops are connected by the remaining connections; and wherein accumulation of these loops within the aneurysm sac substantially occludes the aneurysm.

In an example, the longitudinal lumen can be a removable catheter. In an example, the embolic members can be coils. In an example, a subset of the connections can be disconnected by application of electric current. In an example, a subset of the connections can be disconnected by a cutting mechanism. In an example, the selective disconnection of a subset of connections can cause loops of different sizes and different size loops occlude a greater volume of the aneurysm sac than same size loops. In an example, the selective disconnection of a subset of connections during implantation of the device can enable custom-fitting different size loops to the size and shape of the aneurysm.

We now discuss the specific components of FIGS. 4 through 6 in detail. FIGS. 4 through 6 show three sequential views of the same example of a device and method to occlude an aneurysm. FIG. 4 shows an occlusive device comprising: a longitudinal lumen 404 that has been inserted into the longitudinal blood vessel; a first longitudinal flexible embolic member 401 that travels through lumen 404 into aneurysm sac 1; a second longitudinal flexible embolic member 402 that travels through lumen 404 into aneurysm sac 1; and a plurality of disconnectable and/or detachable connections (including 403) which connect first and second embolic members 401 and 402 at a plurality of locations along their longitudinal lengths. In this example, flexible embolic members 401 and 402 are two different segments (or sides) of the same continuous embolic member. In this example, this continuous member has two parallel segments or sides (comprising embolic members 401 and 402) within longitudinal lumen 404. In another example, embolic member 401 and embolic 402 can be different embolic members that are connected in some other manner at their distal ends.

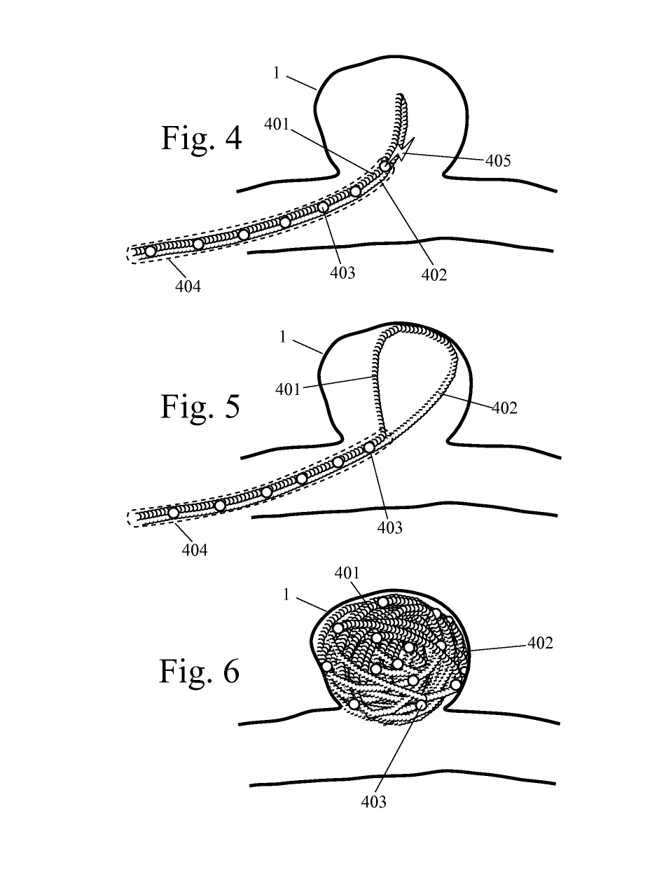

FIG. 4 shows the disconnection and/or detachment of one of the disconnectable and/or detachable connections during the implantable procedure. In this example, this disconnection and/or detachment is done by means of an electric current 405 which melts one of the connections. In this example, connection 403 is among the connections which are not disconnected and/or detached. As shown in FIG. 5, portions of embolic members 401 and 402 which are not connected to each other separate from each other after they exit longitudinal lumen 404 within aneurysm sac 1. Selective disconnection and/or detachment of a subset of the plurality of connections between embolic members 401 and 402 during the implantation procedure can enable the formation of different size loops within the aneurysm sac so as to most efficiently occlude the aneurysm sac.

In an example, the selection of which connections to disconnect and/or detach can be done by a user, in real time, during the implantation procedure. This selection can be informed by real-time imaging concerning the placement of the longitudinal lumen with respect to the aneurysm sac and/or real-time imaging concerning the progressive occlusion of the aneurysm sac. Enabling a user to selectively disconnect and/or detach selected connections during a procedure can avoid having to stock a variety of occluding members with a variety of connection spacing parameters. A standard occluding device with a standard set of connections can be modified by a user, in real time during the implantation procedure, in order to create different size loops in a specific sequence for an aneurysm's specific size and shape.

FIG. 6 shows the accumulation of a plurality of interconnected, contiguous loops within aneurysm sac 1 as some of the connections are disconnected and as flexible longitudinal embolic members 401 and 402 continue to be pushed into aneurysm sac 1. These loops are pair-wise connected to each other by the plurality of remaining connections (including connection 403). As shown in FIG. 3, accumulation of this plurality of loops within aneurysm sac 1 forms an embolic mass which substantially occludes the aneurysm. The interconnected and contiguous nature of these loops can help to reduce the probability of a loop prolapsing out of the aneurysm sac as compared to coils in the prior art which accumulate within the aneurysm sac in a less-controlled spiraling manner. Also, FIG. 6 shows longitudinal lumen 404 as having been removed.

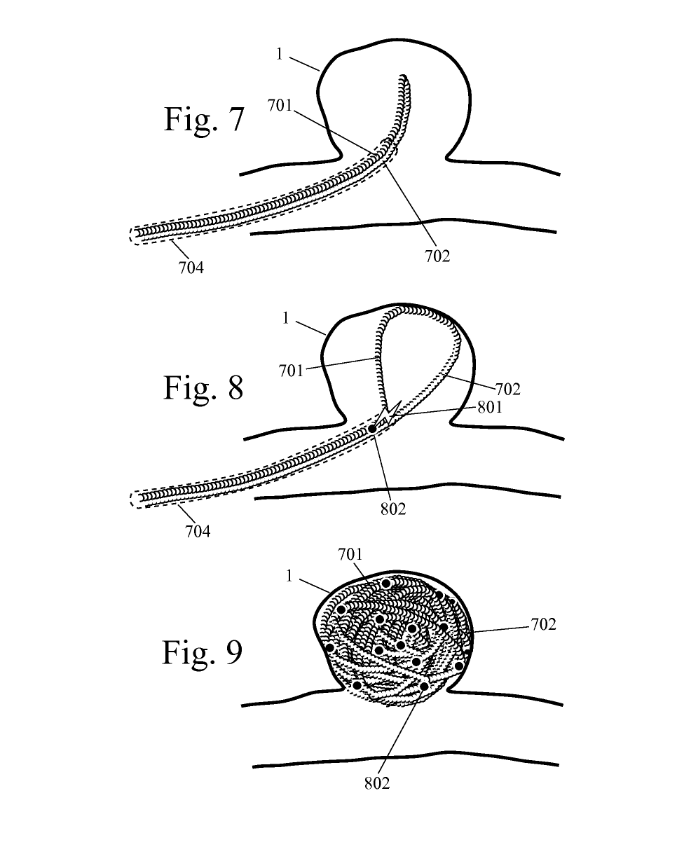

FIGS. 7 through 9 show an example of a device and method to occlude an aneurysm which can be described as an embolic coil with selective connection formation for adjustable-size interconnected loops. More specifically, FIGS. 7 through 9 show an example of a device to occlude an aneurysm comprising: (a) a first longitudinal section of a flexible longitudinal embolic member that is configured to be inserted into an aneurysm; (b) a second longitudinal section of a flexible longitudinal embolic member that is configured to be inserted into the aneurysm, wherein the first and second longitudinal sections are connected and/or are contiguous sections of the same flexible longitudinal embolic member; (c) selectively-formed connections between the first and second sections, wherein these selectively-formed connections connect the first and second longitudinal sections at a plurality of selected locations along their longitudinal axes, and wherein these selectively-formed connections are selectively formed during insertion of the first and second longitudinal sections into the aneurysm; and (d) a longitudinal lumen that is configured to be inserted into a blood vessel, wherein the first and second longitudinal sections travel through the lumen in order to be inserted into the aneurysm; wherein at least a portion of the first and second longitudinal sections are substantially parallel to each other within the lumen; wherein portions of the first and second longitudinal sections which are not connected by selectively-formed connections move apart from each other after exiting the lumen and selectively-formed connections move closer to each other after exiting the lumen in order to form a plurality of loops within the aneurysm; wherein part of the perimeter of a loop is comprised of a portion of the first longitudinal section and part of the perimeter of a loop is comprised of a portion of the second longitudinal section; wherein a loop has a contiguous 360-degree perimeter with ends which are connected to each other; wherein loops are interconnected at the selectively-formed connections; and wherein the loop size of at least one loop is selectively determined by selective formation of a selectively-formed connection during insertion of the first and second longitudinal sections.

FIGS. 7 through 9 also show an example of a device to occlude an aneurysm comprising: (a) a longitudinal lumen that is configured to be inserted into a blood vessel, wherein this blood vessel is the parent vessel from which an aneurysm has formed; (b) a first longitudinal flexible embolic member that is configured to travel through the longitudinal lumen and be inserted into the aneurysm sac; (c) a second longitudinal flexible embolic member that is configured to travel through the longitudinal lumen and be inserted into the aneurysm sac; wherein the longitudinal axes of the first and second embolic members are substantially parallel as these embolic members travel through the longitudinal lumen; and wherein the first and second embolic members are contiguous and/or connected to each other at their distal ends; and (d) a plurality of connections between the first and second embolic members which are formed to connect the first and second embolic members at a plurality of locations along the lengths of the embolic members; wherein these connections are formed during the insertion of the longitudinal members; wherein the segments of the first and second embolic members that are not connected by the connections move away from each other after they exit the longitudinal lumen, thereby forming loops within the aneurysm sac; and wherein accumulation of these loops within the aneurysm sac substantially occludes the aneurysm.

In an example, the longitudinal lumen can be a removable catheter. In an example, the embolic members can be coils. In an example, the connections between the first and second embolic members can be formed by application of electric current. In an example, the connections between the first and second embolic members can be formed by compression, linking, stapling, snapping, latching, or adhesion. In an example, the selective formation of connections between the first and second embolic members can cause loops of different sizes and using different size loops can occlude a greater volume of the aneurysm sac than using same size loops. In an example, the selective formation of connections between the first and second embolic members during implantation of the device can enable custom-fitting different size loops to the size and shape of the aneurysm.

We now discuss the specific components of FIGS. 7 through 9 in detail. FIG. 7 shows an occlusive device comprising: a longitudinal lumen 704 that has been inserted into a longitudinal blood vessel from which an aneurysm has formed; a first longitudinal flexible embolic member 701 that travels through lumen 704 into aneurysm sac 1; and a second longitudinal flexible embolic member 702 that travels through lumen 704 into aneurysm sac 1. In this example, flexible embolic members 701 and 702 are two different segments (or sides) of the same continuous embolic member.

FIG. 8 shows the formation of a connection 802 between embolic members 701 and 702 by application of electric current 801 during the implantable procedure. In other examples, connections between embolic members 701 and 702 can be formed by a means selected from the group consisting of: adhesion, fusing, compressing, pinching, stapling, snapping, linking, twisting, binding, and tying. As shown in FIG. 8, portions of embolic members 701 and 702 which are not connected to each other separate from each other after they exit longitudinal lumen 704 within aneurysm sac 1. Selective formation of connections (such as 802) between embolic members 701 and 702 during the implantation procedure enables the formation of different size loops within the aneurysm sac so as to most efficiently occlude the aneurysm sac.

In an example, the selection of where to form connections along the lengths of embolic members 701 and 702 can be done by a user, in real time, during the implantation procedure. This selection can be informed by real-time imaging concerning the placement of the longitudinal lumen with respect to the aneurysm sac and/or real-time imaging concerning the progressive occlusion of the aneurysm sac. Enabling a user to selectively form connections during a procedure can avoid having to stock a variety of occluding members with a variety of connection spacing parameters. A standard occluding device can be modified by a user, in real time during the implantation procedure, in order to create different size loops in a specific sequence for an aneurysm's specific size and shape.

FIG. 9 shows the accumulation of a plurality of interconnected, contiguous loops within aneurysm sac 1 as connections are formed and flexible longitudinal embolic members 701 and 702 are pushed into aneurysm sac 1. These loops are pair-wise connected to each other by the plurality of connections (including connection 802). The interconnected and contiguous nature of these loops can help to reduce the probability of a loop prolapsing out of the aneurysm sac as compared to coils in the prior art which accumulate within the aneurysm sac in a less-controlled spiraling manner. Also, FIG. 9 shows longitudinal lumen 704 as having been removed.

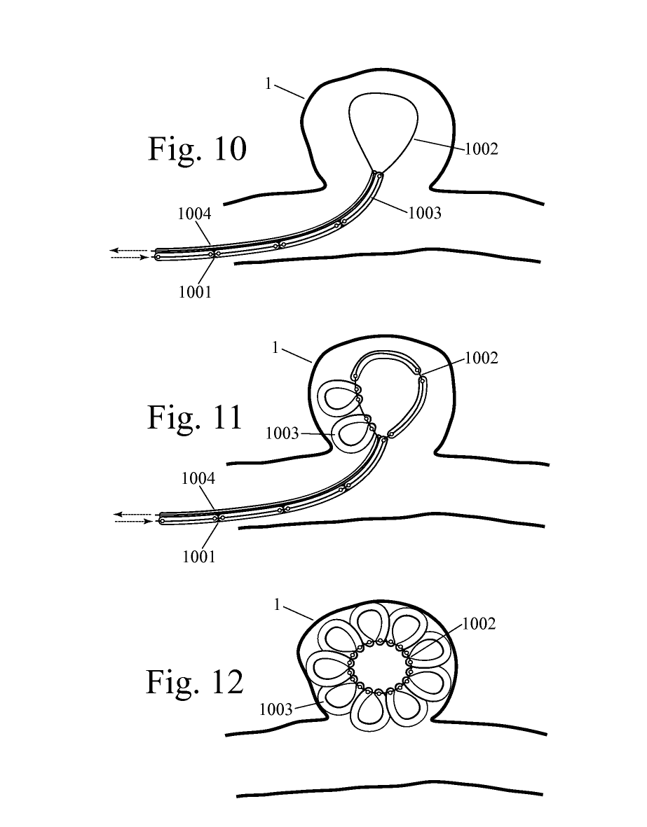

FIGS. 10 through 12 show an example of a device and method to occlude an aneurysm which can be described as connecting and looping embolic members to occlude an aneurysm. More specifically, FIGS. 10 through 12 show an example of a device to occlude an aneurysm comprising: (a) a longitudinal lumen that is configured to be inserted into a blood vessel; (b) a connecting member that travels through the lumen and is configured to be inserted into an aneurysm; wherein this connecting member is longitudinal and flexible; wherein an embolic member has a longitudinal axis that spans from its proximal end to its distal end as it travels through the lumen and a cross-sectional area that is substantively perpendicular to its longitudinal axis; (c) a plurality of embolic members that travel in a series configuration through the lumen and are configured to be inserted into the aneurysm; wherein these embolic members are longitudinal and flexible; wherein these embolic members are connected and/or linked to each other by the connecting member; wherein an embolic member has a longitudinal axis that spans from its proximal end to its distal end as it travels through the lumen and a cross-sectional area that is substantively perpendicular to its longitudinal axis; wherein the longitudinally-central portion of an embolic member bends away from the connecting member after the embolic member exits the lumen due to the two ends of the embolic member being pulled or pushed towards each other; wherein this bending of the longitudinally-central portion of an embolic member causes the embolic member to form a loop within the aneurysm; and wherein the formation of multiple loops within the aneurysm helps to embolize the aneurysm.

FIGS. 10 through 12 also show an example of a device to occlude an aneurysm comprising: (a) a longitudinal lumen that is configured to be inserted into a blood vessel, wherein this blood vessel is the parent vessel from which an aneurysm has formed; (b) a longitudinal flexible member that is configured to travel through the longitudinal lumen and be inserted into the aneurysm sac; (c) a plurality of flexible embolic members that are configured to travel through the longitudinal lumen and be inserted into the aneurysm sac; wherein these flexible embolic members are connected by the longitudinal flexible member; wherein the ends of a flexible embolic member are separated by a first distance as the embolic member travels through the longitudinal lumen; wherein the ends of this flexible embolic member are separated by a second distance after the embolic member exits the longitudinal lumen; wherein the second distance is less than the first distance; and wherein the accumulation of flexible embolic members in the aneurysm sac substantively occludes the aneurysm sac.

In an example, the longitudinal lumen can be a removable catheter. In an example, the longitudinal flexible member can be selected from the group consisting of: wire, string, filament, chain, and coil. In an example, an embolic member can have a longitudinal axis, wherein this longitudinal axis is substantively straight as the embolic member travels through the longitudinal lumen, and wherein this longitudinal axis becomes arcuate after the embolic member exits the longitudinal lumen. In an example, the ends of a flexible embolic member can move closer together after the embolic member exits the longitudinal lumen because the embolic member comprises a material with a shape memory. In an example, the ends of a flexible embolic member can move closer together after the embolic member exits the longitudinal lumen because the embolic member is compressed by the force of other embolic members in the aneurysm sac. In an example, the ends of a flexible embolic member can be slideably connected to the longitudinal flexible member.

We now discuss the specific components of the example that is shown FIGS. 10 through 12 in detail. FIG. 10 shows an example of an occlusive device comprising: a first longitudinal lumen 1001 that is configured to be inserted into a blood vessel, wherein this blood vessel is the parent vessel from which an aneurysm has formed; a second longitudinal lumen 1004 that is configured to be inserted into the blood vessel; a longitudinal flexible member 1002 that is configured to travel through one of the longitudinal lumens and to be inserted into the aneurysm sac; and a plurality of flexible embolic members, including 1003, that are configured to travel through lumen 1001 and be inserted into the aneurysm sac. In this example, flexible member 1002 comprises a loop wherein one side of the loop travels through lumen 1001 and the other side travels through lumen 1004. In another example, a device can only have one lumen and both sides of a loop can travel through this one lumen. Also, as shown in FIG. 10, the flexible embolic members (including 1003) can be connected by longitudinal flexible member 1002.

As shown in FIG. 11, the ends of a flexible embolic member (including 1003) can be separated by a first distance as the embolic member travels through lumen 1001 and the ends of the flexible embolic member are separated by a second distance after the embolic member exits lumen 1001. The second distance is less than the first distance. In this example, the central axis of an embolic member (such as 1003) can change from being relatively straight within lumen 1001 to being relatively arcuate after it exits lumen 1001.

In an example, flexible embolic members (such as 1003) can be relatively straight as they travel through lumen 1001, but they can curve into loops after exiting lumen 1001. In this example, the accumulation of curved, looping embolic members in aneurysm sac 1 forms a looping embolic mass that substantively occludes the aneurysm sac. In this example, the loops look similar to flower petals as they extend outwards from a central circle that is formed by longitudinal flexible member 1002. In this example, this embolic “flower petal” arrangement has a radial-expansion plane which is substantially perpendicular to the plane formed by the central circumference of the aneurysm neck. In an example, an embolic “flower petal” arrangement can have a radial-expansion plane which is substantially parallel to the plane formed by the central circumference of the aneurysm neck.

In an example, both ends of flexible embolic members (such as 1003) can be slideably attached to longitudinal flexible member 1002. In an example, pressure from other flexible embolic members accumulating in the aneurysm sac can push the ends of flexible embolic members (such as 1003) together as they accumulate in the aneurysm sac. In an example, one end of a flexible embolic member (such as 1003) can be slideably attached to longitudinal flexible member 1002, but the other end of the flexible embolic member may not be attached to the longitudinal flexible member.

In an example, the distal portion of longitudinal flexible member 1002 can be fused into a circle and detached from the proximal portion of longitudinal flexible member 1002 after a sufficient number of loops have accumulated in the aneurysm sac to occlude the aneurysm. FIG. 12 shows the distal portion of longitudinal flexible member 1002 having been fused into a circle and detached. FIG. 12 also shows lumens 1001 and 1004 having been removed.

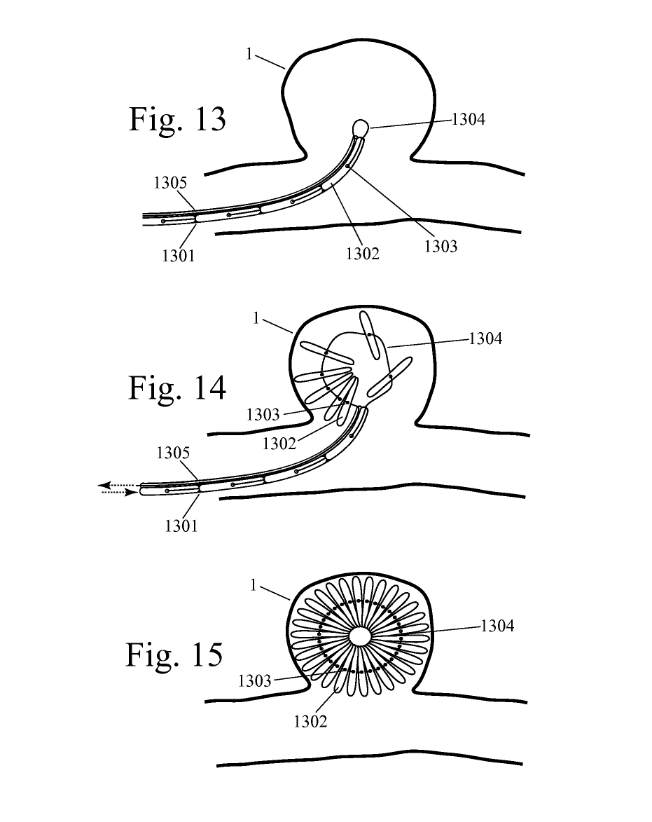

FIGS. 13 through 15 show an example of a device and method to occlude an aneurysm which can be described as inserting and rotating embolic members to occlude an aneurysm. More specifically, FIGS. 13 through 15 show an example of a device to occlude an aneurysm comprising: (a) a longitudinal lumen that is configured to be inserted into a blood vessel; (b) a connecting member that travels through the lumen and is configured to be inserted into an aneurysm; wherein this connecting member is longitudinal and flexible; wherein this connecting member has a longitudinal axis from a proximal end to a distal end as it travels through the lumen and has a cross-sectional area that is substantially perpendicular to its longitudinal axis; and (c) a plurality of embolic members that travel in a series configuration through the lumen and are configured to be inserted into the aneurysm; wherein these embolic members are connected and/or linked to each other by the connecting member; wherein an embolic member has a longitudinal axis that spans from its proximal end to its distal end as it travels through the lumen and a cross-sectional area that is substantively perpendicular to its longitudinal axis; wherein the longitudinal axis of the embolic member has a first orientation with respect to the longitudinal axis of the connecting member as the embolic member travels within the lumen; wherein the longitudinal axis of the embolic member rotates to a second orientation with respect to the longitudinal axis of the connecting member after the embolic member exits the lumen; wherein the second orientation is different than the first orientation; and wherein this second orientation of the longitudinal axis of the embolic member is substantially perpendicular to the longitudinal axis of the connecting member in the vicinity of the embolic member.

FIGS. 13 through 15 also show an example of a device to occlude an aneurysm comprising: (a) a longitudinal lumen that is configured to be inserted into a blood vessel, wherein this blood vessel is the parent vessel from which an aneurysm has formed; (b) a longitudinal flexible member that is configured to travel through the longitudinal lumen and be inserted into aneurysm sac; and (c) a plurality of longitudinal embolic members that are configured to travel through the longitudinal lumen and be inserted into the aneurysm sac; wherein these embolic members are connected by the longitudinal flexible member; wherein the longitudinal axis of an embolic member has a first orientation with respect to the longitudinal axis of the longitudinal flexible member as this embolic member travels through the longitudinal lumen; wherein the longitudinal axis of this embolic member has a second orientation with respect to the longitudinal axis of the longitudinal flexible member after this embolic member exits the longitudinal lumen; wherein the second orientation is closer to perpendicular than the first orientation; and wherein the accumulation of embolic members in the aneurysm sac substantively occludes the aneurysm sac.

In an example, the longitudinal lumen can be a removable catheter. In an example, the longitudinal flexible member can be selected from the group consisting of: wire, string, filament, chain, and coil. In an example, the longitudinal axis of an embolic member can be substantively parallel to the longitudinal axis of the longitudinal flexible member as the embolic member travels through the longitudinal lumen and the longitudinal axis of an embolic member can become substantively perpendicular to the longitudinal axis of the longitudinal flexible member after the embolic member exits the longitudinal lumen. In an example, the longitudinal flexible member can slide through central portions of the embolic members.

We now discuss the specific components of FIGS. 13 through 15 in detail. FIG. 13 shows an example of an occlusive device comprising: a first longitudinal lumen 1301 that is configured to be inserted into a blood vessel from which an aneurysm has formed; a second longitudinal lumen 1305 that is configured to be inserted into the blood vessel; a longitudinal flexible member 1304 that is configured to travel through one or both of these longitudinal lumens and to be inserted into the aneurysm sac; and a plurality of flexible embolic members (such as 1302) that are configured to travel through lumen 1301 and be inserted into the aneurysm sac, wherein each of these embolic members has a central opening (such as 1303) through which it is connected to longitudinal flexible member 1304. In this example, flexible member 1304 comprises a loop wherein one side of the loop travels through lumen 1301 and the other side travels through lumen 1305. In another example, a device can only have one lumen and both sides of a loop can travel through this one lumen.

In this example, the plurality of embolic members (including 1302) are connected by longitudinal flexible member 1304. In this example, the embolic members are slideably connected to the longitudinal flexible member by having the longitudinal flexible member go through openings (such as 1303) in the central portions of the embolic members. In this example, the longitudinal axis of an embolic member (such as 1302) has a first orientation with respect to the longitudinal axis of longitudinal flexible member 1304 as this embolic member travels through lumen 1305 and a second orientation with respect to the longitudinal axis of longitudinal flexible member 1304 after this embolic member exits lumen 1305. In an example, the second orientation can be closer to perpendicular than the first orientation.

As was the case with previous examples, the accumulation of embolic members in the aneurysm sac substantively occludes the aneurysm sac. In an example, the distal portion of longitudinal flexible member 1304 can be fused into a circle and detached from the proximal portion of longitudinal flexible member 1304 after a sufficient number of embolic members have accumulated in the aneurysm sac to occlude the aneurysm. FIG. 15 shows the distal portion of longitudinal flexible member 1304 having been fused into a circle and detached. FIG. 15 also shows lumens 1301 and 1305 having been removed.

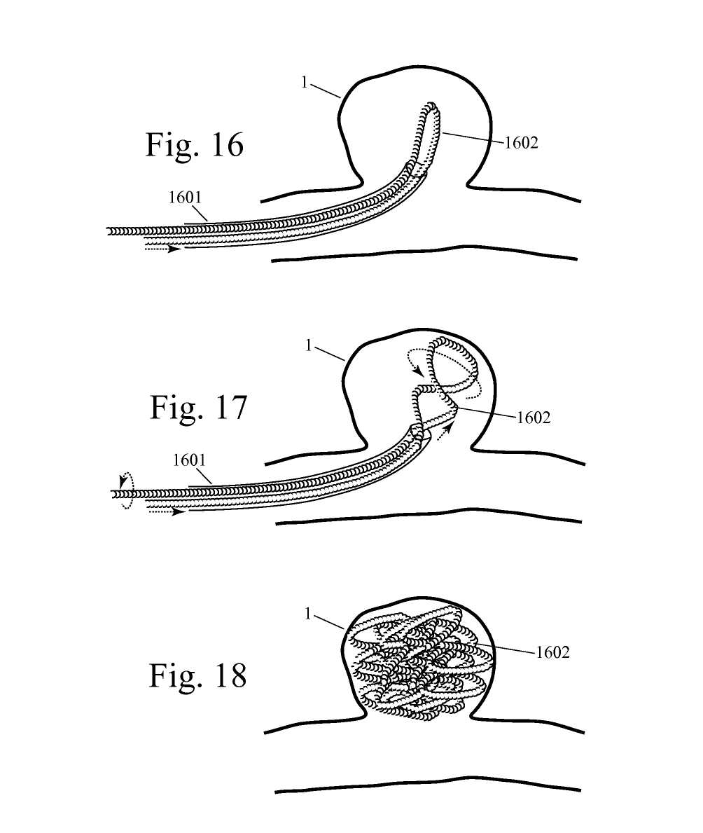

FIGS. 16 through 18 show an example of a device and method to occlude an aneurysm which can be described as coil rotation to form a densely-packed mass within an aneurysm. More specifically, FIGS. 16 through 18 show an example of a device to occlude an aneurysm comprising: (a) a first longitudinal section of a flexible longitudinal embolic member that travels through a lumen that is configured to be inserted into a blood vessel; wherein this embolic member is configured to be inserted into an aneurysm; and (b) a second longitudinal section of a flexible longitudinal embolic member that travels through a lumen that is configured to be inserted into a blood vessel; wherein this embolic member is configured to be inserted into an aneurysm; wherein this second longitudinal section is distally connected to the first longitudinal section; wherein the first longitudinal section is rotated around its longitudinal axis as the second longitudinal section is pushed from the lumen into the aneurysm; and wherein the combination of pushing the second section into the aneurysm and rotating the first section causes the first and second sections to become wound around each other and thereby create a densely-packed mass within the aneurysm.

FIGS. 16 through 18 also show an example of a device to occlude an aneurysm comprising: (a) a longitudinal lumen that is configured to be inserted into a blood vessel, wherein this blood vessel is the parent vessel from which an aneurysm has formed; (b) a first longitudinal flexible embolic member that is configured to travel through the longitudinal lumen and be inserted into the aneurysm sac; and (c) a second longitudinal flexible embolic member that is configured to travel through the longitudinal lumen and be inserted into the aneurysm sac; wherein the first and second embolic members are contiguous and/or connected to each other at their distal ends; and wherein the first embolic member is rotated around its longitudinal axis as the second embolic member is inserted into the aneurysm sac, causing the first and second embolic members to entwine around each other within the aneurysm sac.

In an example, the embolic members can be coils. In an example, both the first and second embolic members can be inserted into an aneurysm sac while the first embolic member is being rotated. In an example, the rate at which a first embolic member and/or a second embolic member is inserted into the aneurysm sac, the rate at which the first embolic member is rotated, or both of these rates can be selected based on the size and shape of an aneurysm sac so that the resulting entwined members substantially occlude the aneurysm sac. In an example, the rate at which a first embolic member and/or a second embolic member is inserted into the aneurysm sac, the rate at which a first embolic member is rotated, or both of these rates can be varied during implantation of the device so that the resulting entwined members substantially occlude an aneurysm sac. In an example, the rate at which the first embolic member is rotated can be adjusted from a faster rate to a slower rate during implantation.

FIGS. 16 through 18 also show an example of a device to occlude an aneurysm comprising: (a) a longitudinal lumen that is configured to be inserted into a blood vessel, wherein this blood vessel is the parent vessel from which an aneurysm has formed; and (b) a longitudinal flexible embolic loop that is configured to travel through the longitudinal lumen and be inserted into the aneurysm sac; wherein a first strand (or side) of the embolic loop is rotated around its longitudinal axis while a second strand (or side) of the embolic loop is pushed into the aneurysm; and wherein the combination of rotating the first strand (or side) of the loop while pushing the second strand (or side) of the loop into the aneurysm causes the first and second strands (or sides) to entwine around each other within the aneurysm sac.

In an example, both the first and second strands (or sides) of the loop can be inserted into the aneurysm sac while the first strand (or side) of the loop is being rotated. In an example, the rate at which the first strand (or side) of the loop and/or second strand (or side) of the loop is inserted into the aneurysm sac, the rate at which the first strand (or side) of the loop is rotated, or both of these rates can be selected based on the size and shape of an aneurysm sac so that the resulting entwined mass substantially occludes the aneurysm sac. In an example, one or both of these rates can be varied during implantation of the device. In an example, the rate at which the first strand (or side) of the loop is rotated can be varied from a faster rate to a slower rate during the implantation.

FIGS. 16 through 18 also show an example of a method for occluding an aneurysm comprising: (a) inserting a first longitudinal section of a flexible longitudinal embolic member through a lumen and into an aneurysm; and (b) rotating this first longitudinal section of a flexible longitudinal embolic member around its longitudinal axis as a second longitudinal section is pushed into the aneurysm; wherein this second longitudinal section is distally connected to the first longitudinal section; and wherein the combination of pushing the second section into the aneurysm and rotating the first section causes the first and second sections to become wound around each other and thereby create a densely-packed mass within the aneurysm.

We now discuss the specific components of FIGS. 16 through 18 in more detail. FIG. 16 shows an occlusive device that comprises: longitudinal lumen 1601 that is configured to be inserted into a blood vessel from which an aneurysm has formed; and longitudinal flexible embolic loop 1602 that is configured to travel through the longitudinal lumen and be inserted into the aneurysm sac. In this example, a first strand (or side) of embolic loop 1602 is rotated around its longitudinal axis while a second strand (or side) of embolic loop 1602 is pushed into the aneurysm. As shown in FIG. 17, the combination of rotating the first strand (or side) of loop 1602 around its longitudinal axis while pushing the second strand (or side) of loop 1602 into the aneurysm causes the first and second strands (or sides) to wind around each other within the aneurysm sac. As shown in FIG. 18, this creates an entwined mass which occludes the aneurysm sac.

In an example, the rate at which the first strand of loop 1602 is rotated, the rate at which one or both strands of loop 1602 are inserted into the aneurysm sac, or both rates can be selected based on the size and shape of an aneurysm sac. In an example, one or both of these rates can be varied during implantation. In an example, the rate at which the first strand of loop 1602 is rotated can vary from a faster rate to a slower rate during implantation. In an example, the rate at which the second strand of loop 1602 is pushed into the aneurysm sac can vary from a slower rate to a faster rate during implantation.

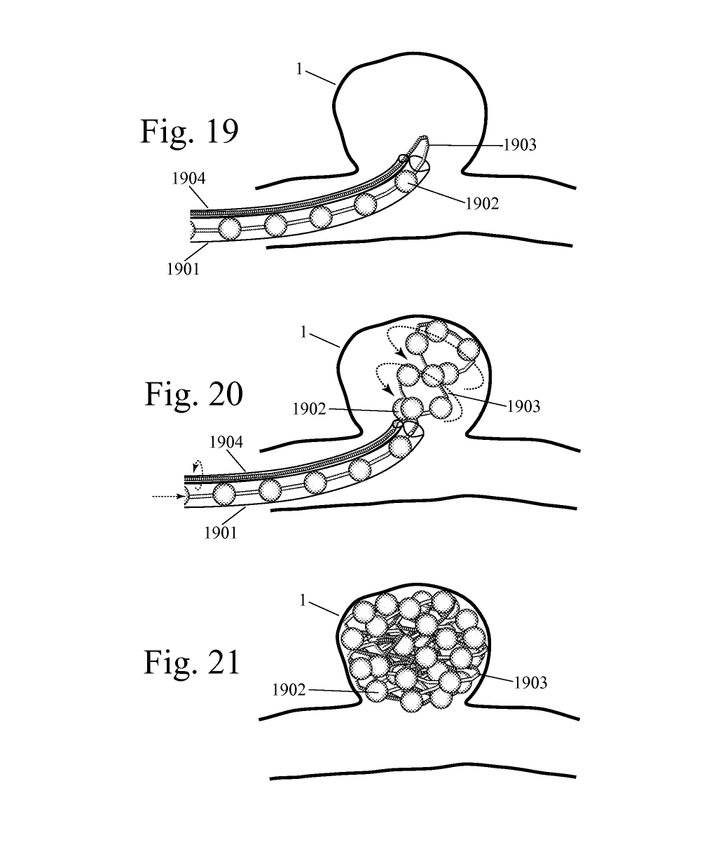

FIGS. 19 through 21 show an example of a device and method to occlude an aneurysm which can be described as “string-of-pearls” rotation to form a densely-packed mass within an aneurysm. More specifically, FIGS. 19 through 21 show an example of a device to occlude an aneurysm comprising: (a) a first longitudinal section of a flexible longitudinal embolic member that travels through a lumen that is configured to be inserted into a blood vessel; wherein this embolic member is configured to be inserted into an aneurysm; (b) a second longitudinal section of a flexible longitudinal embolic member that travels through a lumen that is configured to be inserted into a blood vessel; wherein this embolic member is configured to be inserted into an aneurysm; wherein this second longitudinal section is configured to be pushed from the lumen into the aneurysm; and wherein this second longitudinal section comprises a series of separate embolic members which are linked by a thin flexible central member; wherein this second longitudinal section is distally connected to the first longitudinal section; wherein the first longitudinal section is rotated around its longitudinal axis as the second longitudinal section is pushed from the lumen into the aneurysm; and wherein the combination of pushing the second section into the aneurysm and rotating the first section causes the first and second sections to become wound around each other and thereby create a densely-packed mass within the aneurysm.

FIGS. 19 through 21 also show an example of a device to occlude an aneurysm comprising: (a) a longitudinal lumen that is configured to be inserted into a blood vessel, wherein this blood vessel is the parent vessel from which an aneurysm has formed; (b) a first longitudinal flexible embolic member that is configured to travel through the longitudinal lumen and be inserted into the aneurysm sac; (c) a second longitudinal flexible embolic member that is configured to travel through the longitudinal lumen and be inserted into the aneurysm sac; wherein the first and second embolic members are contiguous and/or connected to each other at their distal ends; and wherein the first embolic member is rotated around its longitudinal axis as the second embolic member is inserted into the aneurysm sac, causing the first and second embolic members to entwine around each other within the aneurysm sac; and (d) a series of embolic members which are configured to travel through the longitudinal lumen and be inserted into the aneurysm sac; wherein these embolic members are connected by the first flexible member, the second flexible member, or both the first and second flexible members.

In an example, the longitudinal lumen can be a removable catheter. In an example, the longitudinal flexible member can be selected from the group consisting of: wire, string, filament, chain, and coil. In an example, the rate at which the first flexible member and/or the second flexible member is inserted into the aneurysm sac, the rate at which the first flexible member is rotated, or both of these rates can be selected based on the size and shape of the aneurysm sac so that the resulting mass substantially occludes the aneurysm sac. In an example, the rate at which the first flexible member and/or the second flexible member is inserted into the aneurysm sac, the rate at which the first flexible member is rotated, or both of these rates can be varied during implantation of the device so that the resulting mass substantially occludes the aneurysm sac. In an example, the rate at which the first flexible member is rotated can be varied from a faster rate to a slower rate during the implantation of the device. In an example, the plurality of embolic members can be evenly-spaced along the longitudinal axis of the first flexible member, the second flexible member, or both the first and second flexible members. In an example, the plurality of embolic members can be unevenly-spaced along the longitudinal axis of the first flexible member, the second flexible member, or both the first and second flexible members and wherein the uneven spacing of the embolic members is selected based on the size and shape of the aneurysm.

FIGS. 19 through 21 also show an example of a device to occlude an aneurysm comprising: (a) a first longitudinal lumen that is configured to be inserted into a blood vessel that is the parent vessel from which an aneurysm has formed; (b) a second longitudinal lumen that is configured to be inserted into the blood vessel; (c) a flexible loop that is configured to travel through the first and/or second longitudinal lumens and be inserted into the aneurysm sac, wherein a first strand (or side) of the loop is rotated around its longitudinal axis while a second strand (or side) of the loop is inserted into the aneurysm sac; and (d) a series of embolic members which are configured to travel through the longitudinal lumen and be inserted into the aneurysm sac; wherein these embolic members are connected by the second strand (or side) of the flexible loop.

In an example, rotation of a first strand (or side) of the flexible loop combined with insertion of a second strand (or side) of the loop (including attached embolic members) into the aneurysm sac can create a entwined embolic mass within the aneurysm sac. In an example, a first strand (or side) of the flexible loop can travel through a first longitudinal lumen and a second strand (or side) of the flexible loop can travel through a second longitudinal lumen. In another example, a device may have only one longitudinal lumen and both strands (or sides) of the flexible loop can travel through the same longitudinal lumen.

FIGS. 19 through 21 also show an example of a method for occluding an aneurysm comprising: (a) inserting a first longitudinal section of a flexible longitudinal embolic member into an aneurysm and rotating this first longitudinal section around its longitudinal axis; and (b) inserting a second longitudinal section of a flexible longitudinal embolic member into the aneurysm; wherein this second longitudinal section comprises a series of linked embolic members; wherein the second longitudinal section is distally connected to the first longitudinal section; and wherein the combination of inserting the second section into the aneurysm and rotating the first section causes the first and second sections to create a densely-packed embolic mass within the aneurysm.

We now discuss the specific components of FIGS. 19 through 21 in detail. FIG. 19 shows an example of an occlusive device that comprises: a first longitudinal lumen 1904 that is configured to be inserted into a blood vessel that is the parent vessel from which an aneurysm has formed; a second longitudinal lumen 1901 that is configured to be inserted into this blood vessel; a flexible loop 1903 that is configured to travel through the first and/or second longitudinal lumens and be inserted into the aneurysm sac, wherein a first strand (or side) of loop 1903 is rotated around its longitudinal axis while a second strand (or side) of loop 1903 is inserted into the aneurysm sac; and a series of embolic members (including 1902) which are configured to travel through longitudinal lumen 1901 and be inserted into the aneurysm sac, wherein these embolic members are connected by the second strand (or side) of flexible loop 1903.

As shown in FIG. 20, rotation of a first strand (or side) of flexible loop 1903 combined with insertion of a second strand (or side) of loop 1903 with attached embolic members (such as 1902) into the aneurysm sac creates an entwined embolic mass within the aneurysm sac. In this example, a first strand (or side) of flexible loop 1903 travels through first longitudinal lumen 1904 and a second strand (or side) of flexible loop 1903 travels through second longitudinal lumen 1901. In another example, an occlusive device may have only one longitudinal lumen and both strands (or sides) of a flexible loop can travel through the same longitudinal lumen.

In an example, the rate at which the first strand of loop 1903 is rotated, the rate at which one or both strands of loop 1903 are inserted into an aneurysm sac, or both rates can be selected based on the size and shape of the aneurysm sac. In an example, one or both of these rates can be varied during implantation. In an example, the rate at which the first strand of loop 1903 is rotated can vary from a faster rate to a slower rate during implantation. In an example, the rate at which the second strand of loop 1903 is pushed into the aneurysm sac can vary from a slower rate to a faster rate during implantation.

In an example, embolic members (including 1902) can be evenly-spaced along the second strand (or side) of flexible loop 1903. In an example, the spacing of embolic members (including 1902) along the second strand (or side) of flexible loop 1903 can be selected based on the size and shape of the aneurysm to be occluded. In an example, the spacing of embolic members (including 1902) along the second strand (or side) of flexible loop 1903 can vary from the distal end of flexible loop 1903 to the proximal end of flexible loop 1903.

As shown in FIG. 21, a distal portion of flexible loop 1903 can be detached and left in the aneurysm sac when a sufficient volume of embolic members has accumulated in the entwined mass. In an example, embolic members (including 1902) can adhere to each other so that the entwined mass that they form is more cohesive and stable. This can reduce the probability of portions of the loop or embolic members prolapsing into the parent blood vessel.

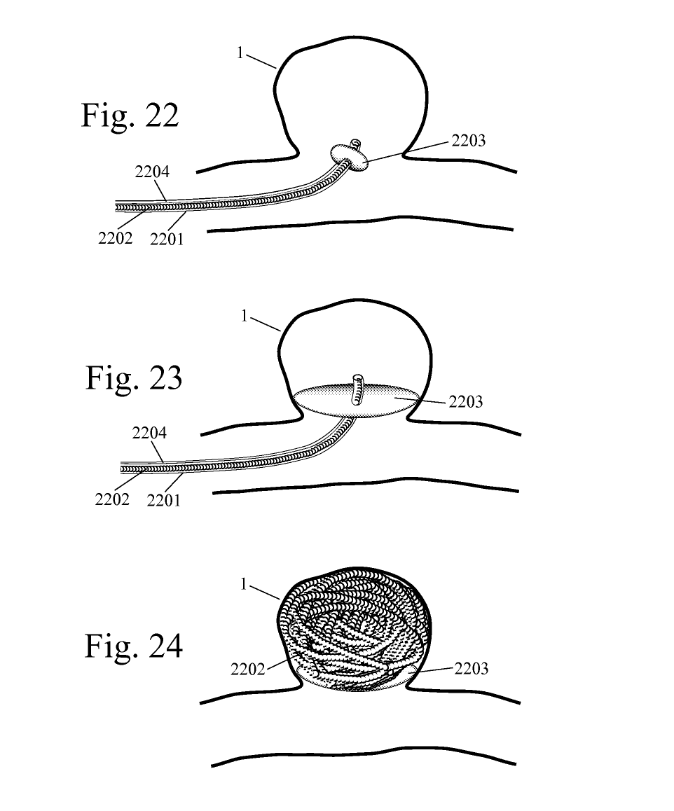

FIGS. 22 through 24 show an example of a device and method to occlude an aneurysm which can be described as aneurysm coil jailing using an expandable torus. More specifically, FIGS. 22 through 24 show an example of a device to occlude an aneurysm comprising: (a) a longitudinal flexible embolic member that is configured to be inserted into an aneurysm, wherein accumulation of this embolic member within the aneurysm occludes the aneurysm; (b) an embolic-member-delivering lumen which is configured to be inserted into a blood vessel, wherein the longitudinal flexible embolic member travels through this lumen in order to be inserted into the aneurysm; and wherein this embolic-member-delivering lumen is withdrawn from the blood vessel after the longitudinal flexible member has accumulated within the aneurysm; (c) a toroidal inflatable member that is configured to be inserted into the aneurysm; wherein this inflatable member substantially occludes the neck of an aneurysm when the inflatable member is inflated after being inserted into the aneurysm; and wherein the longitudinal flexible embolic member is inserted into the aneurysm through the central opening of the toroidal inflatable member after the toroidal member has been inflated; and (d) a flowable-substance-delivering lumen which is configured to be inserted into a blood vessel, wherein a flowable substance travels through this lumen into the toroidal inflatable member in order to inflate the toroidal member after it has been inserted into the aneurysm; and wherein this flowable-substance-delivering lumen is withdrawn from the blood vessel after the toriodal inflatable member has been inflated within the aneurysm.

FIGS. 22 through 24 also show an example of a device to occlude an aneurysm comprising: (a) a longitudinal lumen that is configured to be inserted into a blood vessel, wherein this blood vessel is the parent vessel from which an aneurysm has formed; (b) a longitudinal flexible embolic member that is configured to travel through the longitudinal lumen and be inserted into the aneurysm sac; and (c) an expandable toroidal member that is configured to travel through the longitudinal lumen, be inserted into the aneurysm sac, and to be expanded within the aneurysm sac; wherein this toroidal member is configured to substantially occlude the aneurysm neck after it is expanded; wherein the longitudinal flexible embolic member is inserted into the aneurysm sac through the central opening of the expandable toroidal member; and the expandable toroidal member prevents the longitudinal flexible member from protruding into the parent vessel of the aneurysm.

In an example, the longitudinal lumen can be a removable catheter. In an example, the longitudinal flexible embolic member can be a coil. In an example, the expandable toroidal member can be expanded by inflation with a gas. In an example, the expandable toroidal member can be expanded by filling it with a liquid or gel. In an example, the expandable toroidal member can self expand because it comprises shape memory material. In an example, the expandable toroidal member can remain in the aneurysm sac after implantation of the device. In an example, the expandable toroidal member can be expanded by filling it with a liquid or gel which solidifies after filling and wherein this solidification further helps to keep the expandable toroidal member in the aneurysm sac. In an example, the longitudinal flexible expandable member can adhere to the expandable toroidal member when the two members come into contact with each other and this adhesion can further help to keep the expandable toroidal member within the aneurysm sac.

We now discuss the specific components of the example shown in FIGS. 22 through 24. FIG. 22 shows an occlusive device which comprises: a first longitudinal lumen 2201 that is configured to be inserted into a blood vessel from which an aneurysm sac 1 has formed; a second longitudinal lumen 2204 that is configured to be inserted into this blood vessel; a longitudinal flexible embolic member 2202 that is configured to travel through the first longitudinal lumen 2201 and be inserted into the aneurysm sac; and an expandable toroidal member 2203 that is configured to be inserted into and expanded within aneurysm sac 1.

As shown in FIG. 23, toroidal member 2203 can be expanded by being filled with a gas, liquid, or gel that is delivered via second longitudinal lumen 2204. In an example, toroidal member 2203 can be configured to substantially occlude the aneurysm neck after it is expanded. In an example toroidal member 2203 can be expanded in a radial-expansion plane that is substantively parallel with the plane defined by the central circumference of the aneurysm neck. In an example, toroidal member 2203 can be expanded at a distance from the central circumference of the aneurysm neck from which its radial expansion contacts the aneurysm sac at the sac's maximum circumference. In an example, toroidal member 2203 can be expanded at a distance from the central circumference of the aneurysm neck from which its radial expansion contacts the aneurysm sac at a sac circumference that is greater than the central circumference of the aneurysm neck.

In an example, longitudinal flexible embolic member 2202 can be inserted into aneurysm sac 1 through a central opening in toroidal member 2203. In an example, longitudinal lumen 2201 can fit snugly through this opening so that longitudinal flexible embolic member 2202 does not prolapse out from this opening into the parent vessel. In an example, toroidal member 2203 can prevent longitudinal flexible member 2202 from prolapsing into the parent blood vessel of the aneurysm. In an example, a plurality of embolic members can be inserted into the aneurysm sac through this central opening and toroidal member 2203 can have a sufficiently tight fit with the aneurysm walls that none of the plurality of embolic members escape into the parent blood vessel.

In the example that is shown in FIGS. 22 through 24, longitudinal lumens 2201 and 2204 are parallel catheters which are removed from the body after the aneurysm is occluded. In this example, longitudinal flexible embolic member 2202 is an embolic coil. In this example, toroidal member 2203 is expanded by being filled with a gas, liquid, or gel. In an example, a toroidal member can self expand because it comprises shape memory material, thereby eliminating the need for longitudinal lumen 2204. As shown in FIG. 24, expandable toroidal member 2203 can remain in the aneurysm sac after implantation, but longitudinal lumens 2201 and 2204 can be removed. In an example, longitudinal flexible expandable member 2202 can adhere to toroidal member 2203 to help keep toroidal member from protruding into the blood vessel.

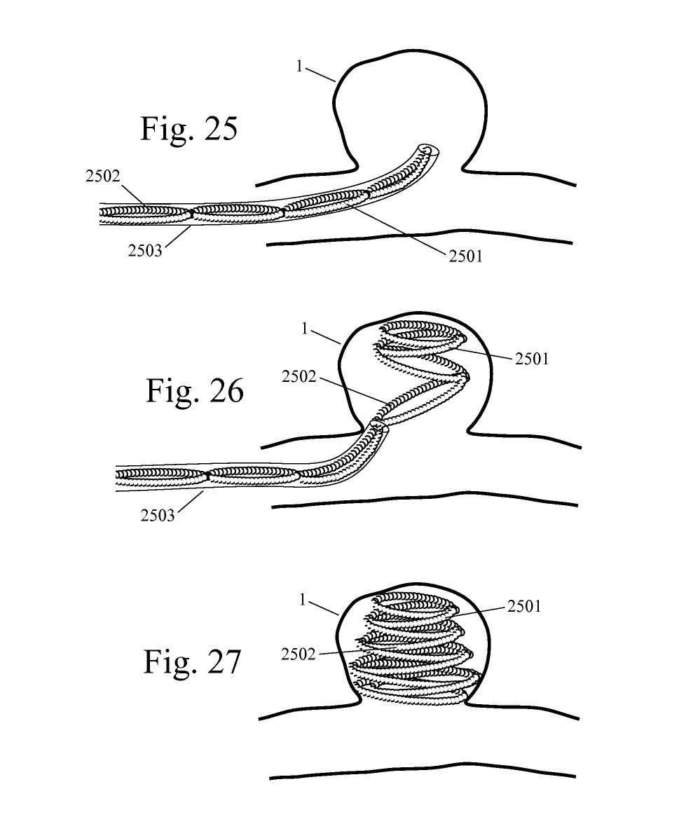

FIGS. 25 through 27 show an example of a device and method to occlude an aneurysm which can be described as aneurysm occlusion using loops which are substantially-parallel to the aneurysm neck. More specifically, FIGS. 25 through 27 show an example of a device to occlude an aneurysm comprising: (a) a longitudinal lumen that is configured to be inserted into a blood vessel; and (b) a flexible longitudinal embolic member that is configured to travel through the lumen in order to be inserted through the lumen into an aneurysm; wherein this flexible longitudinal embolic member comprises a longitudinal series of loops; wherein these loops are laterally compressed as they travel through the lumen and laterally expand within the aneurysm after they exit the lumen; wherein adjacent loops along the longitudinal series are connected to each other by junctions; and wherein loop sizes are selected to be substantially equal to the circumferences of the aneurysm sack at the expected implant locations so that junction locations alternate from one side of the aneurysm to the other side of the aneurysm as successive loops accumulate within the aneurysm.

FIGS. 25 through 27 also show an example of a device to occlude an aneurysm comprising: (a) a longitudinal lumen that is configured to be inserted into a blood vessel; and (b) a flexible longitudinal embolic member that is configured to travel through the lumen in order to be inserted through the lumen into an aneurysm; wherein this flexible longitudinal embolic member comprises a longitudinal series of loops; wherein these loops are laterally compressed as they travel through the lumen and laterally expand within the aneurysm after they exit the lumen; and wherein loop sizes are selected to be substantially equal to the circumferences of the aneurysm sack at the expected implant locations so that the planes formed by loops are substantially within plus or minus 30 degrees of being parallel to the plane formed by the aneurysm neck.

FIGS. 25 through 27 also show an example of a device to occlude an aneurysm comprising: (a) a longitudinal lumen that is configured to be inserted into a blood vessel, wherein this blood vessel is the parent vessel from which an aneurysm has formed; (b) a first longitudinal flexible embolic member that is configured to travel through the longitudinal lumen and be inserted into the aneurysm sac; (c) a second longitudinal flexible embolic member that is configured to travel through the longitudinal lumen and be inserted into the aneurysm sac; wherein the first and second embolic members are contiguous and/or connected to each other at their distal ends; wherein the first and second embolic members are connected at a plurality of locations along the lengths of these embolic members; wherein segments of the first and second embolic members that are not connected move away from each other after they exit the longitudinal lumen, thereby forming loops within the aneurysm sac; and wherein a plane spanned by a loop is substantively parallel to the plane spanned by the central circumference of the aneurysm neck.

In an example, the longitudinal lumen can be a removable catheter. In an example, the embolic members can be coils. In an example, a plane spanned by a loop can be within 30 degrees of being parallel to the plane spanned by the central circumference of the aneurysm neck. In an example, a plane spanned by a loop can be within 45 degrees of being parallel to the plane spanned by the central circumference of the aneurysm neck. In an example, the orientations of sequential loops formed within the aneurysm sac can sequentially alternate from one side of the aneurysm sac to the other side of the aneurysm sac. In an example, the accumulating loops can form a beehive-shaped mass of loops within the aneurysm sac and not prolapse out from the aneurysm neck into the parent vessel.

We now discuss the specific components of the example that is shown in FIGS. 25 through 27. FIG. 25 shows an occlusive device comprising: a longitudinal lumen 2503 that is configured to be inserted into a blood vessel, wherein this blood vessel is the parent vessel from which an aneurysm sac 1 has formed; a first longitudinal flexible embolic member 2501 that is configured to travel through the longitudinal lumen and be inserted into aneurysm sac 1; and a second longitudinal flexible embolic member 2502 that is configured to travel through the longitudinal lumen and be inserted into aneurysm sac 1. In this example, embolic members 2501 and 2502 are contiguous to each other at their distal ends. In this example, embolic members 2501 and 2502 are connected at a plurality of locations along their lengths. As shown in FIG. 26, segments of embolic members 2501 and 2502 that are not connected to each other move away from each other after they exit the longitudinal lumen, thereby forming connected loops within aneurysm sac 1.

In this example, a plane that is spanned by one of these loops can become (in its final configuration) substantively parallel to the plane that is spanned by the central circumference of the aneurysm neck. In an example, this final configuration can occur after multiple such loops have been formed and accumulated in the aneurysm sac. In an example, a plane spanned by a loop can become within plus-or-minus 30 degrees of being parallel to the plane spanned by the central circumference of the aneurysm neck. In an example, virtual extension of the plane spanned by a loop can form an acute angle with the virtual extension of the plane spanned by the central circumference of the aneurysm neck. In an example, this angle can be less than 30 degrees. In an example, a plane spanned by a loop can become within plus-or-minus 45 degrees of being parallel to the plane spanned by the central circumference of the aneurysm neck. In an example, virtual extension of the plane spanned by a loop can form an acute angle with the virtual extension of the plane spanned by the central circumference of the aneurysm neck. In an example, this angle can be less than 45 degrees.

As shown in FIG. 27, the accumulation of sequential loops with sides comprised of embolic members 2501 and 2502 can form an embolic mass with a zigzag pattern within the aneurysm sac. In this example, the acute angles formed between the zigzagging loops are less than 30 degrees. In an example, the orientations of sequential loops formed within aneurysm sac 1 can sequentially alternate from one side of aneurysm sac 1 to the other side of aneurysm sac 1. As shown in FIG. 27, accumulating loops can form a “beehive-shaped” mass of loops within the aneurysm sac. In an example, the probability of having embolic members prolapse into the parent blood vessel can be reduced (as compared to free-form spiraling coils in the prior art) by a combination of: (a) creating loops which frictionally engage the aneurysm sac walls at one or more locations; (b) creating loops with an orientation which is substantially parallel to the plane defined by the central circumference of the aneurysm neck; and (c) creating loops which are contiguous and interconnected.

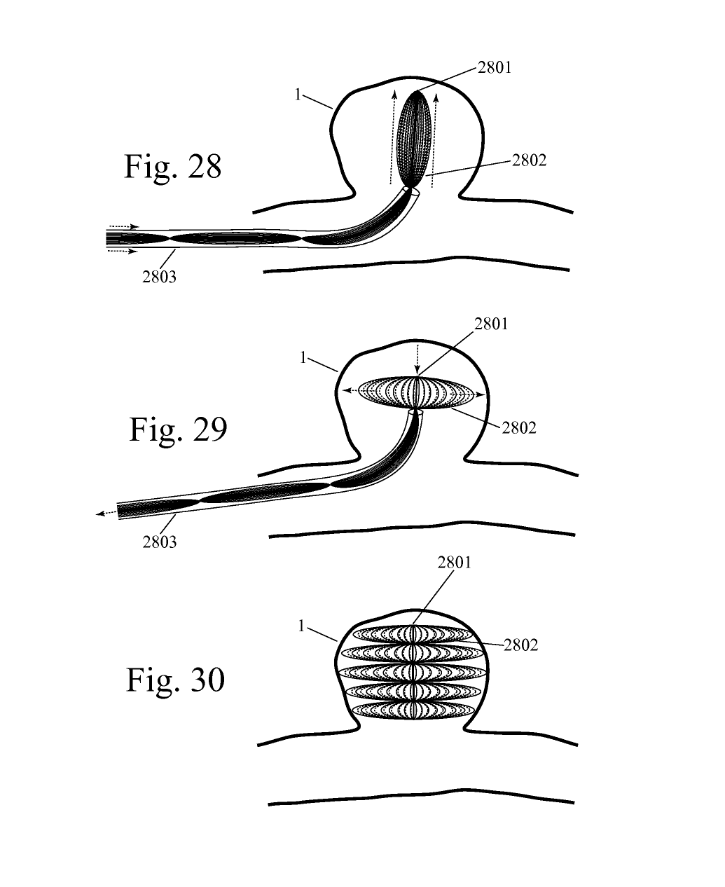

FIGS. 28 through 30 show an example of a device and method to occlude an aneurysm which can be described as aneurysm occlusion using multiple centrally-aligned ellipsoids. More specifically, FIGS. 28 through 30 show an example of a device to occlude an aneurysm comprising: (a) a longitudinal lumen that is configured to be inserted into a blood vessel, wherein this lumen has a longitudinal axis spanning from its proximal end to its distal end and wherein the distal end is first inserted into the blood vessel; and (b) a plurality of longitudinally-linked configuration-changing embolic members which are configured to travel through the longitudinal lumen and to be inserted into an aneurysm; wherein each shape-changing embolic member has its own internally-referenced Z axis, X axis, and Y axis; wherein its Z axis is substantially parallel to the longitudinal axis of the longitudinal lumen as the embolic member travels through the longitudinal lumen, its X axis is substantially perpendicular to its Z axis, and its Y axis is substantially perpendicular to both its Z axis and X axis; wherein each configuration-changing embolic member has a first configuration as the member travels through the longitudinal lumen and a second configuration within the aneurysm after it exits the longitudinal lumen; wherein the distance of the embolic member spanning its Z axis is greater than the distance of the embolic member spanning its X axis or Y axis in the first configuration; wherein the distance of the embolic member spanning its Z axis is less than the distance of the embolic member spanning its X axis or Y axis in the second configuration; wherein the cross-sectional shape of the embolic member in an X-Z plane is substantially elliptical, oval, or another arcuate non-circular shape in the first configuration, with the longer dimension of the ellipse, oval, or another arcuate non-circular shape being along its Z axis; and wherein the cross-sectional shape of the embolic member in the X-Z plane is substantially elliptical, oval, or another arcuate non-circulate shape in the second configuration, with the longer dimension of the ellipse, oval, or another arcuate non-circular shape being along its X axis.

FIGS. 28 through 30 also show an example of a device to occlude an aneurysm comprising: (a) a longitudinal lumen that is configured to be inserted into a blood vessel, wherein this blood vessel is the parent vessel from which an aneurysm has formed; and (b) a series of connected embolic ellipsoids, wherein these embolic ellipsoids are configured to travel in series through the longitudinal lumen and to be inserted into the aneurysm sac; wherein an embolic ellipsoid has a first orientation as it travels through the longitudinal lumen; wherein an embolic ellipsoid has a second orientation after it exits the longitudinal lumen; wherein in the first orientation the longitudinal axis of the ellipsoid is substantively parallel to the longitudinal axis of the longitudinal lumen; wherein in the second orientation the longitudinal axis of the ellipsoid is substantially perpendicular to its prior orientation traveling through the longitudinal lumen.

In an example, the longitudinal lumen can be a removable catheter. In an example, the embolic ellipsoid can be a wire structure. In an example, the embolic ellipsoid can have a first orientation when it exits the aneurysm sac but then be compressed into a second orientation. In an example, the series of connected embolic ellipsoids can form a stack of connected ellipsoids which share a common central axis within the aneurysm sac. In an example, the series of connected embolic ellipsoids can form a stack of connected ellipsoid disks which share a common central axis within the aneurysm sac and fill a greater volume of the aneurysm sac than would be filled by a single hollow mesh structure with a similar size perimeter as the stack of connected ellipsoid disks. In an example, at least one of the connected ellipsoid disks can have a circumference that is larger than the circumference of the aneurysm neck in order to help keep the structure within the aneurysm sac.

We now discuss the components of the example that is shown in FIGS. 28 through 30 in detail. FIG. 28 shows an occluding device that comprises: a longitudinal lumen 2803 that is configured to be inserted into a blood vessel, wherein this blood vessel is the parent vessel from which an aneurysm has formed; and a series of connected embolic ellipsoids (including 2802). In this example, the embolic ellipsoids (including 2802) are configured to travel in series through longitudinal lumen 2803 and be inserted into the aneurysm sac.

As shown in FIG. 29, each embolic ellipsoid (including 2802) can have a first orientation as it travels through lumen 2803 and a second orientation after it exits lumen 2803 inside aneurysm sac 1. In an example, each embolic ellipsoid can have a longitudinal axis such as 2801 for embolic ellipsoid 2802. In an example, in the first orientation, the longitudinal axis (such as 2801) of the ellipsoid (such as 2802) can be substantively-parallel to the longitudinal axis of lumen 2803. In an example, in the second orientation, the longitudinal axis (such as 2801) of the ellipsoid (such as 2802) can be substantially-perpendicular to its prior orientation traveling through lumen 2803.

In an example, an embolic ellipsoid (such as 2802) can be oriented as it travels through lumen 2803 such that its longest axis is substantially-parallel to the longitudinal axis of lumen 2803. In an example, an embolic ellipsoid (such as 2802) can be compressed and/or reoriented after it exits lumen 2803 so that its longest axis becomes substantially-parallel to the plane that is defined by the central circumference of the aneurysm neck. In an example, the longitudinal axes (such as 2801) of the embolic ellipsoids (such as 2802) as these ellipsoids travel through lumen 2803 can become the virtual lateral axes (still 2801) of these embolic ellipsoids (such as 2802) when these ellipsoids are compressed and/or reoriented after they exit lumen 2803.

In example, the longitudinal axes (including 2802) of these ellipsoids (including 2802) can be compressed after the ellipsoids exit lumen 2803. In an example, this compression can be caused by movement of a wire, fiber, or other longitudinal flexible member that is connected to the ellipsoids. In an example, this compression can be caused by contact between the aneurysm wall and the ellipsoids. In an example, the embolic ellipsoids (including 2802) can have a shape memory and a prior shape to which they return after their release from lumen 2803. In an example, their return to a prior shape can cause the change in their orientation and/or compression after they exit lumen 2803. In this example, the embolic ellipsoids (including 2802) are wire structures.

As shown in FIG. 30, a series of connected embolic ellipsoids (including 2802) can form a stack of connected ellipsoids which share a common central axis within aneurysm sac 1. In an example, a series of connected embolic ellipsoids can form a stack of connected ellipsoid disks which share a common central axis within the aneurysm sac. In an example, this stack of connected ellipsoids can fill a greater volume of the aneurysm sac than would be filled by a single hollow-mesh structure (such as a wire-mesh single sphere or ellipsoid that is expanded with an aneurysm sac) with a similar-size perimeter as the combined stack of connected ellipsoid disks. As shown in FIG. 30, at least one of the connected ellipsoids has a circumference that is larger than the circumference of the aneurysm neck in order to help keep the stack within the aneurysm sac.

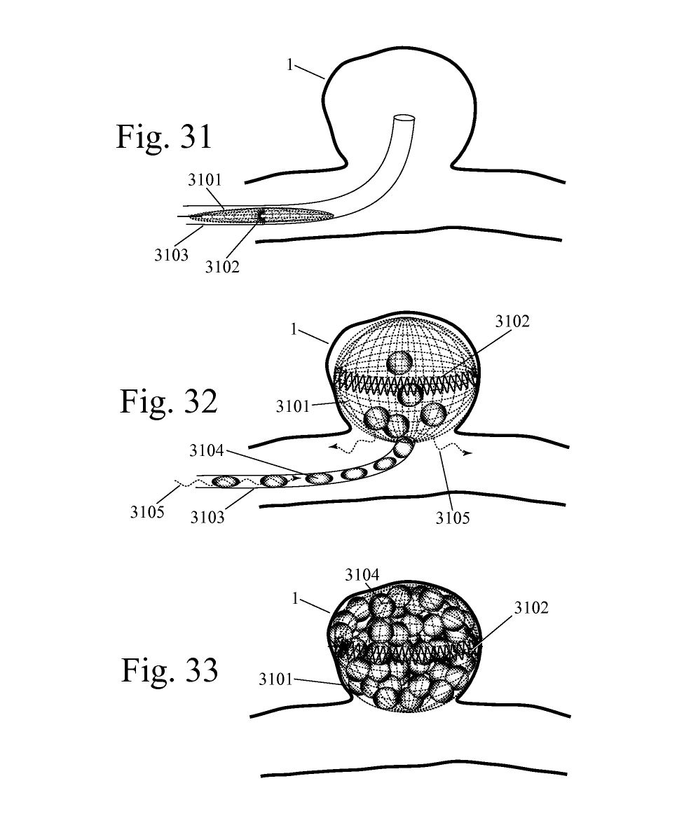

FIGS. 31 through 33 show an example of a device and method to occlude an aneurysm which can be described as a “Saturn-shaped” device for aneurysm occlusion. More specifically, FIGS. 31 through 33 show an example of a device to occlude an aneurysm comprising: (a) a longitudinal lumen that is configured to be inserted into a blood vessel; (b) a flexible expandable member that is configured to travel through the longitudinal lumen, be inserted into an aneurysm, and then be expanded within the aneurysm sack; wherein this flexible expandable member is selected from the group consisting of a net, a mesh, a lattice, and a balloon with holes; wherein this flexible expandable member is sufficiently flexible to substantively conform to the contours of the walls of the aneurysm sack after the flexible expandable member is expanded within the aneurysm; and wherein this flexible expandable member is permeable to liquid; (c) a resilient expandable member that is configured to travel through the longitudinal lumen, be inserted into the aneurysm, and then be expanded within the aneurysm sack; wherein this resilient expandable member resists contraction after it has been expanded; wherein a plane formed by the expanding circumference of this resilient expandable member is substantially parallel to the plane that centrally spans the circumference of the aneurysm neck; wherein a plane formed by the expanding circumference of this resilient expandable member spans the aneurysm sack at the sack's largest circumference parallel to the plane that centrally spans the circumference of the aneurysm neck; and wherein expansion of the resilient expandable member resiliently holds a central portion of the flexible expandable member against the walls of the aneurysm so that the flexible expandable member does not slip out of the aneurysm sack; and (d) a plurality of individual embolic members that are configured to travel through the longitudinal lumen, be inserted into the flexible expandable member within the aneurysm, and accumulate within the flexible expandable member; wherein the flexible expandable member does not allow the embolic members to escape out from the flexible expandable member; and wherein accumulation of the plurality of embolic members inside the flexible expandable member causes the flexible expandable member to expand.

FIGS. 31 through 33 also show an example of a device to occlude an aneurysm comprising: (a) a longitudinal lumen that is configured to be inserted into a blood vessel, wherein this blood vessel is the parent vessel from which an aneurysm has formed; (b) an expandable flexible net or mesh, wherein this expandable flexible net or mesh is configured to travel through the longitudinal lumen and to be inserted into the aneurysm sac, and wherein this net or mesh is sufficiently flexible to substantially conform to the walls of an irregularly shaped aneurysm sac after the net or mesh has been expanded; (c) a plurality of embolic members, wherein these embolic members are configured to travel through the longitudinal lumen and to be inserted into the net or mesh within the aneurysm sac; wherein these embolic members do not escape from the net or mesh; and wherein the net or mesh is expanded by the accumulation of embolic members inside the net or mesh; and (d) an expandable resilient structure, wherein this expandable resilient structure is configured to travel through the longitudinal lumen and to be inserted into the aneurysm sac; wherein this structure comes into engaging contact with the central circumference of the aneurysm sac when this structure is expanded; wherein this structure resists compression after it has been expanded; and wherein expansion of this structure also engages the net or mesh so as to prevent the net or mesh from slipping out from the aneurysm sac.

In an example, the longitudinal lumen can be a removable catheter. In an example, the net or mesh can be a wire net or mesh. In an example, the net or mesh can be a polymer net or mesh. In an example, the expandable resilient structure can be a stent. In an example, the expandable resilient structure can be attached to the net or mesh. In an example, the expandable resilient structure can be inside the net or mesh. In an example, the plurality of embolic members can be conveyed through the longitudinal lumen by means of a liquid flow and the net or mesh can be sufficiently porous so as to let the liquid escape through the net or mesh but does not let the embolic members escape through the net or mesh. In an example, the total volume of an aneurysm sac can be X cubic units, wherein Y cubic units of the volume of the aneurysm would be filled by the largest-volume sphere that can be fitted into the aneurysm without stretching the aneurysm walls, wherein Z cubic units of the volume of the aneurysm can be filled by the net or mesh; and wherein Z>[Y+0.5(X−Y)]. In an example, the total volume of an aneurysm sac can be X cubic units, wherein Y cubic units of the volume of the aneurysm would be filled by the largest-volume ellipsoid that can be fitted into the aneurysm without stretching the aneurysm walls, wherein Z cubic units of the volume of the aneurysm can be filled by the net or mesh; and wherein Z>[Y+0.5(X−Y)].

We now discuss the specific components of the example that is shown FIGS. 31 through 33. FIGS. 31 through 33 show an occlusive device comprising: longitudinal lumen 3103 that is inserted into a blood vessel from which an aneurysm sac 1 has formed; expandable flexible net or mesh 3101 that travels through lumen 3103 into aneurysm sac 1, wherein net or mesh 3101 is sufficiently flexible to substantially conform to the walls of aneurysm sac 1; a plurality of embolic members (including 3104) which travel through lumen 3103 into net or mesh 3101, wherein these embolic members (including 3104) do not escape from net or mesh 3101 and wherein net or mesh 3101 is expanded by the accumulation of embolic members (including 3104) inside net or mesh 3101; and expandable resilient structure 3102 which travels through lumen 3103 and is expanded within aneurysm sac 1.

In an example, a flexible expandable member can be selected from the group consisting of a net, a mesh, and a lattice. In an example, a flexible expandable member can be selected from the group consisting of a balloon, a bag, and a liner. In an example, a flexible expandable member is made of a polymer, a metal, or a combination thereof.

As shown in FIG. 32, structure 3102 comes into engaging contact with the central circumference of aneurysm sac 1 when structure 3102 is expanded. Structure 3102 can be expanded sufficiently to frictionally engage the walls of aneurysm sac 1, but not expanded so much that it risks puncturing the walls of aneurysm sac 1. In an example, structure 3102 can have a rounded perimeter. In an example, structure 3102 can have a bioadhesive coating which adheres to the aneurysm walls to further engage them. Structure 3102 also resists compression after it expands. In this example, expansion of structure 3102 also engages net or mesh 3101 to prevent net or mesh 3101 from slipping out from aneurysm sac 1.

In this example, longitudinal lumen 3103 is a removable catheter. In this example, net or mesh 3101 is a wire net or mesh. In an example, net or mesh 3101 can be a polymer net or mesh. In this example, expandable resilient structure 3102 is integrated with net or mesh 3101. In an example, the plurality of embolic members (including 3104) can be conveyed through lumen 3103 by means of a liquid flow. In an example, net or mesh 3101 can be sufficiently porous so as to let the liquid escape through net or mesh 3101 but not so porous that it lets embolic members (including 3104) escape through net or mesh 3101. In an example, embolic members (including 3104) can be compressed as they travel through lumen 3103 but these embolic members (including 3104) can expand when released from lumen 3103. This can help to prevent embolic members from escaping out of net or mesh 3101.

In an example, this device can also include a closure mechanism which is integrated into net or mesh 3101 to further prevent embolic members (including 3104) from escaping from net or mesh 3101 through the opening by which they were inserted into net or mesh 3101. In an example, this closure mechanism can comprise a one-way valve that automatically lets embolic members into the net or mesh but does not let them out. In an example, this closure mechanism can require action by a user during the procedure to close off the opening. In an example, this closure mechanism can comprise a drawstring, loop, seal, fusible member, adhesive, snap, clip, valve, or cap.

As shown in FIGS. 31 through 33, an aneurysm sac can be irregular in shape. An aneurysm sac with an irregular shape will not be completely filled or spanned by a spherical or ellipsoid mass without stretching the aneurysm walls. In an example, the total volume of an aneurysm sac can be X cubic units (e.g. cubic millimeters). In an example, the maximum volume of the aneurysm which can be filled or spanned by a spherical or ellipsoid mass without stretching the aneurysm walls is Y cubic units (e.g. cubic millimeters). In an example, the device shown in FIGS. 31 through 33 can fill more of the aneurysm than a spherical or ellipsoid mass because the net or mesh is sufficiently flexible to fill or span the irregular perimeter of the aneurysm sac. This can have clinical benefits, such as reducing the chances of recanalization within the aneurysm sac. In an example, this device can fill or span more than 50% of the aneurysm volume which remains unfilled by a sphere or ellipsoid. In an example, this device can fill or span Z cubic units (e.g. cubic millimeters) of the volume of the aneurysm, wherein Z>[Y+0.5(X−Y)].

In an example, net or mesh 3101 can be compressed as it travels through lumen 3103 and then be expanded within aneurysm sac 1 after it is released from lumen 3103. In an example, net or mesh 3101 can be folded as it travels through lumen 3103 and then be unfolded within aneurysm sac 1. In an example, net or mesh 3101 can be relatively loose or relaxed (in a lower-energy state) as it travels through lumen 3103 and then be stretched or tense (in a higher-energy state) within aneurysm sac 1. In an example, net or mesh 3101 can be elastic or stretchable. In an example, net or mesh 3101 can be sufficiently elastic or stretchable that it expands when filled with an accumulation of embolic members (including 3104), but not so elastic or stretchable that it allows embolic members (including 3104) to escape. In an example, net or mesh 3101 can be a balloon with holes, wherein the holes are of sufficient size to let liquid escape, but not so large that they let embolic members escape.

In an example, expandable resilient structure 3102 can be a ring-like expandable stent. In an example, expandable resilient structure 3102 can be a cylindrical expandable stent. In an example, expandable resilient structure can be an ellipsoid expandable stent. In an example, expandable resilient structure 3102 can be a wire mesh stent. In an example, expandable resilient structure 3102 can be centrally-located so as to expand from the center of net or mesh 3101. In an example, expandable resilient structure 3102 can be inside net or mesh 3101 and thereby hold net or mesh against the aneurysm wall when structure 3102 is expanded. In an example, expandable resilient structure 3102 can be attached to net or mesh 3101 and thereby hold net or mesh 3101 within the aneurysm sac when structure 3102 is expanded. In an example the expandable resilient structure 3102 can be radially-expanded in plane which is substantially parallel to the plane that is defined by the central circumference of the aneurysm neck. In an example, expandable resilient structure 3102 can be expanded by a removable balloon. In an example, expandable resilient structure 3102 can self-expand when released from lumen 3103.

In an example, embolic members (including 3104) can be a plurality of soft, compressible members such as microsponges or blobs of gel. In an example, embolic members (including 3104) can be a plurality of hard, uncompressible members such as hard polymer spheres or beads. In an example, embolic members (including 3104) can be conveyed through lumen 3103 in a fluid flow, wherein the fluid escapes out from net or mesh 3101 and the embolic members are retained within net or mesh 3101. In an example, embolic members (including 3104) can be conveyed through lumen 3103 by means of a moving belt or wire loop. In an example, embolic members (including 3104) can be conveyed through lumen 3103 by means of an Archimedes screw.

In an example, the combination of (a) a flexible, non-resilient net or mesh 3101 that spans substantially the entire perimeter of the aneurysm sac 1 and (b) a resilient expandable structure 3102 that only spans a central portion of the circumference of the aneurysm sac 1 can create a device that is sufficiently flexible to substantially fill the entire volume of an irregularly-shaped aneurysm sac, but also sufficiently resilient so as to compress against the aneurysm walls and not slip out of the aneurysm sac.

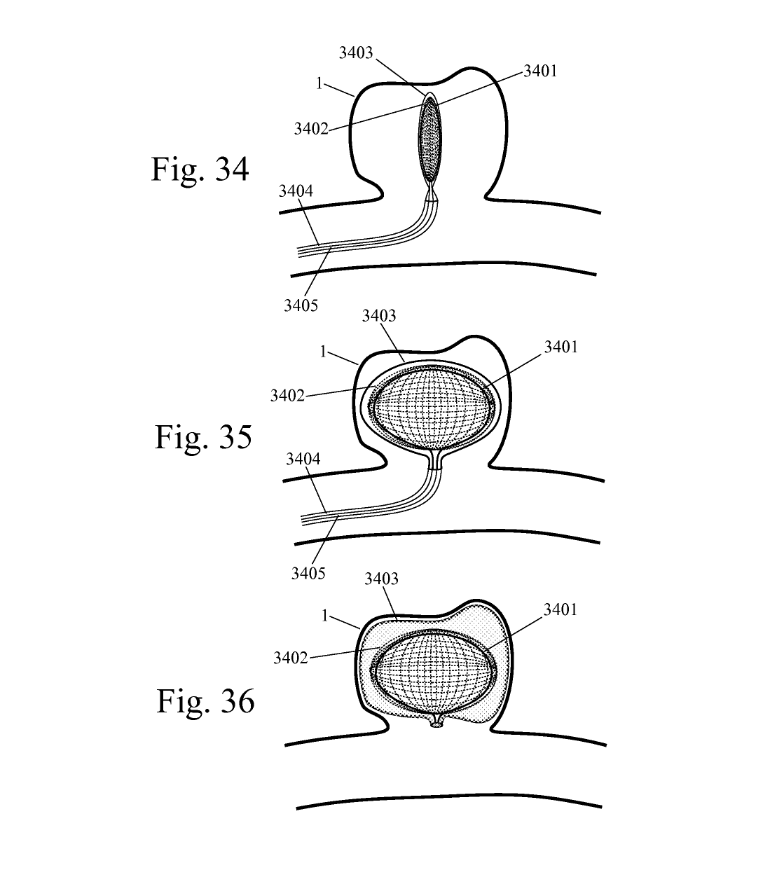

FIGS. 34 through 36 show an example of a device and method to occlude an aneurysm which can be described as using concentric resilient and non-resilient intrasacular members for aneurysm occlusion. More specifically, FIGS. 34 through 36 show an example of a device to occlude an aneurysm comprising: (a) a longitudinal lumen that is configured to be inserted into a blood vessel; (b) a resilient expandable member that is configured to travel through the longitudinal lumen, be inserted into an aneurysm sack, and then be expanded within the aneurysm sack; wherein this resilient expandable member resists contraction after it has been expanded; and wherein this resilient expandable member has a post-expansion shape that is selected from the group consisting of spherical, ellipsoidal, toroidal, compressed-sphere shaped, egg shaped, Saturn shaped, hour-glass shaped, peanut shaped, beehive shaped and geodesic; and (c) a flexible expandable member that is configured to travel through the longitudinal lumen, be inserted into the aneurysm sack, and then be expanded within the aneurysm sack; wherein the resilient expandable member is inside the flexible expandable member; wherein the resilient expandable member is expanded before or while the flexible expandable member is expanded; and wherein the flexible expandable member is sufficiently flexible to substantively conform to the contours of the walls of the aneurysm sack when the flexible expandable member is expanded within the aneurysm.

FIGS. 34 through 36 also show an example of a device to occlude an aneurysm comprising: (a) a longitudinal lumen that is configured to be inserted into a blood vessel, wherein this blood vessel is the parent vessel from which an aneurysm has formed; (b) an expandable flexible member, wherein this expandable flexible member is configured to travel through the longitudinal lumen and to be inserted into the aneurysm sac; and wherein this flexible member is sufficiently flexible to substantially conform to the walls of an irregularly-shaped aneurysm sac after the flexible member has been expanded; and (c) an expandable resilient structure, wherein this expandable resilient structure is configured to travel through the longitudinal lumen and to be inserted into the aneurysm sac; wherein this structure is expanded inside the expandable flexible member; and wherein this structure resists compression after it has been expanded.

In an example, the longitudinal lumen can be a removable catheter. In an example, the expandable flexible member can be a balloon. In an example, the expandable flexible member can be a net or mesh. In an example, the expandable flexible member can be expanded by being filled with a liquid, gel, gas, or embolic members. In an example, the expandable resilient structure can be a stent. In an example, the expandable resilient structure can be substantially spherical or elliptical. In an example, the expandable resilient structure can be expanded before the expandable flexible member is expanded. In an example, the expandable resilient structure and the expandable flexible member can be expanded at substantially the same time. In an example, the total volume of an aneurysm sac can be X cubic units, wherein Y cubic units of the volume of the aneurysm would be filled by the largest-volume sphere that can be fitted into the aneurysm without stretching the aneurysm walls, wherein Z cubic units of the volume of the aneurysm can be filled by the expandable and flexible member; and wherein Z>[Y+0.5(X−Y)]. In an example, the total volume of an aneurysm sac can be X cubic units, wherein Y cubic units of the volume of the aneurysm would be filled by the largest-volume ellipsoid that can be fitted into the aneurysm without stretching the aneurysm walls, wherein Z cubic units of the volume of the aneurysm can be filled by the expandable flexible member; and wherein Z>[Y+0.5(X−Y)].

We now discuss the specific components of the example shown in FIGS. 34 through 36. FIGS. 34 through 36 show an example of an occlusive device comprising: an outer longitudinal lumen 3404 that is configured to be inserted into a blood vessel from which aneurysm sac 1 has formed; an inner longitudinal lumen 3405 within longitudinal lumen 3404; an expandable flexible member 3403 that is inserted and expanded within aneurysm sac 1, wherein flexible member 3403 is sufficiently flexible to substantially conform to the walls of irregularly-shaped aneurysm sac 1; and an expandable resilient structure 3402 that is expanded within flexible member 3403 and resists compression after expansion.

In an example, expandable resilient structure 3402 can be spherical or elliptical. In an example, expandable resilient structure 3402 can be an expandable wire mesh or stent. In an example, expandable resilient structure 3402 can be radially-expanded in plane which is substantially parallel to the plane that is defined by the central circumference of the aneurysm neck. In an example, resilient structure 3402 can be expanded by inflation of a balloon 3401 inside resilient structure 3402. In an example, balloon 3401 can be inflated by a fluid or gas that is delivered via lumen 3404 or lumen 3405. In an example, resilient structure 3402 can self-expand after it exits lumen 3404.

In an example, expandable flexible member 3403 can be an expandable flexible net or mesh. In an example, flexible member 3403 can be a porous fabric net or mesh. In an example, flexible member 3403 can be a porous bag. In an example, expandable flexible member 3403 can be a balloon with holes. In an example, flexible member 3403 can be expanded by being filled with a plurality of embolic members. In an example, embolic members can be delivered into flexible member 3403 through lumen 3404 or lumen 3405. In an alternative example, flexible member 3403 can be non-porous. In an example, flexible member 3403 can be expanded by being filled with liquid or gas. In an example, a liquid or gas can be delivered into flexible member 3403 through lumen 3404 or lumen 3405.

In an example, flexible member 3403 can be compressed as it travels through a longitudinal lumen and then be expanded within aneurysm sac 1 after it is released from the lumen. In an example, flexible member 3403 can be folded as it travels through a lumen and then be unfolded within aneurysm sac 1. In an example, flexible member 3403 can be relatively loose or relaxed (in a lower-energy state) as it travels through a lumen and then be stretched or tense (in a higher-energy state) within aneurysm sac 1. In an example, flexible member 3403 can be elastic or stretchable. In an example, flexible member 3403 can be sufficiently elastic or stretchable that it expands when filled with an accumulation of embolic members, but not so elastic or stretchable that it allows embolic members to escape. In an example, flexible member 3403 can be a balloon with holes, wherein the holes are of sufficient size to let liquid escape, but not so large that they let embolic members escape.

In an example, embolic members for filling flexible member 3403 can be a plurality of soft, compressible members such as microsponges or blobs of gel. In an example, embolic members can be a plurality of hard, uncompressible members such as hard polymer spheres or beads. In an example, embolic members can be conveyed into flexible member 3403 through lumen 3404 or lumen 3405. In various examples, embolic members can be conveyed via a liquid flow, a moving belt, a wire loop, or an Archimedes screw.

In an example, this invention can comprise a method in which resilient structure 3402 is expanded first and flexible member 3403 is expanded second. In an example, this invention can comprise a method in which flexible member 3403 is expanded first and resilient structure 3402 is expanded second. In an example, this invention can comprise a method in which flexible member 3403 and resilient structure 3402 are expanded at substantially the same time.

As shown in FIGS. 34 through 36, an aneurysm sac can be irregular in shape. An aneurysm sac with an irregular shape will not be completely filled or spanned by a spherical or ellipsoid mass without stretching the aneurysm walls. In an example, the total volume of an aneurysm sac can be X cubic units (e.g. cubic millimeters). In an example, the maximum volume of the aneurysm which can be filled or spanned by a spherical or ellipsoid mass without stretching the aneurysm walls is Y cubic units (e.g. cubic millimeters). In an example, the total device shown in FIGS. 34 through 36 can fill more of the aneurysm than a spherical or ellipsoid mass because flexible member 3403 is sufficiently flexible to fill or span the irregular perimeter of the aneurysm sac. This can have clinical benefits, such as reducing the chances of recanalization within the aneurysm sac. In an example, this device can fill or span more than 50% of the aneurysm volume which remains unfilled by a sphere or ellipsoid. In an example, this device can fill or span Z cubic units (e.g. cubic millimeters) of the volume of the aneurysm, wherein Z>[Y+0.5(X−Y)].

In an example, the combination of (a) an outer flexible member 3403 that spans substantially the entire perimeter of the aneurysm sac 1 and (b) an inner resilient structure 3402 can create a device that is sufficiently flexible to substantially fill the entire volume of an irregularly-shaped aneurysm sac, but also sufficiently resilient so as to compress against the aneurysm walls and not slip out of the aneurysm sac.

### B. Longitudinal Parent Vessel Devices and Methods

FIGS. 37 through 57 show a number of parent vessel devices and methods for occluding an aneurysm, wherein these examples involve the insertion of a device into a longitudinal parent vessel from which aneurysm has formed.

FIGS. 37 through 39 show an example of a device and method to occlude an aneurysm which can be described as an aneurysm parent vessel stent with a semi-cylindrical central portion. More specifically, FIGS. 37 through 39 show an example of a device to occlude an aneurysm comprising: (a) a longitudinal lumen that is configured to be inserted into a blood vessel that is the parent vessel from which an aneurysm has formed; and (b) a resilient expandable member that is configured to travel through the longitudinal lumen, be inserted into the parent vessel so as to span the aneurysm neck, and then be expanded within the parent vessel; wherein the resilient expandable member resists contraction after it is expanded; wherein the resilient expandable member is configured to substantively reduce blood flow into the aneurysm when the member is expanded but not substantively reduce blood flow into nearby branching blood vessels when the member is expanded; wherein the expandable member has a proximal end and a distal end; wherein the distal end is first inserted into the parent vessel; wherein the resilient expandable member has a longitudinal axis that spans from its proximal end to its distal end; wherein the resilient expandable member has a proximal portion which longitudinally spans at least 10% of the length of its longitudinal axis and which circumferentially spans 360 degrees of the circumference of the parent vessel when expanded; wherein the resilient expandable member has a distal portion which longitudinally spans at least 10% of the length of its longitudinal axis and which circumferentially spans 360 degrees of the circumference of the parent vessel when expanded; wherein the resilient expandable member has a central portion which longitudinally spans at least 25% of the length of its longitudinal axis and which circumferentially spans at least 90 degrees and no more than 270 degrees of the circumference of the parent vessel when expanded; and wherein the central portion spans the neck of the aneurysm.

FIGS. 37 through 39 also show an example of a device to occlude an aneurysm comprising: (a) a longitudinal lumen that is configured to be inserted into a blood vessel, wherein this blood vessel is the parent vessel from which an aneurysm has formed; and (b) a resilient expandable member that is configured to travel through the longitudinal lumen, be inserted into the blood vessel, and then be expanded within the blood vessel; wherein this expandable member resists compression after it has been expanded; wherein this expandable member has a longitudinal axis spanning from a proximal end to a distal end; wherein the distal end is first inserted into the blood vessel; wherein this expandable member further comprises a first end segment at its proximal end, a second end segment at its distal end, and a central segment between the first and second end segments; wherein the first and second end segments each substantially span the complete circumference of the blood vessel when expanded; wherein the central segment spans between 25% and 75% of the circumference of the blood vessel when expanded; wherein the first and second end segments each span between 5% and 25% of the longitudinal axis of the expandable member; wherein the central segment is configured to span the aneurysm neck.

In an example, the longitudinal lumen can be a removable catheter. In an example, the resilient expandable member can be a stent. In an example, the portion of the circumference of the blood vessel that is spanned by the central segment when the central segment is expanded can be substantially constant over its length. In an example, the central segment can be shaped like a half-cylinder, wherein this half-cylinder is shaped like a cylinder that has been cut in half along its longitudinal axis. In an example, the central segment can be configured so that the portion of its circumference which spans a portion of the circumference of the blood vessel covers the aneurysm neck. In an example, the end segments can be shaped like rings. In an example, the resilient expandable member can self-expand when it is released from the longitudinal lumen. In an example, the resilient expandable member can be expanded by an inflatable member.

We now discuss the specific components of the example shown in FIGS. 37 through 39 in detail. FIGS. 37 through 39 show an example of an aneurysm-occluding device that comprises a resilient expandable member 3701 that is configured to be inserted into a parent blood vessel from which an aneurysm sac has formed and then be expanded within that parent vessel so as to selectively block blood flow to an aneurysm but not to nearby branching blood vessels. In this example, expandable member 3701 resists compression after it has been expanded.

In an example, expandable member 3701 can be an expandable but relatively non-porous wire mesh. In an example, expandable member 3701 can be a multilayer stent with at least one relatively non-porous layer. In an example, expandable member 3701 can comprise an expandable wire mesh that is covered by a stretchable fabric that reduces blood flow. In an example, expandable member 3701 can be positioned within the parent vessel of an aneurysm so that, when expanded, it substantially occludes the aneurysm neck without blocking blood flow to nearby branching blood vessels. In an example, expandable member 3701 can have (radio-opaque) markings which appear in non-invasive body imaging to enable a user to properly position expandable member so as to occlude the aneurysm neck but not nearby branching blood vessels.

Expandable member 3701 can be envisioned to have a longitudinal axis which spans from its proximal end to its distal end, wherein the distal end is first inserted into the blood vessel. As shown in the example in FIGS. 37 through 39, expandable member 3701 can have a first end segment at its proximal end, a second end segment at its distal end, and a central segment that extends between its first and second end segments. As shown in FIGS. 37 through 39, the first and second end segments of expandable member 3701 can each span between 5% and 25% of the longitudinal axis of the expandable member. As shown in these figures, the central segment of expandable member 3701 can be configured to span the remaining length of the expandable member and be positioned to cover the aneurysm neck.

As shown FIGS. 38 through 39, the first and second end segments of expandable member 3701 can each substantially span the complete circumference of the blood vessel when they are expanded and the central segment can span between 25% and 75% of the circumference of the blood vessel when it is expanded. As shown FIGS. 38 through 39, the first and second end segments of expandable member 3701 can each substantially span 360 degrees of the circumference of the blood vessel when they are expanded and the central segment can span between 90 degrees and 270 degrees of the circumference of the blood vessel when it is expanded.

In the example that is shown in FIGS. 37 through 39, expandable member 3701 is expanded by the inflation of balloon 3702 inside expandable member 3701. In this example, balloon 3702 can be inflated by delivery of a liquid or gas through longitudinal lumen 3703. In an example, expandable member 3701, balloon 3702, or both can be delivered to the parent vessel via a longitudinal lumen. In an example, this longitudinal lumen can be removed after expandable member 3701 is expanded within the parent vessel. The expandable member is positioned so as to occlude blood flow to the aneurysm neck, but not occlude blood flow to nearby (diagonally-opposite in this case) blood vessels which branch off from the parent vessel.

As shown in FIGS. 38 and 39, the portion of the circumference of the blood vessel that is spanned by a central segment of expandable member 3701 can be substantially constant over its length. In an example, a central segment of expandable member 3701 can be shaped like a half-cylinder, wherein this half-cylinder is shaped like a cylinder that has been cut in half along its longitudinal axis. As shown in FIGS. 38 and 39, a central segment of expandable member 3701 can be positioned so that the portion of its circumference which spans a portion of the circumference of the blood vessel covers the neck of aneurysm sac 1. In an example, the end segments of expandable member 3701 can be shaped like rings. As shown in FIGS. 38 and 39, expandable member 3701 can be expanded by inflatable member 3702. In an alternative example, expandable member 3701 can self-expand when it is released from a lumen.

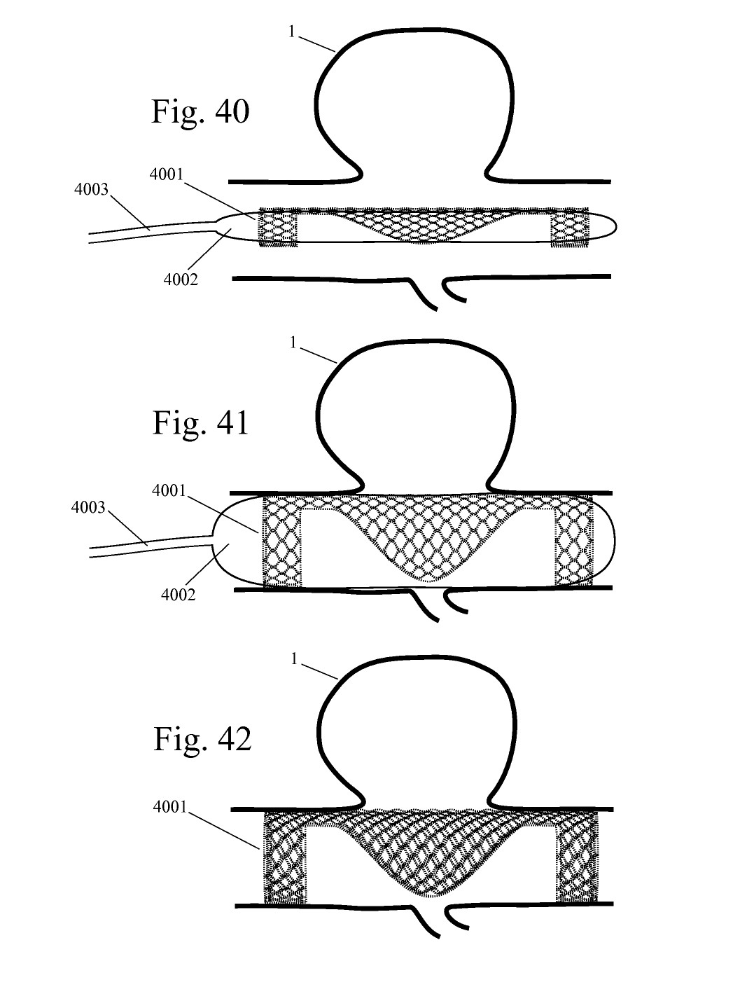

FIGS. 40 through 42 show an example of a device and method to occlude an aneurysm which can be described as an aneurysm parent vessel stent with a saddle-shaped central segment. More specifically, FIGS. 40 through 42 show an example of a device to occlude an aneurysm comprising: (a) a longitudinal lumen that is configured to be inserted into a blood vessel that is the parent vessel from which an aneurysm has formed; and (b) a resilient expandable member that is configured to travel through the longitudinal lumen, be inserted into the parent vessel so as to span the aneurysm neck, and then be expanded within the parent vessel; wherein the resilient expandable member resists contraction after it is expanded; wherein the resilient expandable member is configured to substantively reduce blood flow into the aneurysm when the member is expanded but not substantively reduce blood flow into nearby branching blood vessels when the member is expanded; wherein the expandable member has a proximal end and a distal end; wherein the distal end is first inserted into the parent vessel; wherein the resilient expandable member has a longitudinal axis that spans from its proximal end to its distal end; wherein the resilient expandable member further comprises has a proximal portion which longitudinally spans at least 10% of the length of its longitudinal axis and which circumferentially spans 360 degrees of the circumference of the parent vessel when expanded; wherein the resilient expandable member further comprises has a distal portion which longitudinally spans at least 10% of the length of its longitudinal axis and which circumferentially spans 360 degrees of the circumference of the parent vessel when expanded; wherein the resilient expandable member further comprises a saddle-shaped central portion which longitudinally spans at least 25% of the length of its longitudinal axis; wherein the proximal part of the saddle-shaped central portion spans less than 90 degrees of the circumference of the parent vessel; wherein the middle part of the saddle-shaped central portion spans more than 180 degrees of the circumference of the parent vessel; wherein the distal part of the saddle-shaped central portion spans less than 90 degrees of the circumference of the parent vessel; and wherein the central portion spans the neck of the aneurysm.

FIGS. 40 through 42 also show an example of a device to occlude an aneurysm comprising: (a) a longitudinal lumen that is configured to be inserted into a blood vessel, wherein this blood vessel is the parent vessel from which an aneurysm has formed; and (b) a resilient expandable member that is configured to travel through the longitudinal lumen, be inserted into the blood vessel, and then be expanded within the blood vessel; wherein this expandable member resists compression after it has been expanded; wherein this expandable member has a longitudinal axis spanning from a proximal end to a distal end; wherein the distal end is first inserted into the blood vessel; wherein this expandable member further comprises a first end segment at its proximal end, a second end segment at its distal end, and a central segment between the first and second end segments; wherein the first and second end segments each substantially span the complete circumference of the blood vessel when expanded; wherein the first and second end segments each span between 5% and 25% of the longitudinal axis of the expandable member; wherein the central segment is configured to span the aneurysm neck; and wherein the portion of the circumference of the blood vessel that is spanned by the central segment varies monotonically from under 25% to over 75% and then varies monotonically from over 75% back to under 25%, as one travels along the longitudinal axis of the central segment.

In an example, the longitudinal lumen can be a removable catheter. In an example, the resilient expandable member can be a stent. In an example, the central segment can be shaped like a saddle. In an example, the central segment can be configured so that the portion of its circumference which spans a portion of the circumference of the blood vessel covers the aneurysm neck. In an example, the end segments can be shaped like rings. In an example, the resilient expandable member can self-expand when it is released from the longitudinal lumen. In an example, the resilient expandable member can be expanded by an inflatable member.

We now discuss the components of FIGS. 40 through 42 in detail. FIGS. 40 through 42 show an example of an aneurysm-occluding device which comprises a resilient expandable member 4001 that is inserted and expanded within a parent blood vessel from which an aneurysm sac 1 has formed. In an example, 4001 can be a differential porosity stent. Expandable member 4001 is envisioned to have a virtual longitudinal axis which spans from its proximal end to its distal end. The distal end is the end which is first inserted into the body. In this example, resilient expandable member 4001 further comprises a first (narrow) cylindrical segment at its proximal end, a second (narrow) cylindrical segment its distal end, and a (long) central segment that extends between these first and second end segments.

In this example, each of the two narrow end segments of expandable member 4001 longitudinally spans between 10-15% of the longitudinal length of expandable member 4001 and the central segment longitudinally spans the remaining 70-80% of the length of expandable member 4001. In an example, each of the two narrow end segments of expandable member 4001 can longitudinally span between 5-25% of the longitudinal length of an expandable member and the central segment can longitudinally span the remaining 50-90% of the length of an expandable member. The central segment of an expandable member is positioned to span the aneurysm neck.

In this example, each of the two narrow end segments of expandable member 4001 spans the full circumference of the parent vessel when they are expanded. Alternatively expressing this in angular or polar-coordinate degrees, the two narrow end segments of expandable member 4001 each span the complete 360 degree circumference of the parent vessel when they are expanded. This helps to hold expandable member 4001 in place within the parent vessel.

In this example, the portion of the circumference of parent vessel which is spanned by the central segment varies as one travels in a proximal-to-distal direction along the longitudinal axis of the central segment. In particular, this portion varies in a monotonic manner from under 25% to over 75% and then varies monotonically from over 75% back to under 25%. Alternatively expressing this in angular or polar-coordinate degrees, the portion of the circumference of the parent vessel that is spanned by the central segment varies monotonically from under 90 degrees to over 270 degrees and then varies monotonically from over 270 degrees back to under 90 degrees.

This configuration can be alternatively expressed with respect to wave shapes. As shown in FIGS. 41 through 42, expandable member 4001 has two narrow cylindrical-shaped end segments and a central arcuate segment that has a single-peak sinusoidal or undulating wave shape. This central portion can be compared to the shape of a saddle, wherein the aneurysm neck is in the position of a rider on top of the saddle. The central saddle segment of expandable member 4001 is positioned so that it covers the neck of aneurysm sac 1. Expandable member 4001 is positioned within the parent vessel of an aneurysm so that, when expanded, it substantially occludes the aneurysm neck without blocking blood flow to nearby branching blood vessels. In an example, expandable member 4001 can have (radio-opaque) markings which appear in non-invasive body imaging to enable a user to properly position expandable member 4001 so as to occlude the aneurysm neck but not nearby branching blood vessels.

In an example, expandable member 4001 can be an expandable wire mesh. In an example, expandable member 4001 can be a stent. In an example, expandable member 4001 can have multiple layers, wherein at least one of these layers is relatively impermeable to blood flow. In an example, expandable member 4001 can comprise an expandable wire mesh that is covered by a stretchable fabric or elastic material that reduces blood flow. In an example, the arcuate walls of expandable member 4001 can be relatively impermeable to blood flow, but gaps between its arcuate walls can allow blood flow.

In the example that is shown in FIGS. 40 through 42, expandable member 4001 is expanded by the inflation of balloon 4002 which is inflated by delivery of a liquid or gas through longitudinal lumen 4003. In an alternative example, an expandable member can self-expand when it is released from a lumen. In an example, expandable member 4001, balloon 4002, or both can be delivered to the parent vessel via a longitudinal lumen. In an example, both this longitudinal lumen and balloon 4002 can be removed after expandable member 4001 is expanded within the parent vessel. FIG. 42 shows resilient expandable member 4001 after it has been expanded within the parent vessel and after balloon 4002 has been removed.

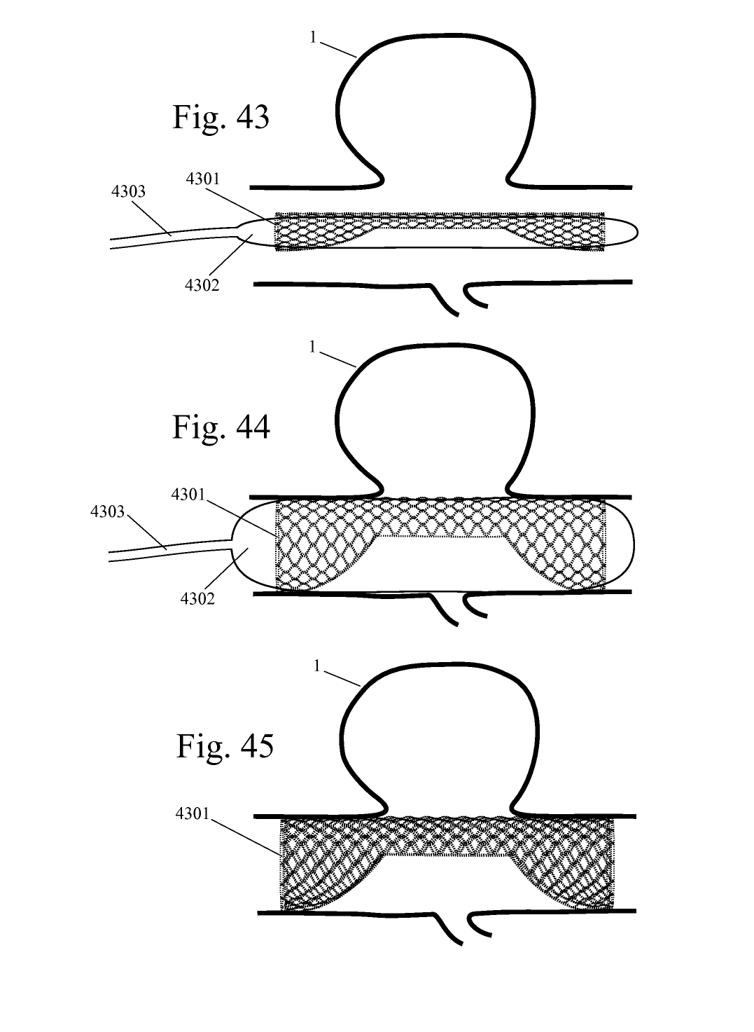

FIGS. 43 through 45 show an example of a device and method to occlude an aneurysm which can be described as a bowtie or barbell shaped stent to occlude an aneurysm. More specifically, FIGS. 43 through 45 show an example of a device to occlude an aneurysm comprising: (a) a longitudinal lumen that is configured to be inserted into a blood vessel that is the parent vessel from which an aneurysm has formed; and (b) a resilient expandable member that is configured to travel through the longitudinal lumen, be inserted into the parent vessel so as to span the aneurysm neck, and then be expanded within the parent vessel; wherein the resilient expandable member resists contraction after it is expanded; wherein the resilient expandable member is configured to substantively reduce blood flow into the aneurysm when the member is expanded but not substantively reduce blood flow into nearby branching blood vessels when the member is expanded; wherein the expandable member has a proximal end and a distal end; wherein the distal end is first inserted into the parent vessel; wherein the portion of the circumference of the parent vessel that is circumferentially spanned by the resilient expandable member varies in an arcuate manner from 360 degrees at its proximal end to 180 degrees or less, remains at 180 degrees or less for a span comprising at least 50% of the length of the member, and then varies in an arcuate manner back to 360 degrees at its distal end.

FIGS. 43 through 45 also show an example of a device to occlude an aneurysm comprising: (a) a longitudinal lumen that is configured to be inserted into a blood vessel, wherein this blood vessel is the parent vessel from which an aneurysm has formed; and (b) a resilient expandable member that is configured to travel through the longitudinal lumen, be inserted into the blood vessel, and then be expanded within the blood vessel; wherein this expandable member resists compression after it has been expanded; wherein this expandable member has a longitudinal axis spanning from a proximal end to a distal end; wherein the distal end is first inserted into the blood vessel; wherein this expandable member further comprises a first end segment at its proximal end, a second end segment at its distal end, and a central segment between the first and second end segments; wherein the first and second end segments each substantially span the complete circumference of the blood vessel when expanded; wherein the central segment is configured to span the aneurysm neck; and wherein the portion of the circumference of the blood vessel that is spanned by the central segment varies monotonically from over 75% to under 50% and then varies monotonically from under 50% back to over 75%, as one travels along the longitudinal axis of the central segment.

In an example, the longitudinal lumen can be a removable catheter. In an example, the resilient expandable member can be a stent. In an example, the resilient expandable member can have a longitudinal cross-sectional shape which looks like a bowtie or barbells cut longitudinally in half. In an example, the resilient expandable member can self-expand when it is released from the longitudinal lumen. In an example, the resilient expandable member can be expanded by an inflatable member.

We now discuss the components of FIGS. 43 through 45 in detail. FIGS. 43 through 45 show an example of an occluding device which comprises a resilient expandable member 4301 that is expanded within a parent blood vessel from which an aneurysm sac 1 has formed. Expandable member 4301 is envisioned to have a virtual longitudinal axis which spans from its proximal end to its distal end. In this example, resilient expandable member 4301 further comprises a first end segment at its proximal end, a second end segment its distal end, and a central segment that extends between these first and second end segments. The central segment of an expandable member is positioned to span the aneurysm neck.

In this example, each of the two end segments of expandable member 4301 spans the full circumference of the parent vessel when they are expanded. Alternatively expressing this in angular or polar-coordinate degrees, the two end segments of expandable member 4301 each span the complete 360 degree circumference of the parent vessel when they are expanded. This helps to hold expandable member 4301 in place within the parent vessel.

In this example, the portion of the circumference of parent vessel which is spanned by the central segment varies as one travels in a proximal-to-distal direction along the longitudinal axis of the central segment. In particular, this portion varies in a monotonic manner from over 75% to under 50% and then varies monotonically from under 50% back to over 75%. Alternatively expressing this in angular or polar-coordinate degrees, the portion of the circumference of the parent vessel that is spanned by the central segment varies monotonically from over 270 degrees to under 180 degrees and then varies monotonically from under 180 degrees back to over 270 degrees.

In this example, the shape of expandable is similar to that of a bowtie or a barbell that has been cut in half longitudinally. Expandable member 4301 is positioned within the parent vessel of an aneurysm so that, when expanded, it substantially occludes the aneurysm neck without blocking blood flow to nearby branching blood vessels. In an example, expandable member 4301 can have (radio-opaque) markings which appear in non-invasive body imaging to enable a user to properly position expandable member 4301 so as to occlude the aneurysm neck but not nearby branching blood vessels.

In an example, expandable member 4301 can be an expandable wire mesh. In an example, expandable member 4301 can be a stent. In an example, expandable member 4301 can have multiple layers, wherein at least one of these layers is relatively impermeable to blood flow. In an example, expandable member 4301 can comprise an expandable wire mesh that is covered by a stretchable fabric or elastic material that reduces blood flow.

In the example that is shown in FIGS. 43 through 45, expandable member 4301 is expanded by the inflation of balloon 4302 which is inflated by delivery of a liquid or gas through longitudinal lumen 4303. In an alternative example, an expandable member can self-expand when it is released from a lumen. In an example, expandable member 4301, balloon 4302, or both can be delivered to the parent vessel via a longitudinal lumen. In an example, both this longitudinal lumen and balloon 4302 can be removed after expandable member 4301 is expanded within the parent vessel. FIG. 45 shows resilient expandable member 4301 after it has been expanded within the parent vessel and after balloon 4302 has been removed.

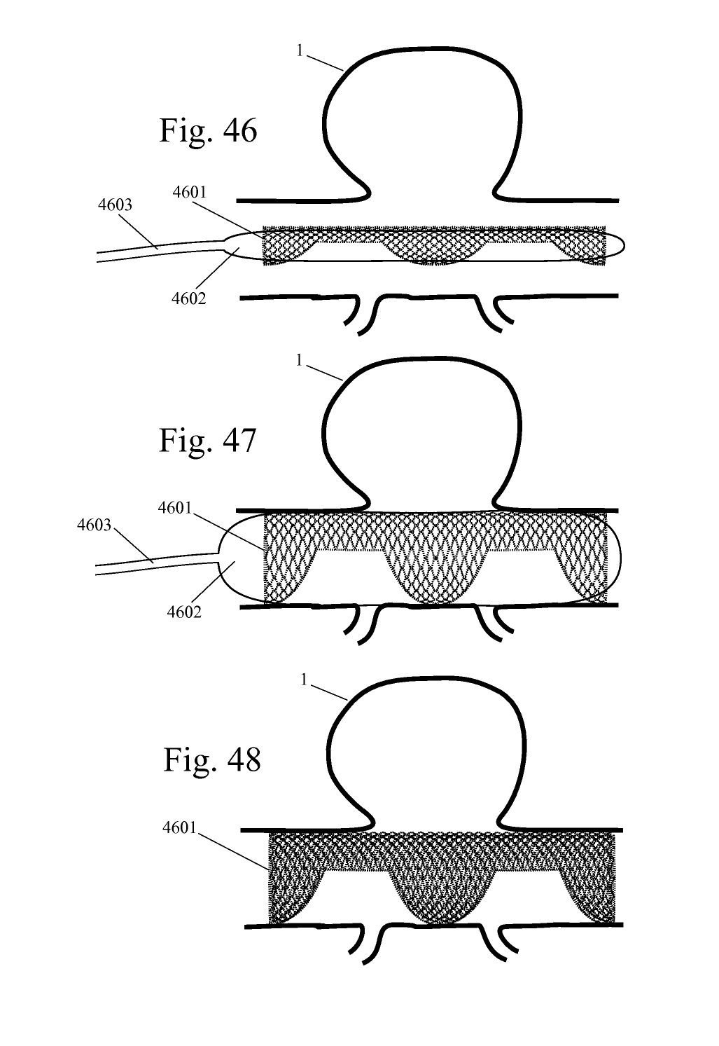

FIGS. 46 through 48 show an example of a device and method to occlude an aneurysm which can be described as an aqueduct-shaped stent to occlude an aneurysm. More specifically, FIGS. 46 through 48 show an example of a device to occlude an aneurysm comprising: (a) a longitudinal lumen that is configured to be inserted into a blood vessel that is the parent vessel from which an aneurysm has formed; and (b) a resilient expandable member that is configured to travel through the longitudinal lumen, be inserted into the parent vessel so as to span the aneurysm neck, and then be expanded within the parent vessel; wherein the resilient expandable member resists contraction after it is expanded; wherein the resilient expandable member is configured to substantively reduce blood flow into the aneurysm when the member is expanded but not substantively reduce blood flow into nearby branching blood vessels when the member is expanded; wherein the expandable member has a proximal end and a distal end; wherein the distal end is first inserted into the parent vessel; wherein the degrees of the circumference of the parent vessel that are circumferentially spanned by the resilient expandable member varies from the proximal end to the distal end in the following manner (1) varies in an arcuate manner from 360 degrees at the proximal end to 180 degrees or less (2) then remains at 180 degrees or less for a span comprising at least 10% of the length of the resilient expandable member (3) then varies in an arcuate manner back to 360 degrees (4) then varies in an arcuate manner from 360 degrees to 180 degrees or less; (5) then remains at 180 degrees or less for a span comprising at least 10% of the length of the resilient expandable member and (6) then varies in an arcuate manner back to 360 degrees at the distal end.

FIGS. 46 through 48 also show an example of a device to occlude an aneurysm comprising: (a) a longitudinal lumen that is configured to be inserted into a blood vessel, wherein this blood vessel is the parent vessel from which an aneurysm has formed; and (b) a resilient expandable member that is configured to travel through the longitudinal lumen, be inserted into the blood vessel, and then be expanded within the blood vessel; wherein this expandable member resists compression after it has been expanded; wherein this expandable member has a longitudinal axis spanning from a proximal end to a distal end; wherein the distal end is first inserted into the blood vessel; wherein this expandable member further comprises a first end segment at its proximal end, a second end segment at its distal end, and a central segment between the first and second end segments; wherein the first and second end segments each substantially span the complete circumference of the blood vessel when expanded; wherein the central segment is configured to span the aneurysm neck; and wherein the portion of the circumference of the blood vessel that is spanned by the central segment monotonically varies from over 75% to under 50% and then from under 50% to over 75%, twice, as one travels along the longitudinal axis of the central segment.

In an example, the longitudinal lumen can be a removable catheter. In an example, the resilient expandable member can be a stent. In an example, the resilient expandable member can have a longitudinal cross-sectional shape which looks similar to an aqueduct. In an example, the resilient expandable member can self-expand when it is released from the longitudinal lumen. In an example, the resilient expandable member can be expanded by an inflatable member.

We now discuss the components of FIGS. 46 through 48 in detail. FIGS. 46 through 48 show an example of an aneurysm-occluding device which comprises a resilient expandable member 4601 that is expanded within a parent blood vessel from which an aneurysm sac 1 has formed. Expandable member 4601 is envisioned to have a virtual longitudinal axis. Resilient expandable member 4601 further comprises a first end segment at its proximal end, a second end segment its distal end, and a central segment that extends between these first and second end segments. The central segment is positioned to span the aneurysm neck.

In this example, each of the two end segments spans the full circumference of the parent vessel when they are expanded. Alternatively expressing this in angular or polar-coordinate degrees, the two end segments each span the complete 360 degree circumference of the parent vessel when they are expanded. This helps to hold expandable member 4601 in place within the parent vessel.

In this example, the portion of the circumference of parent vessel which is spanned by the central segment varies as one travels in a proximal-to-distal direction along the longitudinal axis of the central segment. In particular, this portion varies, twice—from over 75% to under 50% and then from under 50% to over 75%. In other words, this portion varies from over 75% to under 50%, then back to over 75%, then back to under 50%, and then back to over 75%.

Alternatively expressing this in angular or polar-coordinate degrees, the portion of the circumference of the parent vessel that is spanned by the central segment varies, twice—from over 270 degrees to under 180 degrees and then from under 180 degrees to over 270 degrees. In other words, the portion of the circumference of the parent vessel that is spanned by the central segment varies from over 270 degrees to under 180 degrees, then back to over 270 degrees, then back to under 180 degrees, and then back to over 270 degrees.

In this example, the shape of expandable member 4601 is similar to that of an aqueduct, although the upper portion of the arches is straight rather than arcuate. The shape of expandable member 4601 can also be compared to the top row of curtains for a window or for a theater stage, with a series of hanging arcuate loops. Expandable member 4601 is positioned within the parent vessel of an aneurysm so that, when expanded, it substantially occludes the aneurysm neck without blocking blood flow to nearby branching blood vessels. In an example, expandable member 4601 can have (radio-opaque) markings which appear in non-invasive body imaging to enable a user to properly position expandable member 4601 so as to occlude the aneurysm neck but not nearby branching blood vessels.

In an example, expandable member 4601 can be an expandable wire mesh. In an example, expandable member 4601 can be a stent. In an example, expandable member 4601 can have multiple layers, wherein at least one of these layers is relatively impermeable to blood flow. In an example, expandable member 4601 can comprise an expandable wire mesh that is covered by a stretchable fabric or elastic material that reduces blood flow.

In the example that is shown in FIGS. 46 through 48, expandable member 4601 is expanded by the inflation of balloon 4602 which is inflated by delivery of a liquid or gas through longitudinal lumen 4603. In an alternative example, an expandable member can self-expand when it is released from a lumen. In an example, expandable member 4601, balloon 4602, or both can be delivered to the parent vessel via a longitudinal lumen. In an example, both this longitudinal lumen and balloon 4602 can be removed after expandable member 4601 is expanded within the parent vessel. FIG. 48 shows resilient expandable member 4601 after it has been expanded within the parent vessel and after balloon 4602 has been removed.

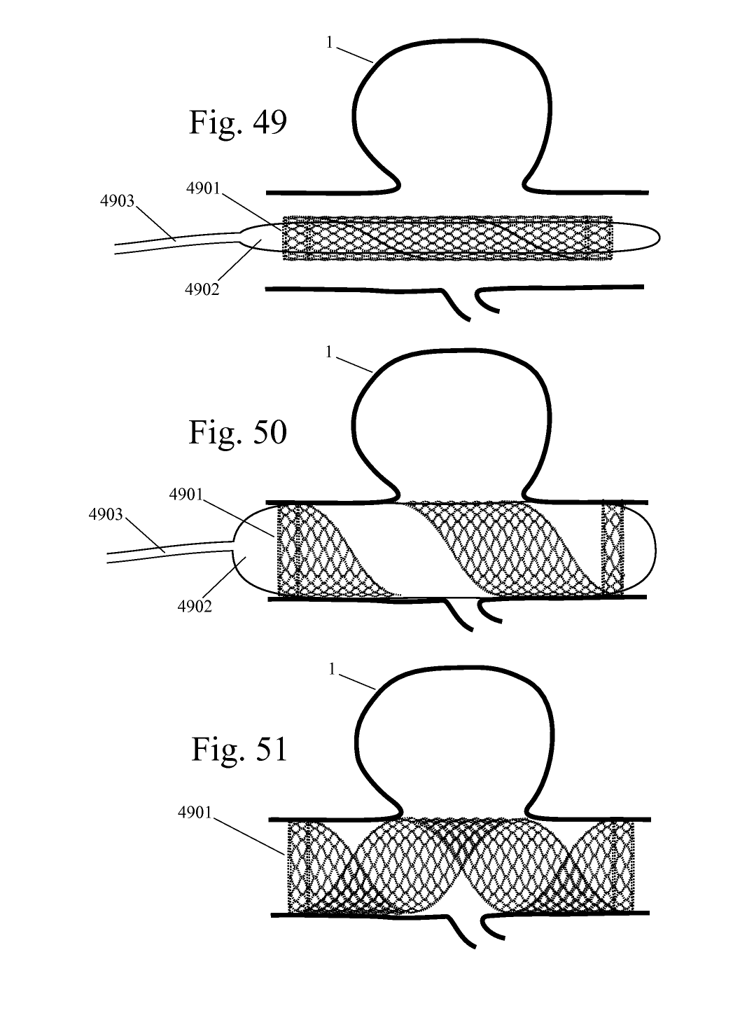

FIGS. 49 through 51 show an example of a device and method to occlude an aneurysm which can be described as a stent with end rings and a central helical segment to occlude an aneurysm. More specifically, FIGS. 49 through 51 show an example of a device to occlude an aneurysm comprising: (a) a longitudinal lumen that is configured to be inserted into a blood vessel that is the parent vessel from which an aneurysm has formed; and (b) a resilient expandable member that is configured to travel through the longitudinal lumen, be inserted into the parent vessel so as to span the aneurysm neck, and then be expanded within the parent vessel; wherein the resilient expandable member resists contraction after it is expanded; wherein the resilient expandable member is configured to substantively reduce blood flow into the aneurysm when the member is expanded but not substantively reduce blood flow into nearby branching blood vessels when the member is expanded; wherein the expandable member has a proximal end and a distal end; wherein the distal end is first inserted into the parent vessel; wherein the resilient expandable member has a longitudinal axis that spans from its proximal end to its distal end; wherein the resilient expandable member further comprises a proximal ring portion which circumferentially spans the full circumference of the parent vessel when expanded; wherein the resilient expandable member further comprises a distal ring portion which circumferentially spans the full circumference of the parent vessel when expanded; wherein the resilient expandable member further comprises a central helical ribbon portion between the proximal ring portion and the distal ring portion; wherein this helical ribbon portion is comprised of a structure selected from the group consisting of a metal mesh, a metal ribbon, a polymer mesh, and a polymer ribbon; wherein the width of the helical ribbon is greater than 20% and less than 60% of the longitudinal axis of the resilient expandable member; wherein a portion of the helical ribbon spans the aneurysm neck when the helical ribbon portion is expanded; and wherein a gap in the helical ribbon spans a branching blood vessel when the helical ribbon portion is expanded.

FIGS. 49 through 51 also show an example of a device to occlude an aneurysm comprising: (a) a longitudinal lumen that is configured to be inserted into a blood vessel that is the parent vessel from which an aneurysm has formed; and (b) a resilient expandable member that is configured to travel through the longitudinal lumen, be inserted into the parent vessel so as to span the aneurysm neck, and then be expanded within the parent vessel; wherein the resilient expandable member resists contraction after it is expanded; wherein the resilient expandable member is configured to substantively reduce blood flow into the aneurysm when the member is expanded but not substantively reduce blood flow into nearby branching blood vessels when the member is expanded; wherein the expandable member has a proximal end and a distal end; wherein the distal end is first inserted into the parent vessel; wherein the resilient expandable member has a longitudinal axis that spans from its proximal end to its distal end; wherein the resilient expandable member further comprises a proximal ring portion which circumferentially spans the full circumference of the parent vessel when expanded; wherein the resilient expandable member further comprises a distal ring portion which circumferentially spans the full circumference of the parent vessel when expanded; wherein the resilient expandable member further comprises a central helical ribbon portion between the proximal ring portion and the distal ring portion; wherein this helical ribbon is comprised of a structure selected from the group consisting of a metal mesh, a metal ribbon, a polymer mesh, and a polymer ribbon; wherein the helical ribbon portion includes at least one 360-degree spiral when expanded; wherein a portion of the helical ribbon spans the aneurysm neck when the helical ribbon portion is expanded; and wherein a gap in the helical ribbon spans a branching blood vessel when the helical ribbon portion is expanded.

FIGS. 49 through 51 also show an example of a device to occlude an aneurysm comprising: (a) a longitudinal lumen that is configured to be inserted into a blood vessel, wherein this blood vessel is the parent vessel from which an aneurysm has formed; and (b) a resilient expandable member that is configured to travel through the longitudinal lumen, be inserted into the blood vessel, and then be expanded within the blood vessel; wherein this expandable member resists compression after it has been expanded; wherein this expandable member has a longitudinal axis spanning from a proximal end to a distal end; wherein the distal end is first inserted into the blood vessel; wherein this expandable member further comprises a first end segment at its proximal end, a second end segment at its distal end, and a central segment between the first and second end segments; wherein the first and second end segments each substantially span the complete circumference of the blood vessel when expanded; wherein the central segment is configured to span the aneurysm neck; and wherein the central segment is a helical ribbon.

In an example, the longitudinal lumen can be a removable catheter. In an example, the resilient expandable member can be a stent. In an example, the width of the helical ribbon can be sufficient to substantially span the aneurysm neck when the resilient expandable member is expanded. In an example, the helical ribbon can comprise between one and two helical rotations in the central segment. In an example, the helical ribbon can comprise one and a half helical rotations in the central segment. In an example, the resilient expandable member can self-expand when it is released from the longitudinal lumen. In an example, the resilient expandable member can be expanded by an inflatable member.

We now discuss the components of FIGS. 49 through 51 in detail. FIGS. 49 through 51 show an example of an aneurysm-occluding device which comprises a partially-helical, resilient, and expandable member 4901 that is expanded within a parent blood vessel from which an aneurysm sac 1 has formed. In this example, resilient expandable member 4901 further comprises a first end segment at its proximal end, a second end segment its distal end, and a central helical ribbon segment that extends between these two end segments. Each of the two end segments spans the full circumference of the parent vessel after they are expanded.

In this example, the width of the central helical ribbon segment of expandable member 4901 is sufficient to substantially span the aneurysm neck after resilient expandable member 4901 is expanded. In an example, the helical ribbon segment of expandable member 4901 can be shaped like the spiral on the surface of a barber-shop pole. In an example, the helical ribbon segment of expandable member 4901 can be shaped like the spiral on the straight portion of a candy cane. In an example, the helical ribbon segment can comprise one and half helical rotations between the end segments. In an example, the helical ribbon can comprise between one and two helical rotations between the end segments. In an example, the width of the arcs comprising the helical ribbon segment can be between 20% and 50% of the length of the longitudinal axis of expandable member 4901.

As shown in FIGS. 50 through 51, there can be gaps between the spiraling arcs of the helical ribbon segment. In an example these gaps can be smaller before expandable member 4901 is expanded and larger after expandable member 4901 is expanded. As shown in FIGS. 50 through 51, expandable member 4901 can be positioned in the parent vessel so that a spiraling arc of the helical ribbon covers the aneurysm neck after the expandable member is expanded. This reduces blood flow to the aneurysm. As also shown in FIGS. 50 through 51, expandable member 4901 can be positioned in the parent vessel so that one of the gaps between the spiraling arcs is aligned with a branching vessel. This allows continued blood flow into the branching vessel. In an example, expandable member 4901 can have (radio-opaque) markings which appear in non-invasive body imaging to enable a user to properly position expandable member 4901 so as to occlude the aneurysm neck, but not block nearby branching blood vessels.

In an example, the helical ribbon segment of expandable member 4901 can be an expandable wire mesh. In an example, the helical ribbon segment can have multiple layers, wherein at least one of these layers is relatively impermeable to blood flow. In an example, the resilient expandable member can be expanded by balloon 4902 which is inflated by delivery of a liquid or gas through lumen 4903. In another example, expandable member 4901 can self-expand when it is released from the longitudinal lumen. In an example, expandable member 4901, balloon 4902, or both can be delivered to the parent vessel via a longitudinal lumen. In an example, both this longitudinal lumen and balloon 4902 can be removed after expandable member 4901 is expanded within the parent vessel. FIG. 51 shows resilient expandable member 4901 after it has been expanded within the parent vessel and after balloon 4902 has been removed.

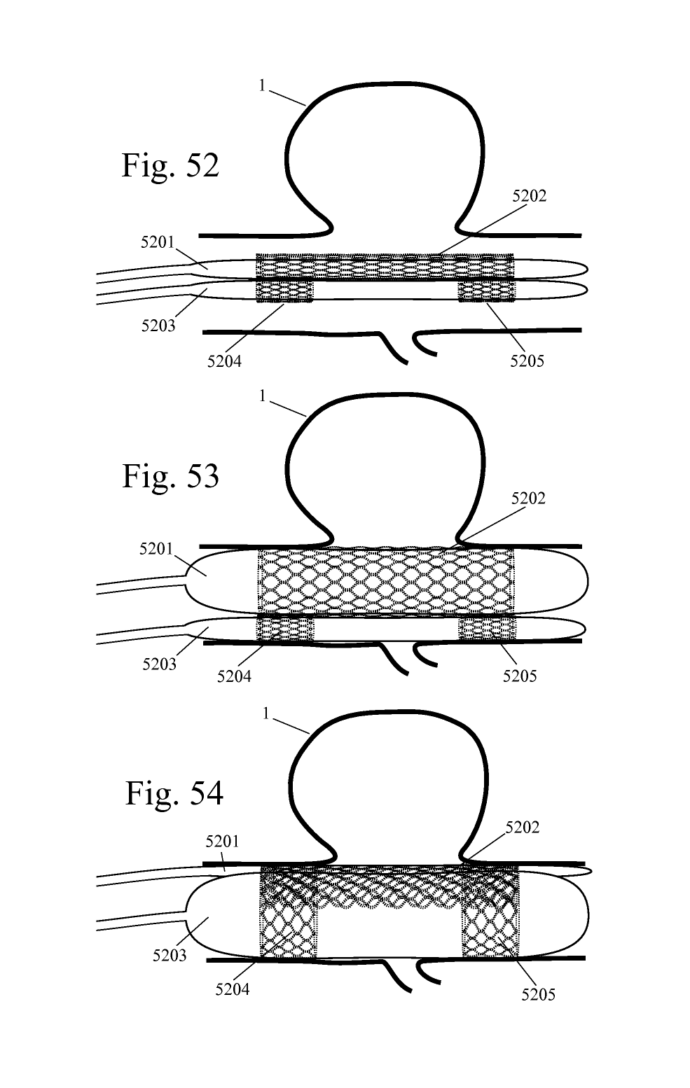

FIGS. 52 through 54 show an example of a device and method to occlude an aneurysm which can be described as a three-stent, two-balloon device for occluding an aneurysm. More specifically, FIGS. 52 through 54 show an example of a device to occlude an aneurysm comprising: (a) a first expandable member that is configured to be inserted into a blood vessel; wherein this blood vessel is the parent vessel from which an aneurysm has formed; wherein a proximal portion of this first expandable member is configured span a portion of the blood vessel which is proximal from the aneurysm neck; wherein a central portion of this first expandable member is configured to span the aneurysm neck; and wherein a distal portion of this first expandable member is configured span a portion of the blood vessel which is distal from the aneurysm neck; (b) a second expandable member that is configured to be inserted into this blood vessel; wherein a portion of this second expandable member longitudinally overlaps the proximal portion of the first expandable member; and wherein this second expandable member resists contraction after it has been expanded; (c) a third expandable member that is configured to be inserted into this blood vessel; wherein a portion of this third expandable member longitudinally overlaps the distal portion of the first expandable member; and wherein this third expandable member resists contraction after it has been expanded; (d) a first inflatable member that is inside the first expandable member; wherein inflation of this first inflatable member expands the first expandable member; and (e) a second inflatable member that is inside the second expandable member and the third expandable; wherein inflation of the second inflatable member expands the second expandable member and the third expandable member; wherein this second inflatable member is inflated after the first inflatable member is inflated; and wherein inflation of the second inflatable member compresses the first expandable member toward the aneurysm neck in order to substantively reduce blood flow to the aneurysm without substantively reducing blood flow to branching vessels on the side of the blood vessel opposite the aneurysm neck.

FIGS. 52 through 54 also show an example of a device to occlude an aneurysm comprising: (a) a first expandable member that is configured to be inserted into a blood vessel that is the parent vessel from which an aneurysm has formed; wherein this first expandable member is configured to be expanded within the blood vessel; (b) a first inflatable member; wherein inflation of this first inflatable member within the first expandable member expands the first expandable member; and wherein a first portion of the circumference of the first expandable member covers the aneurysm neck after inflation of the first inflatable member; (c) a second resilient expandable member that is configured to be inserted into the blood vessel; wherein this second expandable member is configured to be expanded in a location that longitudinally overlaps first expandable member, is proximal from the aneurysm neck, and does not span the aneurysm neck; (e) a third resilient expandable member that is configured to be inserted into the blood vessel; wherein this second expandable member is configured to be expanded in a location that longitudinally overlaps first expandable member, is distal from the aneurysm neck, and does not span the aneurysm neck; and (f) one or more second inflatable members; wherein inflation of these one or more second inflatable members within the second and third resilient expandable members expands the second and third resilient expandable members; and wherein inflation of these one or more second inflatable members also compresses the first expandable member so that a second portion of the circumference of the first expandable member also covers the aneurysm neck; and wherein the combination of the first and second portions is greater than the first portion alone.

In an example, the first expandable member can be a stent. In an example, the first expandable member can be substantially cylindrical after expansion by the first inflatable member but can be compressed into a non-cylindrical shape by the subsequent expansion of the one or more second inflatable members. In an example, the first expandable member can be substantially cylindrical after expansion by the first inflatable member but can be compressed to a substantially half-cylindrical shape by the subsequent expansion of the one or more second inflatable members. In an example, the second and third resilient expandable members can be stents. In an example, the second and third resilient expandable members can have a greater resistance to compression than the first expandable member. In an example, the second and third resilient expandable members can both be expanded by the same second inflatable member.

In an example, the first inflatable member and a second inflatable member can have substantially parallel longitudinal axes. In an example, a second inflatable member may not be left in the blood vessel after implantation but the first inflatable member may be left in the blood vessel after implantation. In an example, both the one or more second inflatable members and the first inflatable member may not be left in the blood vessel after implantation. In an example, the portion of the first expandable member that covers the aneurysm neck can be less porous after inflation of the one or more second inflatable members than this portion was after the inflation of the first inflatable member only.

We now discuss the specific components of the example that is shown in FIGS. 52 through 54. FIGS. 52 through 54 show an example of an occlusive device that comprises: a first expandable member 5202 that is expanded within a parent blood vessel from which an aneurysm has formed; a first inflatable member 5201 whose inflation expands first expandable member 5202, wherein a first portion of the circumference of first expandable member 5202 covers the aneurysm neck after the inflation of first inflatable member 5201; a second resilient expandable member 5204 that is expanded within the parent vessel and longitudinally overlaps first expandable member 5202; a third resilient expandable member 5205 that is expanded within the parent vessel and longitudinally overlaps first expandable member 5202; and one or more second inflatable members 5203 whose inflation within second and third expandable members 5204 and 5205 expands second and third expandable members 5204 and 5205.

In the example shown in FIG. 54, inflation of one or more second inflatable members 5203 also compresses first expandable member 5202 toward the aneurysm neck. This causes a second portion of the circumference of first expandable member 5202 to also cover the aneurysm neck. The combined coverage of the aneurysm neck by both the first and second portions of first expandable member 5202 results in greater and/or less-porous coverage of the aneurysm neck than coverage by the first portion of first expandable member 5202 alone. Inflation of one or more second inflatable members 5203 concentrates occluding mass along the aneurysm neck. Coverage of the aneurysm neck by multiple (compressed) portions of first expandable member 5202 becomes less-porous after inflation of one or more second inflatable members 5203.

As shown in FIG. 53, first expandable member 5202 can be substantially-cylindrical after its expansion by first inflatable member 5201. However, as shown in FIG. 54, first expandable member 5202 can be compressed into a non-cylindrical shape by the subsequent expansion of one or more second inflatable members 5203. In an example, this non-cylindrical shape can be half-cylindrical. In an example, second and third expandable members 5204 and 5205 can help to keep the device in place within the parent vessel while compressed first expandable member 5202 concentrates occlusive mass along the aneurysm neck. In an example, the second and third expandable members can be located in places within the parent vessel which are longitudinally proximal from the aneurysm neck and longitudinally distal from the aneurysm neck.

In an example, the first, second, and third expandable members (5202, 5204, and 5205) can all be stents. However, they can have different characteristics. In an example, second and third resilient expandable members 5204 and 5205 can have high resistance to compression because their primary purpose is to provide support and stability for the device. In an example, first expandable member 5202 can have low resistance to compression and/or low-porosity because its primary function is to occlude blood flow into the aneurysm. In an example, first expandable member 5202 can have multiple layers, wherein at least one of them is impermeable to blood flow.

In an example, first inflatable member 5201 and one or more second inflatable members 5203 can have substantially-parallel longitudinal axes. In an example a single second inflatable member can expand both expandable members 5204 and 5205. In an example, different second inflatable members can expand expandable member 5204 and expandable member 5205. In an example, first inflatable member 5201, one or more second inflatable members 5203, or both can be removed from the parent vessel after implantation. In an example, the first, second, and third expandable members (5202, 5204, and 5205) can have substantially parallel longitudinal axes where they overlap longitudinally.

FIGS. 52 through 54 also show an example of a method to occlude an aneurysm. This method comprises the steps of: inserting a first expandable member into a parent vessel from which an aneurysm has formed; inserting a second expandable member into the parent vessel; inserting a third expandable member into the parent vessel; expanding the first expandable member so that a first portion of the first expandable member covers the aneurysm neck; compressing a second portion of the first expandable member against the aneurysm neck; and expanding the second and third expandable members in order to keep the first expandable member in place within the parent vessel.

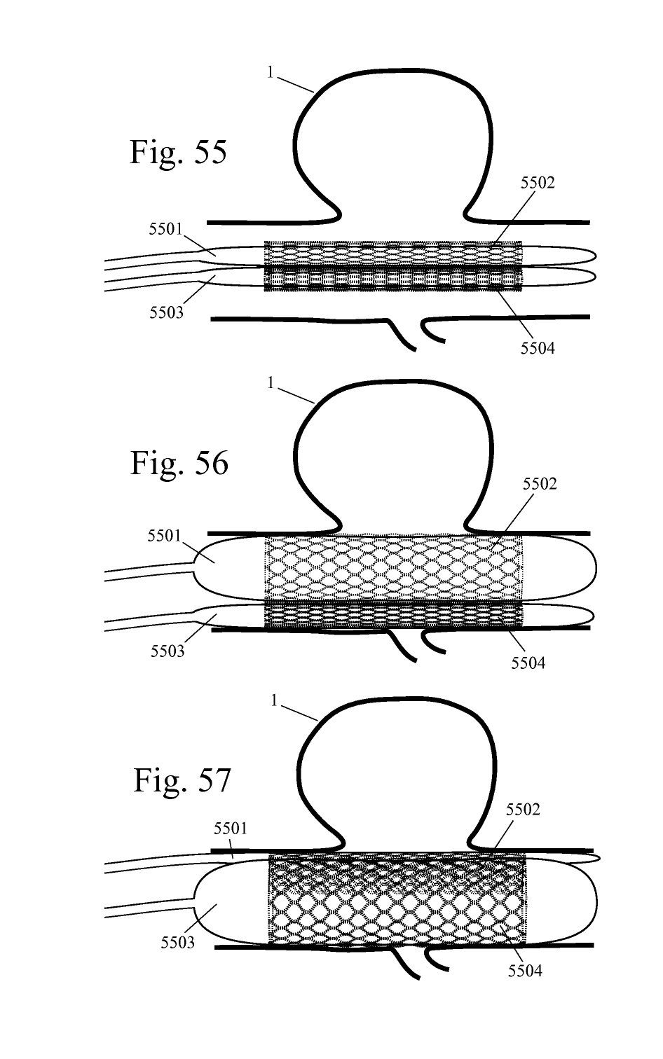

FIGS. 55 through 57 show an example of a device and method to occlude an aneurysm which can be described as a device with two parallel stents for occluding an aneurysm. More specifically, FIGS. 55 through 57 show an example of a device to occlude an aneurysm comprising: (a) a first expandable member that is configured to be inserted into a blood vessel; wherein this blood vessel is the parent vessel from which an aneurysm has formed; wherein a central portion of this first expandable member is configured to span the aneurysm neck; (b) a second expandable member that is configured to be inserted into a blood vessel; wherein this blood vessel is the parent vessel from which an aneurysm has formed; wherein a central portion of this first expandable member is configured to span the aneurysm neck; and wherein this second expandable member resists contraction after it has been expanded; (c) a first inflatable member that is inside the first expandable member; wherein inflation of this first inflatable member expands the first expandable member; and (d) a second inflatable member that is inside the second expandable member; wherein inflation of this second inflatable member expands the second expandable member; wherein this second inflatable member is inflated after the first inflatable member is inflated; and wherein inflation of this second inflatable member compresses the first expandable member toward the aneurysm neck in order to substantively reduce blood flow to the aneurysm without substantively reducing blood flow to branching vessels on the side of the blood vessel opposite the aneurysm neck.

FIGS. 55 through 57 also show an example of a device to occlude an aneurysm comprising: (a) a first expandable member that is configured to be inserted into a blood vessel that is the parent vessel from which an aneurysm has formed; wherein this first expandable member is configured to be expanded within the blood vessel; (b) a first inflatable member; wherein inflation of this first inflatable member within the first expandable member expands the first expandable member; and wherein a portion of the circumference of the first expandable member covers the aneurysm neck after inflation of the first inflatable member; (c) a second expandable member that is configured to be inserted into a blood vessel that is the parent vessel from which an aneurysm has formed; wherein this second expandable member is configured to be expanded within the blood vessel; and (d) a second inflatable member; wherein inflation of this second inflatable member within the second expandable member expands the second expandable member; and wherein a portion of the circumference of the second expandable member covers the aneurysm neck after inflation of the second inflatable member; wherein the longitudinal axis of the second expandable member is parallel to the longitudinal axis of the first expandable member; and wherein the second expandable member is expanded after the first expandable member is expanded.

In an example, the first and second expandable members can be stents. In an example, the first expandable member can be substantially cylindrical after expansion by the first inflatable member but can be compressed into a non-cylindrical shape by the subsequent expansion of the one or more second inflatable member. In an example, the first expandable member can be substantially cylindrical after expansion by the first inflatable member but can be compressed into a substantially half-cylindrical shape by the subsequent expansion of the second inflatable member. In an example, the second expandable member can have a greater resistance to compression than the first expandable member.

We now discuss the specific components of the example that is shown in FIGS. 55 through 57. FIGS. 55 through 57 show an example of an occlusive device that comprises: first expandable member 5502 that is expanded within a parent blood vessel from which an aneurysm has formed; first inflatable member 5501 whose inflation expands first expandable member 5502, wherein a first portion of the circumference of first expandable member 5502 covers the aneurysm neck after the inflation of first inflatable member 5501; second resilient expandable member 5504 that is expanded within the parent vessel and which overlaps substantially the entire longitudinal axis of first expandable member 5502; and second inflatable member 5503 whose inflation expands second expandable member 5504.

In the example that is shown in FIG. 57, inflation of second inflatable member 5503 also compresses first expandable member 5502 toward the aneurysm neck. This causes a second portion of the circumference of first expandable member 5502 to also cover the aneurysm neck. The combined coverage of the aneurysm neck by both the first and second portions of first expandable member 5502 results in greater and/or less-porous coverage of the aneurysm neck than coverage by the first portion of first expandable member 5502 alone. Inflation of second inflatable member 5503 concentrates occluding mass along the aneurysm neck. Coverage of the aneurysm neck by multiple (compressed) portions of first expandable member 5502 becomes less-porous after inflation of one or more second inflatable members 5503.

As shown in FIG. 56, first expandable member 5502 can be substantially-cylindrical after its expansion by first inflatable member 5501. However, as shown in FIG. 57, first expandable member 5502 can be compressed into a non-cylindrical shape by the subsequent expansion of one or more second inflatable members 5503. In an example, this non-cylindrical shape can be half-cylindrical. In an example, second expandable member 5504 can help to keep the device in place within the parent vessel while compressed first expandable member 5502 concentrates occlusive mass along the aneurysm neck.

In an example, first and second expandable members 5502 and 5504 can both be stents. However, they can have different characteristics. In an example, second expandable member 5504 can have high resistance to compression because its primary purpose is to provide support and stability for the device. In an example, first expandable member 5502 can have low resistance to compression and/or low-porosity because its primary function is to occlude blood flow into the aneurysm. In an example, first expandable member 5502 can have multiple layers, wherein at least one of them is impermeable to blood flow.

In an example, first inflatable member 5501 and second inflatable member 5503 can have substantially-parallel longitudinal axes. In an example, first inflatable member 5501, second inflatable member 5503, or both can be removed from the parent vessel after implantation. In an example, first and second expandable members 5502 and 5504 can have substantially parallel longitudinal axes where they overlap longitudinally.

FIGS. 55 through 57 also show an example of a method to occlude an aneurysm. This method comprises the steps of: inserting a first expandable member into a parent vessel from which an aneurysm has formed; inserting a second expandable member into the parent vessel; expanding the first expandable member so that a first portion of the first expandable member covers the aneurysm neck; compressing a second portion of the first expandable member against the aneurysm neck; and expanding the second expandable member in order to keep the first expandable member in place within the parent vessel.

### C. Three-Way Parent Vessel Junction Devices and Methods

FIGS. 58 through 79 show two examples of aneurysm-occluding devices and method for cases wherein the parent vessel of an aneurysm is a three-way blood vessel junction. These examples involve the insertion of a device into a three-way junction of blood vessels. FIGS. 58 through 65 show an example of a device and method to occlude an aneurysm which can be described as a three-stent device (“tri-stent device”) for occluding an aneurysm at a three-way vessel junction.

More specifically, FIGS. 58 through 65 show an example of a device to occlude an aneurysm comprising: (a) a longitudinal lumen that is configured to be inserted into a blood vessel; wherein this blood vessel is the parent vessel from which an aneurysm has formed; (b) a first resilient expandable member; wherein this first resilient expandable member is configured to travel through the longitudinal lumen, be inserted into the blood vessel, and then be expanded within the blood vessel; wherein this member resists contraction after having been expanded; wherein this member has a central axis spanning from the proximal end of the member to the distal end of the member; and wherein the distal end is the end that is first inserted into the blood vessel; (c) a second resilient expandable member; wherein this second resilient expandable member is configured to travel through the longitudinal lumen, be inserted into the blood vessel, and then be expanded within the blood vessel; wherein this member resists contraction after having been expanded; wherein this member has a central axis spanning from the proximal end of the member to the distal end of the member; wherein the distal end is the end that is first inserted into the blood vessel; wherein the central axis of this second resilient expandable member is substantively aligned with the central axis of the first resilient expandable member as these two members travel through the longitudinal lumen; and wherein the central axis of this second resilient expandable member rotates away from alignment with the central axis of the first resilient expandable member after the second resilient expandable member exits the longitudinal lumen; (d) a third resilient expandable member; wherein this third resilient expandable member is configured to travel through the longitudinal lumen, be inserted into the blood vessel, and then be expanded within the blood vessel; wherein this member resists contraction after having been expanded; wherein this member has a central axis spanning from the proximal end of the member to the distal end of the member; wherein the distal end is the end that is first inserted into the blood vessel; wherein the central axis of this third resilient expandable member is substantively aligned with the central axis of the first resilient expandable member as these two members travel through the longitudinal lumen; and wherein the central axis of this third resilient expandable member rotates away from alignment with the central axis of the first resilient expandable member after the third resilient expandable member exits the longitudinal lumen; (e) a first flexible connecting member; wherein this first flexible connecting member connects a portion of the circumference of the first resilient expandable member to a portion of the circumference of the second resilient expandable member; and wherein the portion of the circumference of the second resilient expandable member that is thus connected spans less than three-quarters of the total circumference of the second resilient expandable member; (f) a second flexible connecting member; wherein this second flexible connecting member connects a portion of the circumference of the first resilient expandable member to a portion of the circumference of the third resilient expandable member; and wherein the portion of the circumference of the third resilient expandable member that is thus connected spans less than three-quarters of the total circumference of the third resilient expandable member; and (g) a third flexible connecting member; wherein this third flexible connecting member connects a portion of the circumference of the second resilient expandable member to a portion of the circumference of the third resilient expandable member; wherein the portion of the circumference of the second resilient expandable member that is thus connected spans less than three-quarters of the circumference of the second resilient expandable member; wherein the portion of the circumference of the third resilient expandable member that is thus connected spans less than three-quarters of the circumference of the third resilient expandable member; and wherein this third flexible connecting member is configured to reduce blood flow into the aneurysm.

FIGS. 58 through 65 also show an example of a device to occlude an aneurysm comprising: (a) a first resilient expandable member, wherein this expandable member is configured to be inserted into a blood vessel junction or a blood vessel branch at a blood vessel junction, wherein an aneurysm has formed at the blood vessel junction; (b) a second resilient expandable member, wherein this expandable member is configured to be inserted into the blood vessel junction or a blood vessel branch at the blood vessel junction; (c) a third resilient expandable member, wherein this expandable member is configured to be inserted into the blood vessel junction or a blood vessel branch at the blood vessel junction; (d) a first flexible connecting member, wherein this first flexible connecting member connects a portion of the circumference of the first resilient expanding member to a portion of the circumference of the second resilient expanding member; (e) a second flexible connecting member, wherein this first flexible connecting member connects a portion of the circumference of the first resilient expanding member to a portion of the circumference of the third resilient expanding member; and (f) a third flexible connecting member, wherein this third flexible connecting member connects a portion of the circumference of the second resilient expanding member to a portion of the circumference of the third resilient expanding member, wherein this third flexible connecting member is configured to substantially span the aneurysm neck when the first, second, and third expandable members are expanded.

In an example, the first, second, and third resilient expandable members can be stents. In an example, the first resilient expandable member can be expanded in the blood vessel through which blood comes into the blood vessel junction and wherein the second and third resilient expandable members are expanded in blood vessels through which blood goes out from the blood vessel junction. In an example, the third flexible connecting member can reduce blood flow. In an example, the third flexible connecting member can be impermeable to blood flow. In an example, the third flexible connecting member can connect more than 25% and less than 75% of the circumferences of the second and third resilient expandable members.

In an example, the first resilient expandable member can expand before the second and third resilient expandable members expand. In an example, the first, second, and third resilient expandable members can have longitudinal axes which are substantially aligned as they travel through the longitudinal lumen and can rotate out of alignment after they exit the longitudinal lumen. In an example, the first, second, and third resilient expandable members can be substantially-cylindrical stents. In an example, the first flexible connecting member can be attached to the second resilient expandable member on the side of the second resilient expandable member that is opposite to the side of the second resilient expandable member to which the third flexible connecting member is attached. In an example, the second flexible connecting member can be attached to the third resilient expandable member on the side of the third resilient expandable member that is opposite to the side of the third resilient expandable member to which the third flexible connecting member is attached. In an example, the third flexible connecting member can have a curved semi-cylindrical shape with an arcuate longitudinal axis that spans the aneurysm neck after the device has been implanted.

We now discuss the specific components of the example that is shown in FIGS. 58 through 65. In order to provide anatomical context and to show pre-intervention blood flow dynamics, FIG. 58 shows a three-way blood vessel junction from which an aneurysm 5804 has formed. In this example, the parent vessel of the aneurysm comprises a blood vessel junction with three vessel branches joined at the junction. In some cases, an aneurysm (such as 5804) can form at such a blood vessel junction because of currents of blood flow 5805 which pound against the portion of the junction wall which is opposite from inflow vessel 5801. In this example, blood flow 5805 enters the junction through inflow vessel 5801 and exits the junction through a first outflow vessel 5802 and a second outflow vessel 5803. However, some currents of blood flow 5805 enter (and expand) aneurysm sac 5804. In addition to exerting pressure on the aneurysm sac which can cause the aneurysm to grow and/or hemorrhage, this improper blood flow can also cause undesirable turbulence within the junction and surrounding vessels. In an example, it is desirable to design a device that not only occludes the aneurysm, but also diverts blood flow away from hitting this portion of the junction wall and restores proper blood flow through the blood vessel junction.

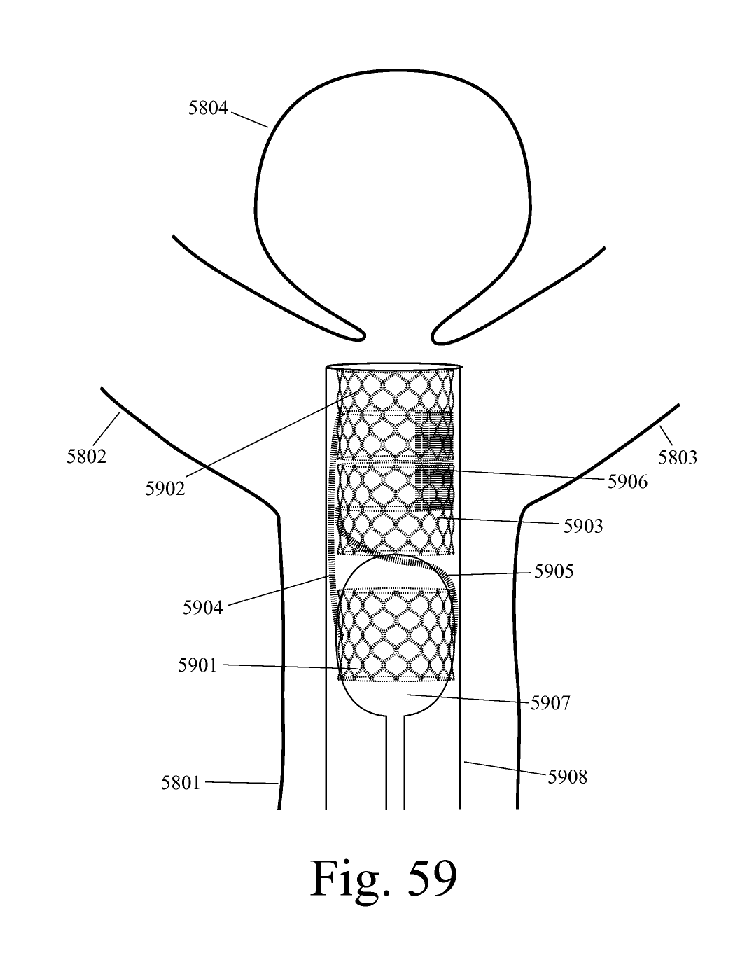

FIGS. 59 through 65 show sequential views of an example of a device to occlude aneurysm 5804 comprising: first resilient expandable member 5901 that is expanded within the blood vessel junction from which aneurysm 5804 has formed or within a vessel branch adjacent to that blood vessel junction; second resilient expandable member 5902 that is expanded within the blood vessel junction from which aneurysm 5804 has formed or within a vessel branch adjacent to the blood vessel junction; third resilient expandable member 5903 that is expanded in the blood vessel junction from which aneurysm 5804 has formed or in a vessel branch adjacent to the blood vessel junction; first flexible connecting member 5904 which connects a portion of the circumference of first resilient expandable member 5901 to a portion of the circumference of second resilient expandable member 5902; second flexible connecting member 5905 which connects a portion of the circumference of first resilient expandable member 5901 to a portion of the circumference of third resilient expandable member 5903; and third flexible connecting member 5906 which connects a portion of the circumference of second resilient expandable member 5902 to a portion of the circumference of third resilient expandable member 5903, wherein third flexible connecting member 5906 is configured to substantially span the aneurysm neck when first, second, and third expandable members 5901, 5902, and 5903 are expanded.

FIG. 59 shows an initial cross-sectional view of a device after it has been inserted into the blood vessel junction, but before the expandable members have been fully positioned and expanded. In this initial view, the three resilient expandable members 5901, 5902, and 5903 are still contained within longitudinal lumen 5908 (such as a removable catheter) which has been used to insert them into the blood vessel junction. In this initial view, the longitudinal axes of the three resilient expandable members 5901, 5902, and 5903 are all aligned. In this initial view, the three resilient expandable members 5901, 5902, and 5903 have not yet been expanded. In addition to showing the three resilient expandable members, FIG. 59 also shows the three connecting members 5904, 5905, and 5906 which connect portions of the circumferences of the three expandable members. In this example, the three connecting members connect portions of the circumferences of the three expandable members at central longitudinal locations on the three expandable members.

In this example, first connecting member 5904 is a relatively narrow spring or elastic member which connects the longitudinal centers of first resilient expandable member 5901 and second resilient expandable member 5902. In the initial view in FIG. 59, first connecting member 5904 is in a stretched configuration. As we will see in subsequent figures, after the three expandable members and the three connecting members are released from lumen 5908, contraction of first connecting member 5904 alters the relative positions and orientations of first expandable member 5901 and second expandable member 5902.

In this example, second connecting member 5905 is a relatively narrow spring or elastic member which connects the longitudinal centers of first resilient expandable member 5901 and third resilient expandable member 5903. In the initial view in FIG. 59, second connecting member 5905 is in a stretched configuration. As we will see in subsequent figures, after the three expandable members and the three connecting members are released from lumen 5908, contraction of second connecting member 5905 alters the relative positions and orientations of first expandable member 5901 and third expandable member 5903.

In this example, third connecting member 5906 is a wider and less-elastic member which connects a wider span of the circumferences of second expandable member 5902 and third expandable member 5903. In this example, third connecting member 5906 spans and connects approximately 33% of the circumferences of the second and third expandable members. In an example, a third connecting member can span and connect between 25% and 75% of the circumferences of the second and third expandable members. Expressing this in angular or polar coordinates, a third connecting member can span and connect between 90 degrees and 270 degrees of the circumferences of the second and third expandable members. Third connecting member 5906 is also relatively non-porous and/or impermeable to blood flow. As we will see in subsequent figures, after the three expandable members and the three connecting members are released from lumen 5908, third connecting member 5906 becomes positioned to cover the neck of aneurysm 5804. This diverts blood flow away from the aneurysm without reducing blood flow to outflow vessels 5802 and 5803.

FIG. 59 also shows first inflatable member 5907 whose inflation will expand first resilient expandable member 5901. In this example, the first, second, and third resilient expandable members are all stents. In an example, one or more of these resilient expandable members can be expandable wire meshes. In an example, one or more of these resilient expandable members can be expandable polymer meshes. In an example, one or more of these resilient expandable members can have multiple layers.

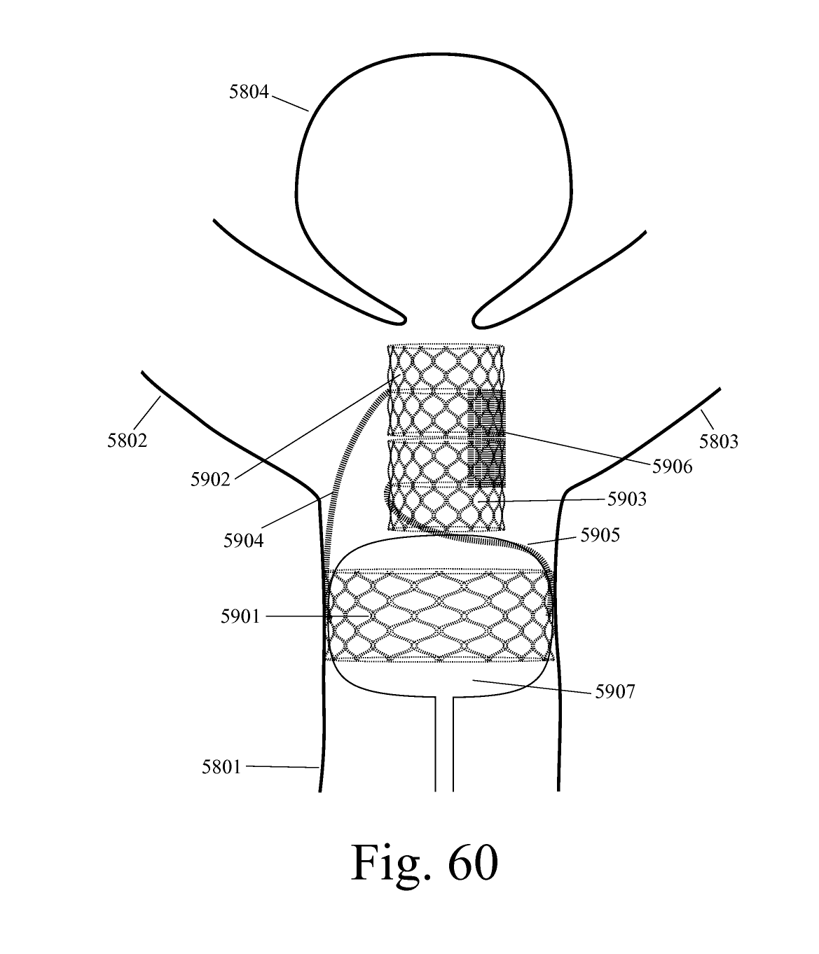

FIG. 60 shows the next sequential view of the same device that was shown in FIG. 59. In FIG. 60, the three resilient expandable members and the three connecting members have exited lumen 5908 and lumen 5908 has been removed. FIG. 60 also shows first expandable member 5901 having been expanded by inflation of inflatable member 5907 within inflow vessel 5801. Expansion of first expandable member 5901 within inflow vessel 5801 helps to hold the device in place during the remainder of the implantation procedure and thereafter.

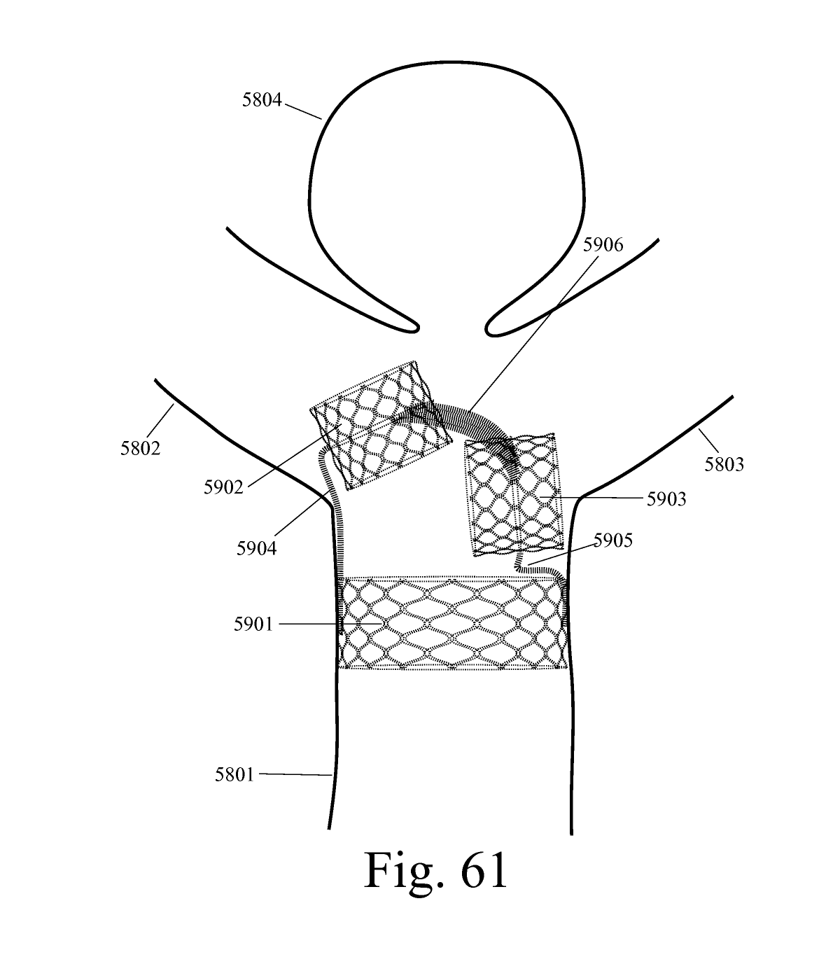

FIG. 61 shows the next sequential view. In FIG. 61, inflatable member 5907 has been deflated and removed. FIG. 61 also shows that the contraction of flexible connecting members 5904 and 5905 is reorienting expandable members 5902 and 5903 so that their longitudinal axes begin to align with the lumens of first and second outflow vessels 5802 and 5803. As part of this reorientation and also due to pressure from blood flow through the junction, third flexible connecting member 5906 begins to billow upwards toward the aneurysm neck.

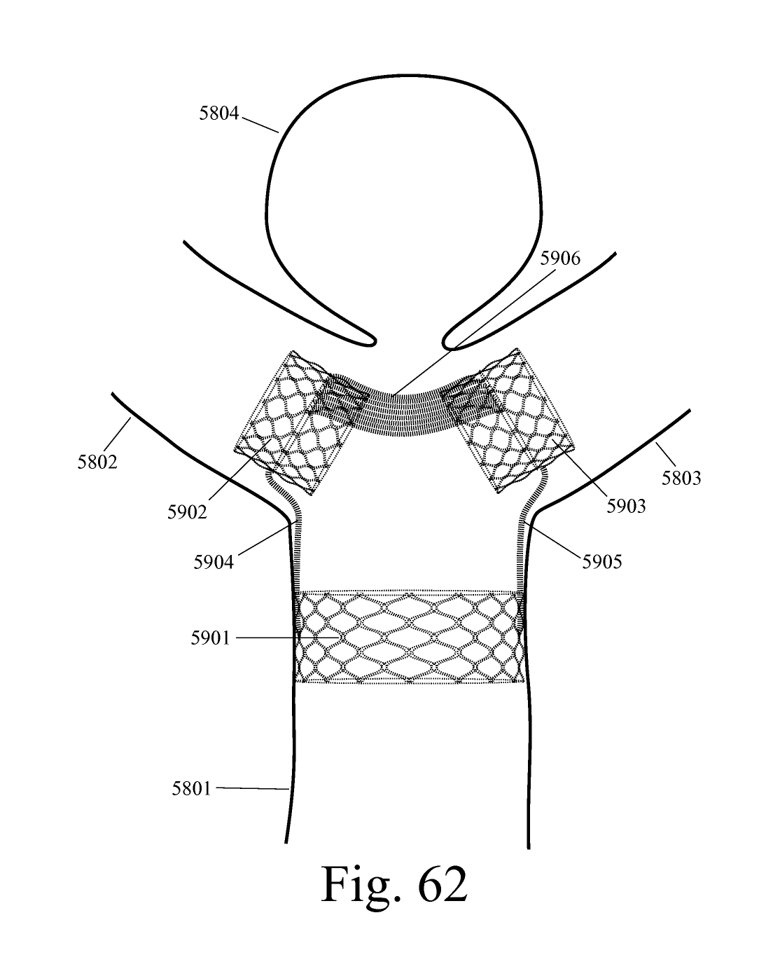

FIG. 62 shows the next sequential view. In FIG. 62, resilient expandable members 5902 and 5903 are positioned for expansion with the lumens of outflow vessels 5802 and 5803, respectively. In an example, this positioning can be partly due to contraction of flexible connecting members 5904 and 5905. In an example, this positioning can be partly due to connecting members 5904 and 5905 having a shape memory and shapes to which they return when released from lumen 5908. In an example, this positioning can be partly due to expandable members 5902 and 5903 being drawn downstream by the force of blood flow through the blood vessel junction. In an example, this positioning can be partly directed by means of a guidewire or interventional member inserted by the person performing the implantation. In an example, resilient expandable members 5902 and 5903 can be moved to these desirable positions by a combination of these causal factors.

FIG. 62 also shows third connecting member 5906 as more-fully spanning and covering the neck of aneurysm 5804. In an example, third connecting member 5906 can be an elastic and non-porous fabric which stretches over the aneurysm neck as resilient expandable members 5902 and 5903 move to their desired positions. In an example, third connecting member 5906 can be a flexible wire mesh which stretches over the aneurysm neck as resilient expandable members 5902 and 5903 move to their desired positions. In this example, third connecting member 5906 spans from a central longitudinal circumference of expandable member 5902 to a central longitudinal circumference of expandable member 5903. In another example, a third connecting member can span from a circumference at a longitudinal end of expandable member 5902 to a circumference at a longitudinal end of expandable member 5903.

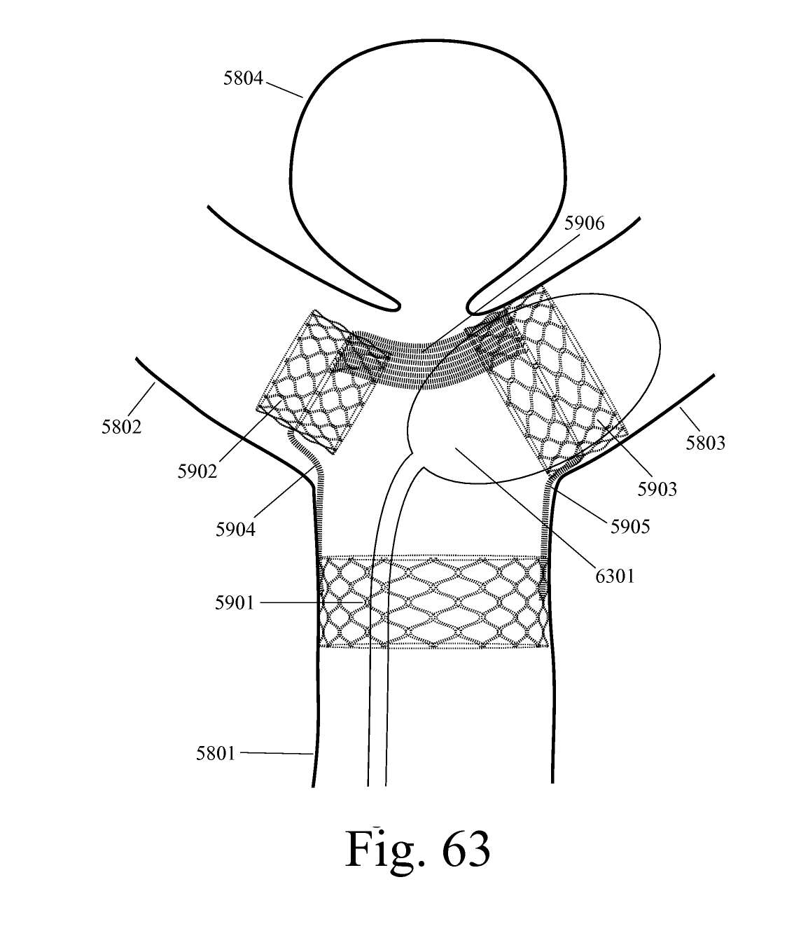

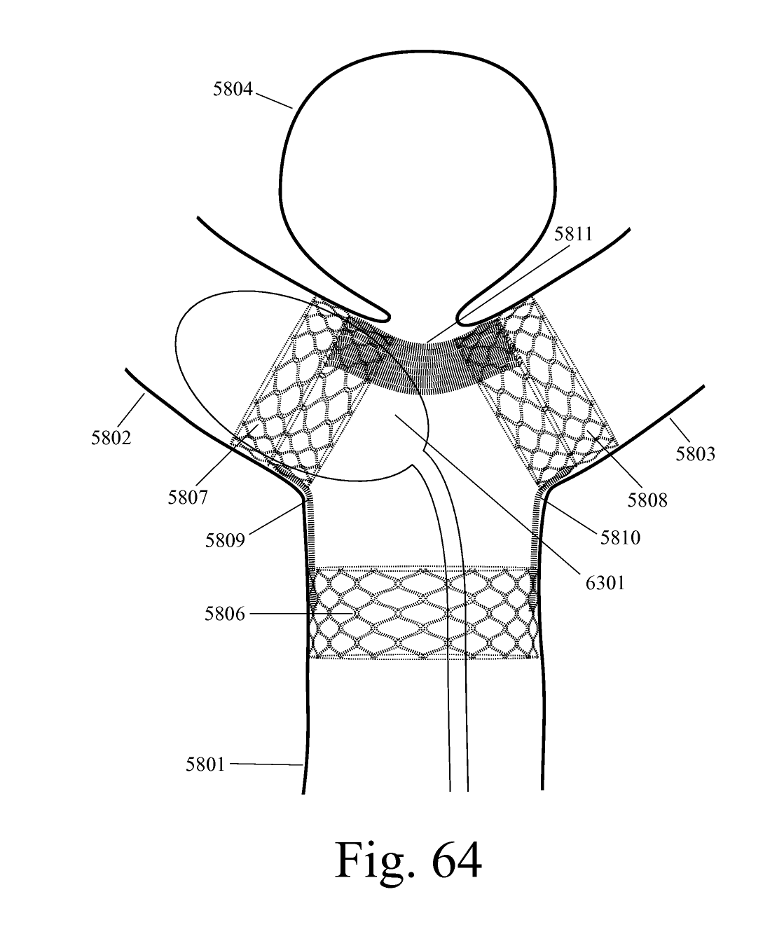

FIG. 63 shows the next sequential view. In FIG. 63, an inflatable member 6301 has been inserted into third resilient expandable member 5903 and this expandable member has been expanded within outflow vessel 5803. FIG. 64 shows a next sequential view. In FIG. 64, inflatable member 6301 has been deflated and removed from expandable member 5903 and then inserted and expanded within second resilient expandable member 5902. This expands second expandable member 5902 within outflow vessel 5802.

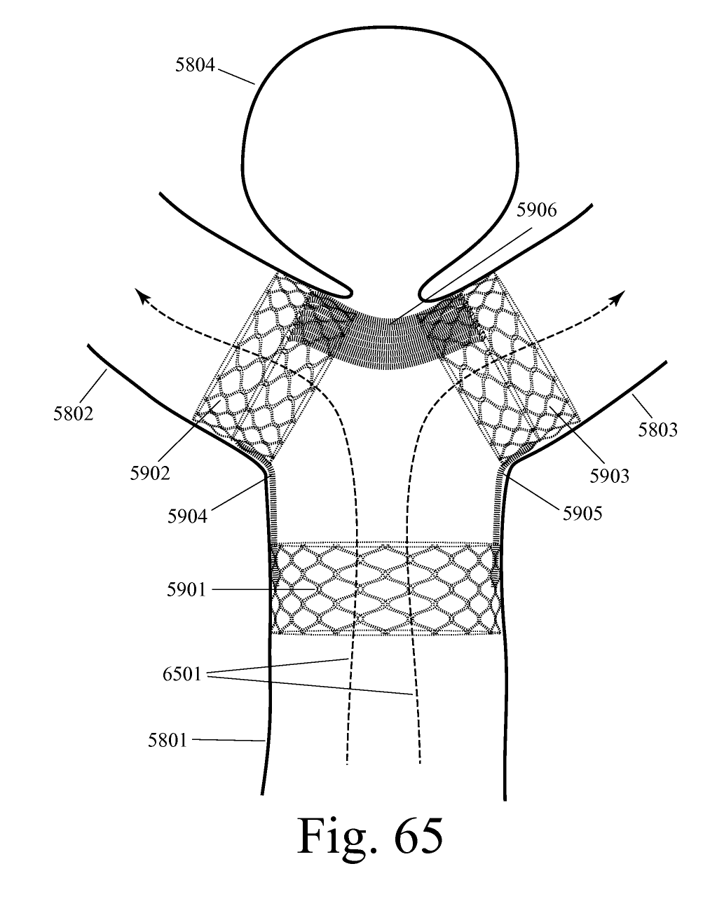

FIG. 65 shows the final view in this sequence. In FIG. 65, the delivery lumen and inflatable members have all been removed and only the occluding device remains in place. FIG. 65 shows how this three-stent device, after proper deployment, diverts blood flow 6501 away from the aneurysm without reducing blood flow to outflow vessels 5802 and 5803. This occludes the aneurysm and restores proper blood flow to the blood vessel junction.

In this example, first resilient expandable member 5901 is expanded before second and third resilient expandable members 5902 and 5903 are expanded. In this example, first, second, and third resilient expandable members 5901, 5902, and 5903 have longitudinal axes which are substantially aligned as they travel through lumen 5908, but which rotate out of alignment after they exit lumen 5908. In this example, first, second, and third resilient expandable members 5901, 5902, and 5903 are substantially-cylindrical stents.

In this example, first connecting member 5904 is attached to second expandable member 5902 on the side of second expandable member 5902 that is opposite to the side of second expandable member 5902 to which third connecting member 5906 is attached. In this example, second connecting member 5902 is attached to third expandable member 5903 on the side of third expandable member 5903 that is opposite to the side of third expandable member 5903 to which third connecting member 5906 is attached. In this example, third connecting member 5906 stretches or bends into a curved semi-cylindrical or saddle shape with an arcuate longitudinal axis that spans aneurysm neck after the device has been fully implanted.

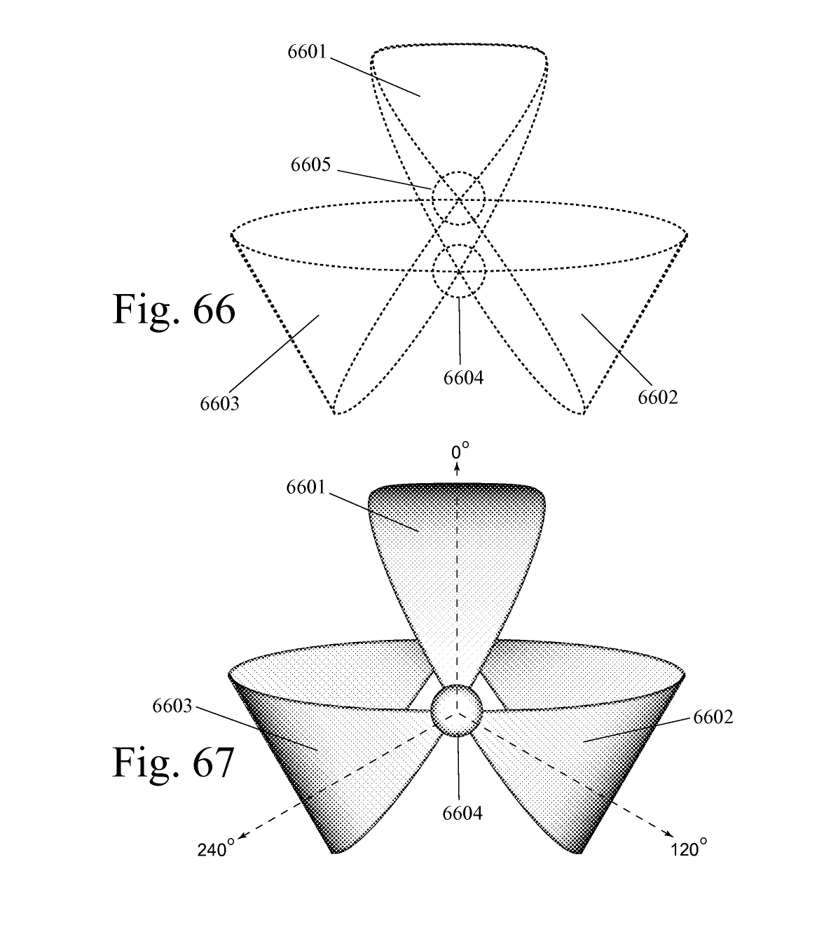

FIGS. 66 through 79 show an example of a device and method to occlude an aneurysm which can be described as a three-petal device (or “tri-petal device”) for occluding an aneurysm at a vessel junction. More specifically, FIGS. 66 through 79 show an example of a tri-petal device to occlude an aneurysm comprising: (a) a longitudinal lumen that is configured to be inserted into a junction of blood vessels; wherein there is an aneurysm extending out from this junction; (b) a resilient expandable member that is configured to travel through the longitudinal lumen, be inserted into the junction, and then be expanded within the junction; wherein first portions of this resilient expandable member have a first porosity to blood; wherein one or more of the first portions are configured to substantially span the aneurysm neck after the resilient expandable member has been expanded; wherein second portions or gaps between the first portions of this resilient expandable member have a second porosity to blood; wherein the second portions are configured to substantially span the lumens of the blood vessels; wherein the first porosity to blood is less than the second porosity to blood; wherein the first portions of the resilient expandable member comprise three arcuate petals that are centrally connected and expand radially outwards from the center of the resilient expandable member as the resilient expandable member is expanded; and (c) an inflatable member that is inflated inside the resilient expandable member; wherein inflation of this inflatable member expands the resilient expandable member.

In an example, the shapes of the three arcuate petals after their expansion can comprise sections of the surface of a three-dimensional arcuate object and this three-dimensional object can be a sphere or an ellipsoid. In an example, two diametrically-opposite poles can be envisioned on the surface of the three-dimensional object. Also, in an example, a plurality of arcuate longitudinal lines can be envisioned which span the surface of the three-dimensional object from one pole to the other pole, in a manner similar to how arcuate lines longitude span a globe of the earth from the North Pole to the South Pole. Each of these longitudinal lines can be associated with a polar coordinate ranging from 0 to 360 degrees around the latitudinal circumference of the three-dimensional object.

In an example, the surface of a first arcuate petal can include an arcuate longitudinal line at a polar coordinate of 0 degrees. The surface of a second arcuate petal can include an arcuate longitudinal line at a polar coordinate of 120 degrees. The surface of a third arcuate petal can include an arcuate longitudinal line at a polar coordinate of 240 degrees. In an example, the surface of the first arcuate petal can include an arcuate longitudinal line at a polar coordinate of 0 degrees. The surface of the second arcuate petal can include an arcuate longitudinal line at a polar coordinate of 100 degrees. The surface of the third arcuate petal can include an arcuate longitudinal line at a polar coordinate of 260 degrees.

In an example, the shapes of three arcuate petals after expansion can comprise sections of the surface of a three-dimensional figure that has a triangular cross-sectional shape in a first plane and an elliptical cross-sectional shape in a second plane that is perpendicular to the first plane. In an example, the shapes of three arcuate petals after expansion can comprise sections of the surface of a geodesic sphere or ellipsoid. In an example, first portions of a resilient expandable member can cover greater than 25% and less than 75% of the surface area of the inflatable member after expansion. In an example, first portions of a resilient expandable member can comprise an expandable resilient mesh, a low-porosity stretchable material, or both an expandable resilient mesh and a layer of low-porosity stretchable material.

FIGS. 66 through 79 also show an example of a tri-petal device to occlude an aneurysm comprising: (a) a resilient expandable member that is configured to be expanded within a three-way junction of blood vessels from which an aneurysm has formed; (b) wherein a virtual diameter-spanning axis can be defined for this resilient expandable member; (c) wherein this virtual diameter-spanning axis spans from a virtual first pole of the resilient expandable member to an diametrically-opposite virtual second pole of the resilient expandable member; (d) wherein virtual arcuate lines of longitude can be defined along the surface of this expandable member; (e) wherein these virtual arcuate lines of longitude span from the first pole to the second pole; (f) wherein these lines of longitude can be assigned polar coordinates ranging from 0 to 360 degrees in a manner similar to the lines of longitude on a globe of the earth; (g) wherein this resilient expandable member further comprises three arcuate radially-expandable petals; (h) wherein a first arcuate radially-expandable petal has a surface that includes the line of longitude at 0 degrees; (i) wherein a second arcuate radially-expandable petal has a surface that includes the line of longitude at 120 degrees; (j) wherein a third arcuate radially-expandable petal has a surface that includes the line of longitude at 240 degrees; (k) wherein one of the three arcuate radially-expandable petals is configured to cover the aneurysm neck when the member is expanded, so as to reduce blood flow to the aneurysm; and (1) wherein gaps between the three arcuate radially-expandable petals are configured to span the three blood vessel lumens joined at the junction the when the member is expanded, so as to allow continued blood flow through these lumens.

In an example, the three radially-expandable petals can be shaped like sections of the surface of a sphere. In an example, the three radially-expandable petals can be shaped like sections of the surface of an ellipsoid. In an example, the three radially-expandable petals can be shaped like sections of the surface of a three-dimensional figure with one cross-section that is triangular and a perpendicular cross-section that is elliptical. In an example, the three radially-expandable petals can be comprised of wire mesh. In an example, the three radially-expandable petals can be comprised of a polymer. In an example, the three radially-expandable petals can be expanded by an inflatable member. In an example, the three radially-expandable petals can self-expand in the junction because they are made from a material with a shape memory.

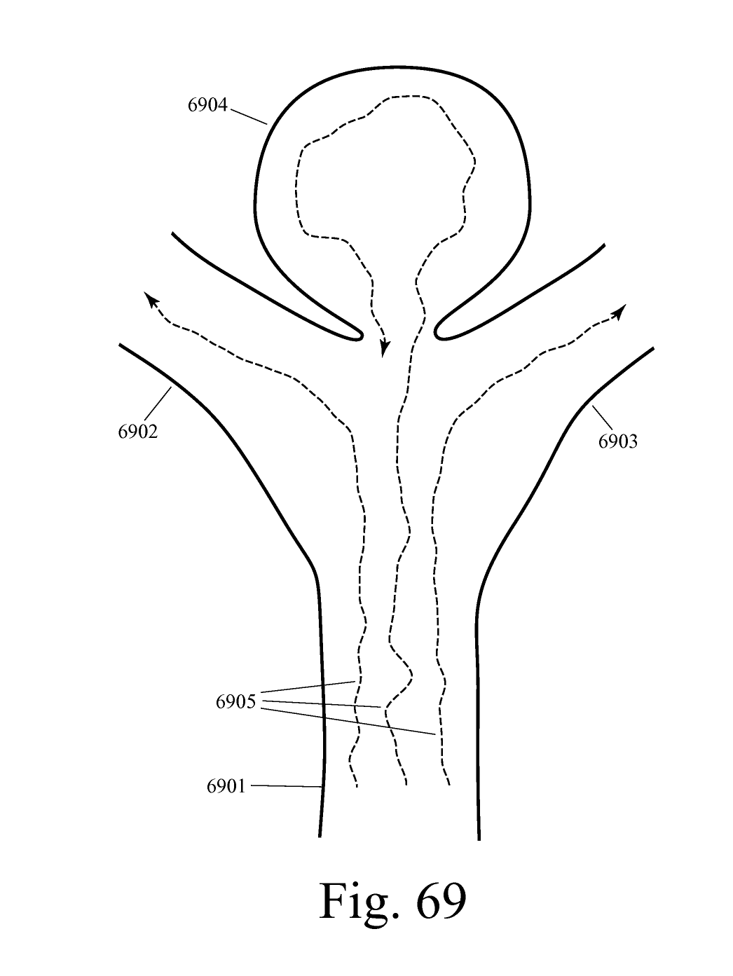

We now discuss the anatomical context and specific components of the tri-petal device that is shown in FIGS. 66 through 79. FIG. 69 shows the anatomical context for this device. The other figures in this range shown the specific components of a tri-petal device to occlude an aneurysm on a three-way vessel junction. This tri-petal device comprises a resilient expandable member with three radially-expandable petals that is configured to be expanded within a three-way junction of blood vessels from which an aneurysm has formed. This tri-petal member is further comprised as follows.

A virtual diameter-spanning axis can be defined for a tri-petal member. This virtual diameter-spanning axis can span from a virtual first pole to a diametrically-opposite virtual second pole of the tri-petal member. FIGS. 66 and 67 show a tri-petal member with a first central hub 6604 located at a virtual first pole and a second central hub 6605 located at a virtual second pole. Virtual lines of longitude can be defined along the surface of this tri-petal member from the first pole (or hub) to the second pole (or hub). Further, these lines of longitude can be assigned polar coordinates ranging from 0 to 360 degrees in a manner similar to the lines of longitude on a globe of the earth. In FIG. 67, virtual lines are shown as straight dotted-line arrows, but these lines could also be envisioned as arcing around a virtual sphere (encompassing the tri-petal member) of which one pole is hub 6604 and a second pole is hub 6605.

As shown in FIGS. 66 and 67, a tri-petal member can comprise two central hubs 6604 and 6605 and three arcuate radially-expandable petals 6601, 6602, and 6603. As shown in FIG. 67, first radially-expandable petal 6601 can have a surface that includes the line of longitude at 0 degrees. Second radially-expandable petal 6602 can have a surface that includes the line of longitude at 120 degrees. Third radially-expandable petal 6603 can have a surface that includes the line of longitude at 240 degrees. In an example, one of the radially-expandable petals (such as 6601) can be configured to cover an aneurysm neck when the tri-petal member is expanded. This reduces blood flow into the aneurysm. Gaps between the three radially-expandable petals can be configured to span the three blood vessel branches joined at the junction when the member is expanded. This allows continued blood flow through these branches.

FIG. 66 shows an outline view of a tri-petal member comprising: first radially-expandable petal 6601, second radially-expandable petal 6602, and third radially-expandable petal 6603. FIG. 67 shows the same tri-petal member in an opaque surface view. In the example shown in FIGS. 66 and 67, the tri-petal member includes central hubs 6604 and 6605, which help to hold together the narrow central portions of three radially-expandable petals 6601, 6602, and 6603. In another example, the narrow portions of three radially-expandable petals can be directly connected without central hubs.

In the example shown in FIGS. 66 and 67, three radially-expandable petals are sections of the surface of a three-dimensional figure with one cross-section that is triangular and a perpendicular cross-section that is elliptical. This three-dimensional figure could be described as a three-dimensional triangle with two convex arcuate sides (in contrast to a two-dimensional triangle with two flat planar sides). In this example, radially-expandable petals 6601, 6602, and 6603 are shaped like sections of the surface of this three-dimensional figure.

In another example, central hubs 6604 and 6605 could be located at diametrically-opposite poles of a virtual sphere which encompasses the tri-petal member. In this other example, the three radially-expandable petals 6601, 6602, and 6603 can be sections of the surface of this virtual sphere. In another example, central hubs 6604 and 6605 could be located at diametrically-opposite poles of a virtual ellipsoid which encompasses the tri-petal member. In this other example, the three radially-expandable petals 6601, 6602, and 6603 can be sections of the surface of this virtual ellipsoid. Such examples will be shown in later figures.

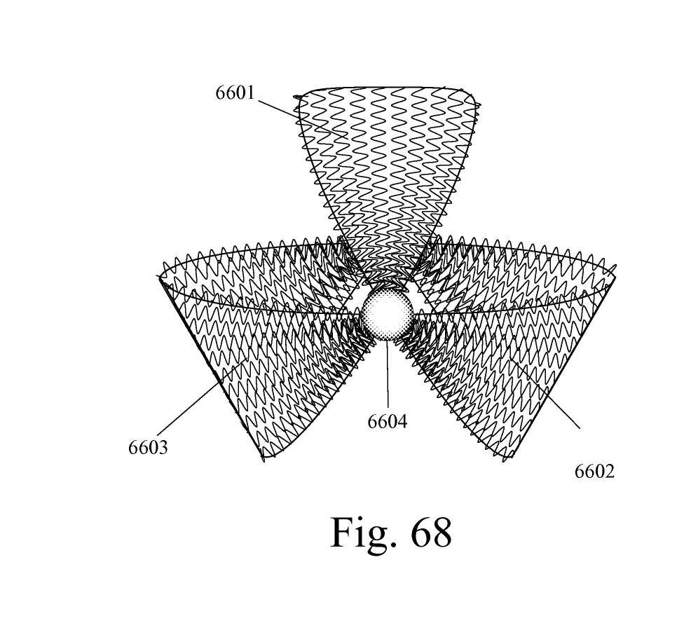

FIG. 68 shows an example of a tri-petal member, like the one introduced in FIGS. 66 and 67, in which radially-expandable petals 6601, 6603, and 6602 are comprised of expandable meshes. In an example, these expandable meshes can be metal. In an example, these expandable meshes can be comprised of wire mesh. In an example, these expandable meshes can be polymer. In an example, expandable meshes can have multiple layers. In an example, they can have a wire mesh layer for structural resistance and a low-porosity fabric layer for blood flow occlusion. In an example radially-expandable petals can be expanded by an inflatable member. In an example, radially-expandable petals can self-expand because they are made from a material with a shape memory.

In order to show the anatomical context into which a tri-petal member (such as the one in FIGS. 65 through 68) is used, FIG. 69 shows a three-way blood vessel junction with adjacent aneurysm 6904. In this example, the parent vessel of the aneurysm comprises a blood vessel junction with three vessel branches. An aneurysm such as 6904 can form at such a blood vessel junction because of currents of blood flow 6905 which pound against the portion of the junction wall which is opposite from inflow vessel 6901. In this example, blood flow 6905 enters the junction through inflow vessel 6901 and exits the junction through a first outflow vessel 6902 and a second outflow vessel 6903. However, some currents of blood flow 6905 enter (and expand) aneurysm sac 6904. In addition to exerting pressure on the aneurysm sac which can cause the aneurysm to grow and/or hemorrhage, this improper blood flow can also cause undesirable turbulence within the junction and surrounding vessels. In an example, it is desirable to design a device that not only occludes the aneurysm, but also diverts blood flow away from hitting this portion of the junction wall and restores proper blood flow through the blood vessel junction.

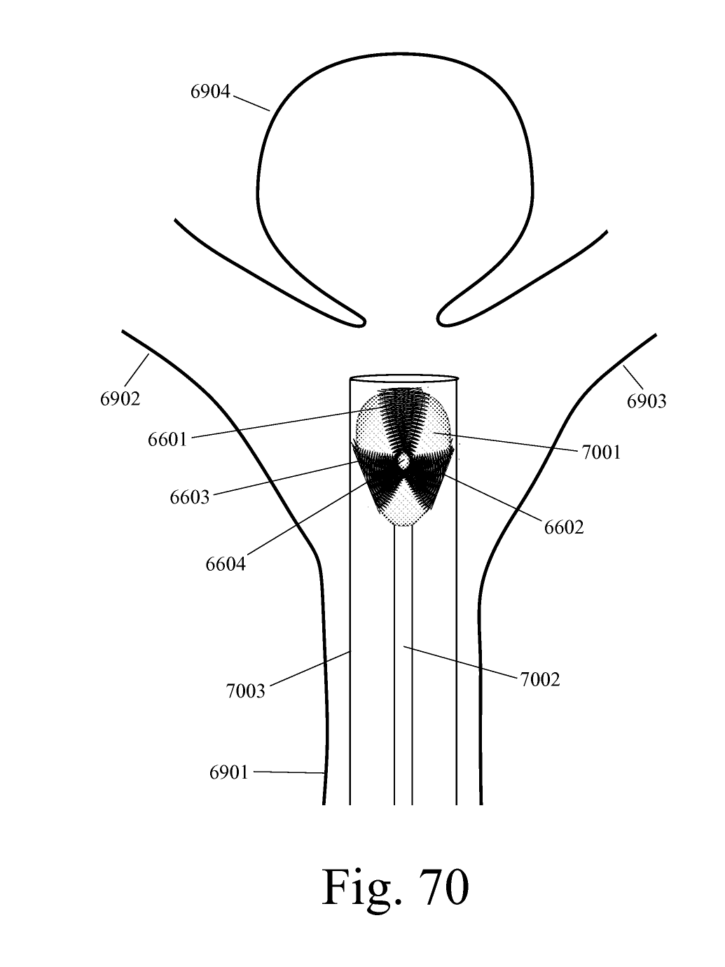

FIG. 70 shows an example of how the tri-petal member with expandable meshes that was introduced in FIG. 68 can be inserted, through longitudinal lumen 7003, into a three-branch vessel junction with an adjacent aneurysm 6904. The tri-petal member in FIG. 70 is comprised of first expandable petal 6601, second expandable petal 6602, third expandable petal 6603, and central hubs 6604 and 6605 (of which only 6604 is visible from this perspective). FIG. 70 also shows inflatable member 7001 which expands all three expandable petals and lumen 7002 which conveys a liquid or gas to inflate inflatable member 7001. FIG. 70 shows the tri-petal metal having been inserted into the blood vessel junction, but not yet having exited lumen 7003 and not yet having been expanded by inflatable member 7001.

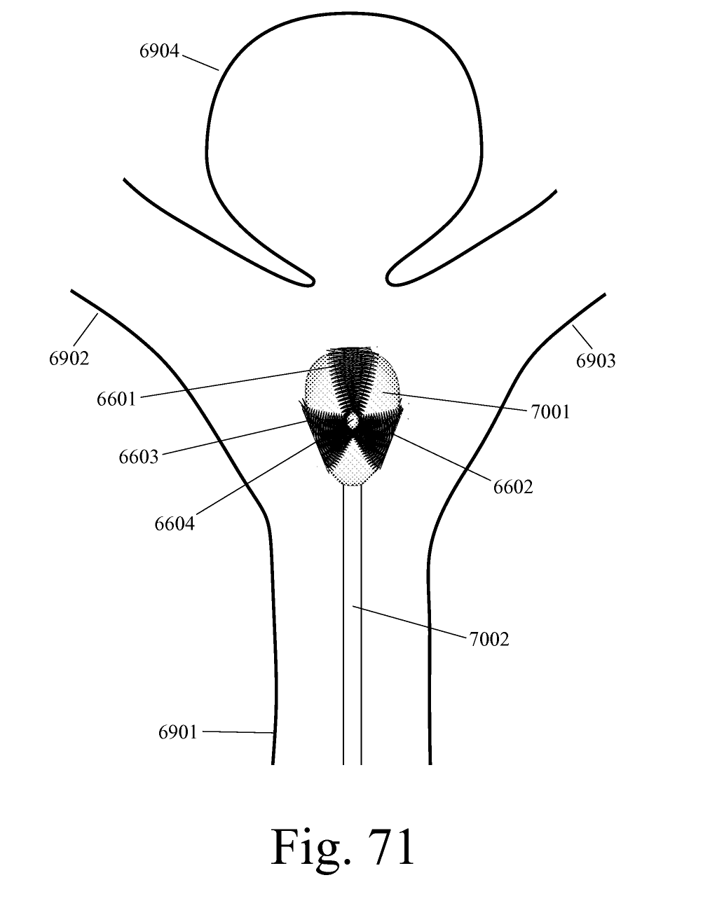

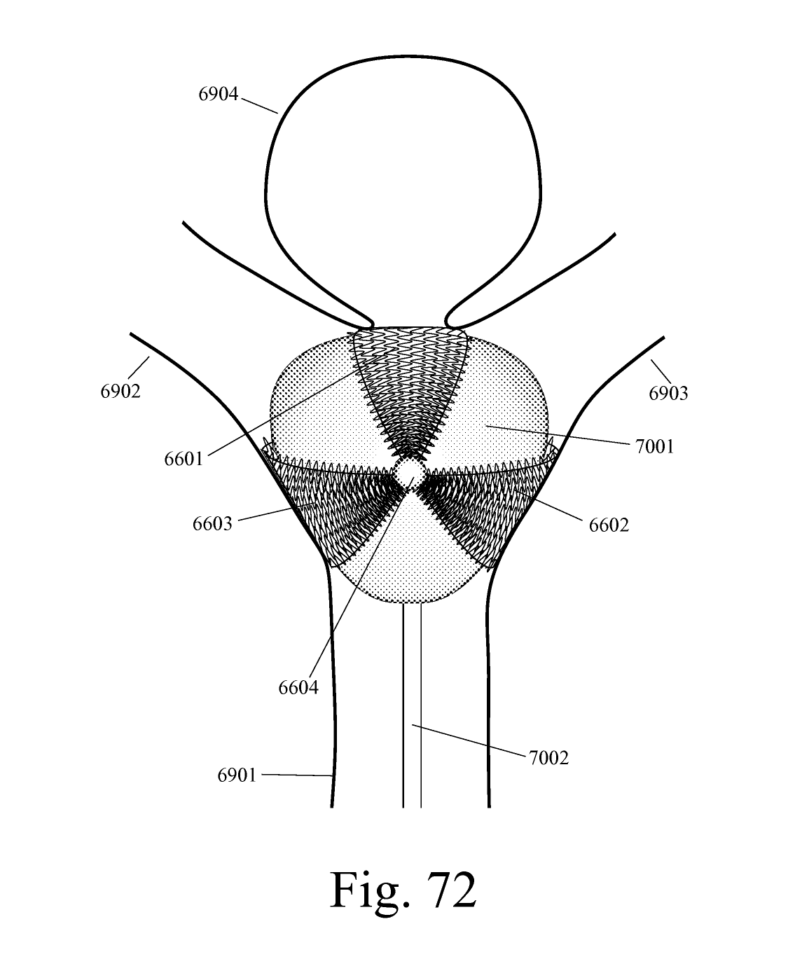

FIG. 71 shows the next sequential view. In FIG. 71, the tri-petal member has exited lumen 7001 and lumen 7001 has been removed, but the tri-petal member has not yet been expanded. FIG. 72 shows the next sequential view. In FIG. 72, the tri-petal member has been expanded by the inflation of inflatable member 7001. In this example, inflatable member 7001 has a rounded, three-dimensional triangular shape when it is inflated. Similarly, in this example, the three expandable petals 6601, 6602, and 6603 are shaped like sections of the surface of a three-dimensional triangle with two arcuate concave sides.

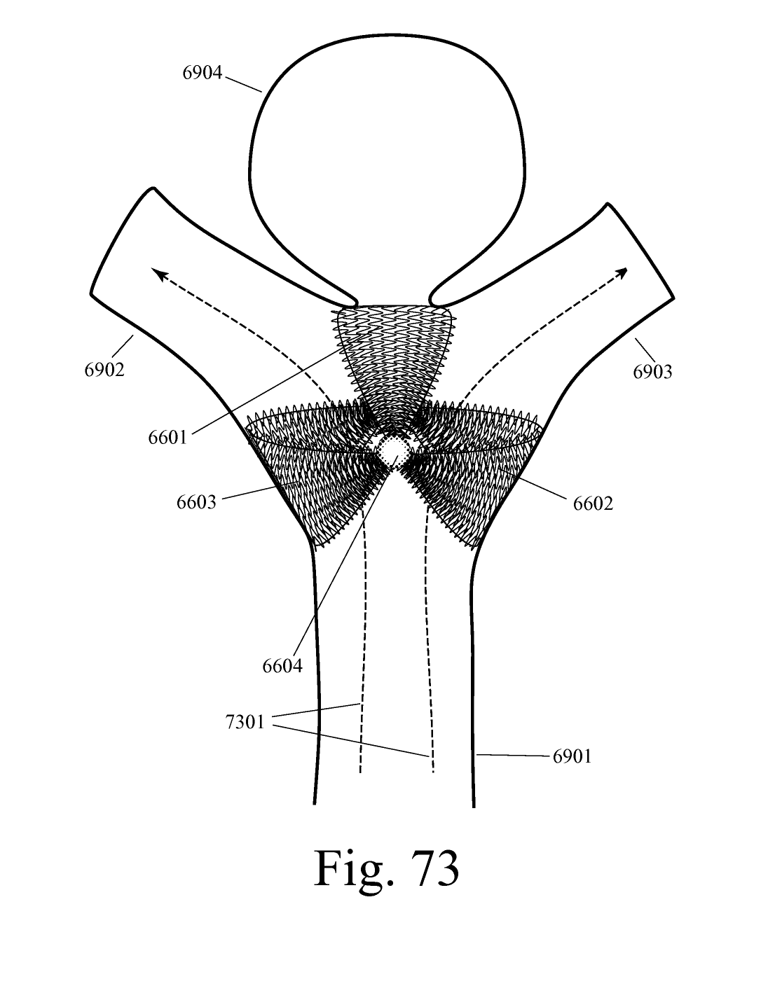

FIG. 73 shows the next sequential view. In FIG. 73, inflatable member 7001 has been deflated and removed, leaving the permanently-implanted tri-petal member in place within the blood vessel junction. In this example, radially-expandable petal 6601 covers the neck of aneurysm 6904 and diverts blood flow away from the neck. In this example, gaps between radially-expandable petals 6601, 6602, and 6603 are aligned with three branches 6901, 6902, and 6903 so that blood flow 7301 through these branches is not reduced. FIG. 73 shows how proper blood flow 7301 has been restored by this device.

FIGS. 74 through 79 show several alternative configurations for a tri-petal device. These configurations are all shown from a “polar” perspective, similar to a polar view of the earth. The configurations all share some common design parameters. In all of these alternative configurations: a first expandable petal spans a longitudinal line at 0 degrees; a second expandable petal spans a longitudinal line at 120 degrees; and a third expandable petal spans a longitudinal line at 240 degrees. For diagrammatic clarity and simplicity, these longitudinal lines are shown as straight dotted-line arrows that sometimes extend past the surfaces of the expandable petals. However, these longitudinal lines can also be envisioned as arcing along the surfaces of the expandable petals to the other pole on the far side of the member.

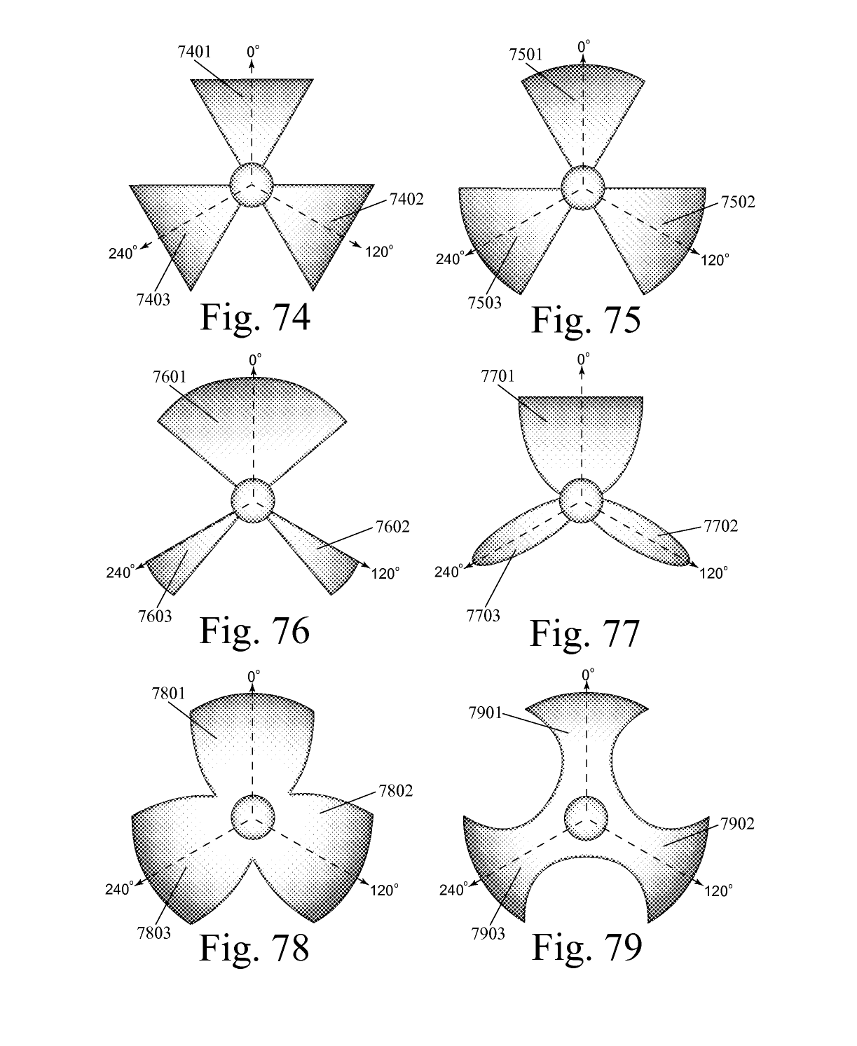

We now briefly discuss each of these tri-petal configurations individually. FIG. 74 shows an example of a tri-petal member whose petals comprise sections of a virtual three-dimensional triangular object with two arcuate convex sides. FIG. 75 shows an example of a tri-petal member whose petals comprise equal sections of a virtual sphere. The petals in FIG. 75 are similar in shape to segments of an orange, wherein the orange is viewed from a polar perspective with the tree attachment nub as the pole. FIG. 76 shows an example of a tri-petal member whose petals comprise sections of a virtual sphere, but wherein these sections are not equal.

FIG. 77 shows an example of a tri-petal member with a saddle-shaped first petal 7701 which spans the aneurysm neck and two smaller petals which span the junction at polar coordinates of 120 degrees and 240 degrees. FIG. 78 shows an example of a tri-petal member whose petals comprise portions of a virtual sphere including a wide common core. FIG. 79 shows an example of a tri-petal member whose petals comprise a virtual sphere with three arcuate “bites” taken of it.

I claim:

1. A device to occlude an aneurysm comprising:
a longitudinal lumen that is configured to be inserted into a blood vessel;
a flexible expandable member that is configured to travel through the longitudinal lumen, be inserted into an aneurysm, and then be expanded within the aneurysm sack; wherein this flexible expandable member is sufficiently flexible to substantively conform to the contours of the walls of the aneurysm sack after the flexible expandable member is expanded within the aneurysm;
a resilient expandable member that is configured to travel through the longitudinal lumen, be inserted into the aneurysm, and then be expanded within the aneurysm sack; wherein this resilient expandable member resists contraction after it has been expanded; and wherein expansion of the resilient expandable member resiliently holds a central portion of the flexible expandable member so that the flexible expandable member does not slip out of the aneurysm sack; and
a plurality of individual embolic members that are configured to travel through the longitudinal lumen, be inserted into the flexible expandable member within the aneurysm, and accumulate within the flexible expandable member; wherein the flexible expandable member does not allow the embolic members to escape out from the flexible expandable member; and wherein accumulation of the plurality of embolic members inside the flexible expandable member causes the flexible expandable member to expand.

2. The device in claim 1 wherein the flexible expandable member is selected from the group consisting of a net, a mesh, and a lattice.

3. The device in claim 1 wherein the flexible expandable member is selected from the group consisting of a balloon, a bag, and a liner.

4. The device in claim 1 wherein the flexible expandable member is made of a polymer, a metal, or a combination thereof.

5. The device in claim 1 wherein the flexible expandable member is elastic and/or stretchable.

6. The device in claim 1 wherein the flexible expandable member has holes which are of sufficient size to let liquid escape, but not so large that they let the plurality of individual embolic members escape.

7. The device in claim 1 wherein the resilient expandable member is a stent.

8. The device in claim 1 wherein the resilient expandable member is an expandable cylinder or ring.

9. The device in claim 1 wherein a plane formed by the expanding circumference of this resilient expandable member is substantially parallel to the plane that centrally spans the circumference of the aneurysm neck.

10. The device in claim 1 wherein a plane formed by the expanding circumference of the resilient expandable member spans the aneurysm sack at the sack's largest circumference parallel to the plane that centrally spans the circumference of the aneurysm neck

11. The device in claim 1 wherein the resilient expandable member is attached to or inside the flexible expandable member.

12. The device in claim 1 wherein the resilient expandable member has a bioadhesive coating which adheres to the aneurysm walls.

13. The device in claim 1 wherein the individual embolic members are compressible members.

14. The device in claim 1 wherein the individual embolic members are microsponges or blobs of gel.

15. The device in claim 1 wherein the individual embolic members are uncompressible members.

16. The device in claim 1 wherein the individual embolic members are hard polymer spheres or beads.

17. The device in claim 1 wherein the individual embolic members are conveyed through the longitudinal lumen by means of a fluid flow.

18. The device in claim 1 wherein the individual embolic members are conveyed through the longitudinal lumen by means of a moving belt or wire loop.

19. The device in claim 1 wherein the individual embolic members are conveyed through the longitudinal lumen by means of an Archimedes screw.

20. A device to occlude an aneurysm comprising:
a longitudinal lumen that is configured to be inserted into a blood vessel, wherein this blood vessel is the parent vessel from which an aneurysm has formed;
an expandable flexible net or mesh, wherein this expandable flexible net or mesh is configured to travel through the longitudinal lumen and to be inserted into the aneurysm sac, and wherein this net or mesh is sufficiently flexible to substantially conform to the walls of an irregularly shaped aneurysm sac after the net or mesh has been expanded;
an expandable resilient structure, wherein this expandable resilient structure is configured to travel through the longitudinal lumen and to be inserted into the aneurysm sac; wherein this structure comes into engaging contact with the central circumference of the aneurysm sac when this structure is expanded; wherein this structure resists compression after it has been expanded; and wherein expansion of this structure also engages the net or mesh so as to prevent the net or mesh from slipping out from the aneurysm sac; and
a plurality of embolic members, wherein these embolic members are configured to travel through the longitudinal lumen and to be inserted into the net or mesh within the aneurysm sac; wherein these embolic members do not escape from the net or mesh; and wherein the net or mesh is expanded by the accumulation of embolic members inside the net or mesh.
